(19) United States
(12) Patent Application Publication
(10) Pub. No.: US 2025/0135020 A1
(43) Pub. Date: May 1, 2025

(54) METHODS FOR TREATING NON-MUSCLE INVASIVE BLADDER CANCER (NMIBC) WITH ANTIBODY DRUG CONJUGATES (ADC) THAT BIND TO 191P4D12 PROTEINS

(71) Applicants: AGENSYS, INC., Northbrook, IL (US); SEAGEN INC., Bothell, WA (US)

(72) Inventors: Christopher CAROSINO, Duvall, WA (US); Sujata NARAYANAN, Newark, CA (US); Amit GARG, Foster City, CA (US)

(73) Assignees: AGENSYS, INC., Northbrook, IL (US); SEAGEN INC., Bothell, WA (US)

(21) Appl. No.: 18/682,886

(22) PCT Filed: Aug. 12, 2022
(86) PCT No.: PCT/US2022/074897

§ 371 Date: Feb. 9, 2024

Related U.S. Application Data
(60) Provisional application No. 63/328,441, filed on Apr. 7, 2022.
(60) Provisional application No. 63/242,380, filed on Sep. 9, 2021.
(60) Provisional application No. 63/233,048, filed on Aug. 13, 2021.

(51) Int. Cl. A61K 47/68; A61K 47/22; A61K 47/26; A61P 35/00

(52) U.S. Cl. CPC A61K 47/6849; A61K 47/22; A61K 47/26; A61K 47/68031; A61K 47/6889; A61P 35/00

(57) ABSTRACT

Provided herein are methods for the intravesical treatment of bladder cancer and methods for treating non-muscle invasive bladder cancer with antibody drug conjugates (ADC) that bind to 191P4D12 protein (Nectin-4).

### CROSS-REFERENCE TO RELATED APPLICATIONS

This application claims the benefit of U.S. Application No. 63/233,048, filed Aug. 13, 2021, U.S. Application No. 63/242,380, filed Sep. 9, 2021, and U.S. Application No. 63/328,441, filed Apr. 7, 2022, the disclosure of each of which is incorporated by reference herein in its entirety.

### REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application is entitled “14369-281-228_SEQ_LISTING.xml”, was created on Aug. 2, 2022, and is 34,743 bytes in size.

### 1. FIELD

Provided herein are methods for treating non-muscle invasive bladder cancer (NMIBC) with antibody drug conjugates (ADC) that bind to 191P4D12 protein (Nectin-4).

### 2. BACKGROUND

Bladder cancer, the most common form of urothelial cancer (UC) and the sixth most common cancer in the United States (U.S.), is estimated to kill nearly 200,000 patients globally on an annual basis, including more than 65,000 in Europe and nearly 18,000 in the U.S. (Bray 2018; Ferlay 2018; Siegel 2019). Annual diagnoses of new cases of bladder cancer were estimated to be more than 549,000 worldwide in 2018. In 2020, there were an estimated 81,400 new cases of bladder cancer in the U.S.; this estimate increased to more than 83,000 cases in 2021 (American Cancer Society (ACS) 2021; National Cancer Institute (NCI) 2021). Bladder cancer incidence and mortality strongly increase with age and will be an increasing problem as the population becomes more elderly.

Approximately 70% to 80% of bladder cancer diagnoses present as non-muscle invasive disease (Chang 2016; Woldu 2017; Kates 2020; Li 2020). Non-muscle invasive bladder cancer (NMIBC) represents a heterogeneous group of cancers that include those that are papillary in nature and limited to the mucosa (Ta), high-grade and flat and confined to the epithelium (Tis or carcinoma in situ [CIS]), and invasive into the submucosa, or lamina propria (T1) (Pasin 2008). Among patients with NMIBC, papillary disease is most common, affecting approximately 70% of patients, while T1 disease and CIS affect approximately 20% and 10% of patients, respectively (Kirkali 2005; Anastasiadis 2012).

The standard of care for treatment of NMIBC involves surgical resection of the bladder tumor via transurethral resection of bladder tumor (TURBT) followed by intravesical administration of therapeutic agents for further antitumor activity (Kawai 2013; Chang 2016; Woldu 2017; Jamil 2019; Kates 2020). Intravesical Bacillus Calmette-Guerin (BCG) is considered a therapeutic agent of choice that triggers local immune responses that appear to correlate with antitumor activity for patients with NMIBC, and especially for patients who have characteristics of high-risk disease (Kassouf 2015; Chang 2016).

BCG-unresponsive disease denotes a subgroup of patients with NMIBC who have failed to respond to adequate treatment with BCG and remain at high risk for disease recurrence and progression to subsequent stages of UC. For these patients, additional treatment with BCG is not an option, and radical cystectomy remains the best available option. Intravesical chemotherapy agents such as gemcitabine, mitomycin, and valrubicin have shown some efficacy; however, current guidelines state that treatments other than radical cystectomy are inferior for treatment of BCG-unresponsive disease (Navai 2016; Taylor 2020) (EAU. Guidelines for Non-muscle-invasive Bladder Cancer. 2020. https://uroweb.org/guideline/non-muscle-invasive-bladdercancer/Accessed Feb. 16, 2021.). While systemic pembrolizumab was recently approved for the treatment of patients with BCG-unresponsive disease with carcinoma in situ (CIS), over half of patients treated with pembrolizumab do not achieve a complete response to therapy; therefore, the need for safe and effective intravesical therapies in this patient population remains.

191P4D12 (which is also known as Nectin-4) is a 66 kDa type I transmembrane protein that belongs to the nectin family of adhesion molecules. It is composed of an extracellular domain (ECD) containing 3 immunoglobulin (Ig)-like subdomains, a transmembrane helix, and an intracellular region (Takai et al., Annu Rev Cell Dev Biol (2008); 24: 309-42). Nectins are thought to mediate Ca²⁺-independent cell-cell adhesion via both homophilic and heterophilic trans-interactions at adherens junctions where they can recruit cadherins and modulate cytoskeletal rearrangements (Rikitake et al., Cell Mol Life Sci (2008); 65(2): 253-63.). Sequence identity of Nectin-4 to other Nectin family members is low and ranges between 25%-30% in the ECD (Reymond et al., Biol Chem (2001); 276(46): 43205-15).

Nectin-4 has been found to be expressed in multiple cancers, particularly urothelial, breast, lung, pancreatic, and ovarian cancers. Higher levels of expression are associated with disease progression and/or poor prognosis (Fabre-Lafay et al., BMC Cancer (2007); 7: 73).

There remains an unmet need for safe and effective intravesical treatments for patients with BCG-unresponsive NMIBC who are unfit or ineligible for radical cystectomy, or for those who have made an informed decision not to undergo radical cystectomy.

### 3. SUMMARY

Provided herein are methods for the treatment of bladder cancer in human subjects via intravesical administration of an antibody drug conjugate (ADC) that binds 191P4D12.

Embodiment 1. A method of treating bladder cancer in a human subject, comprising intravesically administering to the subject an effective amount of an antibody drug conjugate (ADC), wherein the ADC comprises an antibody or antigen binding fragment thereof that binds to 191P4D12 conjugated to one or more units of monomethyl auristatin E (MMAE).

Embodiment 2. The method of embodiment 1, wherein the bladder cancer is non-muscle invasive bladder cancer (NMIBC).

Embodiment 3. The method of embodiment 2, wherein the NMIBC has been histologically confirmed and is carcinoma in situ (CIS).

Embodiment 4. The method of embodiment 3, wherein the subject has papillary disease.

Embodiment 5. The method of embodiment 3, wherein the subject does not have papillary disease.

Embodiment 6. The method of any of embodiments 2 to 5, wherein the NMIBC has been histologically confirmed and wherein the predominant histologic component (>50%) is urothelial (transitional cell) carcinoma.

Embodiment 7. The method of any one of embodiments 1 to 6, wherein the subject has high-risk Bacillus Calmette-Guerin (BCG)-unresponsive disease.

Embodiment 8. The method of any one of embodiments 1 to 7, wherein the subject is ineligible for or refuses to undergo a radical cystectomy.

Embodiment 9. The method of any one of embodiments 1 to 8, wherein all visible papillary Ta/T1 tumors of the subject have completely resected within 60 days prior to the treatment.

Embodiment 10. The method of embodiment 9, wherein the subject has residual pure CIS.

Embodiment 11. The method of embodiment 9, wherein the subject does not have residual pure CIS.

Embodiment 12. The method of any one of embodiments 1 to 11, wherein the subject has an Eastern Cooperative Oncology Group (ECOG) Performance Status score of 0.

Embodiment 13. The method of any one of embodiments 1 to 11, wherein the subject has an Eastern Cooperative Oncology Group (ECOG) Performance Status score of 1.

Embodiment 14. The method of any one of embodiments 1 to 11, wherein the subject has an Eastern Cooperative Oncology Group (ECOG) Performance Status score of 2.

Embodiment 15. The method of embodiment 14, wherein the subject's glomerular filtration rate (GFR) is no less than 50 mL/min and the subject does not have New York Heart Association (NYHA) Class III heart failure.

Embodiment 16. The method of any one of embodiments 1 to 15, wherein the subject has one or more of the conditions selected from the group consisting of:
a. Absolute neutrophil count (ANC)≥1500/μL;
b. Hemoglobin (Hgb)≥10 g/dL;
c. Platelet count≥100,000/μL;
d. Serum bilirubin≤1.5×upper limit of normal (ULN) or ≤3×ULN for subjects with Gilbert's disease;
e. Calculated creatinine clearance (CrCl)≥30 mL/min (GFR can also be used in place of creatinine or CrCl). CrCl should be calculated using the Cockcroft-Gault method or Modification of Diet in Renal Disease (MDRD) equations. Subjects with an ECOG performance status of 2 must have GFR≥50 mL/min;
f. Alanine aminotransferase (ALT) and aspartate aminotransferase (AST)≤3×ULN; or
g. International normalized ratio (INR) or prothrombin time (PT), activated partial thromboplastin time (aPTT) or partial thromboplastin time (PTT)≤51.5 ULN unless subject is receiving anticoagulant therapy as long as PT or aPTT is within therapeutic range of intended use of anticoagulants.

Embodiment 17. The method of embodiment 16, wherein the subject has all of conditions (a) to (g) of embodiment 16.

Embodiment 18. The method of any one of embodiments 1 to 17, wherein the subject's estimated life expectancy is more than 2 years.

Embodiment 19. The method of any one of embodiments 1 to 18, wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23.

Embodiment 20. The method of any one of embodiments 1 to 19,
wherein the antibody or antigen binding fragment thereof comprises CDR-H1 comprising the amino acid sequence of SEQ ID NO:9, CDR-H2 comprising the amino acid sequence of SEQ ID NO:10, CDR-H3 comprising the amino acid sequence of SEQ ID NO:11; CDR-L1 comprising the amino acid sequence of SEQ ID NO:12, CDR-L2 comprising the amino acid sequence of SEQ ID NO:13, and CDR-L3 comprising the amino acid sequence of SEQ ID NO:14, or
wherein the antibody or antigen binding fragment thereof comprises CDR-H1 comprising the amino acid sequence of SEQ ID NO:16, CDR-H2 comprising the amino acid sequence of SEQ ID NO:17, CDR-H3 comprising the amino acid sequence of SEQ ID NO:18; CDR-L1 comprising the amino acid sequence of SEQ ID NO:19, CDR-L2 comprising the amino acid sequence of SEQ ID NO:20, and CDR-L3 comprising the amino acid sequence of SEQ ID NO:21.

Embodiment 21. The method of any one of embodiments 1 to 19,
wherein the antibody or antigen binding fragment thereof comprises CDR-H1 consisting of the amino acid sequence of SEQ ID NO:9, CDR-H2 consisting of the amino acid sequence of SEQ ID NO:10, CDR-H3 consisting of the amino acid sequence of SEQ ID NO:11; CDR-L1 consisting of the amino acid sequence of SEQ ID NO:12, CDR-L2 consisting of the amino acid sequence of SEQ ID NO:13, and CDR-L3 consisting of the amino acid sequence of SEQ ID NO:14, or
wherein the antibody or antigen binding fragment thereof comprises CDR-H1 consisting of the amino acid sequence of SEQ ID NO:16, CDR-H2 consisting of the amino acid sequence of SEQ ID NO:17, CDR-H3 consisting of the amino acid sequence of SEQ ID NO:18; CDR-L1 consisting of the amino acid sequence of SEQ ID NO:19, CDR-L2 consisting of the amino acid sequence of SEQ ID NO:20, and CDR-L3 consisting of the amino acid sequence of SEQ ID NO:21.

Embodiment 22. The method of any one of embodiments 1 to 21, wherein the antibody or antigen binding fragment thereof comprises a heavy chain variable region comprising the amino acid sequence of SEQ ID NO:22 and a light chain variable region comprising the amino acid sequence of SEQ ID NO:23.

Embodiment 23. The method of any one of embodiments 1 to 22, wherein the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

Embodiment 24. The method of any one of embodiments 1 to 23, wherein the antigen binding fragment is an Fab, F(ab′)2, Fv or scFv.

Embodiment 25. The method of any one of embodiments 1 to 24, wherein the antibody is a fully human antibody.

Embodiment 26. The method of any one of embodiments 1 to 25, wherein the antibody is an IgG1 and light chain is a kappa light chain.

Embodiment 27. The method of any one of embodiments 1 to 26, wherein the antibody or antigen binding fragment thereof is recombinantly produced.

Embodiment 28. The method of any one of embodiments 1 to 27, wherein the antibody or antigen binding fragment is conjugated to each unit of MMAE via a linker.

Embodiment 29. The method of embodiment 28, wherein the linker is an enzyme-cleavable linker, and wherein the linker forms a bond with a sulfur atom of the antibody or antigen binding fragment thereof.

Embodiment 30. The method of embodiment 28 or 29, wherein the linker has a formula of: -Aa-Ww-Yy-; wherein -A- is a stretcher unit, a is 0 or 1; -W- is an amino acid unit, w is an integer ranging from 0 to 12; and -Y- is a spacer unit, y is 0, 1, or 2.

Embodiment 31. The method of embodiment 30, wherein the stretcher unit has the structure of Formula (1) below; the amino acid unit is valine-citrulline; and the spacer unit is a PAB group comprising the structure of Formula (2) below:

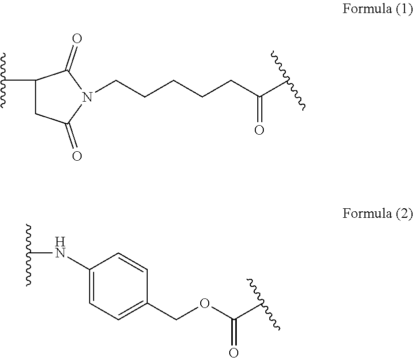 [SMILES: *C(=O)CCCCCN1C(=O)CC(*)C1=O ; *Nc1ccc(COC(*)=O)cc1]

Embodiment 32. The method of embodiment 30 or 31, wherein the stretcher unit forms a bond with a sulfur atom of the antibody or antigen binding fragment thereof, and wherein the spacer unit is linked to MMAE via a carbamate group.

Embodiment 33. The method of any one of embodiments 1 to 32, wherein the ADC comprises from 1 to 20 units of MMAE per antibody or antigen binding fragment thereof.

Embodiment 34. The method of any one of embodiments 1 to 33, wherein the ADC comprises from 1 to 10 units of MMAE per antibody or antigen binding fragment thereof.

Embodiment 35. The method of any one of embodiments 1 to 34, wherein the ADC comprises from 2 to 8 units of MMAE per antibody or antigen binding fragment thereof.

Embodiment 36. The method of any one of embodiments 1 to 35, wherein the ADC comprises from 3 to 5 units of MMAE per antibody or antigen binding fragment thereof.

Embodiment 37. The method of any one of embodiments 1 to 36, wherein the ADC has the following structure:

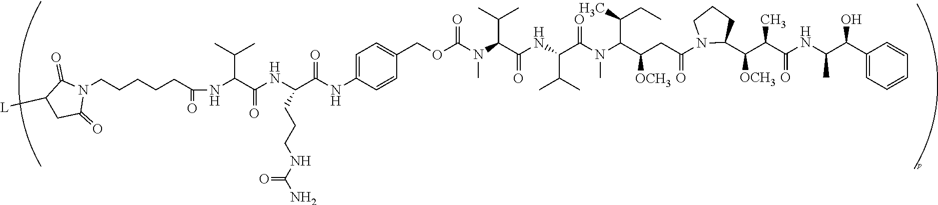

wherein L- represents the antibody or antigen binding fragment thereof and p is from 1 to 10.

Embodiment 38. The method of embodiment 37, wherein p is from 2 to 8.

Embodiment 39. The method of embodiment 37 or 38, wherein p is from 3 to 5.

Embodiment 40. The method of any one of embodiments 37 to 39, wherein p is from 3 to 4.

Embodiment 41. The method of any one of embodiments 37 to 40, wherein p is about 4.

Embodiment 42. The method of any one of embodiments 37 to 40, wherein the average p value of the effective amount of the antibody drug conjugates is about 3.8.

Embodiment 43. The method of any one of embodiments 1 to 42, wherein the ADC is formulated in a pharmaceutical composition comprising L-histidine, polysorbate-20 (TWEEN-20), and trehalose dehydrate.

Embodiment 44. The method of any one of embodiments 1 to 43, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.

Embodiment 45. The method of any one of embodiments 1 to 43, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

Embodiment 46. The method of any one of embodiments 1 to 45, wherein the effective amount of the ADC is a dose of between about 100 mg to about 1000 mg, between about 125 mg to about 950 mg, between about 125 mg to about 900 mg, between about 125 mg to about 850 mg, between about 125 mg to about 800 mg, or between about 125 mg to about 750 mg with a volume of instillation between about 10 mL to about 100 mL.

Embodiment 47. The method of any one of embodiments 1 to 46, wherein the effective amount of the ADC is a dose of between about 125 mg to about 750 mg with a volume of instillation of about 25 mL.

Embodiment 48. The method of any one of embodiments 1 to 47, wherein the effective amount of the ADC is a dose of about 125 mg with a volume of instillation of about 25 mL.

Embodiment 49. The method of any one of embodiments 1 to 47, wherein the effective amount of the ADC is a dose of about 250 mg with a volume of instillation of about 25 mL.

Embodiment 50. The method of any one of embodiments 1 to 47, wherein the effective amount of the ADC is a dose of about 500 mg with a volume of instillation of about 25 mL.

Embodiment 51. The method of any one of embodiments 1 to 47, wherein the effective amount of the ADC is a dose of about 750 mg with a volume of instillation of about 25 mL.

Embodiment 52. The method of any one of embodiments 1 to 51, wherein the maximal dwell time of each intravesical administration is about 90 minutes.

Embodiment 53. The method of any one of embodiments 1 to 51, wherein the maximal dwell time of each intravesical administration is about 120 minutes.

Embodiment 54. The method of any one of embodiments 1 to 51, wherein the dwell time of each intravesical administration is about 30, 40, 50, 60, 70, 80, 90, or 120 minutes.

Embodiment 55. The method of any one of embodiments 1 to 54, wherein the ADC is administered intravesically during two phases, wherein the two phases are an induction phase and a maintenance phase.

Embodiment 56. The method of embodiment 55, wherein the maintenance phase starts between six to ten weeks, between six to nine weeks, or between six to eight weeks after the induction phase.

Embodiment 57. The method of embodiment 55 or 56, wherein the ADC is administered intravesically once a week for six weeks during the induction phase.

Embodiment 58. The method of any one of embodiments 55 to 57, wherein the ADC is administered intravesically once a month for nine months during the maintenance phase.

Embodiment 59. The method of any one of embodiments 1 to 58, wherein the ADC has the following structure:

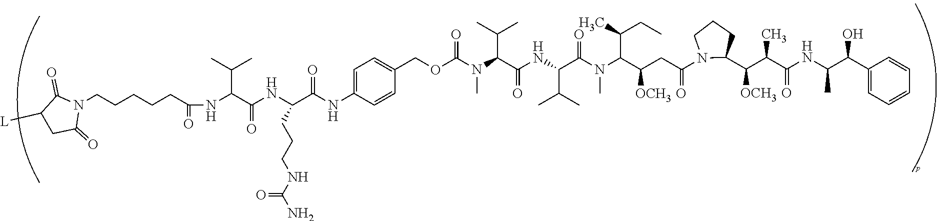

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered intravesically at a dose of about 125 mg with a volume of instillation of about 25 mL and a maximum 90-minute dwell time, wherein the dose is administered intravesically once a week for six weeks during the induction phase and once a month for nine months during the maintenance phase, and wherein the maintenance phase starts between six to ten weeks after the induction phase.

Embodiment 60. The method of any one of embodiments 1 to 58, wherein the ADC has the following structure:

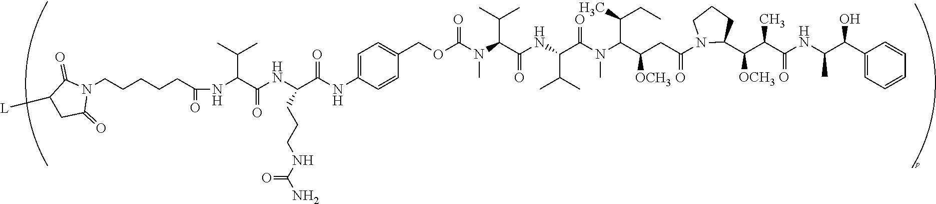

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered intravesically at a dose of about 250 mg with a volume of instillation of about 25 mL and a maximum 90-minute dwell time, wherein the dose is administered intravesically once a week for six weeks during the induction phase and once a month for nine months during the maintenance phase, and wherein the maintenance phase starts between six to ten weeks after the induction phase.

Embodiment 61. The method of any one of embodiments 1 to 58, wherein the ADC has the following structure:

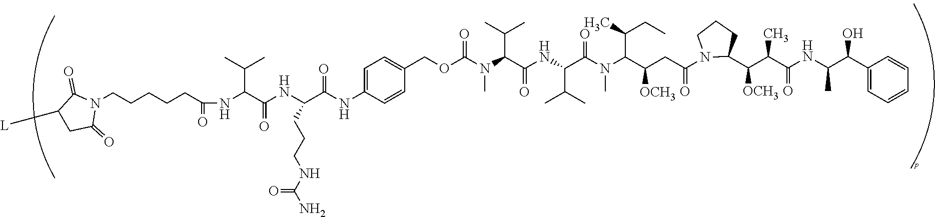

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered intravesically at a dose of about 500 mg with a volume of instillation of about 25 mL and a maximum 90-minute dwell time, wherein the dose is administered intravesically once a week for six weeks during the induction phase and once a month for nine months during the maintenance phase, and wherein the maintenance phase starts between six to ten weeks after the induction phase.

Embodiment 62. The method of any one of embodiments 1 to 58, wherein the ADC has the following structure:

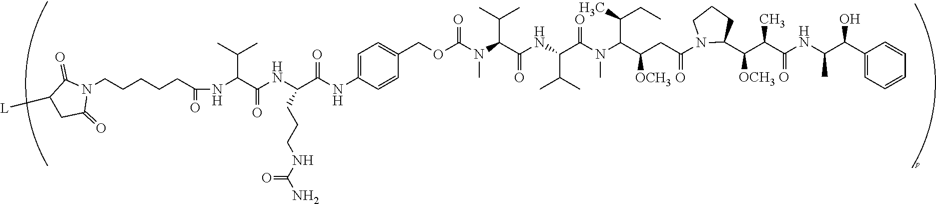

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered intravesically at a dose of about 750 mg with a volume of instillation of about 25 mL and a maximum 90-minute dwell time, wherein the dose is administered intravesically once a week for six weeks during the induction phase and once a month for nine months during the maintenance phase, and wherein the maintenance phase starts between six to ten weeks after the induction phase.

### 4. BRIEF DESCRIPTION OF THE DRAWINGS

FIGS. 1A-1E depict the nucleotide and amino acid sequences of nectin-4 protein (FIG. 1A), the nucleotide and amino acid sequences of the heavy chain (FIG. 1B) and light chain (FIG. 1C) of Ha22-2(2.4)6.1, and the amino acid sequences of the heavy chain (FIG. 1D) and light chain (FIG. 1E) of Ha22-2(2.4)6.1.

FIG. 2 depicts cytotoxic activity of enfortumab vidotin (EV) in vitro in Nectin-4 overexpressing bladder carcinoma cells (i.e., UM-UC-3-hNectin-4⁺) using conditions that mimic intravesical dosing.

FIGS. 3A-3E depict the efficacy of intravesical administration of enfortumab vidotin (EV) in a Nectin-4⁺ bladder orthotopic xenograft mouse model. FIG. 3A depicts the generation of SCID mice orthotopically implanted following chemical abrasion with UM-UC-3-hNectin4⁺-Luc⁺ cells, as well as the dosing schedule for administration of intravesical EV to the mice followed by histological analysis of bladder tissue. FIG. 3B depicts the bioluminescence imaging results that confirm tumor engraftment and EV activity. SWFI, Sterile Water for Injection. FIG. 3C depicts the anti-Nectin-4 immunohistochemistry results that confirm EV activity. The bladder tissue in the five right panels in FIG. 3C were from the five mice treated with intravesical doses of EV in FIG. 3B, respectively. FIG. 3D depicts the quantitative analysis of the bioluminescence imaging results in FIG. 3B. FIG. 3E depicts immunohistochemistry (IHC) staining of Nectin-4 and MMAE in bladder tumor tissue, showing co-localization of Nectin-4 and MMAE.

FIGS. 4A-4B depict free MMAE in the bladder tissue of Sprague-Dawley rats treated with single intravesical dose of EV at varied concentrations and administration volumes.

FIG. 5 depicts intravesical EV systemic exposure.

FIG. 6 depicts free MMAE in the bladder tissue of Sprague-Dawley rats treated with a single intravesical dose of EV for different lengths of dwell time.

FIG. 7 depicts the schema of the clinical trial described in Section 6.1, which is a phase 1, open-label, multicenter, dose-escalation, and dose-expansion study designed to evaluate the safety, tolerability, PK, and antitumor activity of intravesical enfortumab vedotin in adults with NMIBC. (BCG=Bacillus Calmette-Guerin; CIS=carcinoma in situ; EV=enfortumab vedotin; mTPI=modified toxicity probability interval; q3=every 3; q6=every 6; TURBT=transurethral resection of the bladder tumor; wkly=weekly. Safety at a given dose or investigation of a dose level that is lower than or intermediate to the planned dose levels is contemplated.)

### 5. DETAILED DESCRIPTION

Before the present disclosure is further described, it is to be understood that the disclosure is not limited to the particular embodiments set forth herein, and it is also to be understood that the terminology used herein is for describing particular embodiments only, and is not intended to be limiting.

There are a variety of factors that need to be considered in efficient use of intravesicular treatment, including, at least, volume of instillation, dwell time, dose concentration, total dose, the pH of the instillation, the pH of the urine, reduction of urine production before and during treatment, etc. so as to achieve an appropriate balance between efficacy and safety. The present disclosure surprisingly determined that dose concentration and total dose are the most important factors for efficient and safe delivery of an ADC, such as an MMAE ADC disclosed herein. The present disclosure used this knowledge to inform development of various methods employing intravesicular administration of an ADC to treat bladder cancer (e.g., non-muscle invasive bladder cancer (NMIBC)).

### 5.1 Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dubel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)2 fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

The term “monoclonal antibody” refers to an antibody obtained from a population of substantially homogeneous antibodies, that is, the individual antibodies comprising the population are identical except for possible naturally occurring mutations that can be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell, for example, a cancer cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “antigen binding fragment,” “antigen binding domain,” “antigen binding region,” and similar terms refer to that portion of an antibody, which comprises the amino acid residues that interact with an antigen and confer on the binding agent its specificity and affinity for the antigen (e.g., the CDRs). “Antigen-binding fragment” as used herein include “antibody fragment,” which comprise a portion of an intact antibody, such as the antigen-binding or variable region of the intact antibody. Examples of antibody fragments include, without limitation, Fab, Fab′, F(ab′)2, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see, e.g., Woolven et al., 1999, Immunogenetics 50: 98-101; and Streltsov et al., 2004, Proc Natl Acad Sci USA. 101:12444-49); and multispecific antibodies formed from antibody fragments.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well-known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody or antigen binding fragment thereof described herein terms such as “bind to,” “that specifically bind to,” and analogous terms are also used interchangeably herein and refer to binding molecules of antigen binding domains that specifically bind to an antigen, such as a polypeptide. An antibody or antigen binding fragment that binds to or specifically binds to an antigen may be cross-reactive with related antigens. In certain embodiments, an antibody or antigen binding fragment that binds to or specifically binds to an antigen does not cross-react with other antigens. An antibody or antigen binding fragment that binds to or specifically binds to an antigen can be identified, for example, by immunoassays, Octet®, Biacore®, or other techniques known to those of skill in the art. In some embodiments, an antibody or antigen binding fragment binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of an antibody or antigen binding fragment to a “non-target” protein is less than about 10% of the binding of the binding molecule or antigen binding domain to its particular target antigen, for example, as determined by fluorescence activated cell sorting (FACS) analysis or RIA. With regard terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding fragment that binds to an antigen includes one that is capable of binding the antigen with sufficient affinity such that the binding molecule is useful, for example, as a diagnostic agent in targeting the antigen. In certain embodiments, an antibody or antigen binding fragment that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding fragment binds to an epitope of an antigen that is conserved among the antigen from different species (e.g., between human and cyno species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using biolayer interferometry (BLI) or surface plasmon resonance (SPR) assays by Octet®, using, for example, a Octet® QK384 system, or by Biacore®, using, for example, a Biacore® TM-2000 or a Biacore® TM-3000. An “on-rate” or “rate of association” or “association rate” or “kon” may also be determined with the same biolayer interferometry (BLI) or surface plasmon resonance (SPR) techniques described above using, for example, the Octet® QK384, the Biacore® TM-2000, or the Biacore® TM-3000 system.

In certain embodiments, the antibodies or antigen binding fragments can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

In certain embodiments, the antibodies or antigen binding fragments can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate comprising the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

In certain embodiments, the antibodies or antigen binding fragments can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies comprising variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1: 755-68). Also available for the preparation of human monoclonal antibodies are methods described in Cole et al., Monoclonal Antibodies and Cancer Therapy 77 (1985); Boerner et al., 1991, J. Immunol. 147(1):86-95; and van Dijk and van de Winkel, 2001, Curr. Opin. Pharmacol. 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6(5):561-66; Bruggemann and Taussing, 1997, Curr. Opin. Biotechnol. 8(4):455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

In certain embodiments, the antibodies or antigen binding fragments can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies or antigen binding fragments can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well-known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a p sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well-known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Pluckthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted below in Table 1.

TABLE 1

| | Kabat | AbM | Chothia | Contact | IMGT |
|---|---|---|---|---|---|
| CDR-L1 | L24--L34 | L24--L34 | L24--L34 | L30--L36 | L27--L38 |
| CDR-L2 | L50--L56 | L50--L56 | L50--L56 | L46--L55 | L56--L65 |
| CDR-L3 | L89--L97 | L89--L97 | L89--L97 | L89--L96 | L105-L117 |
| CDR-H1 | H31--H35B | H26-- | H26-- | H30-- | H27--H38 |
| | (Kabat | H35B | H32 . . . 34 | H35B | |
| | Numbering | | | | |
| CDR-H1 | H31--H35 | H26--H35 | H26--H32 | H30--H35 | |
| | (Chothia | | | | |
| | Numbering 1 | | | | |
| CDR-H2 | H50--H65 | H50--H58 | H52--H56 | H47--H58 | H56--H65 |
| CDR-H3 | H95--H102 | H95-- | H95-- | H93-- | H105- |
| | | H102 | H102 | H101 | H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule comprising a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations comprising a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

The abbreviation “MMAE” refers to monomethyl auristatin E.

Unless otherwise indicated by context, a hyphen (-) designates the point of attachment to the pendant molecule.

The term “Chemotherapeutic Agent” refers to all chemical compounds that are effective in inhibiting tumor growth. Non-limiting examples of chemotherapeutic agents include alkylating agents; for example, nitrogen mustards, ethyleneimine compounds and alkyl sulphonates; antimetabolites, for example, folic acid, purine or pyrimidine antagonists; mitotic inhibitors, for example, anti-tubulin agents such as vinca alkaloids, auristatins and derivatives of podophyllotoxin; cytotoxic antibiotics; compounds that damage or interfere with DNA expression or replication, for example, DNA minor groove binders; and growth factor receptor antagonists. In addition, chemotherapeutic agents include cytotoxic agents (as defined herein), antibodies, biological molecules and small molecules.

As used herein, the term “conservative substitution” refers to substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al., MOLECULAR BIOLOGY OF THE GENE, The Benjamin/Cummings Pub. Co., p. 224 (4th Edition 1987)). Such exemplary substitutions are preferably made in accordance with those set forth in Table 2 and Table 3. For example, such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g. Table 3 herein; pages 13-15 “Biochemistry” 2nd ED. Lubert Stryer ed (Stanford University); Henikoff et al., PNAS 1992 Vol 89 10915-10919; Lei et al., J Biol Chem 1995 May 19; 270(20):11882-11886). Other substitutions are also permissible and may be determined empirically or in accord with known conservative substitutions.

TABLE 2
Amino Acid Abbreviations

| SINGLE | THREE | |
|---|---|---|
| LETTER | LETTER | FULL NAME |
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gln | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

TABLE 3
Amino Acid Substitution or Similarity Matrix
Adapted from the GCG Software 9.0 BLOSUM62 amino acid substitution matrix
(block substitution matrix). The higher the value, the more likely a
substitution is found in related, natural proteins.

| A | C | D | E | F | G | H | I | K | L | M | N | P | Q | R | S | T | V | W | Y | . |
|---|---|---|---|---|---|---|---|---|---|---|---|---|---|---|---|---|---|---|---|---|
| 4 | 0 | −2 | −1 | −2 | 0 | −2 | −1 | −1 | −1 | −1 | −2 | −1 | −1 | −1 | 1 | 0 | 0 | −3 | −2 | A |
| | 9 | −3 | −4 | −2 | −3 | −3 | −1 | −3 | −1 | −1 | −3 | −3 | −3 | −3 | −1 | −1 | −1 | −2 | −2 | C |
| | | 6 | 2 | −3 | −1 | −1 | −3 | −1 | −4 | −3 | 1 | −1 | 0 | −2 | 0 | −1 | −3 | −4 | −3 | D |
| | | | 5 | −3 | −2 | 0 | −3 | 1 | −3 | −2 | 0 | −1 | 2 | 0 | 0 | −1 | −2 | −3 | −2 | E |
| | | | | 6 | −3 | −1 | 0 | −3 | 0 | 0 | −3 | −4 | −3 | −3 | −2 | −2 | −1 | 1 | 3 | F |
| | | | | | 6 | −2 | −4 | −2 | −4 | −3 | 0 | −2 | −2 | −2 | 0 | −2 | −3 | −2 | −3 | G |
| | | | | | | 8 | −3 | −1 | −3 | −2 | 1 | −2 | 0 | 0 | −1 | −2 | −3 | −2 | 2 | H |
| | | | | | | | 4 | −3 | 2 | 1 | −3 | −3 | −3 | −3 | −2 | −1 | 3 | −3 | −1 | I |
| | | | | | | | | 5 | −2 | −1 | 0 | −1 | 1 | 2 | 0 | −1 | −2 | −3 | −2 | K |
| | | | | | | | | | 4 | 2 | −3 | −3 | −2 | −2 | −2 | −1 | 1 | −2 | −1 | L |
| | | | | | | | | | | 5 | −2 | −2 | 0 | −1 | −1 | −1 | 1 | −1 | −1 | M |
| | | | | | | | | | | | 6 | −2 | 0 | 0 | 1 | 0 | −3 | −4 | −2 | N |
| | | | | | | | | | | | | 7 | −1 | −2 | −1 | −1 | −2 | −4 | −3 | P |
| | | | | | | | | | | | | | 5 | 1 | 0 | −1 | −2 | −2 | −1 | Q |
| | | | | | | | | | | | | | | 5 | −1 | −1 | −3 | −3 | −2 | R |
| | | | | | | | | | | | | | | | 4 | 1 | −2 | −3 | −2 | S |
| | | | | | | | | | | | | | | | | 5 | 0 | −2 | −2 | T |
| | | | | | | | | | | | | | | | | | 4 | −3 | −1 | V |
| | | | | | | | | | | | | | | | | | | 11 | 2 | W |
| | | | | | | | | | | | | | | | | | | | 7 | Y |

The term “homology” or “homologous” is intended to mean a sequence similarity between two polynucleotides or between two polypeptides. Similarity can be determined by comparing a position in each sequence, which can be aligned for purposes of comparison. If a given position of two polypeptide sequences is not identical, the similarity or conservativeness of that position can be determined by assessing the similarity of the amino acid of the position, for example, according to Table 3. A degree of similarity between sequences is a function of the number of matching or homologous positions shared by the sequences. The alignment of two sequences to determine their percent sequence similarity can be done using software programs known in the art, such as, for example, those described in Ausubel et al., Current Protocols in Molecular Biology, John Wiley and Sons, Baltimore, MD (1999). Preferably, default parameters are used for the alignment, examples of which are set forth below. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

The term “homologs” of to a given amino acid sequence or a nucleic acid sequence is intended to indicate that the corresponding sequences of the “homologs” having substantial identity or homology to the given amino acid sequence or nucleic acid sequence.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The term “cytotoxic agent” refers to a substance that inhibits or prevents the expression activity of cells, function of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to auristatins (e.g., auristatin E, auristatin F, MMAE and MMAF), auromycins, maytansinoids, ricin, ricin A-chain, combrestatin, duocarmycins, dolastatins, doxorubicin, daunorubicin, taxols, cisplatin, cc1065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷. Antibodies may also be conjugated to an anti-cancer pro-drug activating enzyme capable of converting the pro-drug to its active form.

The term “effective amount” or “therapeutically effective amount” as used herein refers to the amount of binding molecule (e.g., an antibody) or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., a cancer).

The term “cancer” or “cancer cell” is used herein to denote a tissue or cell found in a neoplasm which possesses characteristics which differentiate it from normal tissue or tissue cells. Among such characteristics include but are not limited to: degree of anaplasia, irregularity in shape, indistinctness of cell outline, nuclear size, changes in structure of nucleus or cytoplasm, other phenotypic changes, presence of cellular proteins indicative of a cancerous or pre-cancerous state, increased number of mitoses, and ability to metastasize. Words pertaining to “cancer” include carcinoma, sarcoma, tumor, epithelioma, leukemia, lymphoma, polyp, and scirrus, transformation, neoplasm, and the like.

As used herein, a “locally advanced” cancer refers to a cancer that has spread from where it started to nearby tissue or lymph nodes.

As used herein, a “metastatic” cancer refers to a cancer that has spread from where it started to different part of the body.

The term “intravesical administration” refers to instillation of a therapeutic agent directly into the bladder via insertion of a urethral catheter.

The term “dwell time” refers to the length of time a therapeutic substance will be retained in certain part or organ (e.g., bladder) of the treated subject.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

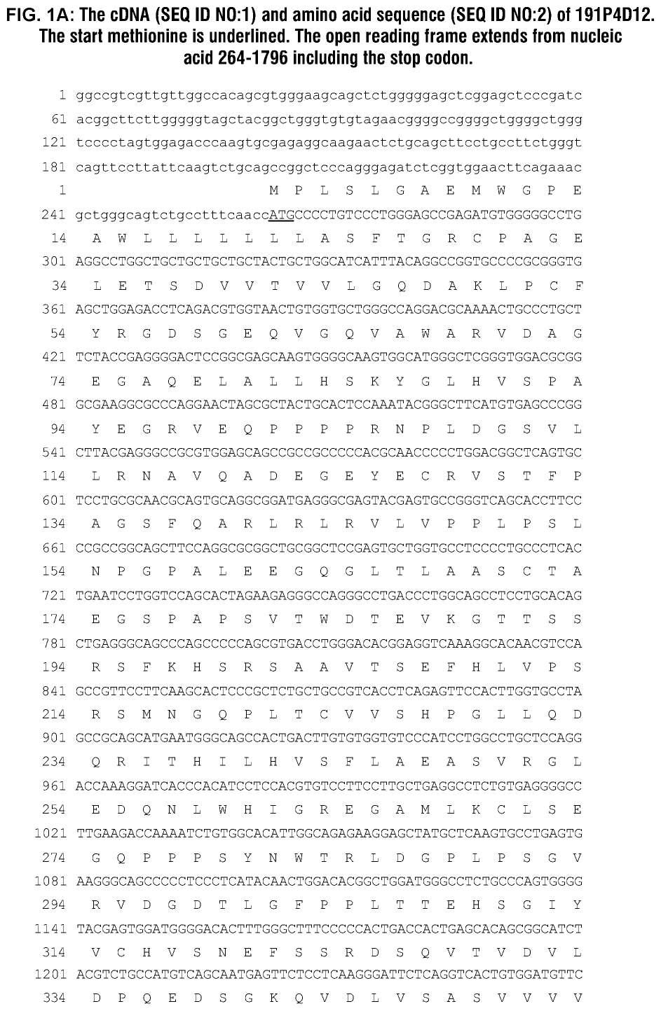

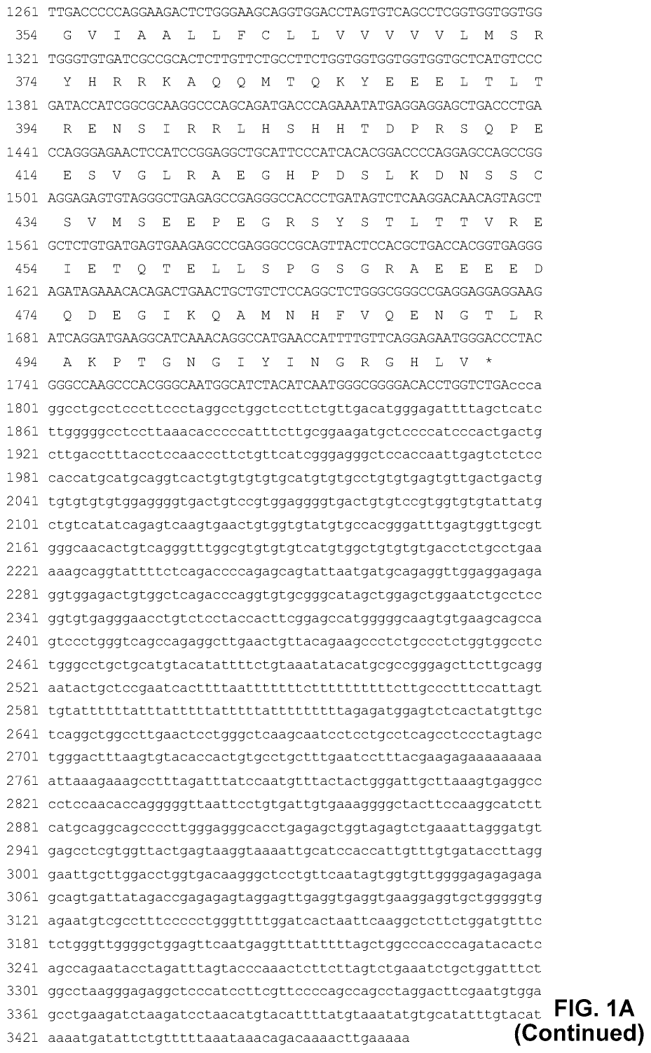

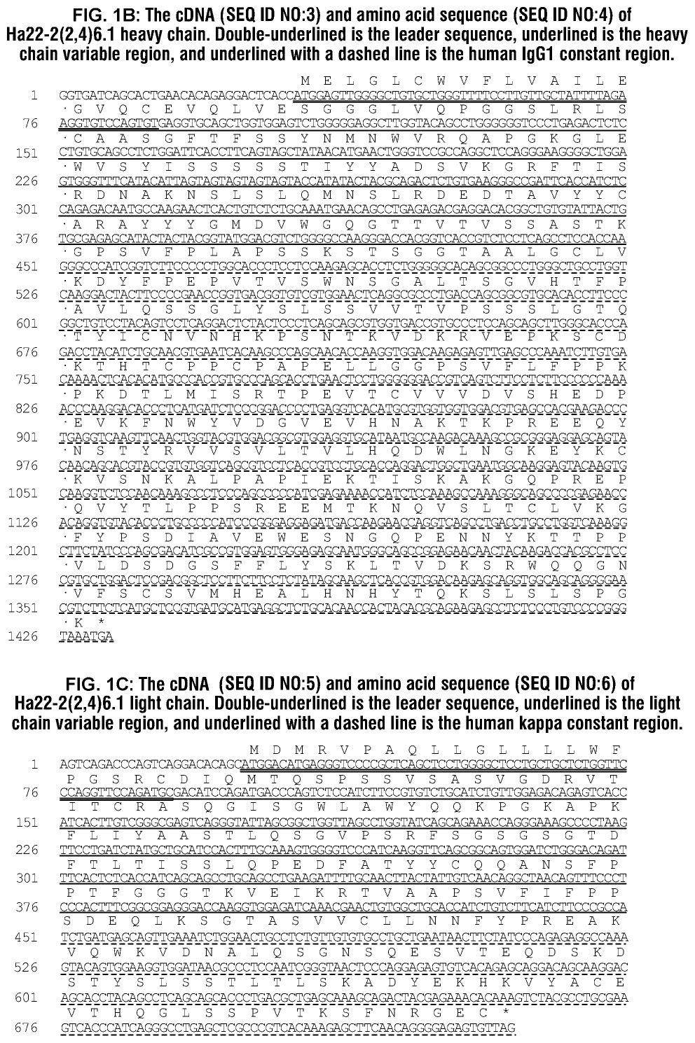

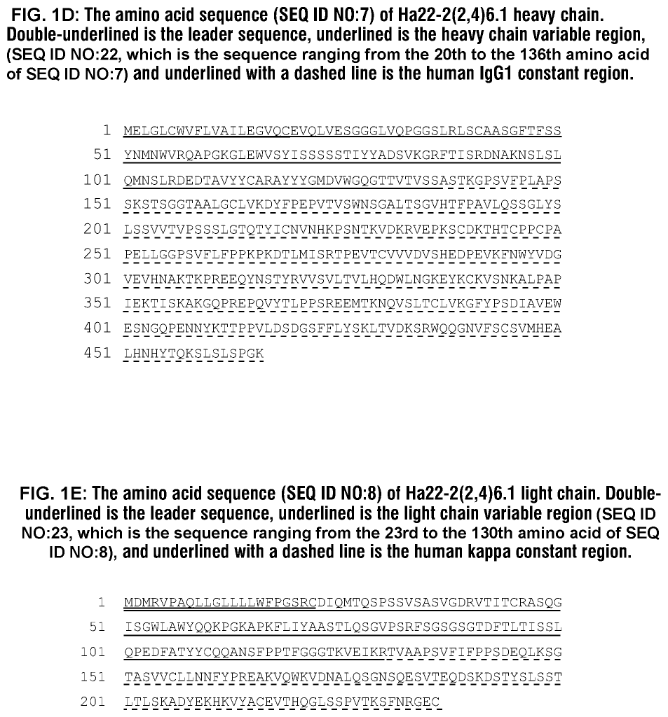

The term “variant” refers to a molecule that exhibits a variation from a described type or norm, such as a protein that has one or more different amino acid residues in the corresponding position(s) of a specifically described protein (e.g. the 191P4D12 protein shown in FIG. 1A.) An analog is an example of a variant protein. Splice isoforms and single nucleotides polymorphisms (SNPs) are further examples of variants.

The “191P4D12 proteins” and/or “191P4D12 related proteins” of the disclosure include those specifically identified herein (see, FIG. 1A), as well as allelic variants, conservative substitution variants, analogs and homologs that can be isolated/generated and characterized without undue experimentation following the methods outlined herein or readily available in the art. Fusion proteins that combine parts of different 191P4D12 proteins or fragments thereof, as well as fusion proteins of a 191P4D12 protein and a heterologous polypeptide are also included. Such 191P4D12 proteins are collectively referred to as the 191P4D12-related proteins, the proteins of the disclosure, or 191P4D12. The term “191P4D12-related protein” refers to a polypeptide fragment or a 191P4D12 protein sequence of 4, 5, 6, 7, 8, 9, 10, 11, 12, 13, 14, 15, 16, 17, 18, 19, 20, 21, 22, 23, 24, 25, or more than 25 amino acids; or, at least 30, 35, 40, 45, 50, 55, 60, 65, 70, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 330, 335, 339 or more amino acids. The term “191P4D12” is used interchangeably with nectin-4.

### 5.2 Methods of Treating Non-Muscle Invasive Bladder Cancer (NMIBC) for Selected Patients

Provided herein are methods for the treatment of bladder cancer in human subjects via intravesical administration of an antibody drug conjugate (ADC) that binds 191P4D12.

In one aspect, provided herein are methods of treating bladder cancer in a human subject, comprising intravesically administering to the subject an effective amount of an antibody drug conjugate (ADC), wherein the ADC comprises an antibody or antigen binding fragment thereof that binds to 191P4D12 conjugated to one or more units of monomethyl auristatin E (MMAE).

In some embodiments, the bladder cancer is non-muscle invasive bladder cancer (NMIBC). In some embodiments, the NMIBC has been histologically confirmed. In some embodiments, the NMIBC is carcinoma in situ (CIS). In some embodiments, the NMIBC has been histologically confirmed and is carcinoma in situ (CIS). In certain embodiments, the subject has papillary disease. In certain embodiments, the subject does not have papillary disease. In certain embodiments, the NMIBC has been histologically confirmed and wherein the predominant histologic component (>50%) is urothelial (transitional cell) carcinoma.

In some embodiments, the human subject treated with the methods provided herein has high-risk Bacillus Calmette-Guerin (BCG)-unresponsive disease. In certain embodiments, the high-risk BCG-unresponsive disease is defined as persistent or recurrent CIS alone or with recurrent Ta/T1 (noninvasive papillary disease/tumor invades the subepithelial connective tissue) disease within 12 months of completion of adequate BCG therapy. In certain embodiments, the adequate BCG therapy is defined as 5 of 6 doses of an initial induction course plus at least 2 of 3 doses maintenance therapy. In certain embodiments, the adequate BCG therapy is defined as 5 of 6 doses of an initial induction course plus at least 2 of 6 doses of a second induction course.

In some embodiments, the human subject treated with the methods provided herein is ineligible for a radical cystectomy. In some embodiments, the human subject treated with the methods provided herein refuses to undergo a radical cystectomy.

In some embodiments, all visible papillary Ta/T1 tumors of the subject have been completely resected within 60 days prior to the treatment. In some embodiments, the subject has residual pure CIS. In some embodiments, the subject does not have residucal pure CIS.

In some embodiments, the human subject treated with the methods provided herein has satisfactory bladder function and the ability to retain the ADC provided herein instillation for a minimum of 1 hour, even with premedication. In some embodiments, the human subject is at least 18-year old. In some embodiments, the human subject's estimated life expectancy is more than 2 years.

In some embodiments, the human subject treated with the methods provided herein has an Eastern Cooperative Oncology Group (ECOG) Performance Status score of 0. In some embodiments, the human subject treated with the methods provided herein has an Eastern Cooperative Oncology Group (ECOG) Performance Status score of 1. In some embodiments, the human subject treated with the methods provided herein has an Eastern Cooperative Oncology Group (ECOG) Performance Status score of 2. In some embodiments, the human subject treated with the methods provided herein has an Eastern Cooperative Oncology Group (ECOG) Performance Status score of 2 and the subject's glomerular filtration rate (GFR) is no less than 50 mL/min and the subject does not have New York Heart Association (NYHA) Class III heart failure.

In further embodiments of the methods provided herein, including the methods of the preceding paragraphs, the human subjects for whom the methods provided herein can be used are human subjects have various other conditions. In some embodiments, the human subject treated with the methods provided herein has the condition of absolute neutrophil count (ANC) no less than 1500/μL. In some embodiments, the human subject treated with the methods provided herein has the condition of hemoglobin (Hgb) no less than 10 g/dL. In some embodiments, the human subject treated with the methods provided herein has the condition of platelet count no less than 100,000/μL. In some embodiments, the human subject treated with the methods provided herein has the condition of serum bilirubin no more than 1.5×upper limit of normal (ULN) or no more than 3×ULN for subjects with Gilbert's disease. In some embodiments, the human subject treated with the methods provided herein has the condition of calculated creatinine clearance (CrCl) no less than 30 mL/min. In some embodiments, CrCl is calculated using the Cockcroft-Gault method or Modification of Diet in Renal Disease (MDRD) equations. In some embodiments, the human subject treated with the methods provided herein has the condition of GFR no less than 30 mL/min. In some embodiments, the human subject treated with the methods provided herein has an ECOG performance status of 2 and has GFR no less than 50 mL/min. In some embodiments, the human subject treated with the methods provided herein has the condition of alanine aminotransferase (ALT) and aspartate aminotransferase (AST) no more than 3×ULN. In some embodiments, the human subject treated with the methods provided herein has the condition of international normalized ratio (INR) or prothrombin time (PT), activated partial thromboplastin time (aPTT) or partial thromboplastin time (PTT) no more than 1.5 ULN unless the human subject is receiving anticoagulant therapy as long as PT or aPTT is within therapeutic range of intended use of anticoagulants. In some embodiments, the human subject treated with the methods provided herein has more than more of the conditions described in this paragraph. In some embodiments, the human subject treated with the methods provided herein has all of the conditions described in this paragraph.

In other embodiments of the methods provided herein, including the methods of the preceding paragraphs, the human subjects for whom the methods provided herein can be used are human subjects that do not have certain conditions. In some embodiments, the human subject treated with the methods provided herein has no current or prior history of muscle-invasive urothelial carcinoma (i.e., T2, T3, or T4 disease) or metastatic disease. In some embodiments, the human subject treated with the methods provided herein has no nodal or metastatic disease as noted on computed tomography (CT) or magnetic resonance imaging (MRI) done within 3 months prior to treatment with the ADC. In some embodiments, the human subject treated with the methods provided herein has no concomitant upper tract urothelial carcinoma as noted on CT or MRI urogram with contrast of abdomen/pelvis performed within 3 months prior to treatment with the ADC. In some embodiments, the human subject treated with the methods provided herein has no prior or concomitant urothelial carcinoma of the prostatic urethra within 6 months prior to treatment with the ADC. In some embodiments, the human subject treated with the methods provided herein has no tumor-related hydronephrosis prior to the administration of the ADC. In some embodiments, the human subject treated with the methods provided herein has not received asystemic anticancer therapy (e.g., chemotherapy, biologic therapy, immunotherapy, targeted therapy, endocrine therapy, investigational agent) within 4 weeks of the first dose of the treatment with the methods provided herein or any intravesical therapy for treatment of NMIBC within 6 weeks prior to the start of the treatment with the methods provided herein. In some embodiments, the human subject treated with the methods provided herein has received a single instillation of cytotoxic agents (e.g., mitomycin C, doxorubicin, and gemcitabine) immediately following a TURBT procedure between 14 and 60 days prior to the start of the treatment with the methods provided herein. In some embodiments, the human subject treated with the methods provided herein has no ongoing symptoms (Grade 2 and higher) secondary to adverse events (AEs) related to prior therapy for NMIBC. In some embodiments, the human subject treated with the methods provided herein has not received prior radiation to the bladder for the treatment of urothelial cancer. In some embodiments, the human subject treated with the methods provided herein has no active infection, wherein the subject has been treated with systemic (e.g., oral or intravenous) antibiotics within 14 days prior to the start of treatment with the ADC. In some embodiments, the human subject treated with the methods provided herein tolerates intravesical dosing or intravesical surgical manipulation. In some embodiments, the human subject treated with the methods provided herein has no history of a malignancy within 3 years prior to the treatment with the methods provided herein, or any evidence of residual disease from a previously diagnosed malignancy. In some embodiments, the human subject treated with the methods provided herein has had a negligible risk of metastasis or death (e.g., 5-year overall survival [OS]≥90%), such as adequately treated CIS of the cervix, non-melanoma skin carcinoma, ductal CIS of the breast, or Stage I uterine cancer. In some embodiments, the human subject treated with the methods provided herein has a history of prostate cancer (T2N0M0 or lower with Gleason score≤7) treated with definitive intent (surgically or with radiation therapy) at least 1 year prior to treatment with the methods provided herein, provided that the subject is considered prostate cancer-free, and the following criteria are met: (1) subjects who have undergone radical prostatectomy must have undetectable prostate-specific antigen (PSA) for >1 year prior to administration of the ADC, and (2) subjects who have had radiation must have a PSA doubling time>1 year (based on at least 3 values determined>1 month apart) and a total PSA value that does not meet Phoenix criteria for biochemical recurrence (i.e., <2.0 ng/mL above nadir). In some embodiments, the human subject treated with the methods provided herein has no previous exposure to Nectin-4-targeted therapy or a monomethyl auristatin E (MMAE)-containing agent. In some embodiments, the human subject treated with the methods provided herein does not have an autoimmune or inflammatory skin disorder. In some embodiments, the human subject treated with the methods provided herein does not have psoriasis or atopic dermatitis. In some embodiments, the human subject treated with the methods provided herein has no ongoing sensory or motor neuropathy Grade 2 or higher. In some embodiments, the human subject treated with the methods provided herein has no positive hepatitis B surface antigen and/or antihepatitis B core antibody. In some embodiments, the human subject treated with the methods provided herein has a negative polymerase chain reaction (PCR) assay of hepatitis B and has appropriate antiviral prophylaxis. In some embodiments, the human subject treated with the methods provided herein does not have active hepatitis C infection or known human immunodeficiency virus (HIV) infection. In some embodiments, the human subject treated with the methods provided herein does not have active tuberculosis. In some embodiments, the human subject treated with the methods provided herein does not have uncontrolled diabetes. In some embodiments, the uncontrolled diabetes is defined as a subject having hemoglobin A1c (HbA1c)≥8% or HbA1c 7% to <8% with associated diabetes symptoms (polyuria or polydipsia). In some embodiments, the human subject treated with the methods provided herein has not had a cerebral vascular event (e.g., a stroke or a transient ischemic attack), unstable angina, myocardial infarction, or cardiac symptoms consistent with NYHA Class III-IV within 6 months prior to a first dose of the ADC. In some embodiments, the human subject treated with the methods provided herein does not have severe (≥Grade 3) hypersensitivity to enfortumab vedotin or to any excipient contained in the drug formulation of enfortumab vedotin (e.g., histidine, trehalose dihydrate, and/or polysorbate 20). In some embodiments, the human subject treated with the methods provided herein does not have active keratitis or corneal ulcerations. In some embodiments, the human subject treated with the methods provided herein has superficial punctate keratitis.

In certain embodiments, the methods provided herein are used for treating subjects having non-muscle invasive bladder cancer (NMIBC) that express 191P4D12 RNA, express 191P4D12 protein, or express both 191P4D12 RNA and 191P4D12 protein.

In some embodiments, the 191P4D12 RNA expression in the cancer is determined by polynucleotide hybridization, sequencing (assessing the relative abundance of the sequences), and/or PCR (including RT-PCR). In some embodiments, the 191P4D12 protein expression in the cancer is determined by IHC, analysis in fluorescence-activated cell sorting (FACS), and/or Western blotting. In some embodiments, the 191P4D12 protein expression in the cancers is determined by more than one method. In some embodiments, the 191P4D12 protein expression in the cancers is determined by two methods of IHC.

In some embodiments, the non-muscle invasive bladder cancer (NMIBC) is confirmed histologically, cytologically, or both histologically and cytologically.

In another aspect, provided herein are methods of treating NMIBC in a human subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has any of the suitable characteristics as provided in Section 6.

In another aspect, provided herein is a method of preventing or treating cancer in a human subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the cancer has any of the suitable markers and/or characteristics as provided in Section 6.

In yet another aspect, provided herein is a method of preventing or treating cancer in a human subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), and wherein the subject has any of the suitable characteristics as provided in Section 6. In a further aspect, provided herein is a method of preventing or treating cancer in a human subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), and wherein the cancer has any of the suitable markers and/or characteristics as provided in Section 6.

In all the methods provided herein and specifically those described in the preceding paragraphs: the ADCs that can be used are described in Sections 3, 5.2, 5.3, 5.4, 5.5, 5.6, and 6, selection of patients for treatment is described herein and exemplified in this Section (Section 5.2) and Sections 3 and 6, dosing regimens and pharmaceutical composition for administering the therapeutic agent are described in Sections 5.4, 5.6 and 6 below, the biomarkers that can be used for identifying the therapeutic agents, selecting the patients, determining the outcome of these methods, and/or serving as criteria in any way for these methods are described herein and exemplified in this Section and Section 6, the biomarkers can be determined as described in Section 5.7 or as known in the art, therapeutic outcomes for the methods provided herein are described in this Section (Section 5.2) and Sections 3 and 6, additional therapeutic outcomes for the methods provided herein can be improvement of the biomarkers described herein, for example, those described and exemplified in this Section (Section 5.2) and Sections 3 and 6, and combination therapies including the ADCs and other therapeutic agents are described in this Section and in Section 5.5. Therefore, a person skilled in the art would understand that the methods provided herein include all permutations and combinations of the patients, therapeutic agents, dosing regiments, biomarkers, and therapeutic outcomes as described above and below.

### 5.3 Antibody Drug Conjugates for the Methods

In various embodiments of the methods provided herein, including the methods provided in Section 5.2, the ADC used in the methods comprises or is an anti-191P4D12 ADC described herein and/or in U.S. Pat. No. 8,637,642, which is herein incorporated in its entirety by reference. In some embodiments, the anti-191P4D12 antibody drug conjugate provided for the methods herein comprises an antibody or antigen binding fragment thereof that binds to 191P4D12 as provided herein, including in Sections 3, 5.3.1, and 6, conjugated to one or more units of cytotoxic agents (drug units, or D) as provided herein, including in Sections 3 and 6 and this Section (Section 5.3) with further disclosures in Sections 5.3.2 and 5.3.4. In certain embodiments, the cytotoxic agents (drug units, or D) can be covalently linked directly or via a linker unit (LU) as provided herein, including in Sections 3 and 6 and this Section (Section 5.3) with further disclosures in Section 5.3.3.

In some embodiments, the antibody drug conjugate compound has the following formula:

L-(LU-D)ₚ (I)
or a pharmaceutically acceptable salt or solvate thereof, wherein:
L is the antibody unit, e.g., the anti-nectin-4 antibody or an antigen binding fragment thereof for example as provided in Sections 3, 5.3.1, and 6, and
(LU-D) is a linker unit-drug unit moiety, wherein:
LU- is a linker unit for example as provided in Sections 3 and 6 and this Section (Section 5.3) with further disclosures in Section 5.3.3, and
D is a drug unit having cytostatic or cytotoxic activity against a target cell for example as provided Sections 3 and 6 and this Section (Section 5.3) with further disclosures in Sections 5.3.2 and 5.3.4; and
p is an integer from 1 to 20 with further examples provided in Sections 3 and 6 and this Section (Section 5.3).

In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20.

In some embodiments, the antibody drug conjugate compound has the following formula:

L-(Aₐ-W_{w}-Y_{y}-D)ₚ (II)
or a pharmaceutically acceptable salt or solvate thereof, wherein:
L is the Antibody unit, e.g., the anti-nectin-4 antibody or an antigen binding fragment thereof for example as provided in Sections 3, 5.3.1, and 6; and
-Aₐ-W_{w}-Y_{y}- is a linker unit (LU), wherein:
-A- is a stretcher unit,
a is 0 or 1,
each -W- is independently an amino acid unit,
w is an integer ranging from 0 to 12,
-Y- is a self-immolative spacer unit,
y is 0, 1 or 2,
each for example as provided in Sections 3 and 6 and this Section (Section 5.3) with further disclosures in Section 5.3.3;
D is a drug units having cytostatic or cytotoxic activity against the target cell for example as provided Sections 3 and 6 and this Section (Section 5.3) with further disclosures in Sections 5.3.2 and 5.3.4; and
p is an integer from 1 to 20 with further examples provided in Sections 3 and 6 and this Section (Section 5.3).

In some embodiments, a is 0 or 1, w is 0 or 1, and y is 0, 1 or 2. In some embodiments, a is 0 or 1, w is 0 or 1, and y is 0 or 1. In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20. In some embodiments, when w is not zero, y is 1 or 2. In some embodiments, when w is 1 to 12, y is 1 or 2. In some embodiments, w is 2 to 12 and y is 1 or 2. In some embodiments, a is 1 and w and y are 0.

In some specific embodiments of the methods provided herein, including the methods provided in Section 5.2, the cytotoxic agent as part of any of the ADCs provided herein for the methods comprises, consists of, or is MMAE.

For compositions comprising a plurality antibodies or antigen binding fragments thereof, the drug loading is represented by p, the average number of drug molecules per antibody unit. Drug loading can range from 1 to 20 drugs (D) per antibody. The average number of drugs per antibody in preparation of conjugation reactions can be characterized by conventional means such as mass spectroscopy, ELISA assay, and HPLC. The quantitative distribution of antibody drug conjugates in terms of p can also be determined. In some instances, separation, purification, and characterization of homogeneous antibody drug conjugates where p is a certain value from antibody drug conjugates with other drug loadings can be achieved by means such as reverse phase HPLC or electrophoresis. In certain exemplary embodiments, p is from 2 to 8.

Additional embodiments of the ADC for the methods provided herein have been described in U.S. Pat. No. 8,637,642 and International Application No. PCT/US2019/056214 (Publication No. WO2020/117373), both of which are hereby incorporated in their entireties by reference.

In some embodiments of the methods provided herein, including in Sections 3, 5.2, and 6 and this Section (Section 5.3), the ADC is enfortumab vedotin. In certain embodiments of the methods provided herein, including in Sections 3, 5.2, and 6 and this Section (Section 5.3), the ADC is a biosimilar of enfortumab vedotin.

### 5.3.1 Anti-191P4D12 Antibodies or Antigen Binding Fragments

In one embodiment, the antibody or antigen binding fragment thereof that binds to nectin-4-related proteins is an antibody or antigen binding fragment that specifically binds to nectin-4 protein comprising amino acid sequence of SEQ ID NO:2 (see FIG. 1A). The corresponding cDNA encoding the 191P4D12 protein has a sequence of SEQ ID NO:1 (see FIG. 1A).

The antibody that specifically binds to nectin-4 protein comprising amino acid sequence of SEQ ID NO:2 includes antibodies that can bind to other nectin-4-related proteins. For example, antibodies that bind nectin-4 protein comprising amino acid sequence of SEQ ID NO:2 can bind nectin-4-related proteins such as nectin-4 variants and the homologs or analogs thereof.

In some embodiments, the anti-nectin-4 antibody provided herein is a monoclonal antibody.

In some embodiments, the antibody comprises a heavy chain comprising an amino acid sequence of SEQ ID NO:4 (cDNA sequence of SEQ ID NO:3), and/or a light chain comprising an amino acid sequence of SEQ ID NO: 6 (cDNA sequence of SEQ ID NO:5), as shown in FIGS. 1B and 1C.

In some embodiments, the anti-nectin-4 antibody or antigen binding fragment thereof comprises a heavy chain variable region comprising complementarity determining regions (CDRs) comprising the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs comprising the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). In certain embodiments, the anti-nectin-4 antibody or antigen binding fragment thereof comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO:22 (which is the amino acid sequence ranging from the 20th amino acid (glutamic acid) to the 136th amino acid (serine) of SEQ ID NO:7) and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO:23 (which is the amino acid sequence ranging from the 23rd amino acid (aspartic acid) to the 130th amino acid (arginine) of SEQ ID NO:8). In some embodiments, the anti-nectin-4 antibody or antigen binding fragment thereof comprises a heavy chain variable region comprising complementarity determining regions (CDRs) consisting of the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs consisting of the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). In certain embodiments, the anti-nectin-4 antibody or antigen binding fragment thereof comprises a heavy chain variable region comprising complementarity determining region 1 (CDR-H1), CDR-H2, and CDR-H3 consisting of the amino acid sequences of the corresponding CDR-H1, CDR-H2, and CDR-H3 in the heavy chain variable region sequence set forth in SEQ ID NO:22 (which is the amino acid sequence ranging from the 20th amino acid (glutamic acid) to the 136th amino acid (serine) of SEQ ID NO:7) and a light chain variable region comprising CDR-L1, CDR-L2, and CDR-L3 consisting of the amino acid sequences of the corresponding CDR-L1, CDR-L2, and CDR-L3 in the light chain variable region sequence set forth in SEQ ID NO:23 (which is the amino acid sequence ranging from the 23rd amino acid (aspartic acid) to the 130th amino acid (arginine) of SEQ ID NO:8). SEQ ID NO: 22, SEQ ID NO:23, SEQ ID NO:7 and SEQ ID NO:8 are as shown in FIGS. 1D and 1E and listed below:

SEQ ID NO: 22
EVQLVESGGGLVQPGGSLRLSCAASGFTFSSYNMNWVRQAPGKGLEWVSY
ISSSSSTIYYADSVKGRFTISRDNAKNSLSLQMNSLRDEDTAVYYCARAY
YYGMDVWGQGTTVTVSS
SEQ ID NO: 23
DIQMTQSPSSVSASVGDRVTITCRASQGISGWLAWYQQKPGKAPKFLIYA
ASTLQSGVPSRFSGSGSGTDFTLTISSLQPEDFATYYCQQANSFPPTFGG
GTKVEIKR
SEQ ID NO: 7
MELGLCWVFLVAILEGVQCEVQLVESGGGLVQPGGSLRLSCAASGFTFSS
YNMNWVRQAPGKGLEWVSYISSSSSTIYYADSVKGRFTISRDNAKNSLSL
QMNSLRDEDTAVYYCARAYYYGMDVWGQGTTVTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEA
LHNHYTQKSLSLSPGK
SEQ ID NO: 8
MDMRVPAQLLGLLLLWFPGSRCDIQMTQSPSSVSASVGDRVTITCRASQG
ISGWLAWYQQKPGKAPKFLIYAASTLQSGVPSRFSGSGSGTDFTLTISSL
QPEDFATYYCQQANSFPPTFGGGTKVEIKRTVAAPSVFIFPPSDEQLKSG
TASVVCLLNNFYPREAKVQWKVDNALQSGNSQESVTEQDSKDSTYSLSST
LTLSKADYEKHKVYACEVTHQGLSSPVTKSFNRGEC

CDR sequences can be determined according to well-known numbering systems. As described above, CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Pluckthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted in Table 1 above.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Kabat numbering and a light chain variable region comprising CDRs comprising the amino acid sequences of the CDRs of the light chain variable region set forth in SEQ ID NO:23 according to Kabat numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to AbM numbering and a light chain variable region comprising CDRs comprising the amino acid sequences of the CDRs of the light chain variable region set forth in SEQ ID NO:23 according to AbM numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Chothia numbering and a light chain variable region comprising CDRs comprising the amino acid sequences of the CDRs of the light chain variable region set forth in SEQ ID NO:23 according to Chothia numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Contact numbering and a light chain variable region comprising CDRs comprising the amino acid sequences of the CDRs of the light chain variable region set forth in SEQ ID NO:23 according to Contact numbering.

In yet other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to IMGT numbering and a light chain variable region comprising CDRs comprising the amino acid sequences of the CDRs of the light chain variable region set forth in SEQ ID NO:23 according to IMGT numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Kabat numbering and a light chain variable region comprising CDRs consisting of the amino acid sequences of the CDRs of the light chain variable region set forth in SEQ ID NO:23 according to Kabat numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to AbM numbering and a light chain variable region comprising CDRs consisting of the amino acid sequences of the CDRs of the light chain variable region set forth in SEQ ID NO:23 according to AbM numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Chothia numbering and a light chain variable region comprising CDRs consisting of the amino acid sequences of the CDRs of the light chain variable region set forth in SEQ ID NO:23 according to Chothia numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Contact numbering and a light chain variable region comprising CDRs consisting of the amino acid sequences of the CDRs of the light chain variable region set forth in SEQ ID NO:23 according to Contact numbering.

In yet other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to IMGT numbering and a light chain variable region comprising CDRs consisting of the amino acid sequences of the CDRs of the light chain variable region set forth in SEQ ID NO:23 according to IMGT numbering.

As described above, the CDR sequences according to different numbering systems can be readily determined, e.g., using online tools such as the one provided by Antigen receptor Numbering And Receptor ClassificatIon (ANARCI). For example, the heavy chain CDR sequences within SEQ ID NO:22, and the light chain CDR sequences within SEQ ID NO:23 according to Kabat numbering as determined by ANARCI are listed in Table 4 below.

TABLE 4

| | VH of SEQ ID NO: 22 | VL of SEQ ID NO: 23 |
|---|---|---|
| CDR1 | SYNMN (SEQ ID NO: 9) | RASQGISGWLA (SEQ ID |
| | | NO: 12) |
| CDR2 | YISSSSSTIYYADSVKG | AASTLQS (SEQ ID NO: |
| | (SEQ ID NO: 10) | 13) |
| CDR3 | AYYYGMDV (SEQ ID NO: | QQANSFPPT (SEQ ID |
| | 11) | NO: 14) |

For another example, the heavy chain CDR sequences within SEQ ID NO:22, and the light chain CDR sequences within SEQ ID NO:23 according to IMGT numbering as determined by ANARCI are listed in Table 5 below.

TABLE 5

| | VH of SEQ ID NO: 22 | VL of SEQ ID NO: 23 |
|---|---|---|
| CDR1 | GFTFSSYN (SEQ ID NO: | QGISGW (SEQ ID NO: |
| | 16) | 19) |
| CDR2 | ISSSSSTI (SEQ ID NO: | AAS (SEQ ID NO: 20) |
| | 17) | |
| CDR3 | ARAYYYGMDV (SEQ ID | QQANSFPPT (SEQ ID |
| | NO: 18) | NO: 21) |

In some embodiments, the antibody or antigen binding fragment thereof comprises CDR-H1 comprising an amino acid sequence of SEQ ID NO:9, CDR-H2 comprising an amino acid sequence of SEQ ID NO:10, CDR-H3 comprising an amino acid sequence of SEQ ID NO:11, CDR-L1 comprising an amino acid sequence of SEQ ID NO:NO:12, CDR-L2 comprising an amino acid sequence of SEQ ID NO:NO:13, and CDR-L3 comprising an amino acid sequence of SEQ ID NO:NO:14.

In some embodiments, the antibody or antigen binding fragment thereof comprises CDR-H1 comprising an amino acid sequence of SEQ ID NO:16, CDR-H2 comprising an amino acid sequence of SEQ ID NO:17, CDR-H3 comprising an amino acid sequence of SEQ ID NO:18, CDR-L1 comprising an amino acid sequence of SEQ ID NO:NO:19, CDR-L2 comprising an amino acid sequence of SEQ ID NO:NO:20, and CDR-L3 comprising an amino acid sequence of SEQ ID NO:NO:21.

In some embodiments, the antibody or antigen binding fragment thereof comprises CDR-H1 consisting of an amino acid sequence of SEQ ID NO:9, CDR-H2 consisting of an amino acid sequence of SEQ ID NO:10, CDR-H3 consisting of an amino acid sequence of SEQ ID NO:11, CDR-L1 consisting of an amino acid sequence of SEQ ID NO:NO:12, CDR-L2 consisting of an amino acid sequence of SEQ ID NO:NO:13, and CDR-L3 consisting of an amino acid sequence of SEQ ID NO:NO:14.

In some embodiments, the antibody or antigen binding fragment thereof comprises CDR-H1 consisting of an amino acid sequence of SEQ ID NO:16, CDR-H2 consisting of an amino acid sequence of SEQ ID NO:17, CDR-H3 consisting of an amino acid sequence of SEQ ID NO:18, CDR-L1 consisting of an amino acid sequence of SEQ ID NO:NO:19, CDR-L2 consisting of an amino acid sequence of SEQ ID NO:NO:20, and CDR-L3 consisting of an amino acid sequence of SEQ ID NO:NO:21.

In some embodiments, the antibody or antigen binding fragment thereof comprises a heavy chain variable region comprising the amino acid sequence of SEQ ID NO:22 and a light chain variable region comprising the amino acid sequence of SEQ ID NO:23.

In some embodiments, the antibody or antigen binding fragment thereof comprises a heavy chain variable region consisting of the amino acid sequence of SEQ ID NO:22 and a light chain variable region consisting of the amino acid sequence of SEQ ID NO:23.

In some embodiments, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

In some embodiments, the antibody comprises a heavy chain consisting of the amino acid sequence ranging from the 20th amino acid (glutamic acid) to the 466th amino acid (lysine) of SEQ ID NO:7 and a light chain consisting of the amino acid sequence ranging from the 23rd amino acid (aspartic acid) to the 236th amino acid (cysteine) of SEQ ID NO:8.

In some embodiments, amino acid sequence modification(s) of antibodies described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art who appreciate that amino acid changes can alter post-translational processes of the antibody, such as changing the number or position of glycosylation sites or altering the membrane anchoring characteristics.

In some embodiments, the antibodies provided herein are chemically modified, for example, by the covalent attachment of any type of molecule to the antibody. The antibody derivatives can include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formulation, metabolic synthesis of tunicamycin, etc. Additionally, the antibody can contain one or more non-classical amino acids.

Variations can be a substitution, deletion, or insertion of one or more codons encoding the single domain antibody or polypeptide that results in a change in the amino acid sequence as compared with the original antibody or polypeptide. Amino acid substitutions can be the result of replacing one amino acid with another amino acid comprising similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions can optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed can be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue comprising a side chain with a similar charge. As described above, families of amino acid residues comprising side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined conservative (e.g., within an amino acid group with similar properties and/or side chains) substitutions can be made, so as to maintain or not significantly change the properties.

Amino acids can be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the antibody also can be substituted, for example, with another amino acid, such as alanine or serine, to improve the oxidative stability of the molecule and to prevent aberrant crosslinking.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the anti-anti-MSLN antibody variant DNA.

Covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

In certain embodiments, the antibody or antigen binding fragment provided herein comprises a heavy chain having certain homology or identity to the heavy chain as set forth in SEQ ID NO:7 and a light chain having certain homology or identity to the light chain as set forth in SEQ ID NO:8. Such embodiments of heavy/light chains with homology or identity are further provided as follows. In some embodiments, the antibody or antigen binding fragment provided herein comprises a heavy chain having more than 70% homology or identity to the heavy chain as set forth in SEQ ID NO:7. In some embodiments, the antibody or antigen binding fragment provided herein comprises a heavy chain having more than 75% homology or identity to the heavy chain as set forth in SEQ ID NO:7. In some embodiments, the antibody or antigen binding fragment provided herein comprises a heavy chain having more than 80% homology or identity to the heavy chain as set forth in SEQ ID NO:7. In some embodiments, the antibody or antigen binding fragment provided herein comprises a heavy chain having more than 85% homology or identity to the heavy chain as set forth in SEQ ID NO:7. In some embodiments, the antibody or antigen binding fragment provided herein comprises a heavy chain having more than 90% homology or identity to the heavy chain as set forth in SEQ ID NO:7. In some embodiments, the antibody or antigen binding fragment provided herein comprises a heavy chain having more than 95% homology or identity to the heavy chain as set forth in SEQ ID NO:7. In certain embodiments, the antibody or antigen binding fragment provided herein comprises a heavy chain having any of the provided homology or identity to the heavy chain as set forth in SEQ ID NO:7, wherein the CDRs (CDR-H1, CDR-H2, and CDR-H3) are identical to the CDRs in the heavy chain as set forth in SEQ ID NO:7. In some embodiments, the antibody or antigen binding fragment provided herein comprises a light chain having more than 70% homology or identity to the light chain as set forth in SEQ ID NO:8. In some embodiments, the antibody or antigen binding fragment provided herein comprises a light chain having more than 75% homology or identity to the light chain as set forth in SEQ ID NO:8. In some embodiments, the antibody or antigen binding fragment provided herein comprises a light chain having more than 80% homology or identity to the light chain as set forth in SEQ ID NO:8. In some embodiments, the antibody or antigen binding fragment provided herein comprises a light chain having more than 85% homology or identity to the light chain as set forth in SEQ ID NO:8. In some embodiments, the antibody or antigen binding fragment provided herein comprises a light chain having more than 90% homology or identity to the light chain as set forth in SEQ ID NO:8. In some embodiments, the antibody or antigen binding fragment provided herein comprises a light chain having more than 95% homology or identity to the light chain as set forth in SEQ ID NO:8. In certain embodiments, the antibody or antigen binding fragment provided herein comprises a light chain having any of the provided homology or identity to the light chain as set forth in SEQ ID NO:8, wherein the CDRs (CDR-L1, CDR-L2, and CDR-L3) are identical to the CDRs in the light chain as set forth in SEQ ID NO:8. In certain embodiments, the antibody or antigen binding fragment provided herein comprises any homologous light chain and any homologous heavy chain as provided in this paragraph in any combination or permutation.

In certain embodiments, the antibody or antigen binding fragment provided herein comprises a heavy chain variable region having certain homology or identity to the heavy chain variable region as set forth in SEQ ID NO:22 and a light chain variable region having certain homology or identity to the light chain variable region as set forth in SEQ ID NO:23. Such embodiments of heavy chain variable regions and light chain variable regions with homology or identity are further provided as follows. In some embodiments, the antibody or antigen binding fragment provided herein comprises a heavy chain variable region having more than 70% homology or identity to the heavy chain variable region as set forth in SEQ ID NO:22. In some embodiments, the antibody or antigen binding fragment provided herein comprises a heavy chain variable region having more than 75% homology or identity to the heavy chain variable region as set forth in SEQ ID NO:22. In some embodiments, the antibody or antigen binding fragment provided herein comprises a heavy chain variable region having more than 80% homology or identity to the heavy chain variable region as set forth in SEQ ID NO:22. In some embodiments, the antibody or antigen binding fragment provided herein comprises a heavy chain variable region having more than 85% homology or identity to the heavy chain variable region as set forth in SEQ ID NO:22. In some embodiments, the antibody or antigen binding fragment provided herein comprises a heavy chain variable region having more than 90% homology or identity to the heavy chain variable region as set forth in SEQ ID NO:22. In some embodiments, the antibody or antigen binding fragment provided herein comprises a heavy chain variable region having more than 95% homology or identity to the heavy chain variable region as set forth in SEQ ID NO:22. In certain embodiments, the antibody or antigen binding fragment provided herein comprises a heavy chain variable region having any of the provided homology or identity to the heavy chain variable region as set forth in SEQ ID NO:22, wherein the CDRs (CDR-H1, CDR-H2, and CDR-H3) are identical to the CDRs in the heavy chain variable region as set forth in SEQ ID NO:22. In some embodiments, the antibody or antigen binding fragment provided herein comprises a light chain variable region having more than 70% homology or identity to the light chain variable region as set forth in SEQ ID NO:23. In some embodiments, the antibody or antigen binding fragment provided herein comprises a light chain variable region having more than 75% homology or identity to the light chain variable region as set forth in SEQ ID NO:23. In some embodiments, the antibody or antigen binding fragment provided herein comprises a light chain variable region having more than 80% homology or identity to the light chain variable region as set forth in SEQ ID NO:23. In some embodiments, the antibody or antigen binding fragment provided herein comprises a light chain variable region having more than 85% homology or identity to the light chain variable region as set forth in SEQ ID NO:23. In some embodiments, the antibody or antigen binding fragment provided herein comprises a light chain variable region having more than 90% homology or identity to the light chain variable region as set forth in SEQ ID NO:23. In some embodiments, the antibody or antigen binding fragment provided herein comprises a light chain variable region having more than 95% homology or identity to the light chain variable region as set forth in SEQ ID NO:23. In certain embodiments, the antibody or antigen binding fragment provided herein comprises a light chain variable region having any of the provided homology or identity to the light chain variable region as set forth in SEQ ID NO:23, wherein the CDRs (CDR-L1, CDR-L2, and CDR-L3) are identical to the CDRs in the light chain variable region as set forth in SEQ ID NO:23. In certain embodiments, the antibody or antigen binding fragment provided herein comprises any homologous light chain variable region and any homologous heavy chain variable region as provided in this paragraph in any combination or permutation.

In some embodiments, the anti-nectin-4 antibody provided herein comprises heavy and light chain CDR regions of an antibody designated Ha22-2(2,4)6.1 produced by a hybridoma deposited under the American Type Culture Collection (ATCC) Accession NO: PTA-11267, or heavy and light chain CDR regions comprising amino acid sequences that are homologous to the amino acid sequences of the heavy and light chain CDR regions of Ha22-2(2,4)6.1, and wherein the antibodies retain the desired functional properties of the anti-nectin-4 antibody designated Ha22-2(2,4)6.1 produced by a hybridoma deposited under the American Type Culture Collection (ATCC) Accession NO: PTA-11267.

In some embodiments, the anti-nectin-4 antibody provided herein comprises heavy and light chain CDR regions (CDR-H1, CDR-H2, CDR-H3, CDR-L1, CDR-L2, and CDR-L3) of an antibody designated Ha22-2(2,4)6.1 produced by a hybridoma deposited under the American Type Culture Collection (ATCC) Accession NO: PTA-11267, or heavy and light chain CDR regions consisting of amino acid sequences that are homologous to the amino acid sequences of the heavy and light chain CDR regions of Ha22-2(2,4)6.1, and wherein the antibodies retain the desired functional properties of the anti-nectin-4 antibody designated Ha22-2(2,4)6.1 produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:
(a) the heavy chain variable region comprises CDRs (CDR-H1, CDR-H2, and CDR-H3) comprising the amino acid sequences of the heavy chain variable region CDRs set forth in the antibody produced by a hybridoma deposited under the American Type Culture Collection (ATCC) Accession NO: PTA-11267;
(b) the light chain variable region comprises CDRs (CDR-L1, CDR-L2, and CDR-L3) comprising the amino acid sequences of the light chain variable region CDRs set forth in the antibody produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:
(a) the heavy chain variable region comprises CDRs (CDR-H1, CDR-H2, and CDR-H3) consisting of the amino acid sequences of the heavy chain variable region CDRs set forth in the antibody produced by a hybridoma deposited under the American Type Culture Collection (ATCC) Accession NO: PTA-11267;
(b) the light chain variable region comprises CDRs (CDR-L1, CDR-L2, and CDR-L3) consisting of the amino acid sequences of the light chain variable region CDRs set forth in the antibody produced by a hybridoma deposited under the American Type Culture Collection (ATCC) Accession NO: PTA-11267.

In some embodiments, the anti-nectin-4 antibody provided herein comprises heavy and light chain variable regions of an antibody designated Ha22-2(2,4)6.1 produced by a hybridoma deposited under the American Type Culture Collection (ATCC) Accession NO: PTA-11267, or heavy and light variable regions comprising amino acid sequences that are homologous to the amino acid sequences of the heavy and light chain variable regions of Ha22-2(2,4)6.1, and wherein the antibodies retain the desired functional properties of the anti-nectin-4 antibody provided herein. In some embodiments, the anti-nectin-4 antibody provided herein comprises heavy and light chain variable regions of an antibody designated Ha22-2(2,4)6.1 produced by a hybridoma deposited under the American Type Culture Collection (ATCC) Accession NO: PTA-11267, or heavy and light variable regions consisting of amino acid sequences that are homologous to the amino acid sequences of the heavy and light chain variable regions of Ha22-2(2,4)6.1, and wherein the antibodies retain the desired functional properties of the anti-nectin-4 antibody provided herein. As the constant region of the antibody of the disclosure, any subclass of constant region can be chosen. In one embodiment, human IgG1 constant region as the heavy chain constant region and human Ig kappa constant region as the light chain constant region can be used.

In some embodiments, the anti-nectin-4 antibody provided herein comprises heavy and light chains of an antibody designated Ha22-2(2,4)6.1 produced by a hybridoma deposited under the American Type Culture Collection (ATCC) Accession NO: PTA-11267, or heavy and light chains comprising amino acid sequences that are homologous to the amino acid sequences of the heavy and light chains of Ha22-2(2,4)6.1, and wherein the antibodies retain the desired functional properties of the anti-nectin-4 antibody provided herein. In some embodiments, the anti-nectin-4 antibody provided herein comprises heavy and light chains of an antibody designated Ha22-2(2,4)6.1 produced by a hybridoma deposited under the American Type Culture Collection (ATCC) Accession NO: PTA-11267, or heavy and light chains consisting of amino acid sequences that are homologous to the amino acid sequences of the heavy and light chains of Ha22-2(2,4)6.1, and wherein the antibodies retain the desired functional properties of the anti-nectin-4 antibody provided herein.

In some embodiments, the antibody or antigen binding fragment thereof provided herein comprises a heavy chain variable region and a light chain variable region, wherein:
(a) the heavy chain variable region comprises an amino acid sequence that is at least 80% homologous or identical to the heavy chain variable region amino acid sequence of the antibody produced by a hybridoma deposited under the American Type Culture Collection (ATCC) Accession NO: PTA-11267; and
(b) the light chain variable region comprises an amino acid sequence that is at least 80% homologous or identical to the light chain variable region amino acid sequence of the antibody produced by a hybridoma deposited under the American Type Culture Collection (ATCC) Accession NO: PTA-11267.

In certain embodiments, the antibody or antigen binding fragment provided herein comprises a heavy chain variable region having certain homology or identity to the heavy chain variable region amino acid sequence of the antibody produced by a hybridoma deposited under the American Type Culture Collection (ATCC) Accession NO: PTA-11267 and a light chain variable region having certain homology or identity to the light chain variable region amino acid sequence of the antibody produced by a hybridoma deposited under the American Type Culture Collection (ATCC) Accession NO: PTA-11267. Such embodiments of heavy chain variable regions and light chain variable regions with homology or identity are further provided as follows. In some embodiments, the heavy chain variable region comprises an amino acid sequence that is at least 85% homologous or identical to the heavy chain variable region amino acid sequence of the antibody produced by a hybridoma deposited under the American Type Culture Collection (ATCC) Accession NO: PTA-11267. In other embodiments, the heavy chain variable region comprises an amino acid sequence that is at least 90% homologous or identical to the heavy chain variable region amino acid sequence of the antibody produced by a hybridoma deposited under the American Type Culture Collection (ATCC) Accession NO: PTA-11267. In yet other embodiments, the heavy chain variable region comprises an amino acid sequence that is at least 95% homologous or identical to the heavy chain variable region amino acid sequence of the antibody produced by a hybridoma deposited under the American Type Culture Collection (ATCC) Accession NO: PTA-11267. In other embodiments, the heavy chain variable region can be 85%, 86%, 87%, 88%, 89%, 90%, 91%, 92%, 93%, 94%, 95%, 96%, 97%, 98% or 99% homologous or identical to the heavy chain variable region amino acid sequence of the antibody produced by a hybridoma deposited under the American Type Culture Collection (ATCC) Accession NO: PTA-11267. In some embodiments, the light chain variable region comprises an amino acid sequence that is at least 85% homologous or identical to the light chain variable region amino acid sequence of the antibody produced by a hybridoma deposited under the American Type Culture Collection (ATCC) Accession NO: PTA-11267. In other embodiments, the light chain variable region comprises an amino acid sequence that is at least 90% homologous or identical to the light chain variable region amino acid sequence of the antibody produced by a hybridoma deposited under the American Type Culture Collection (ATCC) Accession NO: PTA-11267. In yet other embodiments, the light chain variable region comprises an amino acid sequence that is at least 95% homologous or identical to the light chain variable region amino acid sequence of the antibody produced by a hybridoma deposited under the American Type Culture Collection (ATCC) Accession NO: PTA-11267. In other embodiments, the light chain variable region can be 85%, 86%, 87%, 88%, 89%, 90%, 91%, 92%, 93%, 94%, 95%, 96%, 97%, 98% or 99% homologous or identical to the light chain variable region amino acid sequence of the antibody produced by a hybridoma deposited under the American Type Culture Collection (ATCC) Accession NO: PTA-11267. In certain embodiments, the antibody or antigen binding fragment provided herein comprises any homologous light chain variable region and any homologous heavy chain variable region as provided in this paragraph in any combination or permutation.

In other embodiments, the antibody or antigen binding fragment thereof provided herein comprises a heavy chain and a light chain, wherein:
(a) the heavy chain comprises an amino acid sequence that is at least 80% homologous or identical to the heavy chain amino acid sequence of the antibody produced by a hybridoma deposited under the American Type Culture Collection (ATCC) Accession NO: PTA-11267; and
(b) the light chain comprises an amino acid sequence that is at least 80% homologous or identical to the light chain amino acid sequence of the antibody produced by a hybridoma deposited under the American Type Culture Collection (ATCC) Accession NO: PTA-11267.

In certain embodiments, the antibody or antigen binding fragment provided herein comprises a heavy chain having certain homology or identity to the heavy chain amino acid sequence of the antibody produced by a hybridoma deposited under the American Type Culture Collection (ATCC) Accession NO: PTA-11267 and a light chain having certain homology or identity to the light chain amino acid sequence of the antibody produced by a hybridoma deposited under the American Type Culture Collection (ATCC) Accession NO: PTA-11267. Such embodiments of heavy chains and light chains with homology or identity are further provided as follows. In some embodiments, the heavy chain comprises an amino acid sequence that is at least 85% homologous or identical to the heavy chain amino acid sequence of the antibody produced by a hybridoma deposited under the American Type Culture Collection (ATCC) Accession NO: PTA-11267. In other embodiments, the heavy chain comprises an amino acid sequence that is at least 90% homologous or identical to the heavy chain amino acid sequence of the antibody produced by a hybridoma deposited under the American Type Culture Collection (ATCC) Accession NO: PTA-11267. In yet other embodiments, the heavy chain comprises an amino acid sequence that is at least 95% homologous or identical to the heavy chain amino acid sequence of the antibody produced by a hybridoma deposited under the American Type Culture Collection (ATCC) Accession NO: PTA-11267. In other embodiments, the heavy chain can be 85%, 86%, 87%, 88%, 89%, 90%, 91%, 92%, 93%, 94%, 95%, 96%, 97%, 98% or 99% homologous or identical to the heavy chain amino acid sequence of the antibody produced by a hybridoma deposited under the American Type Culture Collection (ATCC) Accession NO: PTA-11267. In some embodiments, the light chain comprises an amino acid sequence that is at least 85% homologous or identical to the light chain amino acid sequence of the antibody produced by a hybridoma deposited under the American Type Culture Collection (ATCC) Accession NO: PTA-11267. In other embodiments, the light chain comprises an amino acid sequence that is at least 90% homologous or identical to the light chain amino acid sequence of the antibody produced by a hybridoma deposited under the American Type Culture Collection (ATCC) Accession NO: PTA-11267. In yet other embodiments, the light chain comprises an amino acid sequence that is at least 95% homologous or identical to the light chain amino acid sequence of the antibody produced by a hybridoma deposited under the American Type Culture Collection (ATCC) Accession NO: PTA-11267. In other embodiments, the light chain can be 85%, 86%, 87%, 88%, 89%, 90%, 91%, 92%, 93%, 94%, 95%, 96%, 97%, 98% or 99% homologous or identical to the light chain amino acid sequence of the antibody produced by a hybridoma deposited under the American Type Culture Collection (ATCC) Accession NO: PTA-11267. In certain embodiments, the antibody or antigen binding fragment provided herein comprises any homologous light chain and any homologous heavy chain as provided in this paragraph in any combination or permutation.

In some embodiments, the antibody or antigen binding fragment thereof provided herein binds to a specific epitope in 191P4D12. In some embodiments, the antibody or antigen binding fragment thereof provided herein binds to VC1 domain of 191P4D12. In some embodiments, the antibody or antigen binding fragment thereof provided herein binds to VC1 domain but not to C1C2 domain of 191P4D12. In some embodiments, the antibody or antigen binding fragment thereof provided herein binds to the 1st to 147th amino acid residues of 191P4D12. In some embodiments, the antibody or antigen binding fragment thereof provided herein binds to an epitope located in the 1st to 147th amino acid residues of 191P4D12. In some embodiments, the antibody or antigen binding fragment thereof provided herein binds to the 1st to 10th amino acid residues of 191P4D12. In some embodiments, the antibody or antigen binding fragment thereof provided herein binds to the 11th to 20th amino acid residues of 191P4D12. In some embodiments, the antibody or antigen binding fragment thereof provided herein binds to the 21st to 30th amino acid residues of 191P4D12. In some embodiments, the antibody or antigen binding fragment thereof provided herein binds to the 31st to 40th amino acid residues of 191P4D12. In some embodiments, the antibody or antigen binding fragment thereof provided herein binds to the 41st to 50th amino acid residues of 191P4D12. In some embodiments, the antibody or antigen binding fragment thereof provided herein binds to the 51st to 60th amino acid residues of 191P4D12. In some embodiments, the antibody or antigen binding fragment thereof provided herein binds to the 61st to 70th amino acid residues of 191P4D12. In some embodiments, the antibody or antigen binding fragment thereof provided herein binds to the 71st to 80th amino acid residues of 191P4D12. In some embodiments, the antibody or antigen binding fragment thereof provided herein binds to the 81st to 90th amino acid residues of 191P4D12. In some embodiments, the antibody or antigen binding fragment thereof provided herein binds to the 91st to 100th amino acid residues of 191P4D12. In some embodiments, the antibody or antigen binding fragment thereof provided herein binds to the 101st to 110th amino acid residues of 191P4D12. In some embodiments, the antibody or antigen binding fragment thereof provided herein binds to the 111th to 120th amino acid residues of 191P4D12. In some embodiments, the antibody or antigen binding fragment thereof provided herein binds to the 121st to 130th amino acid residues of 191P4D12. In some embodiments, the antibody or antigen binding fragment thereof provided herein binds to the 131st to 140th amino acid residues of 191P4D12. In some embodiments, the antibody or antigen binding fragment thereof provided herein binds to the 141st to 147th amino acid residues of 191P4D12. The binding epitopes of certain embodiments the antibodies or antigen binding fragments thereof provided herein have been determined and described in WO 2012/047724, which is incorporated herein in its entirety by reference.

In some embodiments, the antibody or antigen binding fragment thereof provided herein binds to epitopes in 191P4D12 that are common between the 191P4D12 variants observed in human. In some embodiments, the antibody or antigen binding fragment thereof provided herein binds to epitopes in 191P4D12 that are common between the 191P4D12 polymorphism observed in human. In some embodiments, the antibody or antigen binding fragment thereof provided herein binds to epitopes in 191P4D12 that are common between the 191P4D12 polymorphism observed in human cancers. In some embodiments, the antibody or antigen binding fragment thereof provided herein binds to epitopes in 191P4D12 that would bind, internalize, disrupt or modulate the biological function of 191P4D12 or 191P4D12 variants. In some embodiments, the antibody or antigen binding fragment thereof provided herein binds to epitopes in 191P4D12 that would disrupt the interaction between 191P4D12 with ligands, substrates, and binding partners.

Engineered antibodies provided herein include those in which modifications have been made to framework residues within VH and/or VL (e.g. to improve the properties of the antibody). Typically, such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis (e.g., “backmutated” from leucine to methionine). Such “backmutated” antibodies are also intended to be encompassed by the disclosure.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr et al.

In addition or alternative to modifications made within the framework or CDR regions, antibodies of the disclosure can be engineered to include modifications within the Fc region, typically to alter one or more functional properties of the antibody, such as serum half-life, complement fixation, Fc receptor binding, and/or antigen-dependent cellular cytotoxicity. Furthermore, an anti-191P4D12 antibody provided herein can be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the anti-191P4D12 antibody.

In another embodiment, the Fc hinge region of an antibody is mutated to decrease the biological half-life of the anti-191P4D12 antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the anti-191P4D12 antibody is modified to increase its biological half-life. Various approaches are possible. For example, mutations can be introduced as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid specific residues can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

Reactivity of the anti-191P4D12 antibodies with a 191P4D12-related protein can be established by a number of well-known means, including Western blot, immunoprecipitation, ELISA, and FACS analyses using, as appropriate, 191P4D12-related proteins, 191P4D12-expressing cells or extracts thereof. A 191P4D12 antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more 191P4D12 epitopes are generated using methods generally known in the art. Homodimeric antibodies can also be generated by cross-linking techniques known in the art (e.g., Wolff et al., Cancer Res. 53: 2560-2565).

In yet another specific embodiment, the anti-191P4D12 antibody provided herein is an antibody comprising heavy and light chain of an antibody designated Ha22-2(2,4)6.1. The heavy chain of Ha22-2(2,4)6.1 consists of the amino acid sequence ranging from 20^{th} E residue to the 466^{th} K residue of SEQ ID NO:7 and the light chain of Ha22-2(2,4)6.1 consists of amino acid sequence ranging from 23^{rd} D residue to the 236^{th} C residue of SEQ ID NO:8 sequence.

The hybridoma producing the antibody designated Ha22-2(2,4)6.1 was sent (via Federal Express) to the American Type Culture Collection (ATCC), P.O. Box 1549, Manassas, VA 20108 on 18 Aug. 2010 and assigned Accession number PTA-11267.

Additional embodiments of anti-nectin-4 antibody have been described in U.S. Pat. No. 8,637,642 and International Application No. PCT/US2019/056214 (Publication No. WO2020/117373), both of which are hereby incorporated in their entireties by reference.

### 5.3.2 Cytotoxic Agents (Drug Units)

As the ADC used in the methods provided herein comprises an antibody or antigen binding fragment thereof conjugated to a cytotoxic agent, the disclosure further provides various embodiments for the cytotoxic agent as part of the ADC for use in the methods. In various embodiments of the methods provided herein, including the methods provided in Section 5.2, the cytotoxic agent as part of any of the ADCs provided herein for the methods comprises, consists of, or is a tubulin disrupting agent. In one embodiment, the cytotoxic agent is a tubulindisrupting agent. In some embodiments, the tubulin disrupting agent is selected from the group consisting of a dolastatin, an auristatin, a hemiasterlin, a vinca alkaloid, a maytansinoid, an eribulin, a colchicine, a plocabulin, a phomopsin, an epothilone, a cryptophycin, and a taxane. In one specific embodiment, the tubulin disrupting agent is an auristatin. In a further specific embodiment, the auristatin is monomethyl auristatin E (MMAE), monomethyl auristatin F (MMAF), AFP, or auristain T. In yet another specific embodiment, the auristatin is monomethyl auristatin E (MMAE).

In various embodiments of the methods provided herein, including the methods provided in Section 5.2, the cytotoxic agent as part of any of the ADCs provided herein for the methods comprises, consists of, or is any agent selected from the cytotoxic agents described in U.S. Pat. No. 8,637,642 and International Application No. PCT/US2019/056214 (Publication No. WO2020/117373), both of which are hereby incorporated in their entireties by reference

In some embodiments, the auristatin is MMAE (wherein the wavy line indicates the covalent attachment to a linker of an antibody drug conjugate).

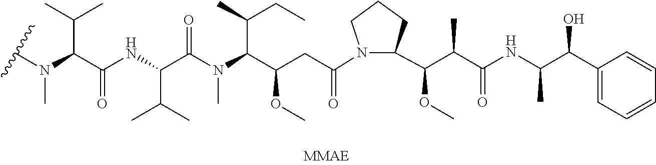 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, an exemplary embodiment comprising MMAE and a linker component (described further herein) has the following structure (wherein L presents the antibody (e.g. anti-nectin-4 antibody or antigen binding fragment thereof) and p ranges from 1 to 12):

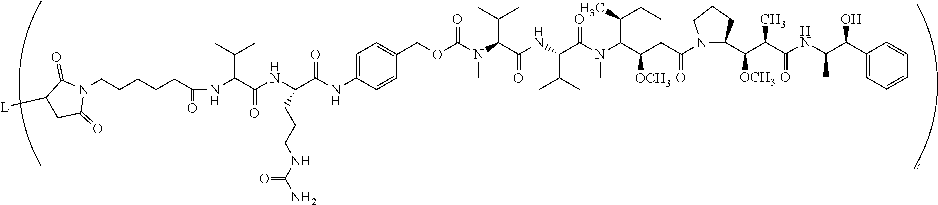

In some embodiments of the formula described in the preceding paragraph, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments of the formula described in the preceding paragraph, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments of the formula described in the preceding paragraph, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments of the formula described in the preceding paragraph, p is about 1. In some embodiments of the formula described in the preceding paragraph, p is about 2. In some embodiments of the formula described in the preceding paragraph, p is about 3. In some embodiments of the formula described in the preceding paragraph, p is about 4. In some embodiments of the formula described in the preceding paragraph, p is about 3.8. In some embodiments of the formula described in the preceding paragraph, p is about 5. In some embodiments of the formula described in the preceding paragraph, p is about 6. In some embodiments of the formula described in the preceding paragraph, p is about 7. In some embodiments of the formula described in the preceding paragraph, p is about 8. In some embodiments of the formula described in the preceding paragraph, p is about 9. In some embodiments of the formula described in the preceding paragraph, p is about 10. In some embodiments of the formula described in the preceding paragraph, p is about 11. In some embodiments of the formula described in the preceding paragraph, p is about 12. In some embodiments of the formula described in the preceding paragraph, p is about 13. In some embodiments of the formula described in the preceding paragraph, p is about 14. In some embodiments of the formula described in the preceding paragraph, p is about 15. In some embodiments of the formula described in the preceding paragraph, p is about 16. In some embodiments of the formula described in the preceding paragraph, p is about 17. In some embodiments of the formula described in the preceding paragraph, p is about 18. In some embodiments of the formula described in the preceding paragraph, p is about 19. In some embodiments of the formula described in the preceding paragraph, p is about 20.

Typically, peptide-based drug units can be prepared by forming a peptide bond between two or more amino acids and/or peptide fragments. Such peptide bonds can be prepared, for example, according to the liquid phase synthesis method (see E. Schroder and K. Lubke, “The Peptides”, volume 1, pp 76-136, 1965, Academic Press) that is well-known in the field of peptide chemistry. The auristatin/dolastatin drug units can be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat Biotechnol 21(7):778-784.

Additional embodiments of cytotoxic agent have been described in U.S. Pat. No. 8,637,642 and International Application No. PCT/US2019/056214 (Publication No. WO2020/117373), both of which are hereby incorporated in their entireties by reference.

### 5.3.3 Linkers

Typically, the antibody drug conjugates comprise a linker unit between the drug unit (e.g., MMAE) and the antibody unit (e.g., the anti-191P4D12 antibody or antigen binding fragment thereof). In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT.

A “linker unit” (LU) is a bifunctional compound that can be used to link a drug unit and an antibody unit to form an antibody drug conjugate. In some embodiments, the linker unit has the formula:

-Aₐ-W_{w}-Y_{y}-
wherein: -A- is a stretcher unit,
a is 0 or 1,
each -W- is independently an amino acid unit,
w is an integer ranging from 0 to 12,
-Y- is a self-immolative spacer unit, and
y is 0, 1 or 2.

In some embodiments, a is 0 or 1, w is 0 or 1, and y is 0, 1 or 2. In some embodiments, a is 0 or 1, w is 0 or 1, and y is 0 or 1. In some embodiments, when w is 1 to 12, y is 1 or 2. In some embodiments, w is 2 to 12 and y is 1 or 2. In some embodiments, a is 1 and w and y are 0. The linker and each of the stretcher unit, the amino acid unit, and the spacer unit have been described in U.S. Pat. No. 8,637,642 and International Application No. PCT/US2019/056214 (Publication No. WO2020/117373), both of which are hereby incorporated in their entireties by reference.

Embodiments of the antibody-drug conjugates can include:

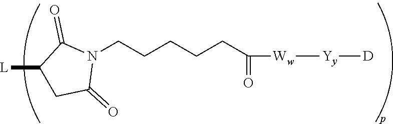

wherein w and y are each 0, 1 or 2, and,

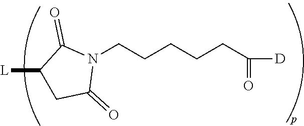

wherein w and y are each 0,

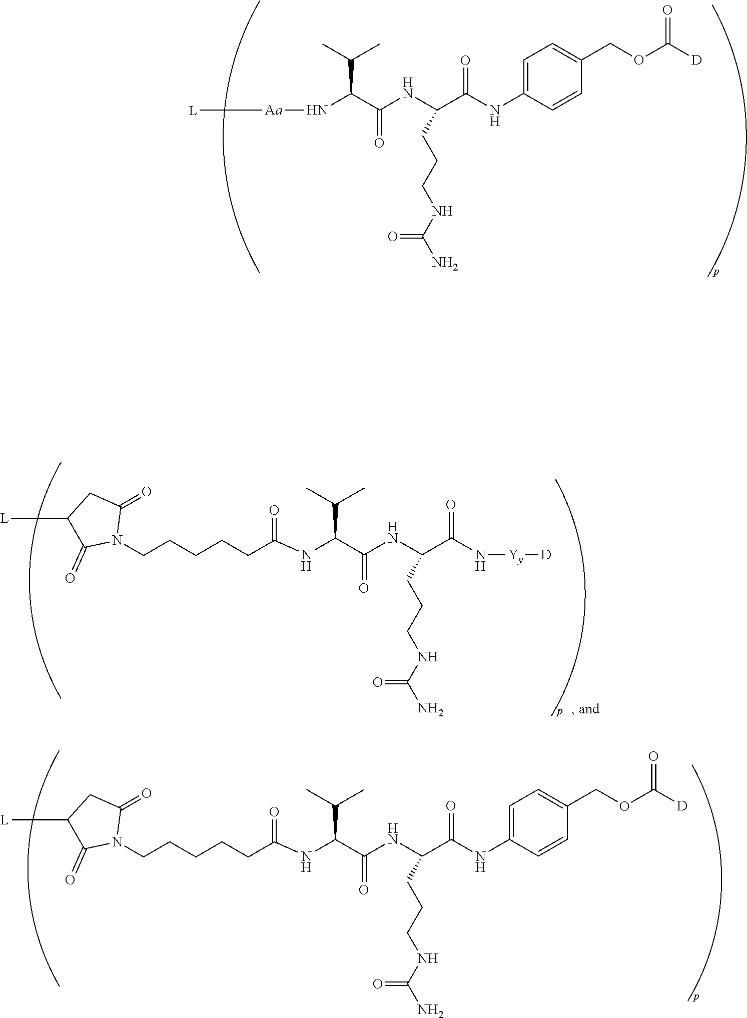

### 5.3.4 Drug Loading

Drug loading is represented by p and is the average number of drug units per antibody in a molecule. Drug loading can range from 1 to 20 drug units (D) per antibody. The ADCs provided herein include collections of antibodies or antigen binding fragments conjugated with a range of drug units, e.g., from 1 to 20. The average number of drug units per antibody in preparations of ADC from conjugation reactions can be characterized by conventional means such as mass spectroscopy and, ELISA assay. The quantitative distribution of ADC in terms of p can also be determined. In some instances, separation, purification, and characterization of homogeneous ADC where p is a certain value from ADC with other drug loadings can be achieved by means such as electrophoresis.

In certain embodiments, the drug loading for an ADC provided herein ranges from 1 to 20. In certain embodiments, the drug loading for an ADC provided herein ranges from 1 to 18. In certain embodiments, the drug loading for an ADC provided herein ranges from 1 to 15. In certain embodiments, the drug loading for an ADC provided herein ranges from 1 to 12. In certain embodiments, the drug loading for an ADC provided herein ranges from 1 to 10. In certain embodiments, the drug loading for an ADC provided herein ranges from 1 to 9. In certain embodiments, the drug loading for an ADC provided herein ranges from 1 to 8. In certain embodiments, the drug loading for an ADC provided herein ranges from 1 to 7. In certain embodiments, the drug loading for an ADC provided herein ranges from 1 to 6. In certain embodiments, the drug loading for an ADC provided herein ranges from 1 to 5. In certain embodiments, the drug loading for an ADC provided herein ranges from 1 to 4. In certain embodiments, the drug loading for an ADC provided herein ranges from 1 to 3. In certain embodiments, the drug loading for an ADC provided herein ranges from 2 to 12. In certain embodiments, the drug loading for an ADC provided herein ranges from 2 to 10. In certain embodiments, the drug loading for an ADC provided herein ranges from 2 to 9. In certain embodiments, the drug loading for an ADC provided herein ranges from 2 to 8. In certain embodiments, the drug loading for an ADC provided herein ranges from 2 to 7. In certain embodiments, the drug loading for an ADC provided herein ranges from 2 to 6. In certain embodiments, the drug loading for an ADC provided herein ranges from 2 to 5. In certain embodiments, the drug loading for an ADC provided herein ranges from 2 to 4. In certain embodiments, the drug loading for an ADC provided herein ranges from 3 to 12. In certain embodiments, the drug loading for an ADC provided herein ranges from 3 to 10. In certain embodiments, the drug loading for an ADC provided herein ranges from 3 to 9. In certain embodiments, the drug loading for an ADC provided herein ranges from 3 to 8. In certain embodiments, the drug loading for an ADC provided herein ranges from 3 to 7. In certain embodiments, the drug loading for an ADC provided herein ranges from 3 to 6. In certain embodiments, the drug loading for an ADC provided herein ranges from 3 to 5. In certain embodiments, the drug loading for an ADC provided herein ranges from 3 to 4.

In certain embodiments, the drug loading for an ADC provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, the drug loading for an ADC provided herein is about 1, about 2, about 3, about 4, about 5, about 6, about 7, about 8, about 9, about 10, about 11, about 12, or more. In some embodiments, the drug loading for an ADC provided herein is about 3.1, about 3.2, about 3.3, about 3.4, about 3.5, about 3.6, about 3.7, about 3.8, or about 3.9.

In some embodiments, the drug loading for an ADC provided herein ranges from 2 to 20, 2 to 19, 2 to 18, 2 to 17, 2 to 16, 2 to 15, 2 to 14, or 2 to 13. In some embodiments, the drug loading for an ADC provided herein ranges from 3 to 20, 3 to 19, 3 to 18, 3 to 17, 3 to 16, 3 to 15, 3 to 14, or 3 to 13. In some embodiments, the drug loading for an ADC provided herein is about 1. In some embodiments, the drug loading for an ADC provided herein is about 2. In some embodiments, the drug loading for an ADC provided herein is about 3. In some embodiments, the drug loading for an ADC provided herein is about 4. In some embodiments, the drug loading for an ADC provided herein is about 3.8. In some embodiments, the drug loading for an ADC provided herein is about 5. In some embodiments, the drug loading for an ADC provided herein is about 6. In some embodiments, the drug loading for an ADC provided herein is about 7. In some embodiments, the drug loading for an ADC provided herein is about 8. In some embodiments, the drug loading for an ADC provided herein is about 9. In some embodiments, the drug loading for an ADC provided herein is about 10. In some embodiments, the drug loading for an ADC provided herein is about 11. In some embodiments, the drug loading for an ADC provided herein is about 12. In some embodiments, the drug loading for an ADC provided herein is about 13. In some embodiments, the drug loading for an ADC provided herein is about 14. In some embodiments, the drug loading for an ADC provided herein is about 15. In some embodiments, the drug loading for an ADC provided herein is about 16. In some embodiments, the drug loading for an ADC provided herein is about 17. In some embodiments, the drug loading for an ADC provided herein is about 18. In some embodiments, the drug loading for an ADC provided herein is about 19. In some embodiments, the drug loading for an ADC provided herein is about 20.

In certain embodiments, fewer than the theoretical maximum of drug units are conjugated to an antibody during a conjugation reaction. An antibody can contain, for example, lysine residues that do not react with the drug-linker intermediate or linker reagent. Generally, antibodies do not contain many free and reactive cysteine thiol groups which can be linked to a drug unit; indeed most cysteine thiol residues in antibodies exist as disulfide bridges. In certain embodiments, an antibody can be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the linker unit or a drug unit is conjugated via a lysine residue on the antibody unit. In some embodiments, the linker unit or a drug unit is conjugated via a cysteine residue on the antibody unit.

In some embodiments, the amino acid that attaches to a linker unit or a drug unit is in the heavy chain of an antibody or antigen binding fragment thereof. In some embodiments, the amino acid that attaches to a linker unit or a drug unit is in the light chain of an antibody or antigen binding fragment thereof. In some embodiments, the amino acid that attaches to a linker unit or a drug unit is in the hinge region of an antibody or antigen binding fragment thereof. In some embodiments, the amino acid that attaches to a linker unit or a drug unit is in the Fc region of an antibody or antigen binding fragment thereof. In other embodiments, the amino acid that attaches to a linker unit or a drug unit is in the constant region (e.g., CH1, CH2, or CH3 of a heavy chain, or CH1 of a light chain) of an antibody or antigen binding fragment thereof. In yet other embodiments, the amino acid that attaches to a linker unit or a drug unit is in the VH framework regions of an antibody or antigen binding fragment thereof. In yet other embodiments, the amino acid that attaches to a linker unit or a drug unit is in the VL framework regions of an antibody or antigen binding fragment thereof.

The loading (drug/antibody ratio) of an ADC can be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug unit reagent, then the resulting product is a mixture of ADC compounds with a distribution of one or more drug unit attached to an antibody unit. The average number of drugs per antibody can be calculated from the mixture by a dual ELISA antibody assay, which is specific for antibody and specific for the drug. Individual ADC molecules can be identified in the mixture by mass spectroscopy and separated by HPLC, e.g. hydrophobic interaction chromatography (see, e.g., Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value can be isolated from the conjugation mixture by electrophoresis or chromatography.

Methods for preparing, screening, and characterizing the antibody drug conjugates are known to a person of ordinary skill in the art, for example, as described in U.S. Pat. No. 8,637,642, which is herein incorporated in its entirety by reference.

In some embodiments, the antibody drug conjugate for the methods provided herein is AGS-22M6E, which is prepared according to the methods described in U.S. Pat. No. 8,637,642 and has the following formula:

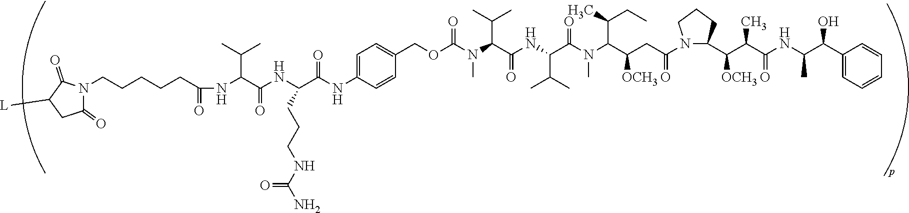

wherein L is Ha22-2(2,4)6.1 and p is from 1 to 20.

In some embodiments, p ranges from 1 to 20, 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is about 1. In other embodiments, p is about 2. In other embodiments, p is about 3. In other embodiments, p is about 4. In other embodiments, p is about 5. In other embodiments, p is about 6. In other embodiments, p is about 7. In other embodiments, p is about 8. In other embodiments, p is about 9. In other embodiments, p is about 10. In some embodiments, p is about 3.1. In some embodiments, p is about 3.2. In some embodiments, p is about 3.3. In some embodiments, p is about 3.4. In some embodiments, p is about 3.5. In other embodiments, p is about 3.6. In some embodiments, p is about 3.7. In some embodiments, p is about 3.8. In some embodiments, p is about 3.9. In some embodiments, p is about 4.0. In some embodiments, p is about 4.1. In some embodiments, p is about 4.2. In some embodiments, p is about 4.3. In some embodiments, p is about 4.4. In some embodiments, p is about 4.5. In other embodiments, p is about 4.6. In some embodiments, p is about 4.7. In some embodiments, p is about 4.8. In some embodiments, p is about 4.9. In some embodiments, p is about 5.0.

In some embodiments, the ADC used in the methods provided herein is enfortumab vedotin. Enfortumab vedotin is an ADC comprised of a fully human immunoglobulin G1 kappa (IgG1_{K}) antibody conjugated to the microtubule-disrupting agent (MMAE) via a protease-cleavable linker (Challita-Eid P M et al, Cancer Res. 2016; 76(10):3003-13]. Enfortumab vedotin induces antitumor activity by binding to 191P4D12 protein on the cell surface leading to internalization of the ADC-191P4D12 complex, which then traffics to the lysosomal compartment where MMAE is released via proteolytic cleavage of the linker. Intracellular release of MMAE subsequently disrupts tubulin polymerization resulting in G2/M phase cell cycle arrest and apoptotic cell death (Francisco J A et al, Blood. 2003 Aug. 15; 102(4):1458-65).

As described above and in in U.S. Pat. No. 8,637,642, AGS-22M6E is an ADC derived from a murine hybridoma cell line. Enfortumab vedotin is a Chinese hamster ovary (CHO) cell line-derived equivalent of AGS-22M6E ADC and is an exemplary product used for human treatment. Enfortumab vedotin has the same amino acid sequence, linker and cytotoxic drug as AGS-22M6E. The comparability between enfortumab vedotin and AGS-22M6E was confirmed through extensive analytical and biological characterization studies, such as binding affinity to 191P4D12, in vitro cytotoxicity, and in vivo antitumor activity.

In one embodiment, the ADC provided herein is enfortumab vedotin, also known as EV, PADCEV, AGS-22M6E, AGS-22C3E, and AGS-22CE. The enfortumab vedotin includes an anti-191P4D12 antibody, wherein the antibody or antigen binding fragment thereof comprises a heavy chain comprising amino acid residue 20 to amino acid residue 466 of SEQ ID NO: 7 and a light chain comprising amino acid residue 23 to amino acid residue 236 of SEQ ID NO:8.

Enfortumab vedotin is a Nectin-4 directed antibody-drug conjugate (ADC) comprised of a fully human anti-nectin-4 IgG1 kappa monoclonal antibody (AGS-22C3) conjugated to the small molecule microtubule disrupting agent, monomethyl auristatin E (MMAE) via a protease-cleavable maleimidocaproyl valine-citrulline (vc) linker (SGD-1006). Conjugation takes place on cysteine residues that comprise the interchain disulfide bonds of the antibody to yield a product with a drug-to-antibody ratio of approximately 3.8:1. The molecular weight is approximately 152 kDa.

Enfortumab vedotin has the following structural formula:

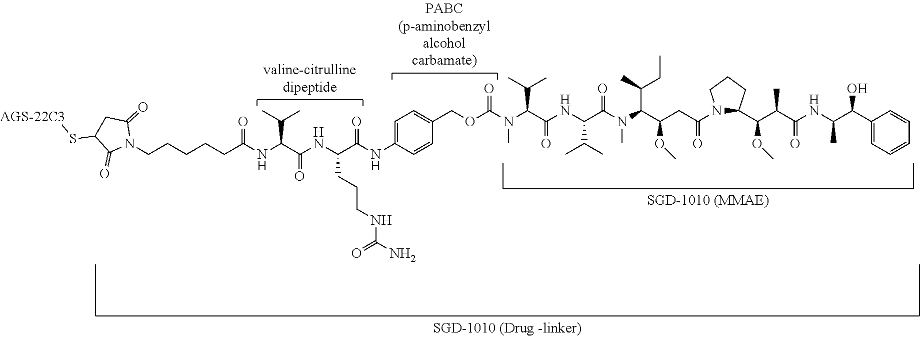

Approximately 4 molecules of MMAE are attached to each antibody molecule. Enfortumab vedotin is produced by chemical conjugation of the antibody and small molecule components. The antibody is produced by mammalian (Chinese hamster ovary) cells and the small molecule components are produced by chemical synthesis.

Enfortumab vedotin injection is provided as a sterile, preservative-free, white to off-white lyophilized powder in single-dose vials for intravenous use. Enfortumab vedotin is supplied as a 20 mg per vial and a 30 mg per vial and requires reconstitution with Sterile Water for Injection, USP, (2.3 mL and 3.3 mL, respectively) resulting in a clear to slightly opalescent, colorless to slightly yellow solution with a final concentration of 10 mg/mL. After reconstitution, each vial allows the withdrawal of 2 mL (20 mg) and 3 mL (30 mg). Each mL of reconstituted solution contains 10 mg of enfortumab vedotin, histidine (1.4 mg), histidine hydrochloride monohydrate (2.31 mg), polysorbate 20 (0.2 mg) and trehalose dihydrate (55 mg) with a pH of 6.0.

### 5.4 Pharmaceutical Compositions

In certain embodiments of the methods provided herein, the ADC used in the methods is provided in “pharmaceutical compositions.” Such pharmaceutical compositions include an antibody drug conjugate provided herein, and one or more pharmaceutically acceptable or physiologically acceptable excipients. In certain embodiments, the antibody drug conjugate are provided in combination with, or separate from, one or more additional agents. Also provided is a composition comprising such one or more additional agents and one or more pharmaceutically acceptable or physiologically acceptable excipients. In particular embodiments, the antibody drug conjugate and an additional agent(s) are present in a therapeutically acceptable amount. The pharmaceutical compositions can be used in accordance with the methods and uses provided herein. Thus, for example, the pharmaceutical compositions can be administered ex vivo or in vivo to a subject in order to practice treatment methods and uses provided herein. Pharmaceutical compositions provided herein can be formulated to be compatible with the intended method or route of administration; exemplary routes of administration are set forth herein.

In some embodiments, provided are pharmaceutical compositions of antibody drug conjugates that modulate a cancer or tumor.

In certain embodiments of the methods provided herein, the pharmaceutical compositions comprising the ADCs can further comprise other therapeutically active agents or compounds disclosed herein or known to the skilled artisan which can be used in the treatment or prevention of various diseases and disorders as set forth herein (e.g., a cancer). As set forth above, the additional therapeutically active agents or compounds can be present in a separate pharmaceutical composition(s).

Pharmaceutical compositions typically comprise a therapeutically effective amount of at least one of the antibody drug conjugates provided herein and one or more pharmaceutically acceptable formulation agents. In certain embodiments, the pharmaceutical composition further comprises one or more additional agents described herein.

In one embodiment, a pharmaceutical composition comprises an antibody drug conjugate provided herein. In some embodiments, a pharmaceutical composition comprises a therapeutically effective amount of an antibody drug conjugate provided herein. In certain embodiments, the pharmaceutical composition comprises a pharmaceutically acceptable excipient.

In some embodiments, the antibody drug conjugate in the pharmaceutical composition provided herein is selected from the antibody drug conjugates described in Section 5.3 above.

In certain embodiments, the pharmaceutical composition comprises the antibody drug conjugate at a concentration of from 0.1-100 mg/mL. In some embodiments, the pharmaceutical composition comprises the antibody drug conjugate at a concentration of from 1 to 20 mg/mL. In other embodiments, the pharmaceutical composition comprises the antibody drug conjugate at a concentration of from 5 to 15 mg/mL. In other embodiments, the pharmaceutical composition comprises the antibody drug conjugate at a concentration of from 8 to 12 mg/mL. In other embodiments, the pharmaceutical composition comprises the antibody drug conjugate at a concentration of from 9 to 11 mg/mL. In some embodiments, the pharmaceutical composition comprises the antibody drug conjugate at a concentration of about 9.5 mg/mL. In some embodiments, the pharmaceutical composition comprises the antibody drug conjugate at a concentration of about 9.6 mg/mL. In some embodiments, the pharmaceutical composition comprises the antibody drug conjugate at a concentration of about 9.7 mg/mL. In some embodiments, the pharmaceutical composition comprises the antibody drug conjugate at a concentration of about 9.8 mg/mL. In some embodiments, the pharmaceutical composition comprises the antibody drug conjugate at a concentration of about 9.9 mg/mL. In yet other embodiments, the pharmaceutical composition comprises the antibody drug conjugate at a concentration of about 10 mg/mL. In yet other embodiments, the pharmaceutical composition comprises the antibody drug conjugate at a concentration of about 10.1 mg/mL. In some embodiments, the pharmaceutical composition comprises the antibody drug conjugate at a concentration of about 10.2 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.4 mg/mL. In some embodiments, the pharmaceutical composition comprises the antibody drug conjugate at a concentration of about 10.5 mg/mL.

In some embodiments, the pharmaceutical composition provided herein comprises

L-histidine, TWEEN-20, and at least one of trehalose dihydrate or sucrose. In some embodiments, the pharmaceutical composition provided herein further comprises hydrochloric acid (HCl) or succinic acid.

In some embodiments, the concentration of L-histidine useful in the pharmaceutical compositions provided herein is in the range of between 5 and 50 mM. In some embodiments, the concentration of L-histidine in the pharmaceutical compositions provided herein is in the range of between 10 and 40 mM. In some embodiments, the concentration of L-histidine in the pharmaceutical compositions provided herein is in the range of between 15 and 35 mM.

In some embodiments, the concentration of L-histidine in the pharmaceutical compositions provided herein is in the range of between 15 and 30 mM. In some embodiments, the concentration of L-histidine in the pharmaceutical compositions provided herein is in the range of between 15 and 25 mM. In some embodiments, the concentration of L-histidine in the pharmaceutical compositions provided herein is in the range of between 15 and 35 mM. In some embodiments, the concentration of L-histidine in the pharmaceutical compositions provided herein is about 16 mM. In some embodiments, the concentration of L-histidine in the pharmaceutical compositions provided herein is about 17 mM. In some embodiments, the concentration of L-histidine in the pharmaceutical compositions provided herein is about 18 mM. In some embodiments, the concentration of L-histidine in the pharmaceutical compositions provided herein is about 19 mM. In some embodiments, the concentration of L-histidine in the pharmaceutical compositions provided herein is about 20 mM. In some embodiments, the concentration of L-histidine in the pharmaceutical compositions provided herein is about 21 mM. In some embodiments, the concentration of L-histidine in the pharmaceutical compositions provided herein is about 22 mM. In some embodiments, the concentration of L-histidine in the pharmaceutical compositions provided herein is about 23 mM. In some embodiments, the concentration of L-histidine in the pharmaceutical compositions provided herein is about 24 mM. In some embodiments, the concentration of L-histidine in the pharmaceutical compositions provided herein is about 25 mM.

In some embodiments, the concentration of TWEEN-20 useful in the pharmaceutical compositions provided herein is in the range of from 0.001 to 0.1% (v/v). In another embodiment, the concentration of TWEEN-20 is in the range of from 0.0025 to 0.075% (v/v). In one embodiment, the concentration of TWEEN-20 is in the range of from 0.005 to 0.05% (v/v). In another embodiment, the concentration of TWEEN-20 is in the range of from 0.0075 to 0.025% (v/v). In another embodiment, the concentration of TWEEN-20 is in the range of from 0.0075 to 0.05% (v/v). In another embodiment, the concentration of TWEEN-20 is in the range of from 0.01 to 0.03% (v/v). In one particular embodiment, the concentration of TWEEN-20 is about 0.01% (v/v). In one particular embodiment, the concentration of TWEEN-20 is about 0.015% (v/v). In one particular embodiment, the concentration of TWEEN-20 is about 0.016% (v/v). In one particular embodiment, the concentration of TWEEN-20 is about 0.017% (v/v). In one particular embodiment, the concentration of TWEEN-20 is about 0.018% (v/v). In one particular embodiment, the concentration of TWEEN-20 is about 0.019% (v/v). In one particular embodiment, the concentration of TWEEN-20 is about 0.02% (v/v). In one particular embodiment, the concentration of TWEEN-20 is about 0.021% (v/v). In one particular embodiment, the concentration of TWEEN-20 is about 0.022% (v/v). In one particular embodiment, the concentration of TWEEN-20 is about 0.023% (v/v). In one particular embodiment, the concentration of TWEEN-20 is about 0.024% (v/v). In one particular embodiment, the concentration of TWEEN-20 is about 0.025% (v/v).

In one embodiment, the concentration of trehalose dihydrate useful in the pharmaceutical compositions provided herein is in the range of between 1% and 20% (w/v). In another embodiment, the concentration of trehalose dihydrate is in the range of 2% and 15% (w/v). In one embodiment, the concentration of trehalose dihydrate is in the range of 3% and 10% (w/v). In another embodiment, the concentration of trehalose dihydrate is in the range of 4% and 9% (w/v). In another embodiment, the concentration of trehalose dihydrate is in the range of 4% and 8% (w/v). In another embodiment, the concentration of trehalose dihydrate is in the range of 4% and 7% (w/v). In another embodiment, the concentration of trehalose dihydrate is in the range of 4% and 6% (w/v). In another embodiment, the concentration of trehalose dihydrate is in the range of 4.5% and 6% (w/v). In another embodiment, the concentration of trehalose dihydrate is about 4.6% (w/v). In another embodiment, the concentration of trehalose dihydrate is about 4.7% (w/v). In another embodiment, the concentration of trehalose dihydrate is about 4.8% (w/v). In another embodiment, the concentration of trehalose dihydrate is about 4.9% (w/v). In another embodiment, the concentration of trehalose dihydrate is about 5.0% (w/v). In another embodiment, the concentration of trehalose dihydrate is about 5.1% (w/v). In another embodiment, the concentration of trehalose dihydrate is about 5.2% (w/v). In another embodiment, the concentration of trehalose dihydrate is about 5.3% (w/v). In another embodiment, the concentration of trehalose dihydrate is about 5.4% (w/v). In another embodiment, the concentration of trehalose dihydrate is about 5.5% (w/v). In another embodiment, the concentration of trehalose dihydrate is about 5.6% (w/v). In another embodiment, the concentration of trehalose dihydrate is about 5.7% (w/v). In another embodiment, the concentration of trehalose dihydrate is about 5.8% (w/v). In another embodiment, the concentration of trehalose dihydrate is about 5.9% (w/v). In another embodiment, the concentration of trehalose dihydrate is about 6.0% (w/v). In another embodiment, the concentration of trehalose dihydrate is about 6.1% (w/v). In another embodiment, the concentration of trehalose dihydrate is about 6.2% (w/v). In another embodiment, the concentration of trehalose dihydrate is about 6.3% (w/v). In another embodiment, the concentration of trehalose dihydrate is about 6.4% (w/v). In another embodiment, the concentration of trehalose dihydrate is about 6.5% (w/v).

In certain embodiments, the molarity of the trehalose dihydrate is from 50 to 300 mM. In other embodiments, the molarity of the trehalose dihydrate is from 75 to 250 mM. In some embodiments, the molarity of the trehalose dihydrate is from 100 to 200 mM. In other embodiments, the molarity of the trehalose dihydrate is from 130 to 150 mM. In some embodiments, the molarity of the trehalose dihydrate is from 135 to 150 mM. In certain embodiments, the molarity of the trehalose dihydrate is about 135 mM. In certain embodiments, the molarity of the trehalose dihydrate is about 136 mM. In certain embodiments, the molarity of the trehalose dihydrate is about 137 mM. In certain embodiments, the molarity of the trehalose dihydrate is about 138 mM. In certain embodiments, the molarity of the trehalose dihydrate is about 139 mM. In certain embodiments, the molarity of the trehalose dihydrate is about 140 mM. In certain embodiments, the molarity of the trehalose dihydrate is about 141 mM. In certain embodiments, the molarity of the trehalose dihydrate is about 142 mM. In certain embodiments, the molarity of the trehalose dihydrate is about 143 mM. In certain embodiments, the molarity of the trehalose dihydrate is about 144 mM. In certain embodiments, the molarity of the trehalose dihydrate is about 145 mM. In certain embodiments, the molarity of the trehalose dihydrate is about 146 mM. In certain embodiments, the molarity of the trehalose dihydrate is about 150 mM. In certain embodiments, the molarity of the trehalose dihydrate is about 151 mM. In certain embodiments, the molarity of the trehalose dihydrate is about 151 mM. In certain embodiments, the molarity of the trehalose dihydrate is about 152 mM. In certain embodiments, the molarity of the trehalose dihydrate is about 153 mM. In certain embodiments, the molarity of the trehalose dihydrate is about 154 mM. In certain embodiments, the molarity of the trehalose dihydrate is about 155 mM.

In one embodiment, the concentration of sucrose useful in the pharmaceutical compositions provided herein is in the range of between 1% and 20% (w/v). In another embodiment, the concentration of sucrose is in the range of 2% and 15% (w/v). In one embodiment, the concentration of sucrose is in the range of 3% and 10% (w/v). In another embodiment, the concentration of sucrose is in the range of 4% and 9% (w/v). In another embodiment, the concentration of sucrose is in the range of 4% and 8% (w/v). In another embodiment, the concentration of sucrose is in the range of 4% and 7% (w/v). In another embodiment, the concentration of sucrose is in the range of 4% and 6% (w/v). In another embodiment, the concentration of sucrose is in the range of 4.5% and 6% (w/v). In another embodiment, the concentration of sucrose is about 4.6% (w/v). In another embodiment, the concentration of sucrose is about 4.7% (w/v). In another embodiment, the concentration of sucrose is about 4.8% (w/v). In another embodiment, the concentration of sucrose is about 4.9% (w/v). In another embodiment, the concentration of sucrose is about 5.0% (w/v). In another embodiment, the concentration of sucrose is about 5.1% (w/v). In another embodiment, the concentration of sucrose is about 5.2% (w/v). In another embodiment, the concentration of sucrose is about 5.3% (w/v). In another embodiment, the concentration of sucrose is about 5.4% (w/v). In another embodiment, the concentration of sucrose is about 5.5% (w/v). In another embodiment, the concentration of sucrose is about 5.6% (w/v). In another embodiment, the concentration of sucrose is about 5.7% (w/v). In another embodiment, the concentration of sucrose is about 5.8% (w/v). In another embodiment, the concentration of sucrose is about 5.9% (w/v). In another embodiment, the concentration of sucrose is about 6.0% (w/v). In another embodiment, the concentration of sucrose is about 6.1% (w/v). In another embodiment, the concentration of sucrose is about 6.2% (w/v). In another embodiment, the concentration of sucrose is about 6.3% (w/v). In another embodiment, the concentration of sucrose is about 6.4% (w/v). In another embodiment, the concentration of sucrose is about 6.5% (w/v).

In certain embodiments, the molarity of the sucrose is from 50 to 300 mM. In other embodiments, the molarity of the sucrose is from 75 to 250 mM. In some embodiments, the molarity of the sucrose is from 100 to 200 mM. In other embodiments, the molarity of the sucrose is from 130 to 150 mM. In some embodiments, the molarity of the sucrose is from 135 to 150 mM. In certain embodiments, the molarity of the sucrose is about 135 mM. In certain embodiments, the molarity of the sucrose is about 136 mM. In certain embodiments, the molarity of the sucrose is about 137 mM. In certain embodiments, the molarity of the sucrose is about 138 mM. In certain embodiments, the molarity of the sucrose is about 139 mM. In certain embodiments, the molarity of the sucrose is about 140 mM. In certain embodiments, the molarity of the sucrose is about 141 mM. In certain embodiments, the molarity of the sucrose is about 142 mM. In certain embodiments, the molarity of the sucrose is about 143 mM. In certain embodiments, the molarity of the sucrose is about 144 mM. In certain embodiments, the molarity of the sucrose is about 145 mM. In certain embodiments, the molarity of the sucrose is about 146 mM. In certain embodiments, the molarity of the sucrose is about 150 mM. In certain embodiments, the molarity of the sucrose is about 151 mM. In certain embodiments, the molarity of the sucrose is about 151 mM. In certain embodiments, the molarity of the sucrose is about 152 mM. In certain embodiments, the molarity of the sucrose is about 153 mM. In certain embodiments, the molarity of the sucrose is about 154 mM. In certain embodiments, the molarity of the sucrose is about 155 mM.

In some embodiments, the pharmaceutical composition provided herein comprises HCl. In other embodiments, the pharmaceutical composition provided herein comprises succinic acid.

In some embodiments, the pharmaceutical composition provided herein has a pH in a range of 5.5 to 6.5. In other embodiments, the pharmaceutical composition provided herein has a pH in a range of 5.7 to 6.3. In some embodiments, the pharmaceutical composition provided herein has a pH of about 5.7. In some embodiments, the pharmaceutical composition provided herein has a pH of about 5.8. In some embodiments, the pharmaceutical composition provided herein has a pH of about 5.9. In some embodiments, the pharmaceutical composition provided herein has a pH of about 6.0. In some embodiments, the pharmaceutical composition provided herein has a pH of about 6.1. In some embodiments, the pharmaceutical composition provided herein has a pH of about 6.2. In some embodiments, the pharmaceutical composition provided herein has a pH of about 6.3.

In some embodiments, the pH is taken at room temperature. In other embodiments, the pH is taken at 15° C. to 27° C. In yet other embodiments, the pH is taken at 4° C. In yet other embodiments, the pH is taken at 25° C.

In some embodiments, the pH is adjusted by HCl. In some embodiments, the pharmaceutical composition comprises HCl, and the pharmaceutical composition has a pH in a range of 5.5 to 6.5 at room temperature. In some embodiments, the pharmaceutical composition comprises HCl, and the pharmaceutical composition has a pH in a range of 5.7 to 6.3 at room temperature. In some more specific embodiments, the pharmaceutical composition comprises HCl, and the pharmaceutical composition has a pH of about of 5.7 at room temperature. In some more specific embodiments, the pharmaceutical composition comprises HCl, and the pharmaceutical composition has a pH of about of 5.8 at room temperature. In some more specific embodiments, the pharmaceutical composition comprises HCl, and the pharmaceutical composition has a pH of about of 5.9 at room temperature. In some more specific embodiments, the pharmaceutical composition comprises HCl, and the pharmaceutical composition has a pH of about of 6.0 at room temperature. In some more specific embodiments, the pharmaceutical composition comprises HCl, and the pharmaceutical composition has a pH of about of 6.1 at room temperature. In some more specific embodiments, the pharmaceutical composition comprises HCl, and the pharmaceutical composition has a pH of about of 6.2 at room temperature. In some more specific embodiments, the pharmaceutical composition comprises HCl, and the pharmaceutical composition has a pH of about of 6.3 at room temperature.

In some embodiments, the pharmaceutical composition comprises HCl, and the pharmaceutical composition has a pH in a range of 5.5 to 6.5 at 15° C. to 27° C. In some embodiments, the pharmaceutical composition comprises HCl, and the pharmaceutical composition has a pH in a range of 5.7 to 6.3 at 15° C. to 27° C. In some more specific embodiments, the pharmaceutical composition comprises HCl, and the pharmaceutical composition has a pH of about of 5.7 at 15° C. to 27° C. In some more specific embodiments, the pharmaceutical composition comprises HCl, and the pharmaceutical composition has a pH of about of 5.8 at 15° C. to 27° C. In some more specific embodiments, the pharmaceutical composition comprises HCl, and the pharmaceutical composition has a pH of about of 5.9 at 15° C. to 27° C. In some more specific embodiments, the pharmaceutical composition comprises HCl, and the pharmaceutical composition has a pH of about of 6.0 at 15° C. to 27° C. In some more specific embodiments, the pharmaceutical composition comprises HCl, and the pharmaceutical composition has a pH of about of 6.1 at 15° C. to 27° C. In some more specific embodiments, the pharmaceutical composition comprises HCl, and the pharmaceutical composition has a pH of about of 6.2 at 15° C. to 27° C. In some more specific embodiments, the pharmaceutical composition comprises HCl, and the pharmaceutical composition has a pH of about of 6.3 at 15° C. to 27° C.

In some embodiments, the pH is adjusted by succinic acid. In some embodiments, the pharmaceutical composition comprises succinic acid, and the pharmaceutical composition has a pH in a range of 5.5 to 6.5 at room temperature. In some embodiments, the pharmaceutical composition comprises succinic acid, and the pharmaceutical composition has a pH in a range of 5.7 to 6.3 at room temperature. In some more specific embodiments, the pharmaceutical composition comprises succinic acid, and the pharmaceutical composition has a pH of about of 5.7 at room temperature. In some more specific embodiments, the pharmaceutical composition comprises succinic acid, and the pharmaceutical composition has a pH of about of 5.8 at room temperature. In some more specific embodiments, the pharmaceutical composition comprises succinic acid, and the pharmaceutical composition has a pH of about of 5.9 at room temperature. In some more specific embodiments, the pharmaceutical composition comprises succinic acid, and the pharmaceutical composition has a pH of about of 6.0 at room temperature. In some more specific embodiments, the pharmaceutical composition comprises succinic acid, and the pharmaceutical composition has a pH of about of 6.1 at room temperature. In some more specific embodiments, the pharmaceutical composition comprises succinic acid, and the pharmaceutical composition has a pH of about of 6.2 at room temperature. In some more specific embodiments, the pharmaceutical composition comprises succinic acid, and the pharmaceutical composition has a pH of about of 6.3 at room temperature.

In some embodiments, the pharmaceutical composition comprises succinic acid, and the pharmaceutical composition has a pH in a range of 5.5 to 6.5 at 15° C. to 27° C. In some embodiments, the pharmaceutical composition comprises succinic acid, and the pharmaceutical composition has a pH in a range of 5.7 to 6.3 at 15° C. to 27° C. In some more specific embodiments, the pharmaceutical composition comprises succinic acid, and the pharmaceutical composition has a pH of about of 5.7 at 15° C. to 27° C. In some more specific embodiments, the pharmaceutical composition comprises succinic acid, and the pharmaceutical composition has a pH of about of 5.8 at 15° C. to 27° C. In some more specific embodiments, the pharmaceutical composition comprises succinic acid, and the pharmaceutical composition has a pH of about of 5.9 at 15° C. to 27° C. In some more specific embodiments, the pharmaceutical composition comprises succinic acid, and the pharmaceutical composition has a pH of about of 6.0 at 15° C. to 27° C. In some more specific embodiments, the pharmaceutical composition comprises succinic acid, and the pharmaceutical composition has a pH of about of 6.1 at 15° C. to 27° C. In some more specific embodiments, the pharmaceutical composition comprises succinic acid, and the pharmaceutical composition has a pH of about of 6.2 at 15° C. to 27° C. In some more specific embodiments, the pharmaceutical composition comprises succinic acid, and the pharmaceutical composition has a pH of about of 6.3 at 15° C. to 27° C.

In some specific embodiments, the pharmaceutical composition provided herein comprises about 20 mM L-histidine, about 0.02% (w/v) TWEEN-20, and at least one of about 5.5% (w/v) trehalose dihydrate or about 5% (w/v) sucrose. In some embodiments, the pharmaceutical composition provided herein further comprises HCl or succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5.5% (w/v) trehalose dihydrate and HCl. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and HCl. In some embodiments, the pH is about 6.0 at room temperature. In some embodiments, the pH is about 6.0 at 25° C.

In other specific embodiments, the pharmaceutical composition provided herein comprises about 20 mM L-histidine, about 0.02% (w/v) TWEEN-20, about 5.5% (w/v) trehalose dihydrate and succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and succinic acid. In some embodiments, the pH is about 6.0 at room temperature. In some embodiments, the pH is about 6.0 at 25° C.

In a specific embodiment, provided herein comprises
(a) an antibody drug conjugate comprising the following structure:

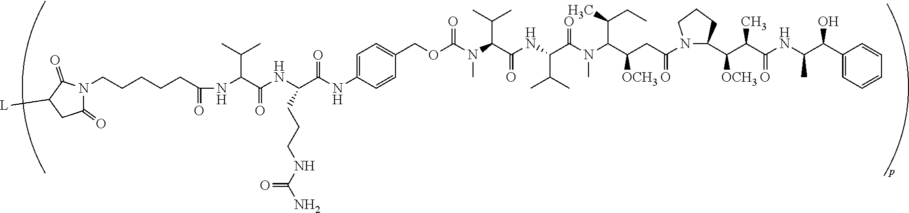

wherein L- represents the antibody or antigen binding fragment (e.g. anti-nectin-4 antibody or antigen binding fragment thereof) thereof and p is from 1 to 10; and
(b) a pharmaceutically acceptable excipient comprising about 20 mM L-histidine, about 0.02% (w/v) TWEEN-20, about 5.5% (w/v) trehalose dihydrate, and HCl, wherein the antibody drug conjugate is at the concentration of about 10 mg/mL, and wherein the pH is about 6.0 at 25° C.

In another specific embodiment, the pharmaceutical composition provided herein comprises:
(a) an antibody drug conjugate comprising the following structure:

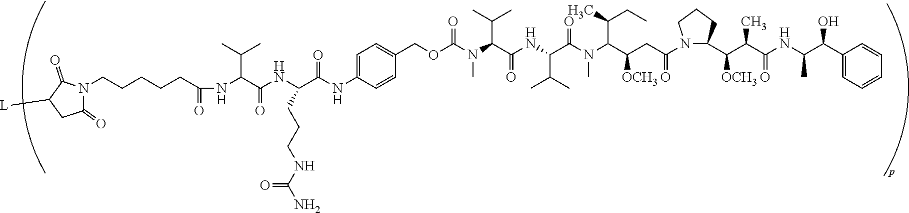

wherein L- represents the antibody or antigen binding fragment thereof (e.g. anti-nectin-4 antibody or antigen binding fragment thereof) and p is from 1 to 10; and
(b) a pharmaceutically acceptable excipient comprising about 20 mM L-histidine, about 0.02% (w/v) TWEEN-20, about 5.5% (w/v) trehalose dihydrate, and succinic acid, wherein the antibody drug conjugate is at the concentration of about 10 mg/mL, and wherein the pH is about 6.0 at 25° C.

In yet another specific embodiment, the pharmaceutical composition provided herein comprises:
(a) an antibody drug conjugate comprising the following structure:

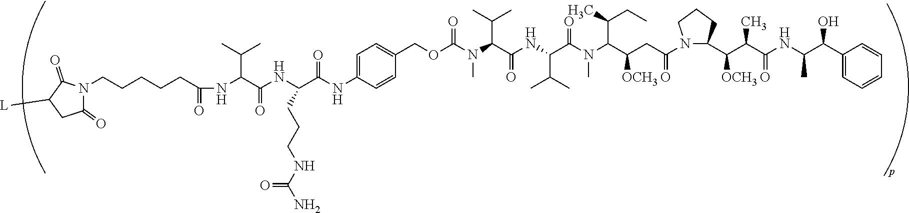

wherein L- represents the antibody or antigen binding fragment thereof (e.g. anti-nectin-4 antibody or antigen binding fragment thereof) and p is from 1 to 10; and
(b) a pharmaceutically acceptable excipient comprising about 20 mM L-histidine, about 0.02% (w/v) TWEEN-20, about 5.0% (w/v) sucrose, and HCl, wherein the antibody drug conjugate is at the concentration of about 10 mg/mL, and wherein the pH is about 6.0 at 25° C.

Although certain numbers (and numerical ranges thereof) are provided, it is understood that, in certain embodiments, numerical values within, e.g., 2%, 5%, 10%, 15% or 20% of said numbers (or numerical ranges) are also contemplated.

A primary solvent in a vehicle can be either aqueous or non-aqueous in nature. In addition, the vehicle can contain other pharmaceutically acceptable excipients for modifying or maintaining the pH, osmolarity, viscosity, sterility or stability of the pharmaceutical composition. In certain embodiments, the pharmaceutically acceptable vehicle is an aqueous buffer. In other embodiments, a vehicle comprises, for example, sodium chloride and/or sodium citrate.

Pharmaceutical compositions provided herein can contain still other pharmaceutically acceptable formulation agents for modifying or maintaining the rate of release of an antibody drug conjugate and/or an additional agent, as described herein. Such formulation agents include those substances known to artisans skilled in preparing sustained-release formulations. For further reference pertaining to pharmaceutically and physiologically acceptable formulation agents, see, for example, Remington's Pharmaceutical Sciences, 18th Ed. (1990, Mack Publishing Co., Easton, Pa. 18042) pages 1435-1712, The Merck Index, 12th Ed. (1996, Merck Publishing Group, Whitehouse, NJ); and Pharmaceutical Principles of Solid Dosage Forms (1993, Technonic Publishing Co., Inc., Lancaster, Pa.). Additional pharmaceutical compositions appropriate for administration are known in the art and are applicable in the methods and compositions provided herein.

In some embodiments, the pharmaceutical composition provided herein is in a liquid form. In other embodiments, the pharmaceutical composition provided herein is lyophilized.

A pharmaceutical composition can be formulated to be compatible with its intended route of administration. Thus, pharmaceutical compositions include excipients suitable for administration by routes including parenteral (e.g., subcutaneous (s.c.), intravenous, intramuscular, or intraperitoneal), intradermal, oral (e.g., ingestion), inhalation, intracavity, intracranial, and transdermal (topical). Other exemplary routes of administration are set forth herein.

Pharmaceutical compositions can be in the form of a sterile injectable aqueous or oleagenous suspension. This suspension can be formulated using suitable dispersing or wetting agents and suspending agents disclosed herein or known to the skilled artisan. The sterile injectable preparation can also be a sterile injectable solution or suspension in a non-toxic parenterally-acceptable diluent or solvent, for example, as a solution in 1,3-butane diol. Acceptable diluents, solvents and dispersion media that can be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil can be employed, including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

In one embodiment, the pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In one embodiment, the pharmaceutical compositions provided herein can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, e.g., Remington, The Science and Practice of Pharmacy, supra).

In one embodiment, the pharmaceutical compositions intended for parenteral administration can include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

In one embodiment, suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

In one embodiment, suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, KS).

In one embodiment, the pharmaceutical compositions provided herein can be formulated for single or multiple dosage administration. The single dosage formulations are packaged in an ampoule, a vial, or a syringe. The multiple dosage parenteral formulations can contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

In one embodiment, the pharmaceutical compositions provided herein can be formulated as immediate or modified release dosage forms, including delayed-, sustained, pulsed-, controlled, targeted-, and programmed-release forms.

Dispersible powders and granules suitable for preparation of an aqueous suspension by addition of water provide the active ingredient in admixture with a dispersing or wetting agent, suspending agent and one or more preservatives. Suitable dispersing or wetting agents and suspending agents are exemplified herein.

Pharmaceutical compositions can also include excipients to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, can be employed. Prolonged absorption of injectable pharmaceutical compositions can be achieved by including an agent that delays absorption, for example, aluminum monostearate or gelatin. Prevention of the action of microorganisms can be achieved by various antibacterial and antifungal agents, for example, parabens, chlorobutanol, phenol, ascorbic acid, thimerosal, and the like.

The pharmaceutical composition provided herein can be stored at −80° C., 4° C., 25° C. or 37° C.

A lyophilized composition can be made by freeze-drying the liquid pharmaceutical composition provided herein. In a specific embodiment, the pharmaceutical composition provided here is a lyophilized pharmaceutical composition. In some embodiments, the pharmaceutical formulations are lyophilized powders, which can be reconstituted for administration as solutions, emulsions and other mixtures. They can also be reconstituted and formulated as solids or gels.

In some embodiments, preparation of the lyophilized formulation provided herein involves batching of the formulated bulk solution for lyophilization, aseptic filtration, filling in vials, freezing vials in a freeze-dryer chamber, followed by lyophilization, stoppering and capping.

A lyophilizer can be used in preparing the lyophilized formulation. For example, a VirTis Genesis Model EL pilot unit can be employed. The unit incorporates a chamber with three working shelves (to a total usable shelf area of ca 0.4 square meters), an external condenser, and a mechanical vacuum pumping system. Cascaded mechanical refrigeration allows the shelves to be cooled to −70° C. or lower, and the external condenser to −90° C. or lower. Shelf temperature and chamber pressure were controlled automatically to +/−0.5° C. and +/−2 microns (milliTorr), respectively. The unit was equipped with a capacitance manometer vacuum gauge, a Pirani vacuum gauge, a pressure transducer (to measure from 0 to 1 atmosphere), and a relative humidity sensor.

The lyophilized powder can be prepared by dissolving an antibody drug conjugate provided herein, or a pharmaceutically acceptable derivative thereof, in a suitable solvent. In some embodiments, the lyophilized powder is sterile. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the antibody drug conjugate. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable excipient. Such amount can be empirically determined and adjusted according to specific needs.

An exemplary reconstitution procedure is illustrated as follows: (1) fit the 5 mL or 3 mL syringe with a with a 18 or 20 Gauge needle and filled the syringe with water of the grade Water for Injection (WFI); (2) measure appropriate amount of WFI using the syringe graduations, ensuring that the syringe was free of air bubbles; (3) inserted the needle through the rubber stopper; (4) dispense the entire contents of the syringe into the container down the vial wall, removed the syringe and needle and put into the sharp container; (4) swirl the vial continuously to carefully solubilize the entire vial contents until fully reconstituted (e.g., about 20-40 seconds) and minimize excessive agitation of the protein solution that could result in foaming.

In some embodiments, the pharmaceutical composition provided herein is supplied as a dry sterilized lyophilized powder or water free concentrate in a hermetically sealed container and can be reconstituted, e.g., with water or saline to the appropriate concentration for administration to a subject. In certain embodiments, the antibody drug conjugate is supplied as a dry sterile lyophilized powder in a hermetically sealed container at a unit dosage of at least 0.1 mg, at least 0.5 mg, at least 1 mg, at least 2 mg, at least 3 mg, at least 5 mg, at least 10 mg, at least 15 mg, at least 25 mg, at least 30 mg, at least 35 mg, at least 45 mg, at least 50 mg, at least 60 mg, at least 75 mg, at least 80 mg, at least 85 mg, at least 90 mg, at least 95 mg, or at least 100 mg. The lyophilized antibody drug conjugate can be stored at between 2 and 8° C. in its original container and the antibody drug conjugate can be administered within 12 hours, such as within 6 hours, within 5 hours, within 3 hours, or within 1 hour after being reconstituted. In an alternative embodiment, the pharmaceutical composition comprising the antibody drug conjugate provided herein is supplied in liquid form in a hermetically sealed container indicating the quantity and concentration of the antibody drug conjugate. In certain embodiments, the liquid form of the antibody drug conjugate is supplied in a hermetically sealed container at least 0.1 mg/ml, at least 0.5 mg/ml, at least 1 mg/ml, at least 5 mg/ml, at least 10 mg/ml, at least 15 mg/ml, at least 25 mg/ml, at least 30 mg/ml, at least 40 mg/ml, at least 50 mg/ml, at least 60 mg/ml, at least 70 mg/ml, at least 80 mg/ml, at least 90 mg/ml, or at least 100 mg/ml.

Additional embodiments for the pharmaceutical compositions have been described in U.S. Pat. No. 8,637,642 and International Application No. PCT/US2019/056214 (Publication No. WO2020/117373), both of which are hereby incorporated in their entireties by reference.

### 5.5 Methods for a Combination Therapy

The method for inhibiting growth of tumor cells using the pharmaceutical composition provided herein may be used in combination with chemotherapy or radiation or both comprises administering the present pharmaceutical composition before, during, or after commencing chemotherapy or radiation therapy, as well as any combination thereof (i.e. before and during, before and after, during and after, or before, during, and after commencing the chemotherapy and/or radiation therapy). Depending on the treatment protocol and the specific patient needs, the method is performed in a manner that will provide the most efficacious treatment and ultimately prolong the life of the patient. Additional embodiments for such combination therapy have been described in U.S. Pat. No. 8,637,642 and International Application No. PCT/US2019/056214 (Publication No. WO2020/117373), both of which are hereby incorporated in their entireties by reference.

### 5.6 Dosage of the ADCs for the Methods

In some embodiments, the amount of a prophylactic or therapeutic agent (e.g., an antibody drug conjugate provided herein), or a pharmaceutical composition provided herein that will be effective in the prevention and/or treatment of a cancer can be determined by standard clinical techniques. In some embodiments, effective doses can be extrapolated from dose-response curves derived from in vitro or animal model test systems. It is to be understood that the precise dose to be employed in the formulation will also depend on the route of administration, and the seriousness of a cancer in a subject, and should be decided according to the judgment of the practitioner and each patient's circumstances.

In some embodiments, the ADC of the methods for which the various dosages are described in this Section (Section 5.6) is enfortumab vedotin (EV).

In some embodiments, the route of administration for a dose of an antibody drug conjugate formulated in the pharmaceutical composition provided herein to a patient is intranasal, intramuscular, intravenous, intravescially, or a combination thereof, but other routes described herein are also acceptable. Each dose may or may not be administered by an identical route of administration. In some embodiments, an antibody drug conjugate formulated in the pharmaceutical composition provided herein can be administered via multiple routes of administration simultaneously or subsequently to other doses of one or more additional therapeutic agents. In some embodiments, the pharmaceutical composition comprising the antibody drug conjugate provided herein is administered intravescially.

For the pharmaceutical composition comprising the antibody drug conjugate provided herein, the effective amount of the ADC is a dose of between about 10 mg to about 1000 mg with a volume of instillation between about 10 mL to about 100 mL. In some embodiments, the effective amount of the ADC is a dose of between about 125 mg to about 950 mg with a volume of instillation between about 10 mL to about 100 mL. In some embodiments, the effective amount of the ADC is a dose of between about 125 mg to about 900 mg with a volume of instillation between about 10 mL to about 100 mL. In some embodiments, the effective amount of the ADC is a dose of between about 125 mg to about 850 mg with a volume of instillation between about 10 mL to about 100 mL. In some embodiments, the effective amount of the ADC is a dose of between about 125 mg to about 800 mg with a volume of instillation between about 10 mL to about 100 mL. In some embodiments, the effective amount of the ADC is a dose of between about 125 mg to about 750 mg with a volume of instillation between about 10 mL to about 100 mL. In some embodiments, the effective amount of the ADC is a dose of between about 125 mg to about 750 mg with a volume of instillation of about 25 mL.

In some embodiments, the effective amount of the ADC is a dose of between about 10 mg to about 1000 mg. In some embodiments, the effective amount of the ADC is a dose of between about 50 mg to about 1000 mg. In some embodiments, the effective amount of the ADC is a dose of between about 100 mg to about 900 mg. In some embodiments, the effective amount of the ADC is a dose of between about 125 mg to about 900 mg. In some embodiments, the effective amount of the ADC is a dose of between about 125 mg to about 850 mg. In some embodiments, the effective amount of the ADC is a dose of between about 125 mg to about 800 mg. In some embodiments, the effective amount of the ADC is a dose of between about 125 mg to about 750 mg.

In some embodiments, the effective amount of the ADC is a dose of about 100 mg. In some embodiments, the effective amount of the ADC is a dose of about 125 mg. In some embodiments, the effective amount of the ADC is a dose of about 150 mg. In some embodiments, the effective amount of the ADC is a dose of about 200 mg. In some embodiments, the effective amount of the ADC is a dose of about 250 mg. In some embodiments, the effective amount of the ADC is a dose of about 300 mg. In some embodiments, the effective amount of the ADC is a dose of about 350 mg. In some embodiments, the effective amount of the ADC is a dose of about 400 mg. In some embodiments, the effective amount of the ADC is a dose of about 450 mg. In some embodiments, the effective amount of the ADC is a dose of about 500 mg. In some embodiments, the effective amount of the ADC is a dose of about 550 mg. In some embodiments, the effective amount of the ADC is a dose of about 600 mg. In some embodiments, the effective amount of the ADC is a dose of about 650 mg. In some embodiments, the effective amount of the ADC is a dose of about 700 mg. In some embodiments, the effective amount of the ADC is a dose of about 750 mg. In some embodiments, the effective amount of the ADC is a dose of about 800 mg. In some embodiments, the effective amount of the ADC is a dose of about 850 mg. In some embodiments, the effective amount of the ADC is a dose of about 900 mg.

In some embodiments, the effective amount of the ADC is a dose of 100 mg. In some embodiments, the effective amount of the ADC is a dose of 125 mg. In some embodiments, the effective amount of the ADC is a dose of 150 mg. In some embodiments, the effective amount of the ADC is a dose of 200 mg. In some embodiments, the effective amount of the ADC is a dose of 250 mg. In some embodiments, the effective amount of the ADC is a dose of 300 mg. In some embodiments, the effective amount of the ADC is a dose of 350 mg. In some embodiments, the effective amount of the ADC is a dose of 400 mg. In some embodiments, the effective amount of the ADC is a dose of 450 mg. In some embodiments, the effective amount of the ADC is a dose of 500 mg. In some embodiments, the effective amount of the ADC is a dose of 550 mg. In some embodiments, the effective amount of the ADC is a dose of 600 mg. In some embodiments, the effective amount of the ADC is a dose of 650 mg. In some embodiments, the effective amount of the ADC is a dose of 700 mg. In some embodiments, the effective amount of the ADC is a dose of 750 mg. In some embodiments, the effective amount of the ADC is a dose of 800 mg. In some embodiments, the effective amount of the ADC is a dose of 850 mg. In some embodiments, the effective amount of the ADC is a dose of 900 mg.

In some embodiments, the volume of instillation is between about 10 mL to about 100 mL. In some embodiments, the volume of instillation is between about 10 mL to about 50 mL. In some embodiments, the volume of instillation is between about 15 mL to about 30 mL. In some embodiments, the volume of instillation is about 10 mL. In some embodiments, the volume of instillation is about 15 mL. In some embodiments, the volume of instillation is about 20 mL. In some embodiments, the volume of instillation is about 25 mL. In some embodiments, the volume of instillation is about 30 mL. In some embodiments, the volume of instillation is about 35 mL. In some embodiments, the volume of instillation is about 40 mL. In some embodiments, the volume of instillation is about 45 mL. In some embodiments, the volume of instillation is about 50 mL. In some embodiments, the volume of instillation is about 55 mL. In some embodiments, the volume of instillation is about 60 mL. In some embodiments, the volume of instillation is about 65 mL. In some embodiments, the volume of instillation is about 70 mL. In some embodiments, the volume of instillation is about 75 mL. In some embodiments, the volume of instillation is about 80 mL. In some embodiments, the volume of instillation is about 85 mL. In some embodiments, the volume of instillation is about 90 mL. In some embodiments, the volume of instillation is about 95 mL. In some embodiments, the volume of instillation is about 100 mL.

In some embodiments, the volume of instillation is 10 mL. In some embodiments, the volume of instillation is 15 mL. In some embodiments, the volume of instillation is 20 mL. In some embodiments, the volume of instillation is 25 mL. In some embodiments, the volume of instillation is 30 mL. In some embodiments, the volume of instillation is 35 mL. In some embodiments, the volume of instillation is 40 mL. In some embodiments, the volume of instillation is 45 mL. In some embodiments, the volume of instillation is 50 mL. In some embodiments, the volume of instillation is 55 mL. In some embodiments, the volume of instillation is 60 mL. In some embodiments, the volume of instillation is 65 mL. In some embodiments, the volume of instillation is 70 mL. In some embodiments, the volume of instillation is 75 mL. In some embodiments, the volume of instillation is 80 mL. In some embodiments, the volume of instillation is 85 mL. In some embodiments, the volume of instillation is 90 mL. In some embodiments, the volume of instillation is 95 mL. In some embodiments, the volume of instillation is 100 mL.

In some embodiments, the effective amount of the ADC is a dose of about 100 mg with a volume of instillation of about 10 mL. In some embodiments, the effective amount of the ADC is a dose of about 125 mg with a volume of instillation of about 10 mL. In some embodiments, the effective amount of the ADC is a dose of about 150 mg with a volume of instillation of about 10 mL. In some embodiments, the effective amount of the ADC is a dose of about 200 mg with a volume of instillation of about 10 mL. In some embodiments, the effective amount of the ADC is a dose of about 250 mg with a volume of instillation of about 10 mL. In some embodiments, the effective amount of the ADC is a dose of about 300 mg with a volume of instillation of about 10 mL. In some embodiments, the effective amount of the ADC is a dose of about 350 mg with a volume of instillation of about 10 mL. In some embodiments, the effective amount of the ADC is a dose of about 400 mg with a volume of instillation of about 10 mL. In some embodiments, the effective amount of the ADC is a dose of about 450 mg with a volume of instillation of about 10 mL. In some embodiments, the effective amount of the ADC is a dose of about 500 mg with a volume of instillation of about 10 mL. In some embodiments, the effective amount of the ADC is a dose of about 550 mg with a volume of instillation of about 10 mL. In some embodiments, the effective amount of the ADC is a dose of about 600 mg with a volume of instillation of about 10 mL. In some embodiments, the effective amount of the ADC is a dose of about 650 mg with a volume of instillation of about 10 mL. In some embodiments, the effective amount of the ADC is a dose of about 700 mg with a volume of instillation of about 10 mL. In some embodiments, the effective amount of the ADC is a dose of about 750 mg with a volume of instillation of about 10 mL. In some embodiments, the effective amount of the ADC is a dose of about 800 mg with a volume of instillation of about 10 mL. In some embodiments, the effective amount of the ADC is a dose of about 850 mg with a volume of instillation of about 10 mL. In some embodiments, the effective amount of the ADC is a dose of about 900 mg with a volume of instillation of about 10 mL.

In some embodiments, the effective amount of the ADC is a dose of 100 mg with a volume of instillation of 10 mL. In some embodiments, the effective amount of the ADC is a dose of 125 mg with a volume of instillation of 10 mL. In some embodiments, the effective amount of the ADC is a dose of 150 mg with a volume of instillation of 10 mL. In some embodiments, the effective amount of the ADC is a dose of 200 mg with a volume of instillation of 10 mL. In some embodiments, the effective amount of the ADC is a dose of 250 mg with a volume of instillation of 10 mL. In some embodiments, the effective amount of the ADC is a dose of 300 mg with a volume of instillation of 10 mL. In some embodiments, the effective amount of the ADC is a dose of 350 mg with a volume of instillation of 10 mL. In some embodiments, the effective amount of the ADC is a dose of 400 mg with a volume of instillation of 10 mL. In some embodiments, the effective amount of the ADC is a dose of 450 mg with a volume of instillation of 10 mL. In some embodiments, the effective amount of the ADC is a dose of 500 mg with a volume of instillation of 10 mL. In some embodiments, the effective amount of the ADC is a dose of 550 mg with a volume of instillation of 10 mL. In some embodiments, the effective amount of the ADC is a dose of 600 mg with a volume of instillation of 10 mL. In some embodiments, the effective amount of the ADC is a dose of 650 mg with a volume of instillation of 10 mL. In some embodiments, the effective amount of the ADC is a dose of 700 mg with a volume of instillation of 10 mL. In some embodiments, the effective amount of the ADC is a dose of 750 mg with a volume of instillation of 10 mL. In some embodiments, the effective amount of the ADC is a dose of 800 mg with a volume of instillation of 10 mL. In some embodiments, the effective amount of the ADC is a dose of 850 mg with a volume of instillation of 10 mL. In some embodiments, the effective amount of the ADC is a dose of 900 mg with a volume of instillation of 10 mL.

In some embodiments, the effective amount of the ADC is a dose of about 100 mg with a volume of instillation of about 15 mL. In some embodiments, the effective amount of the ADC is a dose of about 125 mg with a volume of instillation of about 15 mL. In some embodiments, the effective amount of the ADC is a dose of about 150 mg with a volume of instillation of about 15 mL. In some embodiments, the effective amount of the ADC is a dose of about 200 mg with a volume of instillation of about 15 mL. In some embodiments, the effective amount of the ADC is a dose of about 250 mg with a volume of instillation of about 15 mL. In some embodiments, the effective amount of the ADC is a dose of about 300 mg with a volume of instillation of about 15 mL. In some embodiments, the effective amount of the ADC is a dose of about 350 mg with a volume of instillation of about 15 mL. In some embodiments, the effective amount of the ADC is a dose of about 400 mg with a volume of instillation of about 15 mL. In some embodiments, the effective amount of the ADC is a dose of about 450 mg with a volume of instillation of about 15 mL. In some embodiments, the effective amount of the ADC is a dose of about 500 mg with a volume of instillation of about 15 mL. In some embodiments, the effective amount of the ADC is a dose of about 550 mg with a volume of instillation of about 15 mL. In some embodiments, the effective amount of the ADC is a dose of about 600 mg with a volume of instillation of about 15 mL. In some embodiments, the effective amount of the ADC is a dose of about 650 mg with a volume of instillation of about 15 mL. In some embodiments, the effective amount of the ADC is a dose of about 700 mg with a volume of instillation of about 15 mL. In some embodiments, the effective amount of the ADC is a dose of about 750 mg with a volume of instillation of about 15 mL. In some embodiments, the effective amount of the ADC is a dose of about 800 mg with a volume of instillation of about 15 mL. In some embodiments, the effective amount of the ADC is a dose of about 850 mg with a volume of instillation of about 15 mL. In some embodiments, the effective amount of the ADC is a dose of about 900 mg with a volume of instillation of about 15 mL.

In some embodiments, the effective amount of the ADC is a dose of 100 mg with a volume of instillation of 15 mL. In some embodiments, the effective amount of the ADC is a dose of 125 mg with a volume of instillation of 15 mL. In some embodiments, the effective amount of the ADC is a dose of 150 mg with a volume of instillation of 15 mL. In some embodiments, the effective amount of the ADC is a dose of 200 mg with a volume of instillation of 15 mL. In some embodiments, the effective amount of the ADC is a dose of 250 mg with a volume of instillation of 15 mL. In some embodiments, the effective amount of the ADC is a dose of 300 mg with a volume of instillation of 15 mL. In some embodiments, the effective amount of the ADC is a dose of 350 mg with a volume of instillation of 15 mL. In some embodiments, the effective amount of the ADC is a dose of 400 mg with a volume of instillation of 15 mL. In some embodiments, the effective amount of the ADC is a dose of 450 mg with a volume of instillation of 15 mL. In some embodiments, the effective amount of the ADC is a dose of 500 mg with a volume of instillation of 15 mL. In some embodiments, the effective amount of the ADC is a dose of 550 mg with a volume of instillation of 15 mL. In some embodiments, the effective amount of the ADC is a dose of 600 mg with a volume of instillation of 15 mL. In some embodiments, the effective amount of the ADC is a dose of 650 mg with a volume of instillation of 15 mL. In some embodiments, the effective amount of the ADC is a dose of 700 mg with a volume of instillation of 15 mL. In some embodiments, the effective amount of the ADC is a dose of 750 mg with a volume of instillation of 15 mL. In some embodiments, the effective amount of the ADC is a dose of 800 mg with a volume of instillation of 15 mL. In some embodiments, the effective amount of the ADC is a dose of 850 mg with a volume of instillation of 15 mL. In some embodiments, the effective amount of the ADC is a dose of 900 mg with a volume of instillation of 15 mL.

In some embodiments, the effective amount of the ADC is a dose of about 100 mg with a volume of instillation of about 20 mL. In some embodiments, the effective amount of the ADC is a dose of about 125 mg with a volume of instillation of about 20 mL. In some embodiments, the effective amount of the ADC is a dose of about 150 mg with a volume of instillation of about 20 mL. In some embodiments, the effective amount of the ADC is a dose of about 200 mg with a volume of instillation of about 20 mL. In some embodiments, the effective amount of the ADC is a dose of about 250 mg with a volume of instillation of about 20 mL. In some embodiments, the effective amount of the ADC is a dose of about 300 mg with a volume of instillation of about 20 mL. In some embodiments, the effective amount of the ADC is a dose of about 350 mg with a volume of instillation of about 20 mL. In some embodiments, the effective amount of the ADC is a dose of about 400 mg with a volume of instillation of about 20 mL. In some embodiments, the effective amount of the ADC is a dose of about 450 mg with a volume of instillation of about 20 mL. In some embodiments, the effective amount of the ADC is a dose of about 500 mg with a volume of instillation of about 20 mL. In some embodiments, the effective amount of the ADC is a dose of about 550 mg with a volume of instillation of about 20 mL. In some embodiments, the effective amount of the ADC is a dose of about 600 mg with a volume of instillation of about 20 mL. In some embodiments, the effective amount of the ADC is a dose of about 650 mg with a volume of instillation of about 20 mL. In some embodiments, the effective amount of the ADC is a dose of about 700 mg with a volume of instillation of about 20 mL. In some embodiments, the effective amount of the ADC is a dose of about 750 mg with a volume of instillation of about 20 mL. In some embodiments, the effective amount of the ADC is a dose of about 800 mg with a volume of instillation of about 20 mL. In some embodiments, the effective amount of the ADC is a dose of about 850 mg with a volume of instillation of about 20 mL. In some embodiments, the effective amount of the ADC is a dose of about 900 mg with a volume of instillation of about 20 mL.

In some embodiments, the effective amount of the ADC is a dose of 100 mg with a volume of instillation of 20 mL. In some embodiments, the effective amount of the ADC is a dose of 125 mg with a volume of instillation of 20 mL. In some embodiments, the effective amount of the ADC is a dose of 150 mg with a volume of instillation of 20 mL. In some embodiments, the effective amount of the ADC is a dose of 200 mg with a volume of instillation of 20 mL. In some embodiments, the effective amount of the ADC is a dose of 250 mg with a volume of instillation of 20 mL. In some embodiments, the effective amount of the ADC is a dose of 300 mg with a volume of instillation of 20 mL. In some embodiments, the effective amount of the ADC is a dose of 350 mg with a volume of instillation of 20 mL. In some embodiments, the effective amount of the ADC is a dose of 400 mg with a volume of instillation of 20 mL. In some embodiments, the effective amount of the ADC is a dose of 450 mg with a volume of instillation of 20 mL. In some embodiments, the effective amount of the ADC is a dose of 500 mg with a volume of instillation of 20 mL. In some embodiments, the effective amount of the ADC is a dose of 550 mg with a volume of instillation of 20 mL. In some embodiments, the effective amount of the ADC is a dose of 600 mg with a volume of instillation of 20 mL. In some embodiments, the effective amount of the ADC is a dose of 650 mg with a volume of instillation of 20 mL. In some embodiments, the effective amount of the ADC is a dose of 700 mg with a volume of instillation of 20 mL. In some embodiments, the effective amount of the ADC is a dose of 750 mg with a volume of instillation of 20 mL. In some embodiments, the effective amount of the ADC is a dose of 800 mg with a volume of instillation of 20 mL. In some embodiments, the effective amount of the ADC is a dose of 850 mg with a volume of instillation of 20 mL. In some embodiments, the effective amount of the ADC is a dose of 900 mg with a volume of instillation of 20 mL.

In some embodiments, the effective amount of the ADC is a dose of about 100 mg with a volume of instillation of about 25 mL. In some embodiments, the effective amount of the ADC is a dose of about 125 mg with a volume of instillation of about 25 mL. In some embodiments, the effective amount of the ADC is a dose of about 150 mg with a volume of instillation of about 25 mL. In some embodiments, the effective amount of the ADC is a dose of about 200 mg with a volume of instillation of about 25 mL. In some embodiments, the effective amount of the ADC is a dose of about 250 mg with a volume of instillation of about 25 mL. In some embodiments, the effective amount of the ADC is a dose of about 300 mg with a volume of instillation of about 25 mL. In some embodiments, the effective amount of the ADC is a dose of about 350 mg with a volume of instillation of about 25 mL. In some embodiments, the effective amount of the ADC is a dose of about 400 mg with a volume of instillation of about 25 mL. In some embodiments, the effective amount of the ADC is a dose of about 450 mg with a volume of instillation of about 25 mL. In some embodiments, the effective amount of the ADC is a dose of about 500 mg with a volume of instillation of about 25 mL. In some embodiments, the effective amount of the ADC is a dose of about 550 mg with a volume of instillation of about 25 mL. In some embodiments, the effective amount of the ADC is a dose of about 600 mg with a volume of instillation of about 25 mL. In some embodiments, the effective amount of the ADC is a dose of about 650 mg with a volume of instillation of about 25 mL. In some embodiments, the effective amount of the ADC is a dose of about 700 mg with a volume of instillation of about 25 mL. In some embodiments, the effective amount of the ADC is a dose of about 750 mg with a volume of instillation of about 25 mL. In some embodiments, the effective amount of the ADC is a dose of about 800 mg with a volume of instillation of about 25 mL. In some embodiments, the effective amount of the ADC is a dose of about 850 mg with a volume of instillation of about 25 mL. In some embodiments, the effective amount of the ADC is a dose of about 900 mg with a volume of instillation of about 25 mL.

In some embodiments, the effective amount of the ADC is a dose of 100 mg with a volume of instillation of 25 mL. In some embodiments, the effective amount of the ADC is a dose of 125 mg with a volume of instillation of 25 mL. In some embodiments, the effective amount of the ADC is a dose of 150 mg with a volume of instillation of 25 mL. In some embodiments, the effective amount of the ADC is a dose of 200 mg with a volume of instillation of 25 mL. In some embodiments, the effective amount of the ADC is a dose of 250 mg with a volume of instillation of 25 mL. In some embodiments, the effective amount of the ADC is a dose of 300 mg with a volume of instillation of 25 mL. In some embodiments, the effective amount of the ADC is a dose of 350 mg with a volume of instillation of 25 mL. In some embodiments, the effective amount of the ADC is a dose of 400 mg with a volume of instillation of 25 mL. In some embodiments, the effective amount of the ADC is a dose of 450 mg with a volume of instillation of 25 mL. In some embodiments, the effective amount of the ADC is a dose of 500 mg with a volume of instillation of 25 mL. In some embodiments, the effective amount of the ADC is a dose of 550 mg with a volume of instillation of 25 mL. In some embodiments, the effective amount of the ADC is a dose of 600 mg with a volume of instillation of 25 mL. In some embodiments, the effective amount of the ADC is a dose of 650 mg with a volume of instillation of 25 mL. In some embodiments, the effective amount of the ADC is a dose of 700 mg with a volume of instillation of 25 mL. In some embodiments, the effective amount of the ADC is a dose of 750 mg with a volume of instillation of 25 mL. In some embodiments, the effective amount of the ADC is a dose of 800 mg with a volume of instillation of 25 mL. In some embodiments, the effective amount of the ADC is a dose of 850 mg with a volume of instillation of 25 mL. In some embodiments, the effective amount of the ADC is a dose of 900 mg with a volume of instillation of 25 mL.

In some embodiments, the effective amount of the ADC is a dose of about 100 mg with a volume of instillation of about 30 mL. In some embodiments, the effective amount of the ADC is a dose of about 125 mg with a volume of instillation of about 30 mL. In some embodiments, the effective amount of the ADC is a dose of about 150 mg with a volume of instillation of about 30 mL. In some embodiments, the effective amount of the ADC is a dose of about 200 mg with a volume of instillation of about 30 mL. In some embodiments, the effective amount of the ADC is a dose of about 250 mg with a volume of instillation of about 30 mL. In some embodiments, the effective amount of the ADC is a dose of about 300 mg with a volume of instillation of about 30 mL. In some embodiments, the effective amount of the ADC is a dose of about 350 mg with a volume of instillation of about 30 mL. In some embodiments, the effective amount of the ADC is a dose of about 400 mg with a volume of instillation of about 30 mL. In some embodiments, the effective amount of the ADC is a dose of about 450 mg with a volume of instillation of about 30 mL. In some embodiments, the effective amount of the ADC is a dose of about 500 mg with a volume of instillation of about 30 mL. In some embodiments, the effective amount of the ADC is a dose of about 550 mg with a volume of instillation of about 30 mL. In some embodiments, the effective amount of the ADC is a dose of about 600 mg with a volume of instillation of about 30 mL. In some embodiments, the effective amount of the ADC is a dose of about 650 mg with a volume of instillation of about 30 mL. In some embodiments, the effective amount of the ADC is a dose of about 700 mg with a volume of instillation of about 30 mL. In some embodiments, the effective amount of the ADC is a dose of about 750 mg with a volume of instillation of about 30 mL. In some embodiments, the effective amount of the ADC is a dose of about 800 mg with a volume of instillation of about 30 mL. In some embodiments, the effective amount of the ADC is a dose of about 850 mg with a volume of instillation of about 30 mL. In some embodiments, the effective amount of the ADC is a dose of about 900 mg with a volume of instillation of about 30 mL.

In some embodiments, the effective amount of the ADC is a dose of 100 mg with a volume of instillation of 30 mL. In some embodiments, the effective amount of the ADC is a dose of 125 mg with a volume of instillation of 30 mL. In some embodiments, the effective amount of the ADC is a dose of 150 mg with a volume of instillation of 30 mL. In some embodiments, the effective amount of the ADC is a dose of 200 mg with a volume of instillation of 30 mL. In some embodiments, the effective amount of the ADC is a dose of 250 mg with a volume of instillation of 30 mL. In some embodiments, the effective amount of the ADC is a dose of 300 mg with a volume of instillation of 30 mL. In some embodiments, the effective amount of the ADC is a dose of 350 mg with a volume of instillation of 30 mL. In some embodiments, the effective amount of the ADC is a dose of 400 mg with a volume of instillation of 30 mL. In some embodiments, the effective amount of the ADC is a dose of 450 mg with a volume of instillation of 30 mL. In some embodiments, the effective amount of the ADC is a dose of 500 mg with a volume of instillation of 30 mL. In some embodiments, the effective amount of the ADC is a dose of 550 mg with a volume of instillation of 30 mL. In some embodiments, the effective amount of the ADC is a dose of 600 mg with a volume of instillation of 30 mL. In some embodiments, the effective amount of the ADC is a dose of 650 mg with a volume of instillation of 30 mL. In some embodiments, the effective amount of the ADC is a dose of 700 mg with a volume of instillation of 30 mL. In some embodiments, the effective amount of the ADC is a dose of 750 mg with a volume of instillation of 30 mL. In some embodiments, the effective amount of the ADC is a dose of 800 mg with a volume of instillation of 30 mL. In some embodiments, the effective amount of the ADC is a dose of 850 mg with a volume of instillation of 30 mL. In some embodiments, the effective amount of the ADC is a dose of 900 mg with a volume of instillation of 30 mL.

In some embodiments, the effective amount of the ADC is a dose of about 100 mg with a volume of instillation of about 35 mL. In some embodiments, the effective amount of the ADC is a dose of about 125 mg with a volume of instillation of about 35 mL. In some embodiments, the effective amount of the ADC is a dose of about 150 mg with a volume of instillation of about 35 mL. In some embodiments, the effective amount of the ADC is a dose of about 200 mg with a volume of instillation of about 35 mL. In some embodiments, the effective amount of the ADC is a dose of about 250 mg with a volume of instillation of about 35 mL. In some embodiments, the effective amount of the ADC is a dose of about 300 mg with a volume of instillation of about 35 mL. In some embodiments, the effective amount of the ADC is a dose of about 350 mg with a volume of instillation of about 35 mL. In some embodiments, the effective amount of the ADC is a dose of about 400 mg with a volume of instillation of about 35 mL. In some embodiments, the effective amount of the ADC is a dose of about 450 mg with a volume of instillation of about 35 mL. In some embodiments, the effective amount of the ADC is a dose of about 500 mg with a volume of instillation of about 35 mL. In some embodiments, the effective amount of the ADC is a dose of about 550 mg with a volume of instillation of about 35 mL. In some embodiments, the effective amount of the ADC is a dose of about 600 mg with a volume of instillation of about 35 mL. In some embodiments, the effective amount of the ADC is a dose of about 650 mg with a volume of instillation of about 35 mL. In some embodiments, the effective amount of the ADC is a dose of about 700 mg with a volume of instillation of about 35 mL. In some embodiments, the effective amount of the ADC is a dose of about 750 mg with a volume of instillation of about 35 mL. In some embodiments, the effective amount of the ADC is a dose of about 800 mg with a volume of instillation of about 35 mL. In some embodiments, the effective amount of the ADC is a dose of about 850 mg with a volume of instillation of about 35 mL. In some embodiments, the effective amount of the ADC is a dose of about 900 mg with a volume of instillation of about 35 mL.

In some embodiments, the effective amount of the ADC is a dose of 100 mg with a volume of instillation of 35 mL. In some embodiments, the effective amount of the ADC is a dose of 125 mg with a volume of instillation of 35 mL. In some embodiments, the effective amount of the ADC is a dose of 150 mg with a volume of instillation of 35 mL. In some embodiments, the effective amount of the ADC is a dose of 200 mg with a volume of instillation of 35 mL. In some embodiments, the effective amount of the ADC is a dose of 250 mg with a volume of instillation of 35 mL. In some embodiments, the effective amount of the ADC is a dose of 300 mg with a volume of instillation of 35 mL. In some embodiments, the effective amount of the ADC is a dose of 350 mg with a volume of instillation of 35 mL. In some embodiments, the effective amount of the ADC is a dose of 400 mg with a volume of instillation of 35 mL. In some embodiments, the effective amount of the ADC is a dose of 450 mg with a volume of instillation of 35 mL. In some embodiments, the effective amount of the ADC is a dose of 500 mg with a volume of instillation of 35 mL. In some embodiments, the effective amount of the ADC is a dose of 550 mg with a volume of instillation of 35 mL. In some embodiments, the effective amount of the ADC is a dose of 600 mg with a volume of instillation of 35 mL. In some embodiments, the effective amount of the ADC is a dose of 650 mg with a volume of instillation of 35 mL. In some embodiments, the effective amount of the ADC is a dose of 700 mg with a volume of instillation of 35 mL. In some embodiments, the effective amount of the ADC is a dose of 750 mg with a volume of instillation of 35 mL. In some embodiments, the effective amount of the ADC is a dose of 800 mg with a volume of instillation of 35 mL. In some embodiments, the effective amount of the ADC is a dose of 850 mg with a volume of instillation of 35 mL. In some embodiments, the effective amount of the ADC is a dose of 900 mg with a volume of instillation of 35 mL.

In some embodiments, the effective amount of the ADC is a dose of about 100 mg with a volume of instillation of about 40 mL. In some embodiments, the effective amount of the ADC is a dose of about 125 mg with a volume of instillation of about 40 mL. In some embodiments, the effective amount of the ADC is a dose of about 150 mg with a volume of instillation of about 40 mL. In some embodiments, the effective amount of the ADC is a dose of about 200 mg with a volume of instillation of about 40 mL. In some embodiments, the effective amount of the ADC is a dose of about 250 mg with a volume of instillation of about 40 mL. In some embodiments, the effective amount of the ADC is a dose of about 300 mg with a volume of instillation of about 40 mL. In some embodiments, the effective amount of the ADC is a dose of about 350 mg with a volume of instillation of about 40 mL. In some embodiments, the effective amount of the ADC is a dose of about 400 mg with a volume of instillation of about 40 mL. In some embodiments, the effective amount of the ADC is a dose of about 450 mg with a volume of instillation of about 40 mL. In some embodiments, the effective amount of the ADC is a dose of about 500 mg with a volume of instillation of about 40 mL. In some embodiments, the effective amount of the ADC is a dose of about 550 mg with a volume of instillation of about 40 mL. In some embodiments, the effective amount of the ADC is a dose of about 600 mg with a volume of instillation of about 40 mL. In some embodiments, the effective amount of the ADC is a dose of about 650 mg with a volume of instillation of about 40 mL. In some embodiments, the effective amount of the ADC is a dose of about 700 mg with a volume of instillation of about 40 mL. In some embodiments, the effective amount of the ADC is a dose of about 750 mg with a volume of instillation of about 40 mL. In some embodiments, the effective amount of the ADC is a dose of about 800 mg with a volume of instillation of about 40 mL. In some embodiments, the effective amount of the ADC is a dose of about 850 mg with a volume of instillation of about 40 mL. In some embodiments, the effective amount of the ADC is a dose of about 900 mg with a volume of instillation of about 40 mL.

In some embodiments, the effective amount of the ADC is a dose of 100 mg with a volume of instillation of 40 mL. In some embodiments, the effective amount of the ADC is a dose of 125 mg with a volume of instillation of 40 mL. In some embodiments, the effective amount of the ADC is a dose of 150 mg with a volume of instillation of 40 mL. In some embodiments, the effective amount of the ADC is a dose of 200 mg with a volume of instillation of 40 mL. In some embodiments, the effective amount of the ADC is a dose of 250 mg with a volume of instillation of 40 mL. In some embodiments, the effective amount of the ADC is a dose of 300 mg with a volume of instillation of 40 mL. In some embodiments, the effective amount of the ADC is a dose of 350 mg with a volume of instillation of 40 mL. In some embodiments, the effective amount of the ADC is a dose of 400 mg with a volume of instillation of 40 mL. In some embodiments, the effective amount of the ADC is a dose of 450 mg with a volume of instillation of 40 mL. In some embodiments, the effective amount of the ADC is a dose of 500 mg with a volume of instillation of 40 mL. In some embodiments, the effective amount of the ADC is a dose of 550 mg with a volume of instillation of 40 mL. In some embodiments, the effective amount of the ADC is a dose of 600 mg with a volume of instillation of 40 mL. In some embodiments, the effective amount of the ADC is a dose of 650 mg with a volume of instillation of 40 mL. In some embodiments, the effective amount of the ADC is a dose of 700 mg with a volume of instillation of 40 mL. In some embodiments, the effective amount of the ADC is a dose of 750 mg with a volume of instillation of 40 mL. In some embodiments, the effective amount of the ADC is a dose of 800 mg with a volume of instillation of 40 mL. In some embodiments, the effective amount of the ADC is a dose of 850 mg with a volume of instillation of 40 mL. In some embodiments, the effective amount of the ADC is a dose of 900 mg with a volume of instillation of 40 mL.

In some embodiments, the effective amount of the ADC is a dose of about 100 mg with a volume of instillation of about 45 mL. In some embodiments, the effective amount of the ADC is a dose of about 125 mg with a volume of instillation of about 45 mL. In some embodiments, the effective amount of the ADC is a dose of about 150 mg with a volume of instillation of about 45 mL. In some embodiments, the effective amount of the ADC is a dose of about 200 mg with a volume of instillation of about 45 mL. In some embodiments, the effective amount of the ADC is a dose of about 250 mg with a volume of instillation of about 45 mL. In some embodiments, the effective amount of the ADC is a dose of about 300 mg with a volume of instillation of about 45 mL. In some embodiments, the effective amount of the ADC is a dose of about 350 mg with a volume of instillation of about 45 mL. In some embodiments, the effective amount of the ADC is a dose of about 400 mg with a volume of instillation of about 45 mL. In some embodiments, the effective amount of the ADC is a dose of about 450 mg with a volume of instillation of about 45 mL. In some embodiments, the effective amount of the ADC is a dose of about 500 mg with a volume of instillation of about 45 mL. In some embodiments, the effective amount of the ADC is a dose of about 550 mg with a volume of instillation of about 45 mL. In some embodiments, the effective amount of the ADC is a dose of about 600 mg with a volume of instillation of about 45 mL. In some embodiments, the effective amount of the ADC is a dose of about 650 mg with a volume of instillation of about 45 mL. In some embodiments, the effective amount of the ADC is a dose of about 700 mg with a volume of instillation of about 45 mL. In some embodiments, the effective amount of the ADC is a dose of about 750 mg with a volume of instillation of about 45 mL. In some embodiments, the effective amount of the ADC is a dose of about 800 mg with a volume of instillation of about 45 mL. In some embodiments, the effective amount of the ADC is a dose of about 850 mg with a volume of instillation of about 45 mL. In some embodiments, the effective amount of the ADC is a dose of about 900 mg with a volume of instillation of about 45 mL.

In some embodiments, the effective amount of the ADC is a dose of 100 mg with a volume of instillation of 45 mL. In some embodiments, the effective amount of the ADC is a dose of 125 mg with a volume of instillation of 45 mL. In some embodiments, the effective amount of the ADC is a dose of 150 mg with a volume of instillation of 45 mL. In some embodiments, the effective amount of the ADC is a dose of 200 mg with a volume of instillation of 45 mL. In some embodiments, the effective amount of the ADC is a dose of 250 mg with a volume of instillation of 45 mL. In some embodiments, the effective amount of the ADC is a dose of 300 mg with a volume of instillation of 45 mL. In some embodiments, the effective amount of the ADC is a dose of 350 mg with a volume of instillation of 45 mL. In some embodiments, the effective amount of the ADC is a dose of 400 mg with a volume of instillation of 45 mL. In some embodiments, the effective amount of the ADC is a dose of 450 mg with a volume of instillation of 45 mL. In some embodiments, the effective amount of the ADC is a dose of 500 mg with a volume of instillation of 45 mL. In some embodiments, the effective amount of the ADC is a dose of 550 mg with a volume of instillation of 45 mL. In some embodiments, the effective amount of the ADC is a dose of 600 mg with a volume of instillation of 45 mL. In some embodiments, the effective amount of the ADC is a dose of 650 mg with a volume of instillation of 45 mL. In some embodiments, the effective amount of the ADC is a dose of 700 mg with a volume of instillation of 45 mL. In some embodiments, the effective amount of the ADC is a dose of 750 mg with a volume of instillation of 45 mL. In some embodiments, the effective amount of the ADC is a dose of 800 mg with a volume of instillation of 45 mL. In some embodiments, the effective amount of the ADC is a dose of 850 mg with a volume of instillation of 45 mL. In some embodiments, the effective amount of the ADC is a dose of 900 mg with a volume of instillation of 45 mL.

In some embodiments, the effective amount of the ADC is a dose of about 100 mg with a volume of instillation of about 50 mL. In some embodiments, the effective amount of the ADC is a dose of about 125 mg with a volume of instillation of about 50 mL. In some embodiments, the effective amount of the ADC is a dose of about 150 mg with a volume of instillation of about 50 mL. In some embodiments, the effective amount of the ADC is a dose of about 200 mg with a volume of instillation of about 50 mL. In some embodiments, the effective amount of the ADC is a dose of about 250 mg with a volume of instillation of about 50 mL. In some embodiments, the effective amount of the ADC is a dose of about 300 mg with a volume of instillation of about 50 mL. In some embodiments, the effective amount of the ADC is a dose of about 350 mg with a volume of instillation of about 50 mL. In some embodiments, the effective amount of the ADC is a dose of about 400 mg with a volume of instillation of about 50 mL. In some embodiments, the effective amount of the ADC is a dose of about 450 mg with a volume of instillation of about 50 mL. In some embodiments, the effective amount of the ADC is a dose of about 500 mg with a volume of instillation of about 50 mL. In some embodiments, the effective amount of the ADC is a dose of about 550 mg with a volume of instillation of about 50 mL. In some embodiments, the effective amount of the ADC is a dose of about 600 mg with a volume of instillation of about 50 mL. In some embodiments, the effective amount of the ADC is a dose of about 650 mg with a volume of instillation of about 50 mL. In some embodiments, the effective amount of the ADC is a dose of about 700 mg with a volume of instillation of about 50 mL. In some embodiments, the effective amount of the ADC is a dose of about 750 mg with a volume of instillation of about 50 mL. In some embodiments, the effective amount of the ADC is a dose of about 800 mg with a volume of instillation of about 50 mL. In some embodiments, the effective amount of the ADC is a dose of about 850 mg with a volume of instillation of about 50 mL. In some embodiments, the effective amount of the ADC is a dose of about 900 mg with a volume of instillation of about 50 mL.

In some embodiments, the effective amount of the ADC is a dose of 100 mg with a volume of instillation of 50 mL. In some embodiments, the effective amount of the ADC is a dose of 125 mg with a volume of instillation of 50 mL. In some embodiments, the effective amount of the ADC is a dose of 150 mg with a volume of instillation of 50 mL. In some embodiments, the effective amount of the ADC is a dose of 200 mg with a volume of instillation of 50 mL. In some embodiments, the effective amount of the ADC is a dose of 250 mg with a volume of instillation of 50 mL. In some embodiments, the effective amount of the ADC is a dose of 300 mg with a volume of instillation of 50 mL. In some embodiments, the effective amount of the ADC is a dose of 350 mg with a volume of instillation of 50 mL. In some embodiments, the effective amount of the ADC is a dose of 400 mg with a volume of instillation of 50 mL. In some embodiments, the effective amount of the ADC is a dose of 450 mg with a volume of instillation of 50 mL. In some embodiments, the effective amount of the ADC is a dose of 500 mg with a volume of instillation of 50 mL. In some embodiments, the effective amount of the ADC is a dose of 550 mg with a volume of instillation of 50 mL. In some embodiments, the effective amount of the ADC is a dose of 600 mg with a volume of instillation of 50 mL. In some embodiments, the effective amount of the ADC is a dose of 650 mg with a volume of instillation of 50 mL. In some embodiments, the effective amount of the ADC is a dose of 700 mg with a volume of instillation of 50 mL. In some embodiments, the effective amount of the ADC is a dose of 750 mg with a volume of instillation of 50 mL. In some embodiments, the effective amount of the ADC is a dose of 800 mg with a volume of instillation of 50 mL. In some embodiments, the effective amount of the ADC is a dose of 850 mg with a volume of instillation of 50 mL. In some embodiments, the effective amount of the ADC is a dose of 900 mg with a volume of instillation of 50 mL.

In some embodiments, the maximal dwell time of each intravesical administration is about 120 minutes. In some embodiments, the maximal dwell time of each intravesical administration is about 90 minutes. In some embodiments, the maximal dwell time of each intravesical administration is the subject's tolerated dwell time. In some embodiments, the dwell time of each intravesical administration is between about 30 minutes to about 120 minutes. In some embodiments, the dwell time of each intravesical administration is between about 30 minutes to about 90 minutes. In some embodiments, the dwell time of each intravesical administration is about 30 minutes. In some embodiments, the dwell time of each intravesical administration is about 40 minutes. In some embodiments, the dwell time of each intravesical administration is about 50 minutes. In some embodiments, the dwell time of each intravesical administration is about 60 minutes. In some embodiments, the dwell time of each intravesical administration is about 70 minutes. In some embodiments, the dwell time of each intravesical administration is about 80 minutes. In some embodiments, the dwell time of each intravesical administration is about 90 minutes. In some embodiments, the dwell time of each intravesical administration is about 100 minutes. In some embodiments, the dwell time of each intravesical administration is about 110 minutes. In some embodiments, the dwell time of each intravesical administration is about 120 minutes. In some embodiments, the dwell time of each intravesical administration is 30 minutes. In some embodiments, the dwell time of each intravesical administration is 40 minutes. In some embodiments, the dwell time of each intravesical administration is 50 minutes. In some embodiments, the dwell time of each intravesical administration is 60 minutes. In some embodiments, the dwell time of each intravesical administration is 70 minutes. In some embodiments, the dwell time of each intravesical administration is 80 minutes. In some embodiments, the dwell time of each intravesical administration is 90 minutes. In some embodiments, the dwell time of each intravesical administration is 100 minutes. In some embodiments, the dwell time of each intravesical administration is 110 minutes. In some embodiments, the dwell time of each intravesical administration is 120 minutes.

In some embodiments, the pharmaceutical composition comprising the antibody drug conjugate provided herein is administered intravesically during an induction phase. In some embodiments, the pharmaceutical composition comprising the antibody drug conjugate provided herein is administered intravesically during a maintenance phase. In some embodiments, the pharmaceutical composition comprising the antibody drug conjugate provided herein is administered intravesically during two phases, wherein the two phases are an induction phase and a maintenance phase. In some embodiments, the maintenance phase starts after the induction phase. In some embodiments, the maintenance phase starts between six to ten weeks, between six to nine weeks, or between six to eight weeks after the induction phase. In some embodiments, the maintenance phase starts ten weeks after the induction phase. In some embodiments, the maintenance phase starts nine weeks after the induction phase. In some embodiments, the maintenance phase starts eight weeks after the induction phase. In some embodiments, the maintenance phase starts seven weeks after the induction phase. In some embodiments, the maintenance phase starts six weeks after the induction phase.

In some embodiments, the pharmaceutical composition comprising the antibody drug conjugate provided herein is administered about 1 to about 25 times, wherein the doses can be administered as necessary, e.g., weekly, biweekly, monthly, bimonthly, trimonthly, etc., as determined by a physician. In some embodiments, the pharmaceutical composition comprising the antibody drug conjugate provided herein is administered weekly. In some embodiments, the pharmaceutical composition comprising the antibody drug conjugate provided herein is administered biweekly. In some embodiments, the pharmaceutical composition comprising the antibody drug conjugate provided herein is administered monthly. In some embodiments, the pharmaceutical composition comprising the antibody drug conjugate provided herein is administered bimonthly. In some embodiments, the pharmaceutical composition comprising the antibody drug conjugate provided herein is administered trimonthly. In some embodiments, the pharmaceutical composition comprising the antibody drug conjugate provided herein is administered 25 times, 24 times, 23 times, 22 times, 21 times, 20 times, 19 time, 18 times, 17 times, 16 times, 15 times, 14 times, 13 times, 12 times, 11 times, 10 times, 9 times, 8 times, 7 times, 6 times, 5 times, 4 times, 3 times, 2 times or 1 time to treat NMIBC, wherein the dose is between about 10 mg to about 1000 mg with a volume of instillation between about 10 mL to about 100 mL.

In some embodiments, the pharmaceutical composition comprising the antibody drug conjugate provided herein is administered intravesically once a week for four weeks during the induction phase. In some embodiments, the pharmaceutical composition comprising the antibody drug conjugate provided herein is administered intravesically once a week for five weeks during the induction phase. In some embodiments, the pharmaceutical composition comprising the antibody drug conjugate provided herein is administered intravesically once a week for six weeks during the induction phase. In some embodiments, the pharmaceutical composition comprising the antibody drug conjugate provided herein is administered intravesically once a week for seven weeks during the induction phase. In some embodiments, the pharmaceutical composition comprising the antibody drug conjugate provided herein is administered intravesically once a week for eight weeks during the induction phase.

In some embodiments, the pharmaceutical composition comprising the antibody drug conjugate provided herein is administered intravesically once a month for six months during the maintenance phase. In some embodiments, the pharmaceutical composition comprising the antibody drug conjugate provided herein is administered intravesically once a month for seven months during the maintenance phase. In some embodiments, the pharmaceutical composition comprising the antibody drug conjugate provided herein is administered intravesically once a month for eight months during the maintenance phase. In some embodiments, the pharmaceutical composition comprising the antibody drug conjugate provided herein is administered intravesically once a month for nine months during the maintenance phase. In some embodiments, the pharmaceutical composition comprising the antibody drug conjugate provided herein is administered intravesically once a month for ten months during the maintenance phase. In some embodiments, the pharmaceutical composition comprising the antibody drug conjugate provided herein is administered intravesically once a month for 11 months during the maintenance phase.

In some embodiments, the pharmaceutical composition comprising the antibody drug conjugate provided herein is administered intravesically once a week for six weeks during the induction phase and once a month for nine months during the maintenance phase, wherein the maintenance phase starts between six to ten weeks, between six to nine weeks, or between six to eight weeks after the induction phase.

In some more specific embodiments of the methods provided herein, the ADC has the following structure:

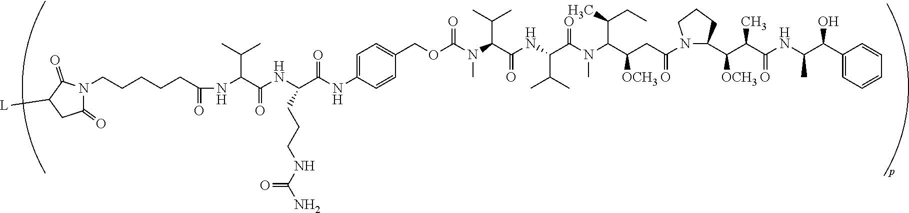

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered intravesically at a dose of about 125 mg with a volume of instillation of about 25 mL and a maximum 90-minute dwell time, wherein the dose is administered intravesically once a week for six weeks during the induction phase and once a month for nine months during the maintenance phase, and wherein the maintenance phase starts between six to ten weeks after the induction phase.

In some more specific embodiments of the methods provided herein, the ADC has the following structure:

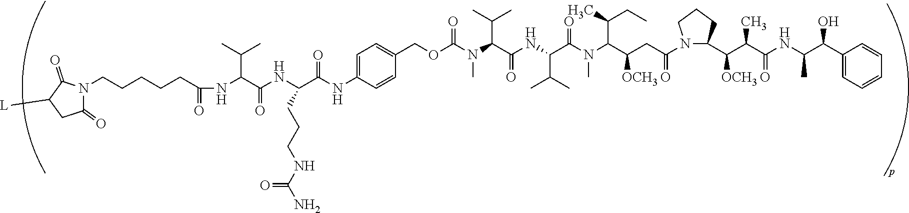

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered intravesically at a dose of about 250 mg with a volume of instillation of about 25 mL and a maximum 90-minute dwell time, wherein the dose is administered intravesically once a week for six weeks during the induction phase and once a month for nine months during the maintenance phase, and wherein the maintenance phase starts between six to ten weeks after the induction phase.

In some more specific embodiments of the methods provided herein, the ADC has the following structure:

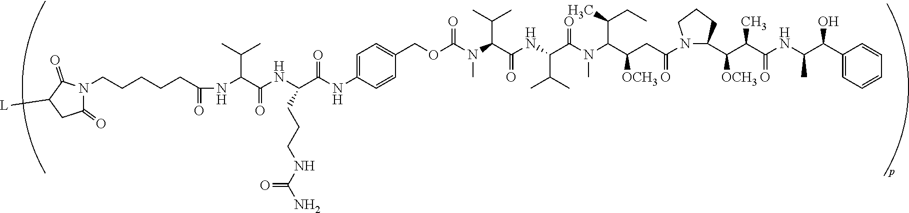

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered intravesically at a dose of about 500 mg with a volume of instillation of about 25 mL and a maximum 90-minute dwell time, wherein the dose is administered intravesically once a week for six weeks during the induction phase and once a month for nine months during the maintenance phase, and wherein the maintenance phase starts between six to ten weeks after the induction phase.

In some more specific embodiments of the methods provided herein, the ADC has the following structure:

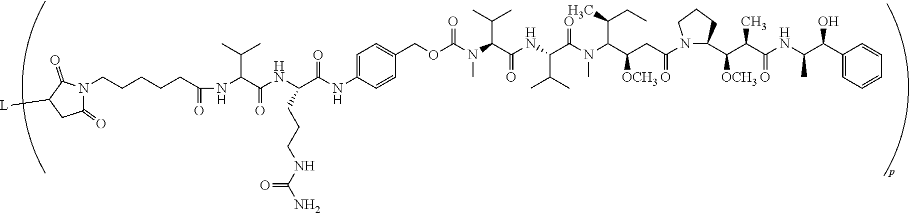

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered intravesically at a dose of about 750 mg with a volume of instillation of about 25 mL and a maximum 90-minute dwell time, wherein the dose is administered intravesically once a week for six weeks during the induction phase and once a month for nine months during the maintenance phase, and wherein the maintenance phase starts between six to ten weeks after the induction phase.

### 5.7 Methods for Determining the Biomarkers

The disclosure provides that the expression of any of the markers provided herein can be determined by various methods known in the field. In some embodiments, the expression of the markers can be determined by the amount or relative amount of mRNA transcribed from the marker genes. In one embodiment, the expression of the marker genes can be determined by the amount or relative amount of the protein products encoded by the marker genes. In another embodiment, the expression of the marker genes can be determined by the level of biological or chemical response induced by the protein products encoded by the marker genes. Additionally, in certain embodiments, the expression of the marker genes can be determined by the expression of one or more genes that correlates with the expression of the marker genes.

As described above, levels or amounts of gene transcripts (e.g. mRNA) of the marker genes can be used as a proxy for the expression levels of markers genes. Numerous different PCR or qPCR protocols are known in the art including those exemplified herein. In some embodiments, the various PCR or qPCR methods are applied or adapted for determining the mRNA level of the various marker genes. Quantitative PCR (qPCR) (also referred as real-time PCR) is applied and adapted in some embodiments as it provides not only a quantitative measurement, but also reduced time and contamination. As used herein, “quantitative PCR (or “qPCR”) refers to the direct monitoring of the progress of PCR amplification as it is occurring without the need for repeated sampling of the reaction products. In quantitative PCR, the reaction products can be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time. When qPCR is applied to determine mRNA expression level, an extra step of reverse-transcription of mRNA to DNA is performed before the qPCR analysis. Examples of PCR methods can be found in the literature (Wong et al., BioTechniques 39:75-85 (2005); D'haene et al., Methods 50:262-270 (2010)), which is incorporated by reference herein in its entirety. Examples of PCR assays can also be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In one specific embodiment, qPCR can be performed to determine or measure the mRNA levels of the marker genes as follows. Briefly, mean Ct (cycle threshold) values (or referred to herein interchangeably as Cq (quantification cycle)) of replicate qPCR reactions for the marker genes and one or more housekeeping genes are determined. Mean Ct values for the marker genes can be then normalized to the Ct values of the housekeeping genes using the following exemplary formula: marker-gene-ΔCt=(mean Ct of marker gene−mean Ct of housekeeping gene A). The relative marker-gene-ΔCt can then be used to determine relative level of marker gene mRNA, for example by using the formula of mRNA expression=2^{-ΔCt}. For a summary of Ct and Cq values, see MIQE guideline (Bustin et al., The MIQE Guidelines: Minimum Information for Publication of Quantitative Real-Time PCR Experiments, Clinical Chemistry 55:4 (2009)).

Other commonly used methods known in the art can also be used for the quantification of RNA transcripts of the marker genes in a sample as the proxy for the expression of the marker genes, including northern blotting and in situ hybridization (Parker & Barnes, Methods in Molecular Biology 106:247-283 (1999)); RNAse protection assays (Hod, Biotechniques 13:852-854 (1992)); microarrays (Hoheisel et al., Nature Reviews Genetics 7:200-210 (2006); Jaluria et al., Microbial Cell Factories 6:4 (2007)); and polymerase chain reaction (PCR) (Weis et al, Trends in Genetics 8:263-264 (1992)). RNA in situ hybridization (ISH) is a molecular biology technique widely used to measure and localize specific RNA sequences, for example, messenger RNAs (mRNAs), long non-coding RNAs (lncRNAs), and microRNAs (miRNAs) within cells, such as circulating tumor cells (CTCs) or tissue sections, while preserving the cellular and tissue context. ISH is a type of hybridization that uses a directly or indirectly labeled complementary DNA or RNA strand, such as a probe, to bind to and localize a specific nucleic acid, such as DNA or RNA, in a sample, in particular a portion or section of tissue or cells (in situ). The probe types can be double stranded DNA (dsDNA), single stranded DNA (ssDNA), single stranded complimentary RNA (sscRNA), messenger RNA (mRNA), micro RNA (miRNA), ribosomal RNA, mitochondrial RNA, and/or synthetic oligonucleotides. The term “fluorescent in situ hybridization” or “FISH” refers to a type of ISH utilizing a fluorescent label. The term “chromogenic in situ hybridization” or “CISH” refers to a type of ISH with a chromogenic label. ISH, FISH and CISH methods are well known to those skilled in the art (see, for example, Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000)). RNA ISH therefore provides for spatial-temporal visualization as well as quantification of gene expression within cells and tissues. It has wide applications in research and in diagnostics (Hu et al., Biomark. Res. 2(1):1-13, doi: 10.1186/2050-7771-2-3 (2014); Ratan et al., Cureus 9(6):e1325. doi: 10.7759/cureus.1325 (2017); Weier et al., Expert Rev. Mol. Diagn. 2(2):109-119 (2002)). Fluorescent RNA ISH utilizes fluorescent dyes and fluorescent microscopes for RNA labeling and detection, respectively. Fluorescent RNA ISH can provides for multiplexing of four to five target sequences.

Alternatively, RNA transcripts of the marker genes in a sample as the proxy for the expression of the marker genes can be determined by sequencing techniques. Representative methods for sequencing-based gene expression analysis include Serial Analysis of Gene Expression (SAGE), and gene expression analysis by massively parallel signature sequencing (MPSS).

In some embodiments, expression of the marker genes can be determined by the relative abundance of the RNA transcripts (including for example mRNA) of the marker genes in a pool of total transcribed RNA. Such relative abundance of the RNA transcripts of the marker genes can be determined by next generation sequencing, which is known as RNA-seq. In one example of the RNA-seq procedure, RNAs from different sources (blood, tissue, cells) are purified, optionally enriched (e.g. with oligo (dT) primers), converted to cDNA, and fragmented. Millions or even billions of short sequence reads are generated from the randomly fragmented cDNA library. See Zhao et al. BMC genomics 16: 97 (2015); Zhao et al. Scientific Reports 8: 4781 (2018); Shanrong Zhao et al., RNA, published in advance Apr. 13, 2020, doi: 10.1261/rna.074922.120, all of which are incorporated herein in their entirety by reference. The expression level of each mRNA transcript of the marker genes is determined by the total number of mapped fragments upon normalization, which is directly proportional to its abundance level. A few normalization schemes are known and used to facilitate the use of the abundance of the RNA transcripts as the parameter for determining gene expression, including RPKM (Reads Per Kilobase Million), FPKM (Fragments Per Kilobase Million), and/or TPM (Transcripts Per Kilobase Million). Briefly, RPKM can be calculated as follows: count up the total reads in a sample and divide that number by 1,000,000—which is the “per million” scaling factor; divide the read counts by the “per million” scaling factor, which normalizes for sequencing depth, giving the reads per million (RPM); and divide the RPM values by the length of the gene, in kilobases, which gives RPKM. FPKM is closely related to RPKM except with fragment replacing read. RPKM was made for single-end RNA-seq, where every read corresponded to a single fragment that was sequenced. FPKM was made for paired-end RNA-seq, in which two reads can correspond to a single fragment, or, if one read in the pair did not map, one read can correspond to a single fragment. TPM is very similar to RPKM and FPKM and is calculated as follows: divide the read counts by the length of each gene in kilobases, which gives the reads per kilobase (RPK); count up all the RPK values in a sample and divide this number by 1,000,000, which gives the “per million” scaling factor; divide the RPK values by the “per million” scaling factor, which gives TPM. See Zhao et al. BMC genomics 16: 97 (2015); Zhao et al. Scientific Reports 8: 4781 (2018); Shanrong Zhao et al., RNA, published in advance Apr. 13, 2020, doi: 10.1261/rna.074922.120, all of which are incorporated herein in their entirety by reference.

In one embodiment, the expression of the marker genes is determined by RNA-seq, for example by TPM, RPKM, and/or FPKM. In some embodiments, the expression of the marker genes is determined by TPM. In some embodiments, the expression of the marker genes is determined by RPKM. In some embodiments, the expression of the marker genes is determined by FPKM.

As described earlier, the expression of the marker genes can be determined in a sample from a subject. In some embodiments, the sample is a blood sample, a serum sample, a plasma sample, bodily fluid (e.g. tissue fluid including cancer tissue fluid), or a tissue (e.g. cancer tissue or the tissue surrounding the cancer). In some embodiments, the sample is a tissue sample. In some embodiments, the tissue sample is tissue fractions isolated or extracted from a mammal, in particular a human. In some embodiments, the tissue sample is a population of cells isolated or extracted from a mammal, in particular a human. In some embodiments, the tissue sample is a sample obtained from a biopsy. In certain embodiments, the samples can be obtained from a variety of organs of a subject, including a human subject. In some embodiments, the samples are obtained from organs of a subject having a cancer. In some embodiments, the samples are obtained from organs having a cancer in a subject having a cancer. In other embodiments, the samples, for example reference samples, are obtained from normal organs from the patient or from a second human subject.

In certain embodiments of the methods provided herein, the tissue includes a tissue from bladder, ureter, breast, lung, colon, rectum, ovary, Fallopian tube, esophagus, cervix, uterine endometrium, skin, larynx, bone marrow, salivary gland, kidney, prostate, brain, spinal cord, placenta, adrenal, pancreas, parathyroid, hypophysis, testis, thyroid, spleen, tonsil, thymus, heart, stomach, small intestine, liver, skeletal muscle, peripheral nerve, mesothelium, or eye.

In further embodiments of the methods provided herein, the expression of the various marker genes can be detected by a variety of immunoassays known in the art, including an immunohistochemistry (IHC) assay, an immunoblotting assay, a FACS assay, and an ELISA.

The expression of the various marker genes can be detected by antibodies against the protein products encoded by the marker genes in a variety of IHC assays. IHC staining of tissue sections has been shown to be a reliable method of assessing or detecting the presence of proteins in a sample. IHC techniques utilize an antibody to probe and visualize cellular antigens in situ, generally by chromogenic or fluorescent methods. Primary antibodies or antisera, such as polyclonal antisera and monoclonal antibodies that specifically target the protein products encoded by the marker genes, can be used to detect expression of the marker genes in an IHC assay. In some embodiments, the tissue sample is contacted with a primary antibody for a specific target for a period of time sufficient for the antibody-target binding to occur. As discussed in detail earlier, the antibodies can be detected by direct labels on the antibodies themselves, for example, radioactive labels, fluorescent labels, hapten labels such as biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. IHC protocols and kits are well known in the art and are commercially available. Automated systems for slide preparation and IHC processing are available commercially. The Leica BOND Autostainer and Leica Bond Refine Detection system is an example of such an automated system.

In some embodiments, an IHC assay is performed with an unlabeled primary antibody in conjunction with a labeled secondary antibody in an indirect assay. The indirect assay utilizes two antibodies for the detection of the protein products encoded by the marker genes in a tissue sample. First, an unconjugated primary antibody was applied to the tissue (first layer), which reacts with the target antigen in the tissue sample. Next, an enzyme-labeled secondary antibody is applied, which specifically recognize the antibody isotype of the primary antibody (second layer). The secondary antibody reacts with the primary antibody, followed by substrate-chromogen application. The second-layer antibody can be labeled with an enzyme such as a peroxidase, which reacts with the chromogen 3, 3′-diaminobenzidine (DAB) to produce brown precipitate at the reaction site. This method is sensitive and versatile due to the potential signal amplification through a signal amplification system.

In certain embodiments to increase the sensitivity of the detection, a signal amplification system can be used. “A signal amplification system”, as used herein, means a system of reagents and methods that can be used to increase the signal from detecting the bound primary or the secondary antibody. A signal amplification system increases the sensitivity of the target protein detection, increases the detected signal, and decreases the lower boundary of the detection limits. There are several types of signal amplification systems including an enzyme labeling system and macrolabeling system. These systems/approaches are not mutually exclusive and can be used in combination for additive effect.

Macrolabels or macrolabeling system are collections of labels numbering in the tens (e.g. phycobiliproteins) to millions (e.g. fluorescent microspheres) attached to or incorporated in a common scaffold. The scaffold can be coupled to a target-specific affinity reagent such as an antibody, and the incorporated labels are thereby collectively associated with the target upon binding. The labels in the macrolabels can be any of the labels described herein such as fluorophores, haptens, enzymes, and/or radioisotopes. In one embodiment of the signal amplification system, a labeled chain polymer-conjugated secondary antibody was used. The polymer technology utilized an HRP enzyme-labeled inert “spine” molecule of dextran to which 1, 2, 3, 4, 5, 6, 7, 8, 9 10, 15, 20, 25, 30, 50 or more molecules of secondary antibodies can be attached, making the system even more sensitive.

Signal amplification system based on an enzyme labeling system utilizes the catalytic activity of enzymes, such as horseradish peroxidase (HRP) or alkaline phosphatase to generate high-density labeling of a target protein or nucleic acid sequence in situ. In one embodiment, tyramide can be used to increase the signal of HRP. In such a system, HRP enzymatically converts the labeled tyramide derivative into highly reactive, short-lived tyramide radicals. The labeled active tyramide radicals then covalently couple to residues (principally the phenol moiety of protein tyrosine residues) in the vicinity of the HRP-antibody-target interaction site, resulting amplification of the number of labels at the site with minimal diffusion-related loss of signal localization. Consequently, the signal can be amplified 1, 2, 3, 4, 5, 6, 7, 8, 9 10, 15, 20, 25, 30, 50, 75, or 100 folds. As known to a person skilled in the art, the labels on the tyramide can be any labels described herein, including fluorophores, enzymes, haptens, radioisotopes, and/or photophores. Other enzyme-based reactions can be utilized to create signal amplification as well. For example, Enzyme-Labeled Fluorescence (ELF) signal amplification is available for alkaline phosphatase, wherein the alkaline phosphatase enzymatically cleaves a weakly blue-fluorescent substrate (ELF 97 phosphate) and converts it into a bright yellow-green-fluorescent precipitate that exhibits an unusually large Stokes shift and excellent photostability. Both tyramide-based signal amplification system and ELF signal amplification are available commercially, for example from ThermoFisher Scientific (Waltham, MA USA 02451).

Thus in some embodiments of the methods provided herein, the expression level of the marker genes is detected with IHC using a signal amplification system. In some embodiments, the specimen is then counterstained to identify cellular and subcellular elements.

In some embodiments, the expression level of the protein products encoded by the marker genes can also be detected with antibodies against the protein products encoded by the marker genes using an immunoblotting assay. In some embodiments of an immunoblotting assay, proteins are often (but do not have to be) separated by electrophoresis and transferred onto membranes (usually nitrocellulose or PVDF membrane). Similar to the IHC assays, primary antibodies or antisera, such as polyclonal antisera and monoclonal antibodies that specifically target the protein products encoded by the marker genes, can be used to detect expression of the marker genes. In some embodiments, the membrane is contacted with a primary antibody for a specific target for a period of time sufficient for the antibody-antigen binding to occur and the bound antibodies can be detected by direct labels on the primary antibodies themselves, e.g. with radioactive labels, fluorescent labels, hapten labels such as biotin, or enzymes such as horseradish peroxidase or alkaline phosphatase. In other embodiments, unlabeled primary antibody is used in an indirect assay as described above in conjunction with a labeled secondary antibody specific for the primary antibody. As described herein, the secondary antibodies can be labeled, for example, with enzymes or other detectable labels such as fluorescent labels, luminescent labels, colorimetric labels, or radioisotopes. Immunoblotting protocols and kits are well known in the art and are commercially available. Automated systems for immunoblotting, e.g. iBind Western Systems for Western blotting (ThermoFisher, Waltham, MA USA 02451), are available commercially. Immunoblotting includes, but is not limited to, Western blot, in-cell Western blot, and dot blot. Dot blot is a simplified procedure in which protein samples are not separated by electrophoresis but are spotted directly onto a membrane. In cell Western blot involves seeding cells in microtiter plates, fixing/permeabilizing the cells, and subsequent detection with a primary labeled primary antibody or unlabelled primary antibody followed by labeled secondary antibody as described herein.

In other embodiments, the expression levels of the protein products encoded by the marker genes can also be detected with the antibodies described herein in a flow cytometry assay, including a fluorescence-activated cell sorting (FACS) assay. Similar to the IHC or immunoblotting assays, primary antibodies or antisera, such as polyclonal antisera and monoclonal antibodies that specifically target the protein products encoded by the marker genes, can be used to detect protein expression in a FACS assay. In some embodiments, cells are stained with primary antibodies against specific target protein for a period of time sufficient for the antibody-antigen binding to occur and the bound antibodies can be detected by direct labels on the primary antibodies, for example, fluorescent labels or hapten labels such as biotin on the primary antibodies. In other embodiments, unlabeled primary antibody is used in an indirect assay as described above in conjunction with a fluorescently labeled secondary antibody specific for the primary antibody. FACS provides a method for sorting or analyzing a mixture of fluorescently labeled biological cells, one cell at a time, based upon the specific light scattering and fluorescent characteristics of each cell. The flow cytometer thus detects and reports the intensity of the fluorichrome-tagged antibody, which indicates the expression level of the target protein. Therefore, the expression level of the protein products encoded by the marker genes can be detected using antibodies against such protein products. Non-fluorescent cytoplasmic proteins can also be observed by staining permeablized cells. Methods for performing FACS staining and analyses are well known to a person skilled in the art and are described by Teresa S. Hawley and Robert G. Hawley in Flow Cytometry Protocols, Humana Press, 2011 (ISBN 1617379506, 9781617379505).

In other embodiments, the expression levels of the protein products encoded by the marker genes can also be detected using immunoassays such as an Enzyme Immune Assay (EIA) or an ELISA. Both EIA and ELISA assays are known in the art, e.g. for assaying a wide variety of tissues and samples, including blood, plasma, serum or bone marrow. A wide range of ELISA assay formats are available, see, e.g., U.S. Pat. Nos. 4,016,043, 4,424,279, and 4,018,653, which are hereby incorporated by reference in their entireties. These include both single-site and two-site or “sandwich” assays of the non-competitive types, as well as in the traditional competitive binding assays. These assays also include direct binding of a labeled antibody to a target protein. Sandwich assays are commonly used assay format. A number of variations of the sandwich assay technique exist. For example, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results can either be qualitative, by simple observation of the visible signal, or can be quantitated by comparing with a control sample containing known amounts of target protein.

In some embodiments of the EIA or ELISA assays, an enzyme is conjugated to the second antibody. In other embodiments, fluorescently labeled secondary antibodies can be used in lieu of the enzyme-labeled secondary antibody to produce a detectable signal an ELISA assay format. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA and ELISA, the fluorescent labeled antibody is allowed to bind to the first antibody-target protein complex. After washing off the unbound reagent, the remaining tertiary complex is then exposed to the light of the appropriate wavelength; the fluorescence observed indicates the presence of the target protein of interest. Immunofluorescence and EIA techniques are both very well established in the art and are disclosed herein.

For the immunoassays described herein, any of a number of enzymes or non-enzyme labels can be utilized so long as the enzymatic activity or non-enzyme label, respectively, can be detected. The enzyme thereby produces a detectable signal, which can be utilized to detect a target protein. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galatoside 6-phosphogalactohydrolase, α-gluosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods of the present disclosure.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for α-glucosidase; and the like. Exemplary fluorogenic substrates include, but are not limited to, 4-(Trifluoromethyl)umbelliferyl phosphate for alkaline phosphatase; 4-Methylumbelliferyl phosphate bis(2-amino-2-methyl-1,3-propanediol), 4-Methylumbelliferyl phosphate bis (cyclohexylammonium) and 4-Methylumbelliferyl phosphate for phosphatases; QuantaBlu™ and QuantaRed™ for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

In some embodiments of the immunoassays, a detectable label can be directly coupled to either the primary antibody or the secondary antibody that detects the unlabeled primary antibody can have. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof, napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene Oreg.); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof, CyDye™ fluorescent dyes (Amersham/GE Healthcare Life Sciences; Piscataway NJ), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

Methods well known to a person skilled in the art such as microscopy or spectroscopy can be utilized to visualize chromogenic or fluorescent detectable signals associated with the bound primary or secondary antibodies.

The methods provided in this Section (Section 5.7) can be used with various cancer models known in the art. In one embodiment, mouse xenograft cancer models are used. Briefly, T-24 and UM-UC-3 cells are purchased from ATCC and cultured using the recommended media conditions. The T-24 hNectin-4 (human nectin-4) and the UM-UC-3 Nectin-4 cells are generated by transducing parental cells with lentivirus containing the human Nectin-4 using the pRCDCMEP-CMV-hNectin-4 EF1-Puro construct and selected using puromycin. The T-24 Nectin-4 (clone 1A9) cells are implanted into nude mice and passaged via trocar, allowed to reach approximately 200 mm3 tumor volume, and subsequently treated with a single intraperitoneal (IP) dose of enfortumab vedotin (3 mg/kg) or non-binding ADC (3 mg/kg) with 7 animals per treatment group. Follow-up ICD studies with this model involve collecting tumors 5 days post treatment for downstream analysis by RNA-seq, flow, immunohistochemistry (IHC), and Luminex. Tumors are fixed in formalin and prepared as FFPE tissue blocks. Blocks are cut at 4 μm and immunohistochemistry is performed using F4/80, CD11c. The immunohistochemically stained slides sections are scanned with a Leica AT2 digital whole slide scanner, and the images are analyzed with Visiopharm software by use of custom-made algorithms for Nectin 4, CD11c and F4/80 staining. The algorithms are optimized on the basis of staining intensity and background staining. Percent positive staining is calculated for Nectin 4 and positive cells per mm2 is calculated for F480 and CD11c.

Sections of tumor are lysed in Cell Lysis Buffer 2 (R&D Systems®, Catalog #895347). The cytokines and chemokines from the tumor samples are measured using the MILLIPLEX MAP mouse cytokine/chemokine magnetic bead panel (Millipore) and read on the LUMINEX MAGPIX system.

For the RNA-seq analysis RNA from flash frozen tumors is isolated using the TRIZOL Plus RNA Purification Kit (Life Technologies) according to the manufacturer's protocol yielding high quality RNA (average RNA integrity number>8). RNA selection method is using Poly(A) selection and the mRNA Library Prep Kit from Illumina and read on the Hi-Seq 2×150 bp, single index (Illumina). The sequence reads are mapped to the human and mouse transcriptome and total reads per million were determined.

The disclosure is generally provided using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

Particular embodiments of this disclosure are described herein, including the best mode known to the inventors for carrying out the disclosure. Upon reading the foregoing description, variations of the disclosed embodiments can become apparent to individuals working in the art, and it is expected that those skilled artisans can employ such variations as appropriate. Accordingly, it is intended that the disclosure be practiced otherwise than as specifically described herein, and that the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All publications, patent applications, accession numbers, and other references cited in this specification are herein incorporated by reference in their entireties as if each individual publication or patent application were specifically and individually indicated to be incorporated by reference.

A number of embodiments of the disclosure have been described. Nevertheless, it will be understood that various modifications can be made without departing from the spirit and scope of the disclosure.

### 6. EXAMPLES

### 6.1 Example 1—Efficacy and Safety Study of Intravesical Administration of Enfortumab Vedotin in Animal Models

### 6.1.1 Efficacy Study in a Nectin-4⁺ Bladder Orthotopic Xenograft Mouse Model

This study aimed to measure the efficacy of intravesical administration of enfortumab vedotin (EV) in a Nectin-4⁺ bladder orthotopic xenograft mouse model. Specifically, and without being limited by any particular mechanism of action, local administration of EV via intravesical administration may enable direct exposure of EV to NMIBC cells, with reduced systemic exposure and an improved safety profile compared to systemic administration of EV.

Table 6 shows dose selection for preclinical experiments in mice and rats. A mouse orthotopic model was developed in SCID mice utilizing the human urothelial bladder cancer cell line, UM-UC-3, that was engineered to express human Nectin-4 and luciferase (i.e., UM-UC-3-hNectin4+-Luc+). Safety evaluations were performed in rats since EV binds with comparable affinity to rat and human Nectin-4 orthologs.

TABLE 6
Dose Selection

| | | Dose | Dose | Dose/Bladder |
|---|---|---|---|---|
| | Total Dose | Concentration | Level^{a} | Surface Area^{b} |
| | (mg) | (mg/mL) | (mg/kg) | (mg/cm²) |
| Mouse | 0.75 | 15 | 30 | 0.5 |
| Rat | 2 | 5 | 10 | 0.4 |
| | 6 | 15 | 30 | 1.2 |
| | 12 | 30 | 60 | 2.5 |
| | 20 | 50 | 100 | 4.1 |
| Human | 125 | | | 0.4 |
| (approved total | | | | |
| IV dose) | | | | |

^{a}Assume 78 kg patient (mean body weight of population pharmacokinetic EV model population), 200 g rat, 25 g mouse.
^{b}Bladder surface area derived from the volume of bladder where V = (4/3)πr³ and SA = 4πr² (Andersson KE, et al. Physiol Rev. 2004; 84:935-986).
Note:
Dose scaling body surface area is not appropriate due to limited systemic exposure and local administration dose route; instead, total dose has been normalized to bladder tissue surface area to ensure relevent preclinical doses (U.S. Food and Drug Administration. Estimating the Maximum Safe Starting Dose in Initial Clinical Trials for Therapeutics in Adult History Volunteers 2005. www.fda.gov/media/72309/download. Accessed Mar. 1, 2022).

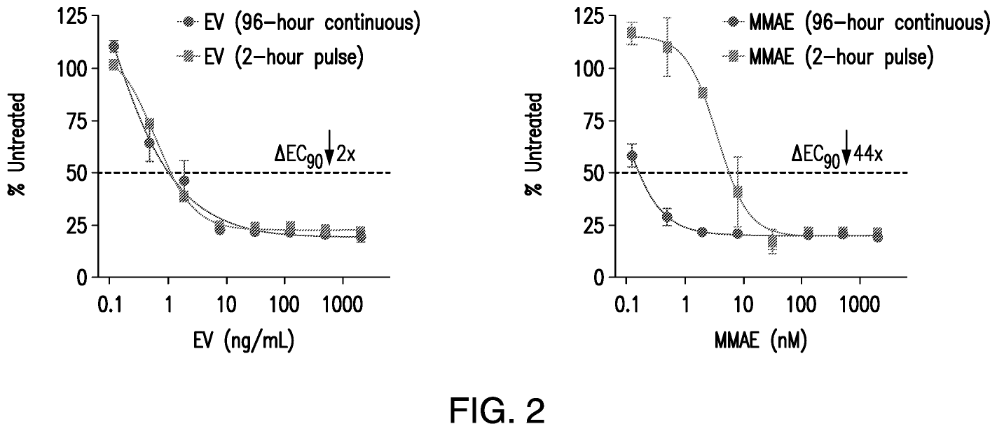

Nectin-4 is highly expressed across all stages of bladder cancer, including non-muscle invasive bladder cancer (NMIBC) and muscle invasive bladder cancer (MIBC) (data not shown). To assess cytotoxic activity of EV in vitro under conditions that mimic intravesical dosing, Nectin-4 overexpressing bladder carcinoma cells (UM-UC-3-hNectin-4⁺) were exposed to either EV, unconjugated anti-Nectin-4 antibody, unconjugated MMAE, or control ADC (FIG. 2). IV dosing was modeled by exposing the cells for 96 hours. Intravesical dosing was modeled with exposures of 2 and 24 hours followed by test article wash out. Cell death was measured using Cell TiterGlo® (Promega Corporation, Madison, WI, USA). Decreasing the exposure from 96 to 2 hours decreased potency (EC₉₀) by 44-fold for unconjugated MMAE but was almost unchanged for EV (2-fold). Control ADC and unconjugated anti-Nectin-4 did not result in sufficient activity to determine an EC₉₀ (data not shown). Thus, EV maintains cytotoxic activity in vitro using conditions that mimic intravesical dosing (FIG. 2).

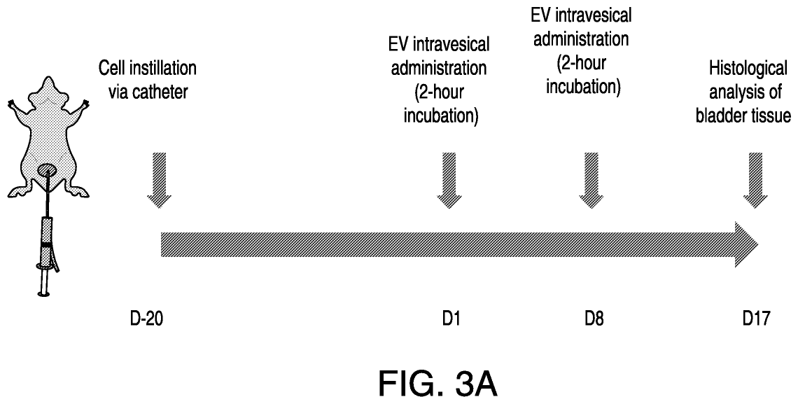

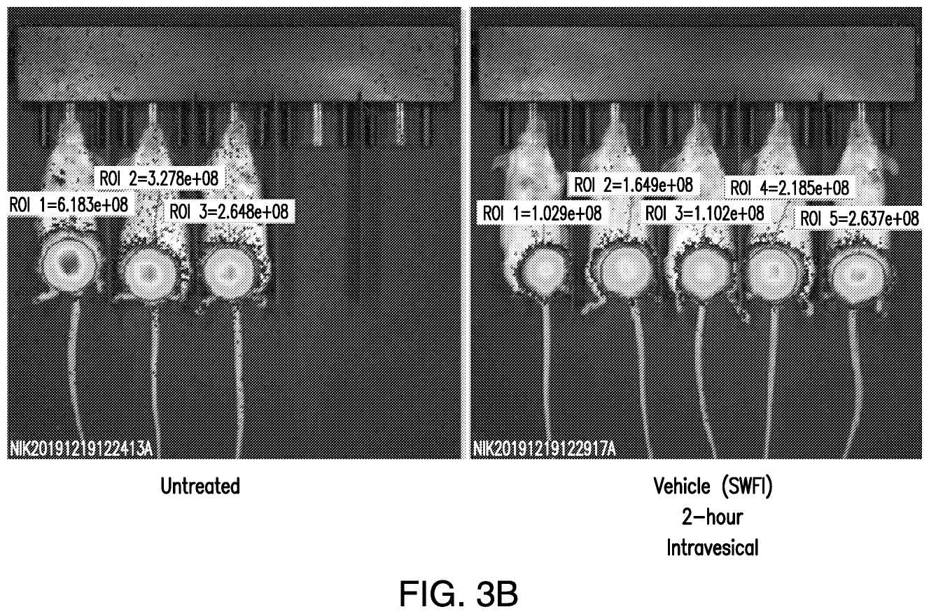

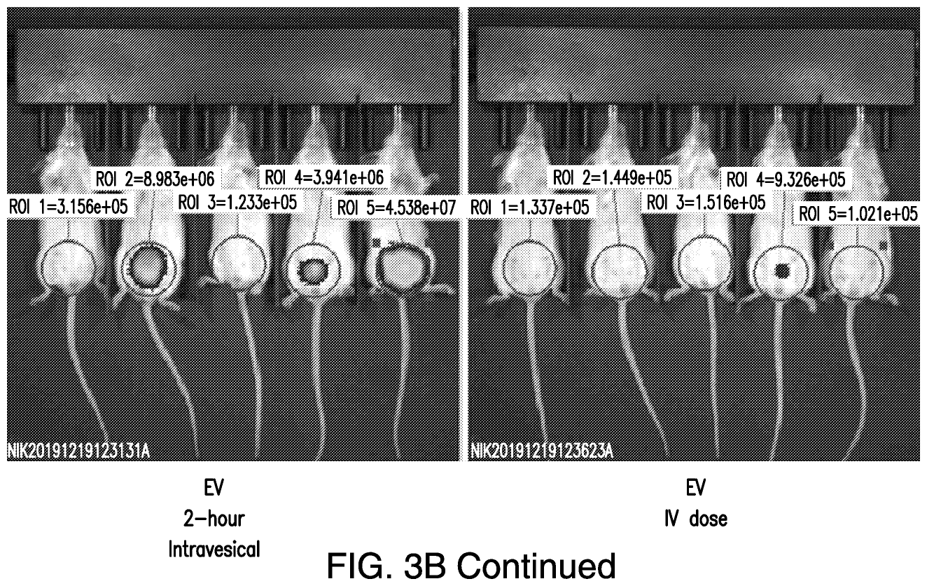

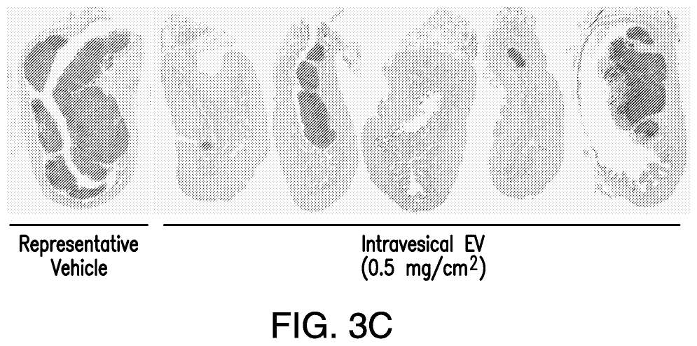

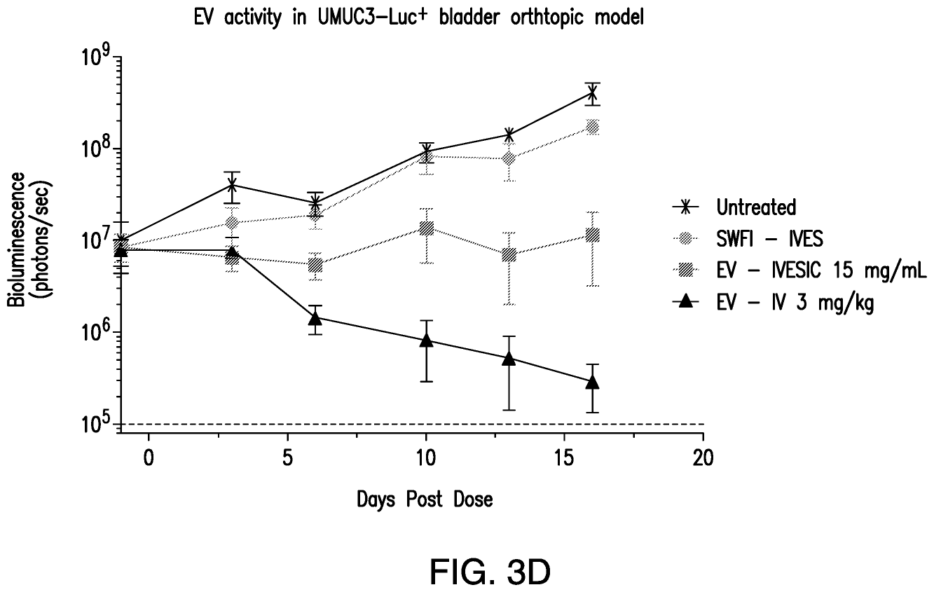

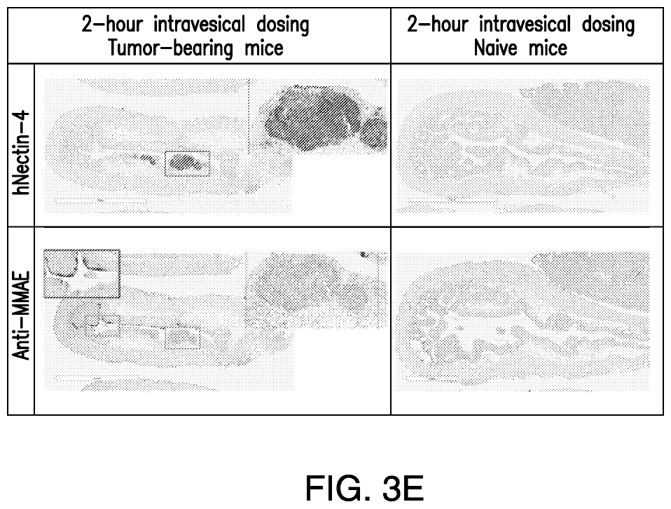

To construct a Nectin-4⁺ bladder orthotopic xenograft mouse model, luciferase-transduced, UM-UC-3-hNectin4+-Luc+, bladder cancer cells were instilled transurethrally via catheter into SCID-beige mice (FIG. 3A). In total 18 mice were used in this study: 3 mice were untreated (FIG. 3B, far left panel entitled “Untreated”); 5 mice were treated once weekly for two weeks with 2-hour intravesical administration of sterile water (FIG. 3B, second panel from left entitled “Vehicle (SWFI) 2-hour Intravesical”); 5 mice were treated once weekly for two weeks with 2-hour intravesical administration of 0.75 mg EV (0.05 mL of EV at 15 mg/mL (note: a dose concentration of 15 mg/ml in mice corresponds to a dose/bladder surface area of 0.5 mg/cm² in mice) (FIG. 3B, third panel from left entitled “EV 2 hour Intravesical”); 5 mice were treated once weekly for two weeks with intravenous administration of 0.75 mg EV (0.25 mL of EV at 3 mg/mL) (FIG. 3B, far right panel entitled “EV IV dose”). The mice were imaged to measure the efficacy of EV. In addition, blood was collected from each of the treated mice 24 hours after the EV administration for pharmacokinetic analysis. Methods involved herein were well-known to those skilled in the art.

As shown in the bioluminescence imaging results in FIG. 3B, the bladder tumors grew large in untreated mice and in mice treated with 2-hour intravesical administration of sterile water (FIG. 3B, far left panel entitled “Untreated” and second panel from left entitled “Vehicle (SWFI) 2-hour Intravesical”). In contrast, the bladder tumors substantially regressed in mice treated with 2-hour intravesical administration of EV, as well as in mice treated with intravenous administration of EV (FIG. 3B, third panel from left entitled “EV 2 hour Intravesical” and far right panel entitled “EV IV dose”). The anti-Nectin-4 immunohistochemistry results shown in FIG. 3C also indicate that the bladder tumors regressed in mice treated with 2-hour intravesical administration of EV. The bladder tissue in the five right panels in FIG. 3C were from the five mice treated with intravesical doses of EV in FIG. 3B, respectively. In addition, the bioluminescence imaging results in FIG. 3B were quantified and summarized in FIG. 3D and Table 7. In mice treated with 2-hour dwell time intravesical administration of EV, tumor growth inhibition (TGI) was 97.1% on day 17 (as measured by analyzing total flux units (photons/second) compared to control) (FIG. 3B, third panel from left entitled “EV 2 hour Intravesical,” FIG. 3D, and Table 7).

TABLE 7
Tumor Growth Inhibition of EV in NMIBC Orthotopic Model

| Treatment | Group | Dose Level | Route | % TGI |
|---|---|---|---|---|
| Untreated | 1 | N/A | N/A | 0.0 |
| Vehicle | 2 | N/A | Intravesical | 57.4 |
| AGS-22C3E | 3 | 15 mg/mL | Intravesical | 97.1 |
| AGS-22C3E | 4 | 3 mg/kg | IV | 99.9 |

Tumor growth inhibition was calculated from the mean tumor bioluminescence signal in comparison to the untreated control group at the end of the study (16 days post-initiation). The intravesical dose of 15 mg/mL was a total dose of 750 μg and represented a dose of 0.5 mg/cm² of bladder surface area. The total dose of EV delivered intravenously was approximately 10-fold lower, 75 μg per 25-gram mouse.

In addition, immunohistochemistry (IHC) staining was performed to assess localization of Nectin-4 and MMAE in bladder tumor tissue (FIG. 3E). In particular, six (6) hours following the first EV dose, tumors were collected and stained for Nectin-4 and anti-MMAE, as shown in FIG. 3E. The IHC analysis showed the presence of Nectin-4 in bladder tumor tissue with co-localization of drug, as detected by a biotin conjugated anti-MMAE primary antibody.

The above results confirm tumor (NMIBC) engraftment and intravesicular EV activity in the Nectin-4⁺ bladder orthotopic xenograft mouse model used herein.

### 6.1.2 Safety Study in Spraque-Dawley Rats

This study aimed to characterize the effects on local tissues, plasma exposures, and tissue drug levels in Sprague-Dawley rats following a single intravesical dose of enfortumab vedotin (EV) at varied concentrations and administration volumes at up to the maximum feasible concentration for reconstituted EV.

Single doses of 0.1, 0.2, or 0.4 mL of EV at 15, 30, or 50 mg/mL were intravesically administered to female Sprague-Dawley rats for two hours according to the study design shown in Table 8.

TABLE 8
Study Design

| | | | | | | | Euthanasia |
|---|---|---|---|---|---|---|---|
| | | Dose | Dose | Total | # of Rats (F) | | Timepoints |
| | Treatment | Concentration | Volume | Dose | 24 | 168 | (hours post- |
| Group | (Single Dose) | (mg/mL) | (mL) | (mg) | hr | hr | dose) |
| 1 | Control | 0 | 0.4 | 0 | 3 | 3 | 24 hrs |
| 2 | Formulation Buffer | 0 | 0.4 | 0 | 3 | 3 | (n = 3/ |
| 3 | Enfortumab vedotin | 15 | 0.1 | 1.5 | 3 | 3 | group); |
| 4 | Enfortumab vedotin | 15 | 0.2 | 3 | 4 | 3 | 168 hrs |
| 5 | Enfortumab vedotin | 15 | 0.4 | 6 | 3 | 3 | (n = 3/ |
| 6 | Enfortumab vedotin | 30 | 0.1 | 3 | 3 | 3 | group) |
| 7 | Enfortumab vedotin | 30 | 0.2 | 6 | 3 | 3 | |
| 8 | Enfortumab vedotin | 30 | 0.4 | 12 | 3 | 3 | |
| 9 | Enfortumab vedotin | 50 | 0.12 | 6 | 3 | 3 | |
| 10 | Enfortumab vedotin | 50 | 0.24 | 12 | 3 | 3 | |
| 11 | Enfortumab vedotin | 50 | 0.4 | 20 | 3 | 3 | |

To determine concentrations of ADC and MMAE in local tissues (kidney, bladder, ureter, and urethra), these tissues were collected from each rat 24 hours after the EV administration for bioanalysis and anti-MMAE immunohistochemistry (IHC) analysis. To determine serum concentrations of ADC and MMAE, 300 μL of blood were collected from each rat at different time points (1 hour, 6 hours, 24 hours, 72 hours, and 168 hours after the EV administration) for bioanalysis. Methods involved herein were well-known to those skilled in the art.

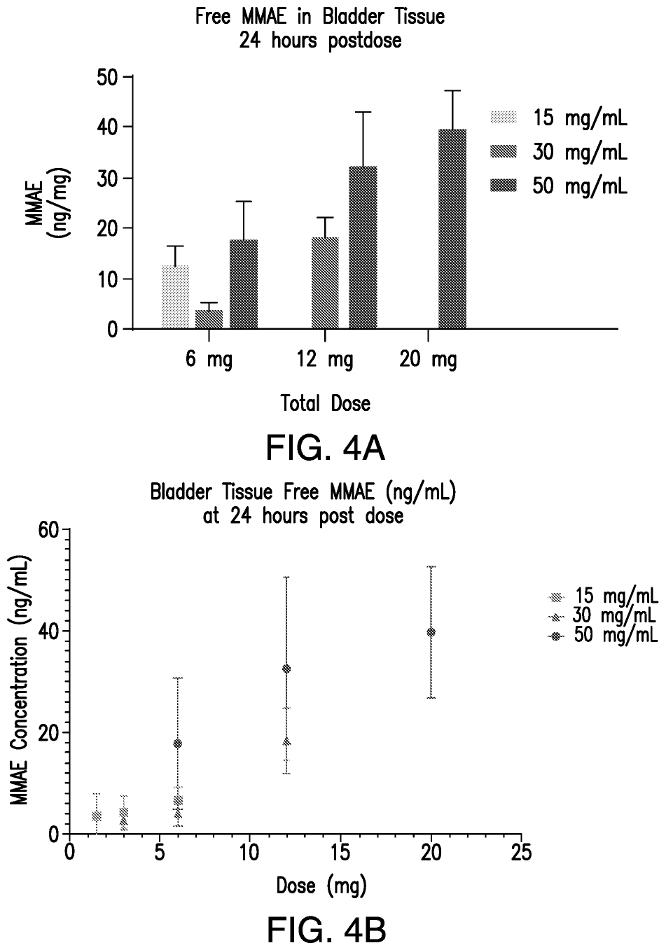

As shown in the bioanalysis results in FIGS. 4A and 4B and anti-MMAE IHC results in Table 9, a higher dose concentration and total dose, more than larger volume of instillation, drove higher MMAE levels in the bladder tissue. Specifically, FIG. 4A shows single intravesical doses of EV that were administered to female rats at doses of 0.3-4.1 mg/cm² at 15-50 mg/mL with a dwell time of 2 hours, representing doses of up to 100 mg/kg on a mass basis. Bladder levels of total MMAE at 24 hours were normalized to bladder tissue weight. MMAE was detected in bladder tissue for up to 7 days with peak concentrations within 24 hours of dose administration. In addition, MMAE was generally undetectable with minimal ADC detected in the serum (<1 ng/mL). There was no meaningful difference in total MMAE levels when concentration was held constant and dwell time varied from 30-120 minutes (data not shown). Thus, delivering a higher total dose and concentration of EV will enhance activity and increase tissue drug levels more so than change to volume instilled or dwell time. The above results indicate that single intravesical doses of 0.1, 0.2, or 0.4 mL of enfortumab vedotin at 15, 30, or 50 mg/mL to female Sprague-Dawley rats were well tolerated. Therefore, intravesical administration of EV offered an effective local administration of EV with limited systemic exposure in Sprague-Dawley rats.

TABLE 9
anti-MMAE IHC in Local Tissues

| Treatment: | | | | | | | | | | | | | | | | |
|---|---|---|---|---|---|---|---|---|---|---|---|---|---|---|---|---|
| water | | | vehicle | | | enfortumab vedotin | | | | | | | | | | |
| total dose (mg): | | | | | | | | | | | | | | | | |
| 0 | | | 0 | | | 1.5 | | | 3 | | | 6 | | | 3 | |
| concentration (mg/ml): | | | | | | | | | | | | | | | | |
| 0 | | | 0 | | | 15 | | | 15 | | | 15 | | | 30 | |
| volume (ml): | | | | | | | | | | | | | | | | |
| 0.4 | | | 0.4 | | | 0.1 | | | 0.2 | | | 0.4 | | | 0.1 | |
| Animal #: | | | | | | | | | | | | | | | | |
| 151 | 152 | 153 | 251 | 252 | 253 | 351 | 352 | 353 | 452 | 453 | 457 | 551 | 552 | 553 | 651 | 652 |

IHC score - transitional cell staining

| | | | | | | | | | | | | | | | | | |
|---|---|---|---|---|---|---|---|---|---|---|---|---|---|---|---|---|---|
| URINARY | — | — | — | — | — | — | 2 | — | — | 1 | — | — | 1 | 3 | 2 | — | 2 |
| BLADDER | | | | | | | | | | | | | | | | | |
| KIDNEY | — | — | — | — | — | — | — | — | — | — | — | — | — | — | — | — | — |
| URETER | — | — | — | — | — | — | — | — | — | — | — | — | 1 | 1 | — | — | — |
| URETHRA | — | — | — | — | — | — | 1 | 1 | — | — | — | 1 | 1 | 1 | 1 | — | — |

| | Treatment: | | | | | | | | | | | | | | | |
|---|---|---|---|---|---|---|---|---|---|---|---|---|---|---|---|---|
| | enfortumab vedotin | | | | | | | | | | | | | | | |
| | total dose (mg): | | | | | | | | | | | | | | | |
| | 3 | 6 | | | 12 | | | 6 | | | 12 | | | 20 | | |
| | concentration (mg/ml): | | | | | | | | | | | | | | | |
| | 30 | 30 | | | 30 | | | 50 | | | 50 | | | 50 | | |
| | volume (ml): | | | | | | | | | | | | | | | |
| | 0.1 | 0.2 | | | 0.4 | | | 0.12 | | | 0.24 | | | 0.4 | | |
| | Animal #: | | | | | | | | | | | | | | | |
| | 653 | 751 | 752 | 753 | 851 | 852 | 853 | 951 | 952 | 953 | ### | ### | ### | ### | ### | ### |
| IHC score - transitional cell staining | | | | | | | | | | | | | | | | |
| URINARY | — | 1 | — | 4 | 4 | 4 | 4 | 2 | 2 | — | 4 | 3 | 4 | 4 | 4 | 3 |
| BLADDER | | | | | | | | | | | | | | | | |
| KIDNEY | — | — | — | — | — | — | — | — | — | — | 1 | — | — | — | — | — |
| URETER | — | 4 | — | 4 | 4 | 4 | 3 | 2 | 1 | — | 3 | 3 | 4 | 3 | 4 | — |
| URETHRA | — | 2 | — | 4 | 4 | 3 | 3 | 2 | 3 | — | 3 | 4 | 4 | 3 | 4 | 1 |

IHC score:
— = <1% transitional cells staining positive
1 = 1-25% positive
2 = 26-50% positive
3 = 50-75% positive
4 = >75% positive

To evaluate the potential for toxicity and characterize the pharmacokinetics of intravesical EV, six (6) weekly intravesical doses of EV or control were administered to female rats. As shown in Table 10, there were no microscopic findings in previously identified target tissues, including the skin and bone marrow. The dose corresponding to the no-effect level of EV is equivalent to >20-fold the approved human IV dose.

TABLE 10
Dose Toxicity Study

| | Clinical Signs, | | |
|---|---|---|---|
| | Clinical Pathology, | | |
| | Organ Weights, | | |
| Weekly | Mortality, | | |
| Intravesical | Macroscopic | Microscopic | |
| Dose Level | Findings | Findings | Conclusions |
| 0.1 mg/cm² | No EV-related | No EV-related | — |
| (3 mg/kg) | findings | findings | |
| 0.4 mg/cm² | No EV-related | No EV-related | No-observed- |
| (10 mg/kg) | findings | findings | effect- level |
| | | | (NOEL) |
| 1.2 mg/cm² | No EV-related | Minimal/Slight | No-observed- |
| (30 mg/kg) | findings | mitotic/apoptotic | adverse-effect-level |
| | | figures in the | (NOAEL) |
| | | transitional | |
| | | epithelium of the | |
| | | kidney and bladder | |

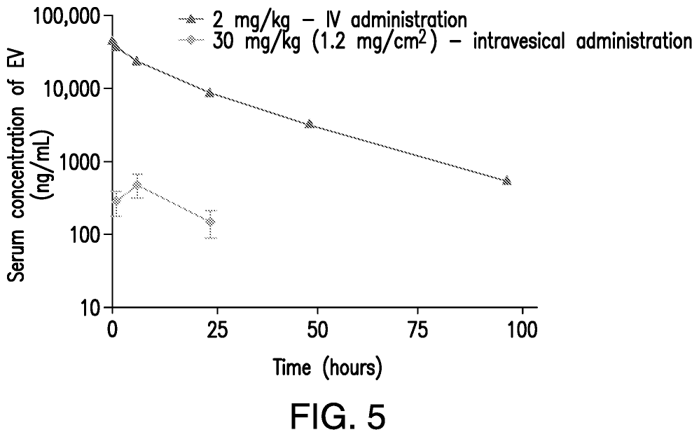

The serum concentration of EV administered intravesically was determined following the first dose of EV using a validated ELISA-based assay (mean±SEM) (FIG. 5). Mean EV Cₘₐₓ was ≤750 ng/mL (i.e., >35-fold lower than the IV Cₘₐₓ of the clinically approved dose) and there was no detectable serum MMAE by the validated mass spectrometry assay. Estimation of the serum area under the time-concentration curve was limited to only the highest EV dose (30 mg/kg) only detectable up to 24 hours post-instillation. Serum concentrations of unconjugated MMAE were below the lower limit of quantitation (<10 μg/mL). Thus, intravesical administration of EV limits systemic exposure, with low and transient systemic absorption.

Therefore, Table 9 and FIG. 5 show that intravesical administration of EV was well tolerated with no detectable local or systemic toxicities, and low and transient systemic absorption. Without being limited by any particular mechanism of action, these data also indicate that low systemic absorption of intravesical EV may lower the incidence of most common adverse events observed with systemically administered EV.

### 6.1.3 Effects of Length of Dwell Time in Spraque-Dawley Rats

This study aimed to determine MMAE levels in bladder tissue in Sprague-Dawley rats following intravesical administration of enfortumab vedotin (EV) for different lengths of dwell time.

Specifically, 24 female Sprague-Dawley rats were randomly grouped into 4 groups (6 rats per group). A single dose of 0.4 mL of EV at 30 mg/mL was intravesically administered to each rat for 30, 60, 90, or 120 minutes. Bladder tissue was collected from each rat 24 hours after the EV administration for bioanalysis to determine concentrations of MMAE in bladder tissues. Methods involved herein were well-known to those skilled in the art.

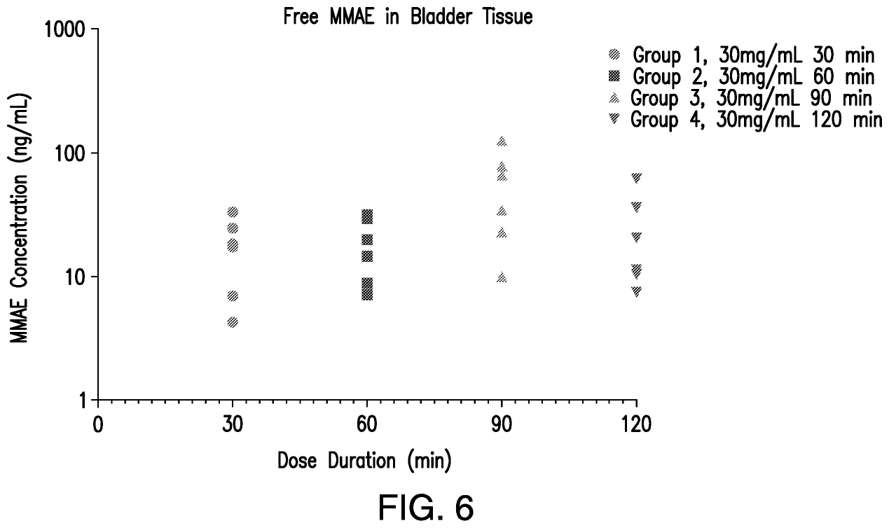

As shown in FIG. 6, there was no substantial difference in bladder tissue MMAE levels in rats intravesically administered EV for different lengths of dwell time ranging from 30 minutes to 120 minutes. In the presence of factors including a mucous layer that can potentially inhibit access of an ADC to targeted cells and control diffusion to a greater degree than with traditional small molecules, such in vivo result is somewhat surprising. Without being bound by theory, it appears that a relatively short dwell time may be sufficient for bladder tissue MMAE to reach saturation in rats.

### 6.2 Hypothetical Example 2—A Phase 1, Open-Label, Multicenter, Dose-Escalation, and Dose-Expansion Study Designed to Evaluate the Safety, Tolerability, PK, and Antitumor Activity of Intravesical Enfortumab Vedotin in Adults with Non-Muscle Invasive Bladder Cancer (NMIBC)

### 6.2.1 Drug for Use in the Clinical Study

Enfortumab vedotin is a Nectin-4 targeted monoclonal antibody (AGS-22C3) covalently linked to the microtubule-disrupting agent monomethyl auristatin E (MMAE). Enfortumab vedotin has three functional subunits:
A fully human IgG1K antibody (AGS-22C3);
The microtubule-disrupting agent MMAE;
A protease-cleavable maleimidocaproyl-valine-citrulline (vc) linker that covalently attaches MMAE to AGS-22C3.

Enfortumab vedotin binds the V domain of Nectin-4 (Challita-Eid et al., Cancer Res (2016); 76(10): 3003-13.). In the presumed mechanism of action, the drug binds Nectin-4 protein on the cell surface and is internalized, causing proteolytic cleavage of the vc linker and intracellular release of MMAE. Free MMAE subsequently disrupts tubulin polymerization and leads to mitotic arrest.

### 6.2.2 Summary of the Study

### 6.2.2.1 Protocol Title

A study of intravesical enfortumab vedotin for treatment of patients with non-muscle invasive bladder cancer (NMIBC)

### 6.2.2.2 Study Objectives

### Primary

To evaluate the safety and tolerability of intravesical enfortumab vedotin in subjects with NMIBC
To identify the maximum tolerated dose (MTD) or recommended dose of intravesical enfortumab vedotin in subjects with NMIBC

### Secondary

To assess the pharmacokinetics (PK) of intravesical enfortumab vedotin
To assess the immunogenicity of intravesical enfortumab vedotin
To assess the antitumor activity of intravesical enfortumab vedotin as measured by complete response (CR) rate
To assess the duration of CR
To assess the rate of cystectomy
To assess progression-free survival (PFS)
To assess cystectomy-free survival (CFS)

### Exploratory

To assess biomarkers in relation to response, toxicity, pharmacodynamics, pharmacokinetic/pharmacodynamics (PK/PD) relationship, or resistance to enfortumab vedotin
To assess subject-reported experience and subject-reported tolerability of treatment

### 6.2.2.3 Study Population

### Inclusion Criteria

1. Subjects must have histologically confirmed, non-muscle invasive urothelial (transitional cell) carcinoma with carcinoma in situ (CIS) (with or without papillary disease). Histological confirmation should occur within 60 days prior to first dose of study treatment.
2. Predominant histologic component (>50%) must be urothelial (transitional cell) carcinoma. Pure variant histologies are excluded.
3. Subjects must have high-risk Bacillus Calmette-Guerin (BCG)-unresponsive disease, defined as:
Persistent or recurrent CIS alone or with recurrent Ta/T1 (noninvasive papillary disease/tumor invades the subepithelial connective tissue) disease within 12 months of completion of adequate BCG therapy.

Adequate BCG therapy is defined as one of the following:
5 of 6 doses of an initial induction course+at least 2 of 3 doses maintenance therapy
5 of 6 doses of an initial induction course+at least 2 of 6 doses of a second induction course

Note: Subjects who are unable to complete an adequate course or receive a reduced dose of BCG therapy due to the worldwide BCG shortage is eligible for enrollment after consultation with the medical monitor.

4. In the opinion of the investigator, subject must be ineligible for or refusing a radical cystectomy.
5. All visible papillary Ta/T1 tumors must be completely resected within 60 days prior to enrollment.Residual pure CIS is permitted.
All subjects noted to have T1 tumors should undergo an additional re-TURBT (transurethral resection of bladder tumor) prior to beginning study treatment. Re-staging TURBT must show uninvolved (no tumor in) muscularis propria.
   6. Subjects must have satisfactory bladder function and the ability to retain study drug instillation for a minimum of 1 hour, even with premedication.
   7. Age 18 years or older.
   8. An Eastern Cooperative Oncology Group (ECOG) Performance Status score of 0, 1, or 2 for conversion of performance status using Karnofsky scale, if applicable).

Subjects with ECOG performance status of 2 must additionally meet the following criteria:
Glomerular filtration rate (GFR)≥50 mL/min
May not have New York Heart Association (NYHA) Class III heart failure
   9. The following baseline laboratory data:
Absolute neutrophil count (ANC)≥1500/μL
Hemoglobin (Hgb)≥10 g/dL
Platelet count≥100,000/μL
Serum bilirubin≤1.5×upper limit of normal (ULN) or ≤3×ULN for subjects with Gilbert's disease
Calculated creatinine clearance (CrCl)≥30 mL/min (GFR can also be used in place of creatinine or CrCl). CrCl should be calculated using the Cockcroft-Gault method or Modification of Diet in Renal Disease (MDRD) equations. Subjects with an ECOG performance status of 2 must have GFR≥50 mL/min.
Alanine aminotransferase (ALT) and aspartate aminotransferase (AST)≤3×ULN
International normalized ratio (INR) or prothrombin time (PT); activated partial thromboplastin time (aPTT) or partial thromboplastin time (PTT)≤1.5 ULN unless subject is receiving anticoagulant therapy as long as PT or aPTT is within therapeutic range of intended use of anticoagulants.
   10. Estimated life expectancy>2 years.
   11. A female subject of childbearing potential is anyone born female who has experienced menarche and who has not undergone surgical sterilization (eg, hysterectomy, bilateral salpingectomy, bilateral oophorectomy) or has not completed menopause. Menopause is defined clinically as 12 months of amenorrhea in a person over age 45 in the absence of other biological, physiological, or pharmacological causes. Female subjects of childbearing potential must meet the following conditions:
Agree not to try to become pregnant during the study and for at least 6 months after the final dose of study drug.
Must have a negative serum or urine pregnancy test (minimum sensitivity 25 mIU/mL or equivalent units of beta human chorionic gonadotropin [β-hCG]) result within 3 days prior to Day 1. Female subjects with false positive results and documented verification of negative pregnancy status are eligible for participation.
If heterosexually active, must consistently use highly effective methods of birth control, with a failure rate of less than 1% starting at screening, throughout the study period, and for at least 6 months after the final dose of study drug.
Female subjects must agree not to breastfeed or donate ova, starting at screening and throughout the study period, and for at least 6 months after the final dose of study drug.
   12. A male subject who can father children is anyone born male who has testes and who has not undergone surgical sterilization (eg, vasectomy followed by a clinical test proving that the procedure was effective). Male subjects who can father children must meet the following conditions:
Must not donate sperm starting at screening and throughout the study period, and for at least 6 months after the final dose of study drug. Male subjects are informed about the negative risk to reproductive function and fertility from the study treatment. Prior to treatment male subjects should be advised to seek information on fertility preservation and sperm cryo conservation.
Must consistently use highly effective methods of birth control, with a failure rate of less than 1% starting at screening and continuing throughout the study period and for at least 6 months after the final dose of study drug.
Male subjects with a pregnant or breastfeeding partner(s) must consistently use one of 2 contraception options for preventing secondary exposure to seminal fluid for the duration of the pregnancy or time partner is breastfeeding throughout the study period and for at least 6 months after the final dose of study drug.
   13. The subject must provide written informed consent.

### Exclusion Criteria

1. Current or prior history of muscle-invasive urothelial carcinoma (ie, T2, T3, or T4 disease) or metastatic disease.
2. Nodal or metastatic disease as noted on computed tomography (CT) or magnetic resonance imaging (MRI) done within 3 months prior to the start of study treatment.
3. Concomitant upper tract urothelial carcinoma as noted on CT or MRI urogram with contrast of abdomen/pelvis performed within 3 months prior to the start of study treatment.
4. Subjects known to have prior or concomitant urothelial carcinoma of the prostatic urethra, as assessed by investigator within 6 months prior to the start of study treatment.
5. Subjects with tumor-related hydronephrosis at screening (subjects with hydronephrosis due to causes other than tumor are allowed if confirmed by the investigator).
6. Subject has received any other systemic anticancer therapy (eg, chemotherapy, biologic therapy, immunotherapy, targeted therapy, endocrine therapy, investigational agent) within 4 weeks of the first dose of study treatment or any intravesical therapy for treatment of NMIBC within 6 weeks prior to the start of study treatment, with the exception of the following:
Cytotoxic agents (eg, mitomycin C, doxorubicin, and gemcitabine) when administered as a single instillation immediately following a TURBT procedure, which is permitted between 14 and 60 days prior to the start of study treatment
   7. Subject has ongoing symptoms (Grade 2 and higher) secondary to adverse events (AEs) related to prior therapy for NMIBC.
   8. Subject has had any prior radiation to the bladder for urothelial cancer.
   9. Active infection requiring systemic (eg, oral or intravenous) antibiotics within 14 days prior to the start of study treatment. Subjects receiving prophylactic antibiotics (eg, for the prevention of a urinary tract infection [UTI] or chronic obstructive pulmonary disease) are eligible.
   10. Subjects who cannot tolerate intravesical dosing or intravesical surgical manipulation.
   11. History of another malignancy within 3 years before the first dose of study drug, or any evidence of residual disease from a previously diagnosed malignancy. Exceptions are malignancies with a negligible risk of metastasis or death (eg, 5-year overall survival [OS]≥90%), such as adequately treated CIS of the cervix, non-melanoma skin carcinoma, ductal CIS of the breast, or Stage I uterine cancer.
A history of prostate cancer (T2N0M0 or lower with Gleason score≤7) treated with definitive intent (surgically or with radiation therapy) at least 1 year prior to study entry is acceptable, provided that the subject is considered prostate cancer-free, and the following criteria are met:
   (i). Subjects who have undergone radical prostatectomy must have undetectable prostate-specificantigen (PSA) for >1 year and at screening.
   (ii). Subjects who have had radiation must have a PSA doubling time>1 year (based on at least 3 values determined>1 month apart) and a total PSA value that does not meet Phoenix criteria forbiochemical recurrence (ie, <2.0 ng/mL above nadir).
      12. Previous exposure to Nectin-4-targeted therapy or a monomethyl auristatin E (MMAE)-containing agent.
      13. Subjects with autoimmune or inflammatory skin disorders, such as psoriasis or atopic dermatitis, that have active disease requiring any treatment.
      14. Subjects with ongoing sensory or motor neuropathy Grade 2 or higher.
      15. Subjects with a positive hepatitis B surface antigen and/or antihepatitis B core antibody. Subjects with a negative polymerase chain reaction (PCR) assay are permitted with appropriate antiviral prophylaxis.
      16. Active hepatitis C infection or known human immunodeficiency virus (HIV) infection. Subjects who have been treated for hepatitis C infection are permitted if they have documented sustained virologic response of ≥12 weeks. No HIV testing is required unless mandated by local health authority.
      17. Known active tuberculosis.
      18. Subjects with uncontrolled diabetes. Uncontrolled diabetes is defined as hemoglobin A1c (HbA1c)≥8% or HbA1c 7% to <8% with associated diabetes symptoms (polyuria or polydipsia) that are not otherwise explained.
      19. Documented history of a cerebral vascular event (stroke or transient ischemic attack), unstable angina, myocardial infarction, or cardiac symptoms consistent with NYHA Class III-IV within 6 months prior to their first dose of enfortumab vedotin.
      20. Subjects who are breastfeeding, pregnant, or planning to become pregnant from time of informed consent until at least 6 months after final dose of study drug.
      21. Known severe (≥Grade 3) hypersensitivity to enfortumab vedotin or to any excipient contained in the drug formulation of enfortumab vedotin (including histidine, trehalose dihydrate, and polysorbate 20).
      22. Subjects with active keratitis or corneal ulcerations. Subjects with superficial punctate keratitis are allowed if the disorder is being adequately treated in the opinion of the investigator.
      23. Other serious underlying medical condition that, in the opinion of the investigator, would impair the subject's ability to receive or tolerate the planned treatment and follow-up.

### 6.2.2.4 Number of Planned Subjects

Approximately 58 subjects is enrolled in this study. This includes up to approximately 18 subjects to be evaluated in dose escalation and approximately 40 subjects to be evaluated in dose expansion (up to 2 cohorts of approximately 20 subjects each).

### 6.2.2.5 Study Design

This is a phase 1, open-label, multicenter, dose-escalation, and dose-expansion study designed to evaluate the safety, tolerability, PK, and antitumor activity of intravesical enfortumab vedotin in adults with NMIBC.

The study is conducted in 2 parts:

Dose escalation: Approximately 18 subjects are treated to evaluate the safety, tolerability, and systemic exposure of intravesical enfortumab vedotin to identify the MTD and/or recommended dose.

Dose expansion: Approximately 40 subjects (up to 2 cohorts of approximately 20 subjects each) are treated at the MTD or recommended dose to further characterize the safety, PK, and antitumor activity of intravesical enfortumab vedotin.

Dose escalation and dose expansion enrolls adult subjects with BCG-unresponsive NMIBC with CIS (with or without papillary disease). All subjects receive enfortumab vedotin, the investigational agent under study, via intravesical administration. Treatment on the study occurs during the induction and maintenance phases, and subjects enter a follow-up period after completion of the maintenance phase.

During the induction phase, subjects receive intravesical instillations of enfortumab vedotin once a week (q1wk) for 6 weeks. Six to 8 weeks following completion of the induction phase, subjects have their first on-study response assessment, after which they enter the maintenance phase. During the maintenance phase, subjects receive intravesical enfortumab vedotin once a month for a total of 9 doses.

After completion of the maintenance phase, subjects enter the follow-up phase of the study. Tumor response assessment via standard of care cystoscopy (ie, cystoscopy±biopsy) and cytology occur every 3 months from the first induction dose for the first 2 years after enrollment, and every 6 months thereafter for 5 years after enrollment until disease recurrence, progression, initiation of subsequent anticancer therapy, or death, whichever occurs first. After discontinuation of study treatment due to disease persistence, recurrence, progression, or initiation of subsequent anticancer therapy, subjects enter the survival follow-up, where survival and subsequent anticancer therapy data are collected every 6 months (±2 weeks) until loss to follow-up, withdrawal of consent, death, or study termination by the sponsor, whichever occurs first, for a maximum of 5 years after enrollment.

Response assessment via cystoscopy and urine cytology must be completed within 14 days prior to the next administration of study drug. Subjects with abnormal cystoscopy, positive, or abnormal urine cytology should undergo additional evaluation (eg, imaging, biopsies, exam under anesthesia) per the investigator's clinical discretion.

Bladder mapping biopsies are required at the Month 12 assessment for the study. In the absence of visible tumor, biopsies should be obtained from all quadrants of the bladder (minimum 4 biopsies required). Biopsy is not required at all other visits but will be considered when clinically indicated for consideration of efficacy.

Subjects who are found to have high-grade T1 disease with or without CIS or stage progression at any on-study evaluation, including the first on-study 3-month evaluation, are discontinued from study treatment. Subjects with persistent CIS or recurrent high-grade Ta disease at the first on-study 3-month evaluation may continue therapy after they are reconsented for continuing study treatment and be assessed again at 6 months. Beginning with the 6-month assessment, any subject with persistent or recurrent high-risk NMIBC or disease progression is discontinued from study treatment. Appearance, presence, or persistence of lower grade tumors are not considered as recurrence. Subjects with only low-grade tumors should continue treatment after resection and histological confirmation.

Subjects who discontinue study treatment due to reasons other than disease persistence, recurrence, or progression remain in the follow-up phase of the study.

### Dose Escalation

Systemic enfortumab vedotin is currently approved in the US at a dose of 1.25 mg/kg (up to a maximum dose of 125 mg) for the treatment of patients with locally advanced or metastatic urothelial cancer who have previously received a PD-1 or PD-L1 inhibitor and a platinum-containing chemotherapy. The safety profile of systemic enfortumab vedotin is well established and is also being evaluated at this dose level in multiple clinical trials. Given this established systemic dose and its safety profile, this study evaluates enfortumab vedotin starting at 125 mg administered intravesically.

The dose escalation portion of the study evaluates escalating concentrations of enfortumab vedotin at 4 planned dose levels (125, 250, 500, and 750 mg) with a 25 mL volume of instillation and a maximum 90-minute dwell time (or subject's tolerated dwell time; actual dwell time will be recorded on the appropriate electronic case report form). Up to approximately 18 subjects are enrolled to evaluate the safety, tolerability, systemic bioavailability, and to identify the MTD and/or recommended dose of intravesical enfortumab vedotin.

Dose escalation is conducted using the modified toxicity probability interval (mTPI) method. Enrollment occurs on a cohort-by-cohort basis.

Enfortumab vedotin is administered intravesically q1wk for 6 weeks during the induction phase and once a month for 9 doses during the maintenance phase at the planned doses shown in the table above. The sponsor and/or safety monitoring committee (SMC) may also recommend investigation of lower and/or intermediate dose levels. If the MTD is not reached, dose levels higher than 750 mg can be explored.

### Dose-Limiting Toxicity

Dose-limiting toxicities (DLTs) are evaluated during the dose escalation portion of the study. The DLT-evaluation period is the time from start of induction to completion of all 6 induction doses plus 1 week. A subject will be considered a DLT-evaluable (DE) subject if they either experienced a DLT or have received a minimum of 5 induction doses of enfortumab vedotin.

A DLT is defined as any of the following if assessed by the investigator to be related to treatment with intravesical enfortumab vedotin. Grading will be according to the NCI CTCAE, Version 5.0:
Occurrence of Grade 3 or higher treatment-emergent AEs of the urinary tract such as hematuria, dysuria, urinary retention, urinary frequency/urgency, or bladder spasm
Grade 3 or higher local skin or mucosal reaction in groin or perineal area
Significant hematuria leading to clot obstruction that is related to the study drug per investigator assessment
Grade 3 or higher hematological toxicity (if it is an increase of ≥2 grades from baseline), including:
   Neutropenic fever
      Grade 3 febrile neutropenia is defined as ANC<1000/mm³ with a single temperature of
      >38.3° C. (101° F.) or a sustained temperature≥38° C. (100.4° F.) for more than 1 hour
      Grade 4 febrile neutropenia is defined as ANC<1000/mm³ with a single temperature of
      >38.3° C. (101° F.) or a sustained temperature≥38° C. (100.4° F.) for more than 1 hour, with life-threatening consequences and urgent intervention indicated
   Grade 4 neutropenia or thrombocytopenia lasting>7 days
   Grade 3 thrombocytopenia with bleeding
Any other Grade 3 or higher nonhematologic AE, unless explained by an underlying medical condition, intercurrent illness, or malignancy
Unresolved treatment-related Grade 2 AE lasting>14 days
Any death not clearly due to the underlying disease or extraneous causes
Cases of Hy's law

The following nonhematological toxicities are not considered DLTs:
Grade 3 nausea/vomiting or diarrhea<72 hours with adequate antiemetic and other supportive care
Grade 3 fatigue<1 week
≥Grade 3 electrolyte or other nonhematologic laboratory abnormality that lasts<72 hours, is not clinically complicated, and resolves spontaneously or responds to conventional medical interventions
≥Grade 3 amylase or lipase that is not associated with symptoms or clinical manifestations of pancreatitis

### Dose Expansion

To further characterize the safety, tolerability, PK, and antitumor activity of intravesical enfortumab vedotin, approximately 40 additional subjects is enrolled in up to 2 dose-expansion cohorts (approximately 20 subjects per cohort). If the MTD is identified in the dose escalation portion of the study, the corresponding dose level is evaluated in dose expansion. The sponsor, in consultation with the SMC, may also evaluate more than 1 dose level in dose expansion if no MTD has been reached, or if different dose levels warrant further evaluation. The opening of these dose-expansion cohorts is determined by the sponsor in consultation with the SMC based on the cumulative safety and activity demonstrated during dose escalation.

### Safety Monitoring Committee

An SMC consisting of the site investigator(s) and representatives from the sponsor (including the study medical monitor, drug safety representative, clinical scientist, and biostatistician) will monitor subject safety and make dosing recommendations throughout dose escalation and dose expansion. The SMC may recommend further evaluation of the safety at a given dose (ie, enrolling additional subjects at a given dose) or investigation of a dose level that is lower than or intermediate to the planned dose levels. The SMC also reviews cumulative safety data to identify safety concerns that may emerge due to cumulative exposure beyond the DLT window. The SMC may also review antitumor activity data to determine whether the benefit-risk profile supports continued investigation or cessation of investigation of a dose or cohort. The SMC provides recommendations and final decisions are made by the sponsor. Further details are documented in an SMC charter.

### 6.2.2.6 Investigational Product, Dose, and Mode of Administration

Enfortumab vedotin is administered intravesically q1wk for 6 weeks during the induction phase, and once a month for 9 doses during the maintenance phase.

### 6.2.2.7 Duration of Treatment

During the induction phase, subjects receive intravesical instillations of enfortumab vedotin q1wk for 6 weeks. Six to 8 weeks following completion of the induction phase, subjects have their first on-study response assessment, after which they enter the maintenance phase. During the maintenance phase, subjects receive intravesical enfortumab vedotin once a month for a total of 9 doses.

### 6.2.2.8 112BResponse/Efficacy Assessments

Tumor response assessment on the study occur via standard of care cystoscopy (ie, cystoscopy±biopsy) and cytology at screening followed by assessments every 3 months from the first induction dose for the first 2 years after enrollment, and every 6 months thereafter for 5 years after enrollment.

Response assessment via cystoscopy and urine cytology must be completed within 14 days prior to the next administration of study drug. Subjects with abnormal cystoscopy, positive, or abnormal urine cytology should undergo additional evaluation (eg, imaging, biopsies, exam under anesthesia) per the investigator's clinical discretion.

Annual upper tract imaging is performed as clinically indicated while the subject is on study.

Bladder mapping biopsies are required at the Month 12 assessment for the study. In the absence of visible tumor, biopsies should be obtained from all quadrants of the bladder (minimum 4 biopsies required). Biopsy is not required at all other visits but are considered when clinically indicated for consideration of efficacy.

Subjects are considered to have a CR when they have all of the following findings:
1. Cystoscopy: normal appearance of bladder. In case of abnormal appearance of bladder on cystoscopy, biopsies should be negative or show low-grade Ta, any grade papillary urothelial neoplasm of low malignant potential, or any grade papilloma. If random bladder biopsies are performed, these biopsies should be negative or show low grade disease.
2. Urine cytology: negative.
   a. Urine cytology that is not conclusive should be evaluated as per protocol
   b. Positive urine cytology should be further evaluated clinically by cystoscopy f biopsy and imaging
3. Imaging (if performed): normal or, if found abnormal, findings should support a CR in the bladder.

Due to the intravesical administration of the study treatment, a subject is considered to have a CR if they have negative cystoscopy with malignant urine cytology if cancer is found in the upper tract or prostatic urethra and random bladder biopsies are negative.

Persistent disease is defined as presence of CIS disease with or without papillary disease (high-grade Ta/T1).

Recurrence is defined as the reappearance of high-grade disease (high-grade Ta, T1, or CIS) after the start of therapy. Recurrence must be confirmed by biopsy.

Progression is defined as the development of any of the following: T1 disease (lamina propria invasion), ≥T2 disease (muscle invasive), lymph node (N1+), distant metastases (M1), or an increase in grade from low to high.

Persistence, appearance, or presence of lower grade disease is not considered recurrence. Subjects with recurrent low-grade papillary disease may undergo resection and continue on study.

### 6.2.2.9 Pharmacokinetic and Immunogenicity Assessments

Blood samples for PK and antitherapeutic antibody (ATA) analyses and urine samples for PK analyses are collected at protocol-defined time points. Dose-related blood PK parameters for enfortumab vedotin to be estimated may include, but are not limited to, area under the concentration-time curve (AUC), maximum concentration (Cₘₐₓ), time to maximum concentration (Tₘₐₓ), apparent terminal half-life (t½), and trough concentration (C_{trough}). Additional analytes are evaluated as necessary.

### 6.2.2.10 Pharmacodynamic and Biomarker Assessments

### Biomarkers in Tumor Tissue

Archival tumor tissue that was collected within 12 months of enrollment is required for all subjects, if available (the most recently available tissue should be used). If freshly cut slides only can be provided, a minimum of 10 to 15 sections is required (if less than 10, contact the sponsor). Biopsy is required at the Month 12 assessment and should be performed as clinically indicated at all other assessments. Tissue from biopsies collected while on treatment is used for biomarker assessments.

To understand the relationship between the biological characteristics of tumors before treatment and subject outcomes, tissue from the TURBT (tumor biopsies) are examined. Biopsies are assessed for specific pharmacodynamic, predictive, and prognostic biomarkers in the tumor. If tissue is available from a standard of care biopsy collected after enrollment, it is also examined to further identify biomarkers of response and mechanism of action and resistance to treatment.

Biomarker assessments in tumor tissue may include, but are not limited to, central assessment of Nectin-4 and PD-L1 expression by immunohistochemistry and next generation sequencing, tumor subtyping, tumor microenvironment analyses, and profiling of somatic mutations or alterations in genes or RNA commonly altered in cancer.

Biomarkers in Blood and/or Urine

The primary effects of enfortumab vedotin on tumor cells may lead to changes in the activation state of local, tumor-associated, and peripheral immune cells. Biomarker assessments in blood and urine samples may include, but may not be limited to, circulating/cell free tumor DNA, enzyme-linked immunosorbent assay (ELISA) assessment of soluble Nectin-4, immunoassays of urinary biomarkers, and markers of immune function, including abundance of immune cell subsets and cytokines.

### 6.2.2.11 Safety Assessments

Safety assessments include the surveillance and recording of AEs including serious adverse events (SAEs), recording of concomitant medications, and measurements of protocol-specified physical examination findings and laboratory tests.

### 6.2.2.12 Other Assessments

The subject perspective are qualitatively assessed through telephone interviews lasting 45 to 60 minutes covering topics around experience and tolerability of treatment. Additional details regarding the topics and questions that are explored during these interviews are described in the Subject Interview Guide document. Interviews are conducted during dose escalation and dose expansion at the following time points: at the end of the induction phase, at the completion of 5 doses during the maintenance phase, and at the end of the maintenance phase.

### 6.2.2.13 Statistical Methods

Dose escalation and identification of the MTD are conducted using the mTPI method. At the end of dose escalation, model-based estimate of the probability of DLT is presented along with the 95% credible intervals. The DE analysis set includes all treated subjects in dose escalation who either experienced a DLT or received at least 5 induction doses of enfortumab vedotin. The DE analysis set is the primary population for determination of the MTD.

Safety and antitumor activity endpoints are summarized using descriptive statistics based on the all-treated subjects analysis set, including all subjects who were treated with any amount of study drug. CR rate at any time on study, CR rates at 3, 6, 12, 18, and 24 months, and rate of cystectomy is summarized along with the exact 95% 2-sided confidence intervals (CIs). Duration of CR, PFS, and cystectomy-free survival is estimated using the Kaplan-Meier method.

No formal hypothesis testing is planned for the expansion cohort. Assuming an observed CR rate in the range of 30% to 50%, the 95% and 80% exact CIs with 20 subjects per cohort are summarized below in Table 11.

TABLE 11

| | 95% Exact CI | 80% Exact CI |
|---|---|---|
| CR Rate | (n = 20) | (n = 20) |
| 30% | (12%, 54%) | (17%, 47%) |
| 40% | (19%, 64%) | (25%, 57%) |
| 50% | (27%, 73%) | (34%, 66%) |

CI = confidence interval;
CR = complete response

### 6.2.3 Objectives

This study evaluates the safety, tolerability, PK, and antitumor activity of intravesical enfortumab vedotin in subjects with NMIBC. Specific objectives and corresponding endpoints for the study are summarized below in Table 12.

TABLE 12

| Primary Objectives | Corresponding Primary Endpoints |
|---|---|
| To evaluate the safety and tolerability | Type, incidence, severity, seriousness, |
| of intravesical enfortumab vedootin | and relatedness of AEs |
| in subjects with NMIBC | Type, incidence, and severity of |
| To identify the MTD or recommended | laboratory abnormalities |
| dose of intravesical enfortumab vedotin | Incidence of DLTs and cumulative |
| in subjects with NMIBC | safety by dose level |
| Secondary Objectives | Corresponding Secondary Endpoints |
| To assess the PK of intravesical | Estimates of selected PK parameters |
| enfortumab vidotin | including AUC, Cₘₐₓ, Tₘₐₓ, t½, |
| To assess the immunogenicity of | C_{trough} |
| intravesical enfotumab vidotin | Incidence of ATAs |
| To assess the antitumor activity of | CR rate at any time on study and CR |
| intravesical enfortumab vedotin as | rates at 3, 6, 12, 18, and 24 months |
| measured by CR rate | Duration of CR |
| To assess the duration of CR | Rate of cystectomy |
| To assess the rate of cystectomy | Progression-free survival |
| To assess progression-free survival | Cystectomy-free survival |
| To assess cystectomy-free survival | |
| Exploratory Objectives | Corresponding Exploratory Endpoints |
| To assess biomarkers in relation to | Correlative analyses of pharmacodynamics |
| response, toxicity, pharmacodynamics, | measurements and PK, response, toxicity, |
| PK/PD relationship, or resistance to | and resistance assessments |
| enfortumab vidotin | Assess Nectin-4 and PD-L1 expression |
| To assess subject-reported experience | levels tumor cells as exploratory predictive |
| and subject-reported tolerability | biomarker of clinical activity |
| of treatment | Biomarkers of enfortumab vedotin-mediates |
| | pharmacodynamic effects including but |
| | not limited to level of cell-free tumor DNA, |
| | soluble Nectin-4, cytokines |
| | Subject experience as assessed by subject |
| | interviews |

AE = adverse event;
ATA = antitherapeutic antibody;
AUC = area under the concentration-time curve;
Smax = maximum concentration;
CR = complete response;
Ctrough = trough concentration;
DLT = dose-limiting toxicity;
MRD = maximum tolerated dose;
NMIBC = non-muscle invasive bladder cancer;
PD-L1 = programmed death-ligand 1;
PK = pharmacokinetics;
PK//PD = pharmacokinetic/pharmacodynamics;
Tmax = time at which the maximum concentration occurs;
t½ = half life

### 6.2.4 Investigational Plan

### 6.2.4.1 Summary of Study Design

This is a phase 1, open-label, multicenter, dose-escalation, and dose-expansion study designed to evaluate the safety, tolerability, PK, and antitumor activity of intravesical enfortumab vedotin in adults with NMIBC.

The study is conducted in 2 parts:
Dose escalation: Approximately 18 subjects are treated to evaluate the safety, tolerability, and systemic exposure of intravesical enfortumab vedotin to identify the maximum tolerated dose (MTD) and/or recommended dose.
Dose expansion: Approximately 40 subjects (up to 2 cohorts of approximately 20 subjects each) are treated at the MTD or recommended dose to further characterize the safety, PK, and antitumor activity of intravesical enfortumab vedotin.

Dose escalation and dose expansion enrolls adult subjects with BCG-unresponsive NMIBC with CIS (with or without papillary disease). All subjects receive enfortumab vedotin, the investigational agent under study, via intravesical administration. Treatment on the study occurs during the induction and maintenance phases, and subjects enter a follow-up period after completion of the maintenance phase.

During the induction phase, subjects receive intravesical instillations of enfortumab vedotinonce a week (q1wk) for 6 weeks. Six to 8 weeks following completion of the induction phase, subjects have their first on-study response assessment, after which they enter the maintenance phase. During the maintenance phase, subjects receive intravesical enfortumab vedotin once a month for a total of 9 doses.

After completion of the maintenance phase, subjects enter the follow-up phase of the study. Tumor response assessment via standard of care cystoscopy (i.e., cystoscopy±biopsy) and cytology occurs every 3 months from the first induction dose for the first 2 years after enrollment, and every 6 months thereafter for 5 years after enrollment until disease recurrence, progression, initiation of subsequent anticancer therapy, or death, whichever occurs first. After discontinuation of study treatment due to disease persistence, recurrence, progression, or initiation of subsequent anticancer therapy, subjects enter the survival follow-up, where survival and subsequent anticancer therapy data is collected every 6 months (±2 weeks) until loss to follow-up, withdrawal of consent, death, or study termination by the sponsor, whichever occurs first, for a maximum of 5 years after enrollment.

Response assessment via cystoscopy and urine cytology must be completed within 14 days prior to the next administration of study drug. Subjects with abnormal cystoscopy, positive, or abnormal urine cytology should undergo additional evaluation (eg, imaging, biopsies, exam under anesthesia) per the investigator's clinical discretion.

Bladder mapping biopsies are required at the Month 12 assessment for the study. In the absence of visible tumor, biopsies should be obtained from all quadrants of the bladder (minimum biopsies required). Biopsy is not required at all other visits but is considered when clinically indicated for consideration of efficacy.

Subjects who are found to have high-grade T1 disease with or without CIS or stage progression at any on-study evaluation, including the first on-study 3-month evaluation, are discontinued from study treatment. Subjects with persistent CIS or recurrent high-grade Ta disease at the first on-study 3-month evaluation may continue therapy after they are reconsented for continuing study treatment and be assessed again at 6 months. Beginning with the 6-month assessment, any subject with persistent or recurrent high-risk NMIBC or disease progression is discontinued from study treatment. Appearance, presence, or persistence of lower grade tumors is not considered to be recurrence. Subjects with only low-grade tumors should continue treatment after resection and histological confirmation.

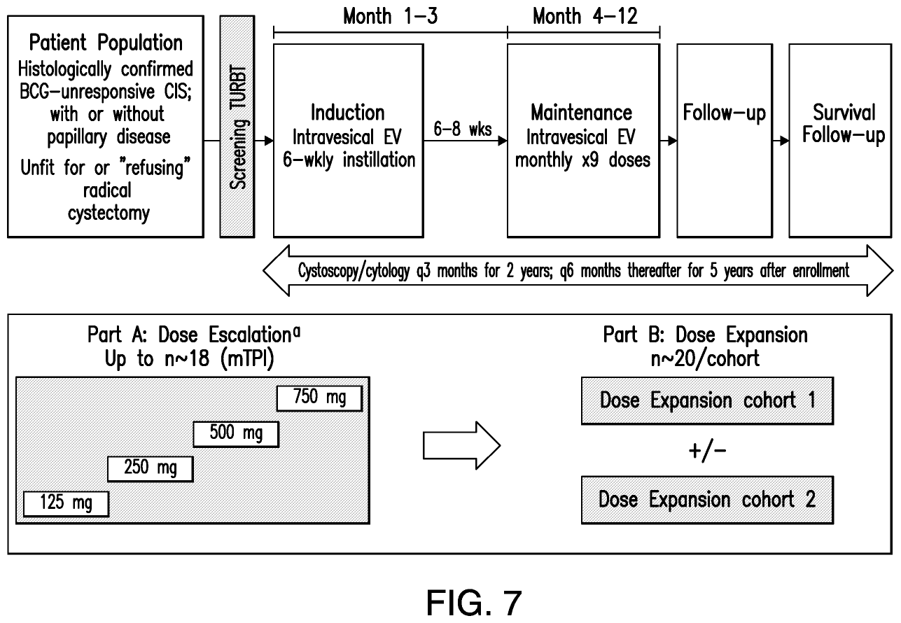

Subjects who discontinue study treatment due to reasons other than disease persistence, recurrence, or progression remain in the follow-up phase of the study. FIG. 7 presents the overall study design.

A safety monitoring committee (SMC) consisting of the site investigator(s) and representatives from the sponsor (including the study medical monitor, drug safety representative, clinical scientist, and biostatistician) monitor subject safety and make dosing recommendations throughout dose escalation and dose expansion. The SMC may recommend further evaluation of the safety at a given dose (i.e., enrolling additional subjects at a given dose) or investigation of a dose level that is lower than or intermediate to the planned dose levels. The SMC also reviews cumulative safety data to identify safety concerns that may emerge due to cumulative exposure beyond the dose-limiting toxicity (DLT) window. The SMC may also review antitumor activity data to determine whether the benefit-risk profile supports continued investigation or cessation of investigation of a dose or cohort. The SMC provides recommendations and final decisions will be made by the sponsor. Further details are documented in an SMC charter.

### (i) Dose-Escalation Cohort

Approximately 18 subjects are enrolled in dose escalation.

Dose escalation is conducted using the modified toxicity probability interval (mTPI) method (Ji 2010) to evaluate the safety and tolerability and to identify the MTD and/or recommended dose of intravesical enfortumab vedotin. Safety, PK, pharmacodynamics, biomarker analyses, and preliminary antitumor activity is used to determine a recommended dose and schedule.

The mTPI method uses a Bayesian model to compute the posterior probabilities of 3 intervals that reflect the relative distance between the toxicity rate of each dose level to the target DLT rate. Dosing-decision rules are determined for a target DLT rate of 25% with a 5% margin. The 3 intervals are (0%, 20%), (20%, 30%), and (30%, 100%), and the corresponding dosing decision rules are
1. Escalate if the current dose DLT rate is likely<20%,
2. Stay if the current dose DLT rate is likely between 20% and 30%,
3. De-escalate if the current dose DLT rate is likely>30%.

Dose-finding decisions are shown in Table 13. “E” represents escalating the dose, “S” represents staying at the same dose, and “D” represents de-escalating the dose. Decision “DU” means that the current dose level is unacceptable because of high toxicity. A dose is defined as having unacceptable toxicity if the posterior probability that the DLT rate is higher than 25% is more than 95%.

TABLE 13
Dose finding spreadsheet for mTPI design

| | Number of DLT-Evaluable Subjects at Current Dose | | | | | | | | | | | | | | |
|---|---|---|---|---|---|---|---|---|---|---|---|---|---|---|---|
| | | 2 | 3 | 4 | 5 | 6 | 7 | 8 | 9 | 10 | 11 | 12 | 13 | 14 | 15 |
| Number | 0 | E | E | E | E | E | E | E | E | E | E | E | E | E | E |
| of | 1 | S | S | S | S | E | E | E | E | E | E | E | E | E | E |
| DLTs | 2 | DU | D | D | S | S | S | S | S | S | S | S | S | E | E |
| | 3 | | DU | DU | DU | D | S | S | S | S | S | S | S | S | S |
| | 4 | | | DU | DU | DU | DU | DU | D | S | S | S | S | S | S |
| | 5 | | | | DU | DU | DU | DU | DU | DU | D | S | S | S | S |
| | 6 | | | | | DU | DU | DU | DU | DU | DU | DU | DU | D | S |
| | 7 | | | | | | DU | DU | DU | DU | DU | DU | DU | DU | DU |
| | 8 | | | | | | | DU | DU | DU | DU | DU | DU | DU | DU |
| | 9 | | | | | | | | DU | DU | DU | DU | DU | DU | DU |
| | 10 | | | | | | | | | DU | DU | DU | DU | DU | DU |
| | 11 | | | | | | | | | | DU | DU | DU | DU | DU |
| | 12 | | | | | | | | | | | DU | DU | DU | DU |
| | 13 | | | | | | | | | | | | DU | DU | DU |
| | 14 | | | | | | | | | | | | | DU | DU |
| | 15 | | | | | | | | | | | | | | DU |

D = de-escalate to the next lower dose; DLT = dose-limiting toxicity; DU = current dose is unacceptably toxic; E = escalate to the nexthigher dose; mTPI = modified toxicity probability interval; S = stay at the current dose.

Enrollment occurs on a cohort-by-cohort basis. Decisions on dose escalation and subsequent cohort size (minimum of 2 subjects) are made by the sponsor in consultation with the SMC after completion of each cohort. Subjects in the current cohort must be observed for the full duration of the DLT period before the next cohort is enrolled. As a precaution, for the first 2 subjects in the study there is a 72-hour observation period after each subject receives their first dose of study drug before the next subject can be dosed. At doses above dose level 1, a 72-hour observation period is required after the first subject receives their first dose of intravesical enfortumab vedotin prior to dosing subsequent subjects at that dose level. Subjects who are considered not evaluable for DLT are replaced. A minimum of 6 DLT-evaluable (DE) subjects is observed at the estimated MTD before the dose escalation is stopped. The MTD is estimated based on data from all subjects across all evaluated doses. If the MTD is not reached, safety, PK, pharmacodynamics, and biomarker data, as well as preliminary antitumor activity, are used to determine a recommended dose.

De-escalation to a lower or intermediate dose level is performed at any time by the sponsor in consultation with the SMC.

The dose escalation portion of the study evaluates escalating concentrations of enfortumab vedotin at 4 planned dose levels (125, 250, 500, and 750 mg; see Table 3) with a 25 mL volume of instillation and a maximum 90-minute dwell time (or subject's tolerated dwell time; actual dwell time is recorded on the appropriate electronic case report form [eCRF]). Up to approximately 18 subjects are enrolled in the dose escalation portion of the study. Enfortumab vedotin is administered intravesically q1wk for 6 weeks during the induction phase and once a month for 9 doses during the maintenance phase at the planned doses shown in Table 14. The sponsor and/or SMC may also recommend investigation of lower and/or intermediate dose levels. If the MTD is not reached, dose levels higher than 750 mg is explored.

TABLE 14
Dose escalation schema

| | Dose |
|---|---|
| Dose Level^{a} | (mg) |
| 1 | 125 |
| 2 | 250 |
| 3 | 500 |
| 4 | 750 |

q1 wk = once a week;
SMC = safety monitoring committee;
Dose frequency: induction q1 wk for 6 weeks; maintenance: once a month for 9 doses; 25 mL volume of instillation.
^{a}The SMC may recommand investigation of lower or intermediate dose levels based on emerging clinical data.

### (ii) Dose Expansion Cohorts

To further characterize the safety, tolerability, PK, and antitumor activity of intravesical enfortumab vedotin, approximately 40 additional subjects is enrolled in up to 2 dose-expansion cohorts (approximately 20 subjects per cohort). If the MTD is identified in the dose escalation portion of the study, the corresponding dose level is evaluated in dose expansion. The sponsor, in consultation with the SMC, may also evaluate more than 1 dose level in dose expansion if no MTD has been reached, or if different dose levels warrant further evaluation. The opening of these dose-expansion cohorts are determined by the sponsor in consultation with the SMC based on the cumulative safety and activity demonstrated during dose escalation.

(iii) Duration of Treatment

During the induction phase, subjects receive intravesical instillations of enfortumab vedotin q1wk for 6 weeks. Six to 8 weeks following completion of the induction phase, subjects have their first on-study response assessment, after which they enter the maintenance phase. During the maintenance phase, subjects receive intravesical enfortumab vedotin once a month for a total of 9 doses.

### (iv) Dose-Limiting Toxicity

DLTs are evaluated during the dose escalation portion of the study. The DLT-evaluation period is the time from start of induction to completion of all 6 induction doses plus 1 week. A subject is considered a DE subject if they either experienced a DLT or have received a minimum of 5 induction doses of enfortumab vedotin.

A DLT is defined as any of the following if assessed by the investigator to be related to treatment with intravesical enfortumab vedotin. Grading is according to the National Cancer Institute Common Terminology Criteria for Adverse Events (NCI CTCAE), Version 5.0:
Occurrence of Grade 3 or higher treatment-emergent AEs of the urinary tract such as hematuria, dysuria, urinary retention, urinary frequency/urgency, or bladder spasm
Grade 3 or higher local skin or mucosal reaction in groin or perineal area
Significant hematuria leading to clot obstruction that is related to the study drug per investigator assessment
Grade 3 or higher hematological toxicity (if it is an increase of ≥2 grades from baseline), including:
   Neutropenic fever
      Grade 3 febrile neutropenia is defined as absolute neutrophil count (ANC)
      <1000/mm³ with a single temperature of >38.3° C. (101° F.) or a sustained temperature≥38° C. (100.4° F.) for more than 1 hour
      Grade 4 febrile neutropenia is defined as ANC<1000/mm³ with a single temperature of >38.3° C. (101° F.) or a sustained temperature≥38° C. (100.4° F.) for more than 1 hour, with life-threatening consequences and urgent intervention indicated
   Grade 4 neutropenia or thrombocytopenia lasting>7 days
   Grade 3 thrombocytopenia with bleeding
Any other Grade 3 or higher nonhematologic AE, unless explained by an underlying medical condition, intercurrent illness, or malignancy
Unresolved treatment-related Grade 2 AE lasting>14 days
Any death not clearly due to the underlying disease or extraneous causes
Cases of Hy's law

The following nonhematological toxicities are not considered as DLTs:
Grade 3 nausea/vomiting or diarrhea<72 hours with adequate antiemetic and other supportive care
Grade 3 fatigue<1 week
≥Grade 3 electrolyte or other nonhematologic laboratory abnormality that lasts
<72 hours, is not clinically complicated, and resolves spontaneously or responds to conventional medical interventions
≥Grade 3 amylase or lipase that is not associated with symptoms or clinical manifestations of pancreatitis

### (v) Stopping Criteria

### (a) Enrollment Pause at the Cohort Level

If a death is considered to be related to enfortumab vedotin by the sponsor, enrollment is paused at that dose and all higher doses until:
1. The case is reviewed by the investigator, the sponsor, and the SMC, and
2. The sponsor has notified applicable regulatory authorities of the outcome of the safety assessment and justification for restarting enrollment in the affected cohorts, and has received approval to resume, if required by local regulations.

### (b) Enrollment Halt for the Entire Study

Enrollment in the entire study is halted by the sponsor if the overall benefit-risk balance is considered negative.

Safety is continuously monitored throughout the study by the sponsor and the SMC, with consideration for enrollment halt if the incidence and/or the severity of toxicity leads to a risk-benefit assessment that is unacceptable to the study population. The sponsor consults with the SMC to consider whether to allow subjects already receiving treatment to continue, to consider modifications to the protocol to continue enrollment, or to terminate the study.

If enrollment is halted due to safety concerns, enrollment can only be restarted after appropriate amendments and notifications to Regulatory Authorities, with approval to resume, if required by local regulations.

### (vi) End of Study

The study is closed approximately 5 years after the last subject is enrolled, or when no subjects remain in follow-up, whichever occurs first. In addition, the sponsor may terminate the study at any time.

(vii) Safety Monitoring Committee

The SMC monitors the safety of enfortumab vedotin throughout dose escalation and dose expansion. The SMC is composed of the site investigator(s) and representatives from the sponsor (including the study medical monitor, drug safety representative, clinical scientist, and biostatistician). The committee monitors the safety of participants in this study, through regular and/or ad hoc meetings that include review of data pertaining to dose escalation decisions and treatment-emergent (TE) toxicities. At a minimum, SMC meetings are held during the dose-escalation part of the study after all subjects in a cohort have completed the DLT-evaluation period. The SMC reviews clinical data from enrolled subjects for safety assessment and for DLT determination. The SMC makes recommendations on dose level and cohort size in conjunction with the mTPI decision rules chart. Multiple cohorts is enrolled at a dose level following the mTPI model. The SMC also meets approximately quarterly throughout dose expansion for cumulative subject data review.

In addition to determination of DLTs and dose-escalation recommendations, the SMC may make one or more of the following recommendations during the study, as applicable:
Further evaluation of safety at a given dose
Evaluation of alternative approaches to study drug administration (e.g., increasing the duration of administration or requiring pre-medication prior to administration)
Evaluation of a lower or intermediate dose level during dose escalation
Recommendation on the dose(s) to be evaluated in the expansion cohorts based inactivity and tolerability of dose levels during escalation
Recommendation on the single-agent dose and schedule for intravesical enfortumabvedotin based on the safety and activity data
If the MTD is not reached, evaluation of higher dose levels during dose escalation

Further details are documented in an SMC charter.

### 6.2.4.2 Discussion and Rationale for Study Design

This first-in-human (FIH) study is a phase 1, dose-escalation, and dose-expansion study to evaluate the safety and tolerability of intravesical enfortumab vedotin administered q1wk for 6 weeks during the induction phase and monthly for a total of 9 doses during the maintenance phase, and to estimate the MTD and/or determine a recommended dose and schedule in subjects with NMIBC. Initial clinical development of intravesical enfortumab vedotin involves its evaluation in subjects who are BCG-unresponsive and who have no appropriate standard therapeutic options (e.g., are ineligible for or refuse to undergo a radical cystectomy) and are candidates for enfortumab vedotin intravesical administration in the opinion of the treating physician.

Dose escalation is used to estimate the MTD and/or determine a recommended dose of intravesical enfortumab vedotin. Once dose escalation is complete and initial safety of the drug is demonstrated, up to 2 expansion cohorts of approximately 20 subjects each are enrolled to further evaluate the safety and antitumor activity of intravesical enfortumab vedotin. The expansion cohorts allow for the collection of additional information about the safety, tolerability, PK, and antitumor activity of intravesical enfortumab vedotin. This information will provide the basis for further development of intravesical enfortumab vedotin.

(i) Rationale for Using mTPI During Dose Escalation

The mTPI dose-escalation method was chosen for this study because of the potential advantages it has over the traditional “3+3” approach for dose finding. These advantages include the ability to target any pre-specified DLT rate, treatment of fewer subjects above the MTD thereby improving safety, and allowing for flexible cohort sizes (Ji 2010). The mTPI method also uses information from all subjects treated at all dose levels for estimation of the MTD to improve the accuracy of estimation.

### (ii) Method of Assigning Subjects to Treatment Groups

During dose escalation, subject allocation to a dose level is determined by mTPI decision rules as illustrated in Table 13, and occur upon approval of subject enrollment by the sponsor.

During dose expansion, subjects are enrolled in up to 2 cohorts of approximately 20 subjects each. If the MTD is identified in the dose escalation portion of the study, the corresponding dose level is evaluated in dose expansion. The sponsor, in consultation with the SMC, may also evaluate more than 1 dose level in dose expansion if no MTD has been reached, or if different dose levels warrant further evaluation. The opening of these dose-expansion cohorts will be determined by the sponsor in consultation with the SMC based on the cumulative safety and activity demonstrated during dose escalation.

The medical monitor assesses the safety of enfortumab vedotin intravesical administration throughout the trial and, for safety reasons, may override the model's allocation of a subject to a particular dose level.

(iii) Rationale for Selection of Doses

Systemic enfortumab vedotin is currently approved in the US at a dose of 1.25 mg/kg (up to a maximum dose of 125 mg) for the treatment of patients with locally advanced or metastatic UC who have previously received a PD-1 or PD-L1 inhibitor and a platinum-containing chemotherapy in the neoadjuvant/adjuvant, locally advanced, or metastatic setting and is also being evaluated at this dose in clinical trials. The safety profile of enfortumab vedotin at this dose level has been well established in systemic solid tumor trials, and specifically in bladder cancer (EV-101, EV-201, EV-301; see Investigator's Brochure for further detail). Given the preclinical evidence of tolerability, low systemic absorption, and the known safety profile of systemic enfortumab vedotin at 125 mg, this study will evaluate enfortumab vedotin starting at 125 mg administered intravesically.

### (iv) Blinding and Unblinding

This is an open-label study.

### 6.2.5 Study Population

Subjects must meet all of the enrollment criteria to be eligible for this study. Eligibility criteria may not be waived by the investigator and are subject to review in the event of a good clinical practice audit and/or health regulatory authority inspection.

### 6.2.5.1 Inclusion and Exclusion Criteria

See Section 6.2.2.

### 6.2.5.2 Childbearing Potential

A person of childbearing potential is anyone born female who has experienced menarche and who has not undergone surgical sterilization (eg, hysterectomy, bilateral salpingectomy, bilateral oophorectomy) or has not completed menopause. Menopause is defined clinically as 12 months of amenorrhea in a person over 45 years old in the absence of other biological, physiological, or pharmacological causes.

A person who can father children is anyone born male who has testes and who has not undergone surgical sterilization (eg, vasectomy followed by a clinical test proving that the procedure was effective).

6.2.5.3 Removal of Subjects from Study Treatment or Assessments

The sponsor of the study must be notified if a subject is withdrawn from study treatment or from the study. The reason(s) for withdrawal of a subject must be documented in the subject's medical records and case report form (CRF).

### (i) Discontinuation of Study Treatment

A subject's study treatment is discontinued for any of the following reasons:
Completed treatment per protocol
Persistent, recurrent, or progressive disease
   Subjects who are found to have high-grade T1 disease with or without CIS at anyon-study evaluation, including the first on-study 3-month evaluation, are discontinued from study treatment
   Subjects with persistent CIS or recurrent high-grade Ta disease at the first on-study 3-month evaluation may continue therapy after they are reconsented and continue on study treatment and be assessed again at 6 months
   Beginning with the 6-month assessment, any subject with persistent or recurrent high-risk NMIBC or disease progression are discontinued from study treatment.
   Appearance, presence, or persistence of lower grade tumors will not be considered as recurrence. Subjects with only low-grade tumors should continue treatment after resection and histological confirmation
Adverse event (AE)
Pregnancy
Investigator decision
Subject decision, non-AE
Study termination by sponsor
Other, non-AE

Subjects who discontinue from study treatment will remain on study for follow-up unless they withdraw consent.

### (ii) Treatment Discontinuation Recommendations Related to Liver Safety

In the absence of an explanation for increased liver function tests, such as viral hepatitis, preexisting or acute liver disease, or exposure to other agents associated with liver injury, the subject is discontinued from the study treatment. The investigator may determine that it is not in the subject's best interest to continue study treatment. Discontinuation of treatment shouldbe considered if:
ALT or AST>8×ULN
ALT or AST>5×ULN for more than 2 weeks
ALT or AST>3×ULN and total bilirubin>2×ULN or INR>1.5 (if INR testing inapplicable/evaluated)
ALT or AST>3×ULN with the appearance of symptoms suggestive of liver injury (eg, right upper quadrant pain or tenderness) and/or eosinophilia (>5%)

These treatment discontinuation recommendations are based on the FDA Guidance for Industry (Drug-Induced Liver Injury: Premarketing Clinical Evaluation, July 2009). The recommendations are a basic guide to the investigator based on accumulated clinical experience with drugs in development and are not specific to clinical experience with enfortumab vedotin.

(iii) Subject Withdrawal from Study

Any subject is discontinued from the study for any of the following reasons:
Completed study per protocol
Subject withdrawal of consent
Study termination by sponsor
Lost to follow-up
Death
Other

### 6.2.6 Treatments

### 6.2.6.1 Treatments Administered

All subjects receive enfortumab vedotin, the investigational agent under study in this protocol, via intravesical administration.

### 6.2.6.2 Investigational Product

Detailed information describing the preparation, administration, and storage of enfortumab vedotin is located in the Pharmacy Instructions.

### (i) Description

Enfortumab vedotin is generated by conjugation of a chemical intermediate that contains both the MMAE and linker subunits to cysteine residues of the antibody. The resulting ADC contains an average of 3.8 drug molecules per antibody. The enfortumab vedotin drug product is a sterile, preservative free, white to off-white lyophilized powder to be reconstituted for intravesical administration. Enfortumab vedotin is supplied in 30 mg single-dose vials.

### (ii) Dose and Administration

Enfortumab vedotin is administered intravesically q1wk for 6 weeks during the induction phase, and once a month for 9 doses during the maintenance phase.

The dose escalation portion of the study evaluates escalating concentrations of enfortumab vedotin at 4 planned dose levels (125, 250, 500, and 750 mg) with a 25 mL volume of instillation and a maximum 90-minute dwell time (or subject's tolerated dwell time; actual dwell time will be recorded on the appropriate eCRF).

(iii) Dose Modifications

Dose delays or dose modifications for toxicity should be made by the investigator in consultation with the medical monitor on a per-subject basis. During the dose escalation portion of the study, subjects are discontinued from study treatment if they experience any toxicity meeting the DLT criteria during the DLT evaluation period, unless toxicity is adequately managed, the investigator considers resumption of study treatment to be appropriate, and there is approval from the medical monitor. The type and severity of the AE observed is taken into consideration to inform the decision.

In the event of dose delay, subjects should continue to undergo response assessments every 3 months as per standard of care and the Schedule of Events.

### (a) Induction Phase

Dose delays up to 14 days are permitted during the induction phase with medical monitor approval. During the induction phase, the investigator may skip only 1 scheduled induction treatment due to unresolved toxicity. Subjects requiring more than 1 scheduled induction treatment to be skipped are discontinued from study treatment.

Subjects who experience an AE meeting DLT criteria during the induction phase should not receive further treatment with intravesical enfortumab vedotin unless the toxicity is managed adequately, the investigator considers resumption of intravesical enfortumab vedotin appropriate, and the medical monitor approves resumption. The type and severity of the AE observed are taken into consideration to inform the decision.

Subjects may resume treatment at the same dose level or a reduced dose level after discussion with the medical monitor. If a subject continues treatment after an AE that qualifies as a DLT and the same AE recurs, treatment must be permanently discontinued. Subjects who experience AEs that meet the criteria for permanent discontinuation of intravesical enfortumab vedotin may not resume study treatment, including at a lower or modified dose.

For the second occurrence of the same Grade 3 AE, intravesical enfortumab vedotin should be discontinued permanently.

Contact the medical monitor in the event of any other treatment-unrelated AE that requires a dose delay≥14 days.

### (b) Maintenance Phase

During the maintenance phase, subjects with unresolved treatment-related Grade 2 or higher AEs requiring dose delay may hold the scheduled maintenance dose until the AE returns to Grade 1 or baseline severity. Treatment delays>28 days for any unresolved AEs should be discussed with the medical monitor to determine if there is ongoing clinical benefit for the subject to continue on trial. Maintenance doses should be delayed rather than skipped. Delaying 2 consecutive maintenance doses due to unresolved treatment-related toxicity is not permitted and treatment must be permanently discontinued.

Subjects may resume treatment at the same dose level or a reduced dose level after discussion with the medical monitor. If a subject continues treatment after an AE that qualifies as a DLT and the same AE recurs, treatment must be permanently discontinued. Subjects who experience AEs that meet the criteria for permanent discontinuation of intravesical enfortumab vedotin may not resume study treatment, including at a lower or modified dose.

For the second occurrence of the same Grade 3 AE, intravesical enfortumab vedotin will be discontinued permanently.

### (c) Dose Modifications for Treatment-Associated Toxicity

Dose modification recommendations for enfortumab vedotin-associated hematologic and nonhematologic toxicity are presented in Table 15 and Table 16, respectively.

TABLE 15
Recommended dose modifications for enfortumab
vedotin-associated hematologic toxicity

| Grade 1 | Grade 2 | Grade 3 | Grade 4 |
|---|---|---|---|
| Continue at same | Continue at same | Discontinue | Discontinue |
| dose | dose | treatment. | treatment. |
| level. | level. | | |
| | For Grade 2 | | |
| | thrombocytopenia, | | |
| | withhold dose until | | |
| | toxicity is ≤Grade 1 | | |
| | or has returned to | | |
| | baseline, then | | |
| | resume treatment at | | |
| | the same dose level. | | |

TABLE 16
Recommended dose modifications for enfortumab vedotin-
associatednonhematologic toxicity

| Toxicity | Grade 1 | Grade 2 | Grade 3 | Grade 4 |
|---|---|---|---|---|
| Skin rash not | May continue | For Grade, | Discontinue | Discontinue |
| related to | at same dose | 2 skin rash, | treatment. | treatment. |
| accidental local | level. | consider with- | | |
| urine or drug | | holding | | |
| exposure | | enfortumab | | |
| | | vedotin until | | |
| | | toxicity is | | |
| | | ≤Grade 1 or | | |
| | | has returned | | |
| | | to baseline, | | |
| | | then resume | | |
| | | treatment | | |
| | | at the same | | |
| | | dose level | | |
| Ocular events | For Grade 1 | Discontinue | Discontinue | Discontinue |
| | ocular events, | treatment. | treatment. | treatment. |
| | ocular | | | |
| | symptoms | | | |
| | and/or changes | | | |
| | in vision, if | | | |
| | identified, | | | |
| | should be | | | |
| | evaluated by a | | | |
| | qualified | | | |
| | optometrist | | | |
| | or ophthalmologist. | | | |
| Neuropathy | May continue at | Discontinue | Discontinue | Discontinue |
| | | treatment. | treatment. | treatment. |
| Hyperglycemia | same dose level. | May continue | For Grade 3 | For Grade 4 |
| | | at same dose | hyperglycemia | hyperglycemia |
| | | level. | or blood glucose | or blood glucose |
| | | | >250 mg/dL, | >500 mg/dL |
| | | | withhold | that is considered |
| | | | enfortumab | related to study |
| | | | vedotin treatment. | treatment, |
| | | | Resume treatment | withhold |
| | | | once hyper- | enfortumab |
| | | | glycemia/blood | vedotin |
| | | | glucose is ≤250 | treatment and |
| | | | mg/dL and subject | undertake a full |
| | | | is clinically and | evaluation of the |
| | | | metabolically | hyperglycemia |
| | | | stable. | to determine the |
| | | | | underlying |
| | | | | diagnosis. Once |
| | | | | hyperglycemia/ |
| | | | | elevated blood |
| | | | | glucose has |
| | | | | improved to <250 |
| | | | | mg/dL, dosing |
| | | | | may resume with |
| | | | | close monitoring |
| | | | | after consultation |
| | | | | with the medical |
| | | | | monitor. |

Throughout the study treatment, Grade 3/4 electrolyte imbalances/laboratory abnormalities, except hyperglycemia, that are not associated with clinical sequelae and are corrected with supplementation/appropriate management within 72 hours of their onset do not require discontinuation (e.g., Grade 4 hyponatremia).

### (iv) Storage and Handling

Refrigeration should be set at 2-8° C. for storage of vials and solutions containing enfortumab vedotin. The controlled location must be accessible only to the pharmacist, the investigator, or a duly designated person. Study drug must be reconstituted before administration.

The effect of light on the study drug has not been assessed; therefore, it is recommended that vials of enfortumab vedotin lyophilized powder, reconstituted drug product and/or dosing solutions be protected from light until the time of use.

Do not shake reconstituted vials of the study drug.

Any partially used vials or prepared dosing solutions should be destroyed by the site according to institutional drug disposal procedures. Unused vials should be destroyed by the site or returned to the sponsor after authorization by the sponsor.

### 6.2.6.3 Required Premedication and Post Medication

There are no required premedications or postmedications for intravesical enfortumab vedotin.

Use of local skin barrier ointments prior to instillation around the external genitalia is considered to prevent skin contact or irritation.

Subjects, especially females, should be instructed to wash the genitalia after each urination on the day of instillation and use barrier ointments as needed.

### 6.2.6.4 Concomitant Therapy

All treatments that the investigator considers necessary for a subject's welfare is administered at the discretion of the investigator in keeping with the community standards of medical care except for those medications identified as prohibited.

All concomitant medications and blood products administered are recorded from Day 1 (predose) through the safety reporting period. Any concomitant medication given for a study protocol-related AE should be recorded from the time of informed consent.

### (i) Required Concomitant Therapy

There are no required concomitant therapies.

### (ii) Allowed Concomitant Therapy

Concomitant chronic prednisone (or equivalent) is used at a dose of ≤20 mg/day. Higher doses of prednisone (or equivalent) are permitted for limited duration to treat acute conditions that arise during the study as medically indicated. The use of anti-emetics is permitted.

Subjects who are receiving strong cytochrome P450 (CYP) 3A4 inhibitors or P-glycoprotein (P-gp) inhibitors concomitantly with enfortumab vedotin should be closely monitored for adverse reactions.

Routine prophylaxis with vaccines is permitted; it is recommended that vaccines used do not contain live micro-organisms.

Subjects with a positive hepatitis B surface antigen and/or antihepatitis B core antibody and a negative PCR assay at baseline should receive appropriate antiviral prophylaxis or regular surveillance monitoring as per local or institutional guidelines.

Prophylactic antibiotics are permitted as clinically indicated to minimize the risk of urinary tract infections. If a subject has a symptomatic UTI, they is treated with a full course of antibiotics and study drug will be withheld until resolution.

Anticholinergics is prescribed for subjects who have symptoms of frequency, urgency, or incontinence.

All treatments that the investigator considers necessary for a subject's welfare is administered at the discretion of the investigator in keeping with the community standards of medical care.

(iii) Prohibited Concomitant Therapy

Diuretics should not be taken the night before a dosing day, or on the day of dosing prior to study drug administration.

To the extent feasible, fluid consumption should be avoided 4 to 6 hours prior to instillation of intravesical enfortumab vedotin.

Subjects may not receive other investigational drugs, radiotherapy, intravesical, or systemic anti-neoplastic therapy during the study.

### 6.2.6.5 Management of Treatment-Emergent Adverse Events

### (i) Management of Hyperglycemia

Investigators should monitor blood glucose levels and are advised to perform additional assessments if any symptoms of hyperglycemia are observed, including a thorough evaluation for infection. In addition, if steroids are used to treat any other condition, blood glucose levels may require additional monitoring. If elevated blood glucose levels are observed, subjects should be treated according to local standard of care and referral to endocrinology is considered.

Subjects, especially those with a history of or ongoing diabetes mellitus or hyperglycemia, should be advised to immediately notify their physician if their glucose level becomes difficult to control or if they experience symptoms suggestive of hyperglycemia such as frequent urination, increased thirst, blurred vision, fatigue, and headache.

Subjects who enter the study with an elevated HbA1c (≥6.5%) at baseline should be referred to an appropriate provider during the induction phase for glucose management. Blood glucose should be checked prior to each dosing and dose should be withheld for blood glucose>250 mg/dL. Dosing may continue once the subject's blood glucose has improved to ≤250 mg/dL and subject is clinically and metabolically stable. The use of insulin is permitted as part of standard of care. Blood glucose>500 mg/dL considered related to enfortumab vedotin requires drug interruption and a full evaluation of the hyperglycemia to determine the underlying diagnosis. Once hyperglycemia/elevated blood glucose has improved to ≤250 mg/dL, dosing may resume with close monitoring after consultation with medical monitor. If a subject experiences new onset of diabetes mellitus, evaluate with a metabolic panel, urine ketones, HbA1c, C-peptide, to assess new onset of type 1 diabetes in the setting of prior checkpoint inhibitor therapy.

(ii) Management of Rash with Enfortumab Vedotin

Enfortumab vedotin is a Nectin-4 directed antibody drug conjugate. Nectin-4 is a cell adhesion molecule that is highly expressed in urothelial carcinoma. Low to moderate levels of Nectin-4 are also expressed on normal tissues, including skin keratinocytes, sweat glands and hair follicles; thus, skin reactions are anticipated events. As such, skin reactions are adverse events of interest in all clinical studies with enfortumab vedotin.

A cumulative review of post-marketing safety data from 18 Dec. 2019 (the approval date of enfortumab vedotin in the US) through 22 Oct. 2020 identified reports of severe cutaneous adverse reactions in 15 patients receiving IV enfortumab vedotin, some of whom had fatal outcomes. These reactions occurred predominantly during the first cycle of treatment. AEs reported in these cases included Stevens-Johnson syndrome (SJS) (5 cases), blister (3 cases), dermatitis bullous (3 cases), symmetrical drug-related intertriginous and flexural exanthema (SDRIFE; 2 cases), and 1 case each of dermatitis exfoliative, exfoliative rash, epidermal necrosis, oropharyngeal blistering, stomatitis, and toxic epidermal necrolysis (TEN).

In systemic enfortumab vedotin monotherapy studies of urothelial carcinoma, serious adverse events (SAEs) of severe cutaneous adverse reactions were reported in 11 of 749 subjects (1.5%) and included dermatitis bullous (0.4%), drug eruption (0.4%), blister (0.1%), conjunctivitis (0.1%), SJS (0.1%), stomatitis (0.1%), and toxic skin eruption (0.1%).

Subjects should also be advised to contact the investigator immediately if they have signs and symptoms of skin reactions, oral mucosal and ocular abnormalities including mucositis or conjunctivitis. Starting in the first cycle and throughout treatment, closely monitor subjects for skin reactions. For mild to moderate skin reactions, consider appropriate treatment, such as topical corticosteroids and antihistamines as clinically indicated. For worsening Grade 2 rash or skin reactions, consider withholding enfortumab vedotin. For Grade 3 or 4 rash or skin reactions or suspected or confirmed SJS or TEN, permanently discontinue enfortumab vedotin and consider referral for specialized care.

### (a) Local Skin Care

Safety of enfortumab vedotin when exposure occurs via skin or mucosal surfaces has not been evaluated in ongoing clinical trials or preclinical studies. Care should be taken to avoid accidental exposure to any skin, eye, or mucosal surface with the drug during instillation or urine post-instillation.

Since there is inadvertent skin exposure during instillation, dwell time or voiding, the treating investigator should examine the local genital and perineal area for any skin irritation or breakdown prior to each instillation. For minimal skin irritation or breakdown, local barrier ointments or dressings should be applied to prevent skin exposure during these times. For significant skin changes or breakdown noted prior to instillation, necessary precautions and/or dose hold should be considered in addition to medical monitor consultation. Visual inspection of the genital and perineal area, or other affected skin areas should be done post-void when feasible or if the subject reports any accidental exposure, or symptoms related to skin irritation.

Subjects should wash hands after voiding. If subjects have any accidental exposure, the affected skin area should be immediately washed with soap and water. The affected area should be observed in the following days for the development of any redness, itchiness, swelling or other symptoms. Any symptoms must be reported to the treating investigator immediately for appropriate care and follow up.

(iii) Allergic/Hypersensitivity Reaction

Allergic/hypersensitivity reactions are characterized by adverse local or general responses from exposure to an allergen (NCI CTCAE version 5.0). Allergic/hypersensitivity reactions may manifest as a combination of signs or symptoms including itching, various types of rash, urticaria, nausea, vomiting, back or abdominal pain.

Allowed measures include dose modifications (Table 16) and concomitant medications.

### (iv) Management of Overdose

In the event of an overdose>10% of enfortumab vedotin, the site should notify the sponsor as soon as they are aware of the overdose. The patient should be closely monitored for adverse reactions. Supportive care per institutional standards should be administered.

### 6.2.6.6 Treatment Compliance

Study drug administration will be performed by study site staff and documented in source documents and the CRF.

### 6.2.7 Study Activities

### 6.2.7.1 Schedule of Events

AEs and concomitant medications are recorded from Day 1 (predose) through the safety reporting period. Any study protocol-related AE as well as any concomitant medications given for treatment of the AE, should be recorded from the time of informed consent.

Clinical laboratory assessments (serum chemistry panel, complete blood count [CBC] with differential [manual differential if clinically indicated], and urinalysis [with reflexive microscopy for abnormal results]), physical examination, weight, and ECOG performance status is performed within 3 days prior to administration of study drug on Day 1, Week 1 of the induction phase. The results from all relevant clinical laboratory assessments must be reviewed prior to dosing. A Schedule of Events is provided in Table 17.

TABLE 17
Schedule of Events

| | | Screening/ | | | Induction Phase^{A} | | | | |
|---|---|---|---|---|---|---|---|---|---|
| | | Baseline | | Enrollment | (Month 1 to Month 3) | | | | |
| | | Treatment Visit | | | | | | | |
| | | | | | | | | Week 2 | |
| | | | | Within | Week 1 | | | through 6 | |
| | | D −28 | D −7 | 7 Dof 1st | Day | Day | Day | Day | Day |
| | | to 1 | to 1 | dose | 1 | 2^{E} | 4^{E} | 1 | 2^{E} |
| | | Visit window | | | | | | | |
| | | | | | | | | ±1 d | |
| Screening/ | Informed consent | X | | Submit | | | | | |
| Baseline | Inclusion/ | X | | eligibility | X | | | | |
| Assessments | Exclusion, | | | confirmation | | | | | |
| | medical history | | | tosponsor | | | | | |
| | Pregnancy test | | X | prior to | X^{G} | | | X^{G} | |
| | (serum orurine; | | | treatment | | | | | |
| | subjects of | | | | | | | | |
| | childbearing | | | | | | | | |
| | potential) | | | | | | | | |
| Safety | Physical | | X | | X^{I} | X^{J} | | X | X^{J} |
| Assessments | examination | | | | | | | | |
| | Height^{K} | | X | | | | | | |
| | Weight | | X | | X^{I} | | | X | |
| | CBC with | | X | | X^{I} | | | X | |
| | differential^{L} | | | | | | | | |
| | Serum chemistry | | X | | X^{I, M} | | | X^{M} | |
| | panel^{L, AA} | | | | | | | | |
| | HbA1c | | X | | | | | | |
| | Glucose | | X | | X^{Z} | | | X^{Z} | |
| | Serology | | X | | | | | | |
| | (hepatitis | | | | | | | | |
| | B and C) | | | | | | | | |
| | PT/PTT/INR | | X | | X^{N} | | | X^{N} | |
| | Urinalysis with | | X | | X^{I} | | | X | |
| | reflexive- | | | | | | | | |
| | microscopic | | | | | | | | |
| | analysis | | | | | | | | |
| | Vital signs | | X | | X^{O} | | | X^{O} | |
| | ECOG performance | | X | | X^{I} | | | | |
| | status | | | | | | | | |
| | ECG | X | | | | | | | |
| | Imaging | X^{P} | | | | | | | |
| | Complete eye exam | X^{BB} | | | | | | | |
| | Subject | | | | | | | | X^{Q} |
| | interview^{Q} | | | | | | | | |
| | Concomitant | Related to study | | | Collect from Day 1 predose through safety | | | | |
| | medications | procedures^{R} | | | reporting period of study drug | | | | |
| | Adverse event | | | | | | | | |
| | collection | | | | | | | | |
| Treatment | Study drug | | | | X | | | X | |
| | administration | | | | | | | | |
| PK/ | Initiate collection | X^{T} | | | See Table 18 PK, ATA, and Biomarker Collections (Blood) and Table 19 PK | | | | |
| Immunogenicity/ | of (archived) tumor | | | | and Biomarker Collections (Urine) tables forsample collection details | | | | |
| Biomarker | specimen | | | | | | | | |
| | Blood/urine/ | X | | | | | | | |
| | tissue sample | | | | | | | | |
| | collection | | | | | | | | |
| Response | Cystoscopy^{W, DD} | X | | | | | | | |
| Assessment | Cytology^{W} | X | | | | | | | |
| | Biopsy^{W} | X^{N} | | | | | | | |
| | Disease status, | | | | | | | | |
| | survival status, | | | | | | | | |
| | collection of first | | | | | | | | |
| | subsequent therapy | | | | | | | | |

| | | | | | | | | Survival |
|---|---|---|---|---|---|---|---|---|
| | | Induction Phase^{A} | | Maintenance Phase^{B} | | EOT | Follow- | Follow- |
| | | (Month 1 to Month 3) | | (Month 4 to Month 12) | | Within | up^{D} | up |
| | | Treatment Visit | | | | | | |
| | | | | Week 12-14^{B} to | | | | |
| | | | | Week 52 | | | | |
| | | Week 2 | | (monthly for 9 doses) | | | | |
| | | through 6 | | | Day 15^{F} | 30-37 D | | |
| | | Day | Week 9 | | (phone | of last | | Every 6 |
| | | 4^{E} | Day 1 | Day 1 | call) | dose^{C} | | months^{Y} |
| | | Visit window | | | | | | |
| | | | ±3 d | ±3 d | ±3 d | | ±1 wk | ±2 weeks |
| Screening/ | Informed consent | | | X^{CC} | | | | |
| Baseline | Inclusion/ | | | | | | | |
| Assessments | Exclusion, | | | | | | | |
| | medical history | | | | | | | |
| | Pregnancy test | | X^{G} | X^{G} | | X | X^{H} | |
| | (serum orurine; | | | | | | | |
| | subjects of | | | | | | | |
| | childbearing | | | | | | | |
| | potential) | | | | | | | |
| Safety | Physical | | | X | | X | X | |
| Assessments | examination | | | | | | | |
| | Height^{K} | | | | | | | |
| | Weight | | | X | | | | |
| | CBC with | | | X | | X | | |
| | differential^{L} | | | | | | | |
| | Serum chemistry | | | X | | X | | |
| | panel^{L, AA} | | | | | | | |
| | HbA1c | | | | | | | |
| | Glucose | | | X^{Z} | | X | | |
| | Serology | | | | | | | |
| | (hepatitis | | | | | | | |
| | B and C) | | | | | | | |
| | PT/PTT/INR | | | X^{N} | | | | |
| | Urinalysis with | | | X | | X | X | |
| | reflexive- | | | | | | | |
| | microscopic | | | | | | | |
| | analysis | | | | | | | |
| | Vital signs | | | X^{O} | | X | | |
| | ECOG performance | | | X | | X | | |
| | status | | | | | | | |
| | ECG | | | | | | | |
| | Imaging | | | | | | X^{N} | |
| | Complete eye exam | | | | | X^{BB} | | |
| | Subject | | | | X^{Q} | | | |
| | interview^{Q} | | | | | | | |
| | Concomitant | Collect from Day 1 predose through safety | | | | | | |
| | medications | reporting period of study drug | | | | | | |
| | Adverse event | | | | | | | |
| | collection | | | | | | | |
| Treatment | Study drug | | | X^{S} | | | | |
| | administration | | | | | | | |
| PK/ | Initiate collection | See Table 18 PK, ATA, and Biomarker Collections (Blood) and Table 19 PK | | | | | | |
| Immunogenicity/ | of (archived) tumor | and Biomarker Collections (Urine) tables forsample collection details | | | | | | |
| Biomarker | specimen | | | | | | | |
| | Blood/urine/ | | | | | | | |
| | tissue sample | | | | | | | |
| | collection | | | | | | | |
| Response | Cystoscopy^{W, DD} | | | X^{U, V} | | | X | |
| Assessment | Cytology^{W} | | | X^{U} | | | X | |
| | Biopsy^{W} | | | X^{X} | | | X^{N} | |
| | Disease status, | | | | | X | | X^{Y} |
| | survival status, | | | | | | | |
| | collection of first | | | | | | | |
| | subsequent therapy | | | | | | | |

AE = adverse event;
ALT = alanine aminotransferase;
AST = aspartate aminotransferase;
ATA = antitherapeutic antibody;
CBC = complete blood count;
CIS = carcinoma in situ;
CT = computed tomography;
D/d = Day;
ECOG = Eastern Cooperative Oncology Group;
ECG = electrocardiogram;
EOT = end of treatment;
HbA1c = hemoglobin A1c;
MRI = magnetic resonance imaging;
PK = pharmacokinetic;
PT/PTT/INR = prothrombin time/partial thromboplastin time/international normalized ratio;
q1wk = once weekly;
TURBT = transurethral resection of bladder tumor;
Wk = week
^{A}Procedures will be conducted q1wk on Day 1 and Day 2 of each week during the induction phase.
^{B}After their first on-study response assessment (which will occur 6 to 8 weeks following completion of the induction phase), subjects will enter the maintenance phase. Procedures will be conducted monthly on Day 1 and Day 15 during the maintenance phase.
^{C}EOT evaluations should be obtained before the initiation of non-protocol anticancer treatment. If EOT evaluations are completed before 30 days following the last study treatment, conduct a phone screen 30-37 days following the subject's last study treatment to ensure that no changes in AE profile have occurred.
^{D}Subjects who discontinue study treatment due to reasons other than disease persistence, recurrence, or progression and subjects who complete the maintenance phase will enter the follow-up phase of the study. Follow-up will occur every 3 months from the first induction dose for the first 2 years after enrollment, and every 6 months thereafter for 5 years after enrollment until disease recurrence, progression, initiation of subsequent anticancer therapy, or death, whichever occurs first.
^{E}Day 2 visits during the induction phase is conducted in the clinic or via a telehealth/phone call visit. Day 4 visits to occur at Weeks 1, 2, and 6 only. It is possible for PK samples on Day 2 and Day 4 to be collected via home visits by an external home health aide vendor.
^{F}The Day 15 visit is required during the first 3 maintenance doses and will be conducted via telehealth/phone call to assess for AEs and concomitant medications.
^{G}Not required if performed within 7 days prior to Day 1. Pregnancy test is required on study Day 1 of Weeks 1, 3, 6, and 9 during induction, and on study Day 1 of each month during the maintenance phase.
^{H}Collected monthly for 6 months after the last received dose of enfortumab vedotin.
^{I}Not required if performed within 3 days prior to Day 1.
^{J}To be conducted if visit is performed on site.
^{K}Measurements of height obtained within the prior 12 months is utilized.
^{L}Is conducted at any point during the study if clinically indicated.
^{M}Serum chemistry panel required at Weeks 1, 3, and 6 only during the induction phase.
^{N}If clinically indicated.
^{O}Vital signs will be collected and AEs will be recorded predose and 2 hours after the first void.
^{P}A diagnostic quality CT of the chest with IV contrast is required unless medically contraindicated. If the subject is unable to tolerate IV contrast, non-contrast chest CT is acceptable. For imaging of the upper tract, abdomen, and pelvis, CT or MRI urogram with contrast (unless medically contraindicated) is acceptable. Previous imaging with similar modality is used if performed within 3 months prior to start of study treatment.
^{Q}Interviews will be conducted during dose escalation and dose expansion at the following time points (+7-day window): at the end of the induction phase, at the completion of 5 doses during the maintenance phase, and at the end of the maintenance phase. Subjects who discontinue prior to Week 6 of the induction phase will undergo an exit interview at their final visit.
^{R}From time of informed consent.
^{S}During the maintenance phase, enfortumab vedotin will be administered intravesically once a month for a total of 9 doses.
^{T}Archived tumor specimen or tissue collected from the most recent TURBT collected within 12 months of enrollment is required, if available. The most recently available tissue should be used. If freshly cut slides only can be provided, a minimum of 10 to 15 sections is required (ifless than 10, contact the sponsor).
^{U}Response assessment via cystoscopy and urine cytology must be completed within 14 days prior to the next administration of study drug. Subjects with abnormal cystoscopy, positive, or abnormal urine cytology should undergo additional evaluation (eg, imaging, biopsies, exam under anesthesia) per the investigator's clinical discretion.
^{V}Subjects must have a cystoscopy prior to entering the maintenance phase.
^{W}All visible papillary Ta/T1 tumors must be completely resected within 60 days prior to enrollment. Residual pure CIS is permitted. Subjects with T1 disease should undergo repeat TURBT prior to starting study. Re-staging TURBT must show uninvolved muscularis propria. Tumor response assessment via standard of care cystoscopy (ie, cystoscopy ± biopsy) and cytology will occur every 3 months from the first induction dose for the first 2 years after enrollment, and every 6 months thereafter for 5 years after enrollment until disease recurrence, progression, initiation of subsequent anticancer therapy, or death, whichever occurs first. For all subjects, additional evaluation (such as imaging, biopsy) should be considered as clinically indicated based on findings during tumor assessment such as abnormal cystoscopy, positive urine cytology, and having 2 or more sequential urine cytology specimens with suspicious or indeterminate findings.
^{X}Bladder mapping biopsies are required at the Month 12 assessment for the study. In the absence of visible tumor, biopsies should be obtained from all quadrants of the bladder (minimum 4 biopsies required). Biopsy is not required at all other visits but will be considered when clinically indicated for consideration of efficacy.
^{Y}After discontinuation of study treatment due to disease persistence, recurrence, progression, or initiation of subsequent anticancer therapy, subjects will enter the survival follow-up. Survival and subsequent anticancer therapy data will be collected every 6 months (±2 weeks) until loss to follow-up, withdrawal of consent, death, or study termination by the sponsor, whichever occurs first, for a maximum of 5 years after enrollment.
^{Z}Verify blood glucose is <250 mg/dL prior to dosing either by blood draw or fingerstick.
^{AA}The serum chemistry panel is to include the following tests: albumin, alkaline phosphatase, ALT, AST, bicarbonate, blood urea nitrogen, calcium, creatinine, chloride, lactate dehydrogenase, phosphorus, potassium, sodium, total bilirubin, amylase, lipase, glucose, and uric acid.
^{BB}Complete eye examination performed by a qualified optometrist or ophthalmologist, including but not limited to visual acuity, slit lamp, tonometry examination, and dilated fundus examination. Subsequent eye examinations are to be conducted as clinically indicated. EOT slit lamp examinations are required for subjects who experience corneal AEs during the study and must be performed at least 4 weeks from last dose.
^{CC}Month 4 only. Subjects with persistent CIS or recurrent high-grade Ta disease at the first on-study 3-month evaluation may continue therapy after they are reconsented for continuing study treatment and be assessed again at 6 months.
^{DD}All known sites of disease must be documented at screening and re-assessed at each subsequent tumor evaluation.

TABLE 18
PK, ATA, AND BIOMARKER COLLECTIONS (BLOOD)

| | | | | | | | | | | PBMC | | Plasma | |
|---|---|---|---|---|---|---|---|---|---|---|---|---|---|
| | Study | | | | | | PK | | Serum | Immuno- | Plasmal | 2 | Tumor |
| Study Phase | Day | Time | | Window | | Relative Time | (blood) | ATA | Nectin-4 | phenotype | Cytokine | cfDNA | specimen^{A} |
| Screening | −28 to 1 | N/A | | N/A | | N/A | | | | | | XB | XC |
| Induction | Day 1 | Predose | | 24 | hr | START of | X | X | X | X | X | X | |
| Weeks 1, | | | | | | instillation | | | | | | | |
| 2, and 6 | | Post-void | | +15 | min | END of dwell | X | | | | | | |
| | | 2 | hr | ±15 | min | time | X | | | | | | |
| | Day 2^{D} | 24 | hr | ±4 | hr | END of dwell | X | | | | | | |
| | Day 4^{D} | 72 | hr | ±4 | hr | time | X | | | | | | |

| Induction | Day 1 | Predose | 24 | hr | START of | X | X | X |
|---|---|---|---|---|---|---|---|---|
| Weeks 3, | | | | | instillation | | | |
| 4 and 5 | | Post-void | +15 | min | END of dwell | X | | |

| | | | | | |
|---|---|---|---|---|---|
| 2 | hr | ±15 | min | time | X |

| Maintenance | Day 1 | Predose | 24 | hr | START of | X | X | X | XE |
|---|---|---|---|---|---|---|---|---|---|
| (Month 4 to | | | | | instillation | | | | |
| Month 12) | | Post-void | +15 | min | END of dwell | X | | | |

| | | | | | |
|---|---|---|---|---|---|
| 2 | hr | ±15 | min | time | X |

| | | | | |
|---|---|---|---|---|
| EOT | X | X | X | X^{A} |
| Follow-up^{F} | | | X | X^{A} |

ATA = antitherapeutic antibody;
cfDNA = circulating free DNA;
EOT = end of treatment;
N/A = not applicable;
PBMC = peripheral blood mononuclear cell;
PK = pharmacokinetic;
TURBT = transurethral resection of bladder tumor
^{A}If a tumor sample is obtained as part of standard of care during the study, a part of that sample should be submitted to the sponsor for biomarker testing if available. Tissue from biopsies collected while on treatment is used for biomarker assessments.
BIf TURBT is performed within the screening window, collection for biomarker assessments at screening should be collected prior to or at the time of TURBT.
CArchived tumor specimen or tissue collected from the most recent TURBT collected within 12 months of enrollment is required, if available. The most recently available tissue should be used. If freshly cut slides only can be provided, a minimum of 10 to 15 sections is required (if less than 10, contact the sponsor).
^{D}It is possible for PK samples on Day 2 and Day 4 to be collected via home visits by an external home health aide vendor.
EBladder mapping biopsies are required at the Month 12 assessment for the study. In the absence of visible tumor, biopsies should be obtained from all quadrants of the bladder (minimum 4 biopsies required). Biopsy is not required at all other visits but will be considered when clinically indicated for consideration of efficacy.
^{F}Follow-up will occur every 3 months from the first induction dose for the first 2 years after enrollment, and every 6 months thereafter for 5 years after enrollment until disease recurrence, progression, initiation of subsequent anticancer therapy, or death, whichever occurs first.

TABLE 19
PK AND BIOMARKER COLLECTIONS (URINE)

| | | | | | | | | | Urine |
|---|---|---|---|---|---|---|---|---|---|
| | Study | | | | | | PK | Urine | Nectin-4 |
| Study Phase | Day | Time | | Window | | Relative Time | (urine) | cfDNA | Cytokine |
| Screening | −28 to 1 | N/A | | N/A | | N/A | | X^{A} | X^{A} |
| Induction Weeks | Day 1 | Predose | | 24 | hr | START of | X | X | X |
| 1, 2, and 6 | | | | | | instillation | | | |
| | | Void^{B} | | None^{B} | | END of | X | | |
| | | 2 | hr | ±15 | min^{D} | dwell time^{C} | X | | |
| | Day 2^{E} | 24 | hr | ±4 | hr | END of | X | | |
| | Day 4^{E} | 72 | hr | ±4 | hr | dwell time | X | | |
| Induction Weeks | Day 1 | Predose | | 24 | hr | START of | X | X | X |
| 3, 4, and 5; and | | | | | | instillation | | | |
| Maintenance Phase | | Void^{B} | | None^{B} | | END of | X | | |
| Month 4 to 12 | | 2 | hr | ±15 | min^{D} | dwell time^{C} | X | | |

| | | | |
|---|---|---|---|
| EOT | X | X | X |
| Follow-up^{F} | | X | X |

cfDNA = circulating freeDNA;
EOT = end of treatment;
N/A = not applicable;
PK = pharmacokinetic;
TURBT = transurethral resection of bladder tumor
^{A}If TURBT is performed within the screening window, collection for biomarker assessments at screening should be collected prior to or at the time of TURBT.
^{B}Voided urine immediately after completion of the dwell time should be collected. Urine collection will be the void of urine following the maximum 90-minute dwell time (or end of subject's tolerated dwell time) for study drug.
^{C}After instillation of study drug, subjects will hold study drug for a maximum 90-minute dwell time, or until the end of the subject's tolerated dwell time.
^{D}Second urine collection after completion of maximum 90-minute dwell time void (or end of subject's tolerated dwell time void).
^{E}It is possible for PK samples on Day 2 and Day 4 to be collected via home visits by an external home health aide vendor.
^{F}Follow-up will occur every 3 months from the first induction dose for the first 2 years after enrollment, and every 6 months thereafter for 5 years after enrollment until disease recurrence, progression, initiation of subsequent anticancer therapy, or death, whichever occurs first.

### 6.2.7.2 Screening Visit (Day˜28 to Day 1)

Informed consent
Study eligibility per inclusion/exclusion criteria
Medical history
Electrocardiogram (ECG)
Imaging (previous imaging with similar modality is used if performed within 3 months prior to start of study treatment;):
   A diagnostic quality CT of the chest with IV contrast is required unless medically contraindicated. If the subject is unable to tolerate IV contrast, non-contrast chest CT is acceptable.
   For imaging of the upper tract, abdomen, and pelvis, CT or MRI urogram with contrast (unless medically contraindicated) is acceptable.
Complete eye exam
Initiate collection of archived tumor biopsy specimen for submission upon enrollment
Cystoscopy and cytology (all known sites of disease must be documented at screening and re-assessed at each subsequent tumor evaluation. Subjects with T1 disease should undergo repeat TURBT prior to starting study. Re-staging TURBT must show uninvolved muscularis propria)
Biopsy (if clinically indicated)
Blood and urine sample collection for biomarker assessments (if possible and if applicable, to be collected prior to or at time of TURBT)

### 6.2.7.3 Baseline Visit (Day˜7 to Day 1)

Physical examination
Height and weight (measurements of height obtained within the prior 12 months is utilized)
Vital signs
ECOG performance status
a Blood and urine sample collection for:
   CBC with differential
   Serum chemistry panel (including glucose)
   HbA1c
   Serology (hepatitis B and C)
   Prothrombin time/partial thromboplastin time/international normalized ratio(PT/PTT/INR)
   Urinalysis (with reflexive microscopy for abnormal results)
   Serum or urine β-hCG pregnancy test for subjects of childbearing potential

### 6.2.7.4 Induction Phase (Month 1 to Month 3)

(i) Day 1 of Week 1 to Week 6 (±1 Day after Week 1 Only)
Prior to enfortumab vedotin intravesical administration:
   Confirm subject eligibility per inclusion/exclusion criteria; record medical history (Week 1 only)
   Physical examination; discuss new or ongoing symptoms*
   Weight*
   ECOG performance status (Week 1 only)*
   Vital signs
   Collect AEs and concomitant medications (if applicable)
   Blood and/or urine sample collection for:
      Serum chemistry panel (Weeks 1, 3, and 6 only)*
      Glucose should be verified as <250 mg/dL prior to dosing either by blood draw or fingerstick
      CBC with differential*
      PT/PTT/INR (if clinically indicated)
      PK/antitherapeutic antibody (ATA) assessments
      Biomarker assessments
      Serum or urine β-hCG pregnancy test (Weeks 1, 3, and 6) (only subjects of childbearing potential) (not required if collected within 7 days prior to dosing)
      Urinalysis (with reflexive microscopy for abnormal results)*
Results from clinical laboratory assessments must be reviewed and must confirm eligibility prior to study drug administration (Week 1 only)
Enfortumab vedotin intravesical administration (to be administered q1wk for 6 weeks)
After completion of enfortumab vedotin intravesical administration:
   Vital signs (to be collected 2 hours [±15 minutes] after the first void)
   Observation for 2 hours after the first void
   Collect AEs and concomitant medications (if applicable)
   Blood and urine samples for PK assessments
On Day 1 of Week 1 only, indicated assessments is collected within 3 days prior to dosing.

### (ii) Day 2 of Week 1 to Week 6

The Day 2 visits during the induction phase is conducted in the clinic or via a telehealth/phone call visit. For subjects who do not have an in-clinic visit, blood and urine samples is collected via a home health provider.
Physical examination (to be conducted if visit is performed on site)
Subject interview (+7-day window; Week 6 only; subjects who discontinue prior to Week 6 of the induction phase will undergo an exit interview at their final visit)
Collect AEs and concomitant medications (if applicable)
Blood and urine samples for PK assessments (±4 hours; Weeks 1, 2, and 6 only)
   (iii) Day 4 of Week 1 to Week 6

For subjects who do not have an in-clinic visit, blood and urine samples is collected via a home health provider.
Collect AEs and concomitant medications (if applicable)
Blood and urine samples for PK assessments (±4 hours; Weeks 1, 2, and 6 only)

### (iv) Day 1 of Week 9 (±3 Days)

Serum or urine β-hCG pregnancy test (only subjects of childbearing potential)

### 6.2.7.5 Maintenance Phase (Month 4 to Month 12)

### (i) Day 1 (±3 Days)

Prior to enfortumab vedotin intravesical administration:
   Informed consent (Month 4 only; subjects with persistent CIS or recurrent high-grade Ta disease at the first on-study 3-month evaluation may continue therapy after they are reconsented for continuing study treatment and be assessed again at 6 months)
   Physical examination; discuss new or ongoing symptoms
   Weight
   ECOG performance status
   Vital signs
   Collect AEs and concomitant medications (if applicable)
   Blood and/or urine sample collection for:
      Serum chemistry panel
      Glucose should be verified as <250 mg/dL prior to dosing either by blood draw or fingerstick
      CBC with differential
      PT/PTT/INR (if clinically indicated)
      PK/ATA assessments
      Biomarker assessments
      Serum or urine β-hCG pregnancy test (only subjects of childbearing potential)(not required if collected within 7 days prior to dosing)
      Urinalysis (with reflexive microscopy for abnormal results)
   Cystoscopy and cytology (Subjects must have a cystoscopy prior to entering the maintenance phase and every 3 months from the first induction dose for the first 2 years after enrollment; must be completed within 14 days prior to administration of study drug)
   Bladder mapping biopsy (required at the Month 12 assessment. In the absence of visible tumor, biopsies should be obtained from all quadrants of the bladder [minimum 4 biopsies required]. Biopsy is not required at all other visits but will be considered when clinically indicated for consideration of efficacy)
Enfortumab vedotin intravesical administration (to be administered monthly for a total of 9 doses)
After completion of enfortumab vedotin intravesical administration:
   Vital signs (to be collected 2 hours [±15 minutes] after the first void)
   Observation for 2 hours after the first void
   Collect AEs and concomitant medications (if applicable)
   Blood and urine samples for PK assessments

### (ii) Day 15 (±3 Days)

The Day 15 visit is required during the first 3 maintenance doses and will be conducted via telehealth/phone call to assess for AEs and concomitant medications.
Subject interview (to be conducted at the completion of 5 doses during the maintenance phase and at the end of the maintenance phase [+7-day window])

### 6.2.7.6 Response Assessments

Tumor response assessment on the study occur via standard of care cystoscopy (ie, cystoscopy±biopsy) and cytology at screening followed by assessments every 3 months from the first induction dose for the first 2 years after enrollment, and every 6 months thereafter for 5 years after enrollment.

Response assessment via cystoscopy and urine cytology must be completed within 14 days prior to the next administration of study drug. Subjects with abnormal cystoscopy, positive, or abnormal urine cytology should undergo additional evaluation (eg, imaging, biopsies, exam under anesthesia) per the investigator's clinical discretion.

Bladder mapping biopsies are required at the Month 12 assessment for the study. In the absence of visible tumor, biopsies should be obtained from all quadrants of the bladder (minimum 4 biopsies required). Biopsy is not required at all other visits but are considered when clinically indicated for consideration of efficacy.

6.2.7.7 End of Treatment Visit (30 to 37 Days after Last Dose of Study Drug)

End of treatment (EOT) visits should occur 30 to 37 days after the last dose of study drug unless delayed due to an AE. Note: The time to EOT visit is longer than 37 days for subjects who discontinue treatment after the Month 3 cystoscopy; however, EOT evaluations must be performed before initiation of a new anticancer treatment. If EOT evaluations are completed before 30 days after the last study treatment, the subject is contacted 30 to 37 days following the last treatment to assess for AEs.
Physical examination
Vital signs
ECOG performance status
Complete eye examination, if applicable (EOT slit lamp examinations are required for subjects who experience corneal AEs during the study and must be performed at least 4 weeks from last dose)
Collect AEs and concomitant medications (if applicable)
Blood and/or urine sample collection for:
   Serum chemistry panel (including glucose)
   CBC with differential
   PK/ATA assessments
   Biomarker assessments
   Serum or urine β-hCG pregnancy test (only subjects of childbearing potential)
   Urinalysis (with reflexive microscopy for abnormal results)
Disease status, survival status, collection of first subsequent therapy (if applicable)

### 6.2.7.8 Follow-Up (±1 Week)

Subjects who discontinue study treatment due to reasons other than disease persistence, recurrence, or progression and subjects who complete the maintenance phase enter the follow-up phase of the study. In the follow-up phase, tumor response assessment via standard of care cystoscopy (ie, cystoscopy±biopsy) and cytology occur every 3 months from the first induction dose for the first 2 years after enrollment, and every 6 months thereafter for 5 years after enrollment until disease recurrence, progression, initiation of subsequent anticancer therapy, or death, whichever occurs first.

The following assessments is performed every 3 months from the first induction dose for the first 2 years after enrollment, and every 6 months thereafter for 5 years after enrollment until disease recurrence, progression, initiation of subsequent anticancer therapy, or death, whichever occurs first.
Physical examination
Serum or urine β-hCG pregnancy test (only subjects of childbearing potential) (collected monthly for 6 months after the last received dose of enfortumab vedotin)
Urinalysis (with reflexive microscopy for abnormal results)
Imaging (if clinically indicated)
Blood and urine samples for biomarker assessments
Cystoscopy and cytology
Biopsy (if clinically indicated)
Collection of AEs if serious and considered to be related to study treatment

### 6.2.7.9 Survival Follow-Up (t2 Weeks)

After discontinuation of study treatment due to disease persistence, recurrence, progression, or initiation of subsequent anticancer therapy, subjects enter survival follow-up. Disease status, survival, and subsequent anticancer therapy data are collected every 6 months (±2 weeks) until loss to follow-up, withdrawal of consent, death, or study termination by the sponsor, whichever occurs first, for a maximum of 5 years after enrollment.

### 6.2.7.10 End of Study/End of Follow-Up

The date the subject met criteria for study discontinuation and the reason for study discontinuation will be recorded.

### 6.2.8 Study Assessments

### 6.2.8.1 Screening/Baseline Assessments

Only subjects who meet the eligibility criteria are enrolled in this study.

Subject medical history includes a thorough review of significant past medical history, current conditions, any treatment for prior malignancies and response to prior treatment, and any concomitant medications.

Physical examinations should include assessments of the following body parts/systems: abdomen, extremities, head, heart, lungs, neck, and neurological. Weight and height are also measured; measurements of height obtained within the prior 12 months is utilized.

A complete eye exam is conducted at screening.

Blood and urine tests include CBC with differential, serum chemistry panel, HbA1c, glucose, serology (hepatitis B and C), PT/PTT/INR, and urinalysis (with reflexive microscopy for abnormal results). A serum or urine pregnancy test is conducted for subjects of childbearing potential.

Blood and urine samples are collected for biomarker assessments. Archived tumor specimen or tissue collected from the most recent TURBT collected within 12 months of enrollment is required, if available. See Table 18 and Table 19 for further details.

An ECG is performed at screening for all subjects.

Chest CT and upper tract, abdomen, and pelvis imaging with CT or MRI urography is required within 3 months prior to start of study treatment. For chest CT, imaging with contrast is preferred. If the subject is unable to tolerate IV contrast, non-contrast CT is acceptable. For imaging of the upper tract, abdomen, and pelvis, CT or MRI urogram with contrast (unless medically contraindicated) is acceptable.

### 6.2.8.2 Response/Antitumor Activity Assessments

Tumor response assessment on the study occurs via standard of care cystoscopy (ie, cystoscopy±biopsy) and cytology at screening followed by assessments every 3 months from the first induction dose for the first 2 years after enrollment, and every 6 months thereafter for 5 years after enrollment.

Response assessment via cystoscopy and urine cytology must be completed within 14 days prior to the next administration of study drug. Subjects with abnormal cystoscopy, positive or abnormal urine cytology should undergo additional evaluation (eg, imaging, biopsies, exam under anesthesia) per the investigator's clinical discretion.

Annual upper tract imaging is performed as clinically indicated while the subject is on study.

White-light cystoscopy, narrow band imaging cystoscopy, or fluorescent (blue-light) are acceptable for assessments on study. As much as possible, after initiating study treatment, the same cystoscopy method should be used for disease surveillance for each subject throughout the study and captured in the study CRF.

Bladder mapping biopsies are required at the Month 12 assessment for the study. In the absence of visible tumor, biopsies should be obtained from all quadrants of the bladder (minimum 4 biopsies required). Biopsy is not required at all other visits but is considered when clinically indicated for consideration of efficacy.

Subjects are considered to have a CR when they have all of the following findings:
1. Cystoscopy: normal appearance of bladder. In case of abnormal appearance of bladder on cystoscopy, biopsies should be negative or show low-grade Ta, any grade papillary urothelial neoplasm of low malignant potential, or any grade papilloma. If random bladder biopsies are performed, these biopsies should be negative or show low grade disease.
2. Urine cytology: negative.
   a. Urine cytology that is not conclusive should be evaluated
   b. Positive urine cytology should be further evaluated clinically by cystoscopy±biopsy and imaging
3. Imaging (if performed): normal or, if found abnormal, findings should support a CR in the bladder.

Due to the intravesical administration of the study treatment, a subject is considered to have a CR if they have negative cystoscopy with malignant urine cytology if cancer is found in the upper tract or prostatic urethra and random bladder biopsies are negative.

### (i) Interpretation of Urine Cytology

If a subject has a positive urine cytology while on study, further evaluation must be undertaken by the investigator with cystoscopy and/or upper tract imaging, as clinically indicated.
Positive urine cytology cannot be used alone as an indicator of disease progression, and supportive clinical studies must be performed.
If the urine cytology results are Unsatisfactory, Atypical Cell, Suspicious, or Indeterminate, then a repeat cytology must be performed within the next 21 days. Time between samples should be >24 hours at minimum.
If 2 consecutive repeat urine cytology results are Suspicious or Indeterminate, these results require further clinical evaluation by cystoscopy and/or upper tract imaging as determined by the treating investigator.
If the subject has a Suspicious or Indeterminate urine cytology and the biopsy is negative, then the result is interpreted as negative.
If 2 consecutive results are unsatisfactory or atypical, the overall result would be considered negative.

Persistent disease is defined as presence of CIS disease with or without papillary disease (high-grade Ta/T1).

Recurrence is defined as the reappearance of high-grade disease (high-grade Ta, T1, or CIS) after the start of therapy. Recurrence must be confirmed by biopsy.

Progression is defined as the development of any of the following: T1 disease (lamina propria invasion), ≥T2 disease (muscle invasive), lymph node (N1+), distant metastases (M1), or an increase in grade from low to high.

Persistence, appearance, or presence of lower grade disease is not considered recurrence. Subjects with recurrent low-grade papillary disease may undergo resection and continue on study.

Subjects' clinical data must be available for CRF source verification.

### 6.2.8.3 Pharmacokinetic and Immunogenicity Assessments

Blood and urine samples for PK and ATA analyses are collected throughout the study per the sample collection schedule provided in Table 18 and Table 19. Enfortumab vedotin, ac-MMAE, total antibody (TAb), and unconjugated MMAE concentrations will be determined using validated assays. The assays may include enzyme-linked immunosorbent assays (ELISA), 1% as well as other assays if further characterization is required. PK samples are archived for possible analysis of other enfortumab vedotin related species. An electrochemiluminescence assay are used to determine levels of ATA against enfortumab vedotin in serum. Dose related blood PK parameters for enfortumab vedotin to be estimated may include, but are not limited to, area under the concentration-time curve (AUC), maximum concentration (Cmax), time to maximum concentration (Tmax), apparent terminal half-life (t½), and trough concentration (Ctrough).

Additional analytes is evaluated as necessary.

### 6.2.8.4 Pharmacodynamic and Biomarker Assessments

Biomarker assessments are performed in urine, peripheral blood, and tumor tissue as outlined in this section and the Schedule of Events. Biomarker assessments are not used for subject selection. Exploratory, predictive, and prognostic biomarkers associated with response, resistance, or safety observations are monitored before and during treatment with enfortumab vedotin.

The primary effects of enfortumab vedotin on tumor cells may lead to changes in the activation state of local, tumor-associated, and peripheral immune cells. Biomarker assessments in blood and urine samples may include, but may not be limited to, circulating/cell free tumor DNA, ELISA assessment of soluble Nectin-4, immunoassays of urinary biomarkers, and markers of immune function, including abundance of immune cell subsets and cytokines.

Archival tumor tissue that was collected within 12 months of enrollment is required for all subjects, if available (the most recently available tissue should be used). If freshly cut slides only can be provided, a minimum of 10 to 15 sections is required (if less than 10, contact the sponsor). Biopsy is required at the Month 12 assessment and should be performed as clinically indicated at all other assessments. If a tumor sample is obtained as part of standard of care during the study, a part of that sample should be submitted to the sponsor for biomarker testing.

Biopsies should be collected by appropriately trained clinical site personnel. It is recommended that a pathologist be present during biopsies when feasible to ensure sufficient tumor content of the biopsy location, and to confirm that biopsy acquisition and processing techniques are optimal (Ferry-Galow 2018).

To understand the relationship between the biological characteristics of tumors before treatment and subject outcomes, tissue from the TURBT (tumor biopsies) are examined. Biopsies are assessed for specific pharmacodynamic, predictive, and prognostic biomarkers in the tumor. If tissue is available from a standard of care biopsy collected after enrollment, it may also be examined to further identify biomarkers of response and mechanism of action and resistance to treatment.

Biomarker assessments in tumor tissue may include, but are not limited to, central assessment of Nectin-4 and PD-L1 expression by immunohistochemistry and next generation sequencing, tumor subtyping, tumor microenvironment analyses, and profiling of somatic mutations or alterations in genes or RNA commonly altered in cancer.

### 6.2.8.5 Biospecimen Repository

In the US only, for subjects who provide additional consent, remaining de-identified unused blood and/or tissue is retained by the study sponsor and used for future research, including but not limited to the evaluation of targets for novel therapeutic agents, the biology of ADC sensitivity and resistance mechanisms, and the identification of biomarkers of ADCs. Blood and tissue samples donated for future research are retained for a period of up to 25 years. If additional consent is not provided, any remaining biological samples are destroyed after the study has been completed and all applicable regulatory obligations have been met.

### 6.2.8.6 Subject Interview

The subject perspective is qualitatively assessed through telephone interviews lasting 45 to 60 minutes covering topics around experience and tolerability of treatment. Additional details regarding the topics and questions that are explored during these interviews are described in the Subject Interview Guide. Interviews are conducted during dose escalation and dose expansion at the following time points: at the end of the induction phase, at the completion of 5 doses during the maintenance phase, and at the end of the maintenance phase.

### 6.2.8.7 Safety Assessments

The assessment of safety during the course of this study consists of the surveillance and recording of AEs including SAEs, recording of concomitant medications, and measurements of protocol-specified physical examination findings and laboratory tests.

### (i) Adverse Events

### (a) Definitions

### Adverse Event

According to the International Council for Harmonisation (ICH) E2A guideline Definitions and Standards for Expedited Reporting, and 21 CFR 312.32, IND Safety Reporting, an AE is any untoward medical occurrence in a subject or clinical investigational subject administered a medicinal product and which does not necessarily have a causal relationship with this treatment.

The following information should be considered when determining whether or not to record a test result, medical condition, or other incident on the Adverse Events CRF:
From the time of informed consent through the day prior to study Day 1, only study protocol-related AEs should be recorded. A protocol-related AE is defined as an untoward medical event occurring as a result of a protocol mandated procedure.
All medical conditions present or ongoing predose on study Day 1 that increase in NC ICTCAE grade should be recorded.
Medical conditions present or ongoing predose on study Day 1 that worsen in severity, increase in frequency, become related to study drug, or worsen in any other way but do not meet the threshold for increase in NCI CTCAE grade should be recorded.
All AEs (regardless of relationship to study drug) should be recorded from study Day 1(during and post-dose) through the end of the safety reporting period. Complications that occur in association with any procedure (eg, biopsy) should be recorded as AEs whether or not the procedure was protocol mandated.
In general, an abnormal laboratory value should not be recorded as an AE unless it is associated with clinical signs or symptoms, requires an intervention, results in a SAE, or results in study termination or interruption/discontinuation of study treatment. When recording an AE resulting from a laboratory abnormality, the resulting medical condition rather than the abnormality itself should be recorded (eg, record “anemia” rather than “low hemoglobin”).

### Serious Adverse Events

An AE should be classified as an SAE if it meets one of the following criteria:
Fatal: AE results in death
Life threatening: The AEs places the subject at immediate risk of death. This classification does not apply to an AE that hypothetically might cause death if it were more severe.
Hospitalization: The AE results in hospitalization or prolonged an existing inpatient hospitalization. Hospitalizations for elective medical or surgical procedures or treatments planned before the signing of informed consent in the study or routine check-ups are not SAEs by this criterion. Admission to a palliative unit or hospice care facility is not considered to be a hospitalization. Hospitalizations or prolonged hospitalizations for scheduled therapy of the underlying cancer or study target disease need not be captured as SAEs.
Disabling/incapacitating: An AE that results in a persistent or significant incapacity or substantial disruption of the subject's ability to conduct normal life functions.
Congenital anomaly or birth defect: An adverse outcome in a child or fetus of a subject who is exposed to the molecule or study treatment regimen before conception or during pregnancy.
Medically significant: The AE does not meet any of the above criteria, but may jeopardize the subject and might require medical or surgical intervention to prevent one of the outcomes listed above or involves suspected transmission via a medicinal product of an infectious agent. Potential DILI also is considered a medically significant event.

### Adverse Event Severity

AE severity should be graded using the NCI CTCAE, Version 5.0. These criteria are provided in the study manual.

AE severity and seriousness are assessed independently. ‘Severity’ characterizes the intensity of an AE. ‘Serious’ is a regulatory definition and serves as a guide to the sponsor for defining regulatory reporting obligations (see definition for SAEs, above).

### Relationship of the Adverse Event to Study Treatment

The relationship of each AE to enfortumab vedotin should be evaluated by the investigator using the following criteria:
Related: There is evidence to suggest a causal relationship between the drug and the AE, such as:
   A single occurrence of an event that is uncommon and known to be strongly associated with drug exposure (eg, angioedema, hepatic injury, Stevens-Johnson Syndrome) One or more occurrences of an event that is not commonly associated with drug exposure, but is otherwise uncommon in the population exposed to the drug (eg, tendon rupture)
Unrelated: Another cause of the AE is more plausible (eg, due to underlying disease or occurs commonly in the study population), or a temporal sequence cannot be established with the onset of the AE and administration of the study treatment, or a causal relationship is considered biologically implausible

### (b) Procedures for Eliciting and Recording Adverse Events

Investigator and study personnel reports all AEs and SAEs whether elicited during subject questioning, discovered during physical examination, laboratory testing and/or other means byre cording them on the CRF and/or SAE form, as appropriate.

### Eliciting Adverse Events

An open-ended or non-directed method of questioning should be used at each study visit to elicit the reporting of AEs.

### Recording Adverse Events

The following information should be recorded on the Adverse Events CRF:
Description including onset and resolution dates
Whether it met SAE criteria
Severity
Relationship to study treatment or other causality
Outcome
   Diagnosis Vs. Signs or Symptoms

In general, the use of a unifying diagnosis is preferred to the listing out of individual symptoms. Grouping of symptoms into a diagnosis should only be done if each component sign and/or symptom is a medically confirmed component of a diagnosis as evidenced by standard medical textbooks. If any aspect of a sign or symptom does not fit into a classic pattern of the diagnosis, the individual symptom is reported as a separate AE.

### Recording Serious Adverse Events

For SAEs, the event(s) is recorded on both the CRF and an SAE form. The following should be considered when recording SAEs:
Death is an outcome of an event. The event that results in the death should be recorded and reported on both an SAE form and CRF.
For hospitalizations, surgical, or diagnostic procedures, the illness leading to the surgical or diagnostic procedure should be recorded as the SAE, not the procedure itself. The procedure should be captured in the narrative as part of the action taken in response to the illness.

### Progression of Underlying Malignancy

Since progression of underlying malignancy is being assessed as an efficacy variable, it should not be reported as an AE or SAE. The terms “Disease Progression”, “Progression of Disease”, or “Malignant disease progression” and other similar terms should not be used to describe an AE or SAE. However, clinical symptoms of progression is reported as AEs or SAEs if the symptom cannot be determined as exclusively due to progression of the underlying malignancy or does not fit the expected pattern of progression for the disease under study. In addition, complications from progression of the underlying malignancy should be reported as AEs or SAEs.

### Pregnancy

### Notification to Drug Safety

A Pregnancy Report Form is completed for all pregnancies that occur from the time of first study drug dose until 6 months after the last dose of study drug(s) including any pregnancies that occur in the partner of a study subject who is able to father a child. Only pregnancies that occur in a subject's partner are reported if the estimated date of conception is after the subject's first study drug dose. An email or fax is sent to the sponsor's Drug Safety Department within 48 hours of becoming aware of a pregnancy. All pregnancies are monitored for the full duration; all perinatal and neonatal outcomes should be reported. Infants should be followed for a minimum of 8 weeks.

### Collection of Data on the CRF

All pregnancies (as described above) that occur within 30 days of the last dose of study drug(s) are also be recorded on the Adverse Events CRF.

Abortion, whether accidental, therapeutic, or spontaneous, should be reported as an SAE. Congenital anomalies or birth defects, as defined by the ‘serious’ criterion above should be reported as SAEs.

### Corneal Adverse Events

Corneal ulcer or keratitis AEs≥Grade 2 should be graded within their respective NCI CTCAE categories. Grade 1 corneal ulcer or keratitis AEs should be graded per “Eye disorders—Other, specify” criteria. Other corneal AEs should be recorded and graded per “Eye disorders—Other, specify” criteria.

### Diabetes and Hyperglycemia

Grading for diabetes should be based on the NCI CTCAE v5.0 event term of glucose intolerance. Grading for hyperglycemia should be based on the NCI CTCAE v5.0 event term of hyperglycemia.

### Potential Drug-Induced Liver Injury

Hy's Law is used to estimate severity and the likelihood that a study drug may cause an increased incidence of severe hepatotoxicity.

The absence of hepatotoxicity in clinical trials provides a limited predictive value for potential drug-induced liver injury (DILI) in the clinical setting(s) being studied. However, finding 1 Hy's Law case in clinical trials is ominous; finding 2 cases is highly predictive of a potential for severe DILI.

### Definition

Briefly, potential Hy's Law cases include the following 3 components:
1. Aminotransferase (ALT and/or AST) elevation>3×ULNAND
2. Total bilirubin>2×ULN, without initial findings of cholestasis (ie, elevated serum alkalinephosphatase),

### AND

3. No other immediately apparent possible causes of aminotransferase elevation and hyperbilirubinemia, including, but not limited to, viral hepatitis, pre-existing chronic or acute liver disease, or the administration of other drug(s) known to be hepatotoxic.

### Reporting Requirements

Any potential Hy's Law case should be handled as an SAE and reported promptly to the sponsor.

Reporting should include all available information and should initiate close follow-up until complete resolution of the problem and completion of all attempts to obtain supplementary data.

### Follow-Up for Abnormal Laboratory Results Suggesting Potential DILI

In general, an increase of serum ALT or AST to >3×ULN should be followed by repeat testing within 48 to 72 hours of serum ALT, AST, alkaline phosphatase, and total bilirubin, to confirm the abnormalities and to determine whether they are worsening.

Appropriate medical assessment should be initiated to investigate potential confounding factors and alternative causes of hepatotoxicity. During this investigation, consider withholding study drug.

### (c) Reporting Periods for Adverse Events and Serious Adverse Events

The safety reporting period for all AEs and SAEs is from study Day 1 (predose) through 30 days after the last study treatment. However, all study protocol-related AEs are recorded from the time of informed consent. All SAEs that occur after the safety reporting period and are considered study treatment-related in the opinion of the investigator should also be reported to the sponsor.

SAEs are followed until significant changes return to baseline, the event stabilizes (recovering/resolving) or is no longer considered clinically significant by the investigator, or the subject dies or withdraws consent. All non-serious AEs are followed through the safety reporting period. Certain non-serious AEs of interest is followed until resolution, return to baseline, or study closure.

### (d) Serious Adverse Events Require Immediate Reporting

Within 24 hours of observing or learning of an SAE, investigators report the event to the sponsor, regardless of the relationship of the event to the study treatment regimen.

For initial SAE reports, available case details are recorded on an SAE form. At a minimum, the following should be included:
Subject number
Date of event onset
Description of the event
Study treatment, if known
Investigator causality assessment

The completed SAE form is emailed or faxed to the sponsor's Drug Safety Department within 24 hours (see email or fax number specified on the SAE report form).

Relevant follow-up information is submitted to the sponsor as soon as it becomes available.

### (e) Sponsor Safety Reporting to Regulatory Authorities

Investigators are required to report all SAEs to the sponsor. The sponsor reports all SAEs, including suspected unexpected serious adverse reactions (SUSARs) to regulatory authorities as required per local legislation or regulatory reporting requirements.

### (f) Adverse Events of Special Interest

Certain non-serious adverse events of special interest (AESIs) is followed (including collection of relevant concomitant medications) until resolution, return to baseline, subject withdrawal, study closure, or the events become chronic to the extent that they are adequately characterized.

AESIs related to enfortumab vedotin for this purpose include but may not be limited to those events in the list below:
Skin Reactions
Peripheral neuropathy
Corneal event
Hyperglycemia

AESIs should be promptly reported by electronic data capture (EDC). If an event meets serious criteria it should be reported as a serious event within 24 hours.

### (ii) Vital Signs

Vital signs measures include heart rate, systolic and diastolic blood pressure, and temperature. Vital sign values are recorded, and any diagnosis associated with clinically significant abnormal vital signs are recorded as an AE or pre-existing condition.

(iii) Clinical Laboratory Tests

Samples are drawn for local labs. Local laboratory testing includes institutional standard tests for evaluating safety and making clinical decisions. The following laboratory assessments are performed by local laboratories to evaluate safety at scheduled time points (see Schedule of Events) during the course of the study:
The serum chemistry panel includes the following tests: albumin, alkaline phosphatase, ALT, AST, bicarbonate, blood urea nitrogen, calcium, creatinine, chloride, lactate dehydrogenase (ALDH), phosphorus, potassium, sodium, total bilirubin, amylase, lipase, glucose, and uric acid.
HbA1c
Glucose (must be verified to be <250 mg/dL prior to dosing either by blood draw or fingerstick).
The CBC with differential includes the following tests: white blood cell count with five-part differential (neutrophils, lymphocytes, monocytes, eosinophils, and basophils), platelet count, Hgb, and hematocrit.
Calculated CrCl (GFR can also be used in place of creatinine or CrCl). CrCl should be calculated using the Cockcroft-Gault method or MDRD equations.

The following laboratory assessment(s) is performed by local laboratories at scheduled timepoints (see Schedule of Events) during the course of the study:
Serology (hepatitis B and C)
PT/PTT/INR
Urinalysis
   Standard urinalysis (with reflexive microscopy, if abnormal)
   A serum or urine β-hCG pregnancy test for subjects of childbearing potential

### (iv) Physical Examination

Physical examinations should include assessments of the following body parts/systems: abdomen, extremities, head, heart, lungs, neck, and neurological. Weight and height are also measured. Measurements of height obtained within the prior 12 months is utilized.

Prior to each intravesical enfortumab vedotin instillation, suggested questions addressed with the subject may include any new or ongoing symptoms such as abdominal pain (especially in the lower abdomen, flank, or urethra), fevers, chills, symptoms suggestive of obstruction, skin rash, and check for any signs of bleeding (minimal or gross hematuria, clots). If the overall clinical evaluation is suggestive of an AE, further treatment should be held until symptoms resolve per the treating investigator's discretion.

### (v) ECOG Performance Status

ECOG performance status will be evaluated at protocol-specified timepoints.

### (vi) Electrocardiograms

All subjects receive 12-lead ECGs in triplicate at screening. Additional ECGs should be conducted if clinically indicated. ECGs are performed after the subject has been in a supine position for at least 5 minutes. The ECG should be performed prior to obtaining biomarker samples, if possible.

Waiting periods between each ECG are not required. Electronic or paper copies of the tracings is submitted to the sponsor's designee for possible central assessment.

(vii) Pregnancy Testing

For subjects of childbearing potential, a serum or urine β-hCG pregnancy test with sensitivity of at least 25 mIU/mL is performed at baseline, on study Day 1 of Weeks 1, 3, 6, and 9 during induction, on study Day 1 of each month during the maintenance phase, at the EOT visit, and monthly for 6 months after the last received dose of enfortumab vedotin. A negative pregnancy result is required before the subject may receive study drug. Pregnancy tests may also be repeated as requested per Institutional review board independent ethics committee (IRB/IEC) or if required by local regulations.

(viii) Imaging

Subjects undergo a CT or MRI urogram of the upper tract, abdomen, and pelvis and imaging of the chest at screening. If the subject is unable to tolerate IV contrast, non-contrast CT is acceptable. For imaging of the upper tract, abdomen, and pelvis, CT or MRI urogram with contrast (unless medically contraindicated) is acceptable. Previous imaging with similar modality is used if performed within 3 months prior to the start of study treatment.

### (ix) Complete Eye Examination

Subjects have a complete eye examination at screening performed by a qualified optometrist or ophthalmologist, including but not limited to visual acuity, slit lamp, tonometry examination, and dilated fundus examination. Subsequent eye examinations are conducted as clinically indicated. EOT slit lamp examinations are required for subjects who experience corneal AEs during the study and must be performed at least 4 weeks from last dose.

### 6.2.8.8 Post-Treatment Assessments

### (i) Follow-Up Assessments

Subjects who discontinue study treatment due to reasons other than disease persistence, recurrence, or progression and subjects who complete the maintenance phase enter the follow-up phase of the study. Physical examination, urinalysis, and pregnancy test are performed as indicated in the Schedule of Events. Imaging is performed if clinically indicated. During follow-up, tumor response assessment via standard of care cystoscopy (i.e., cystoscopy f biopsy) and cytology will occur every 3 months from the first induction dose for the first 2 years after enrollment, and every 6 months thereafter for 5 years after enrollment until disease recurrence, progression, initiation of subsequent anticancer therapy, or death, whichever occurs first.

### (ii) Survival Follow-Up Assessments

After discontinuation of study treatment due to disease persistence, recurrence, progression, or initiation of subsequent anticancer therapy, subjects enter the survival follow-up. Survival and subsequent anticancer therapy data is collected every 6 months (±2 weeks) until loss to follow-up, withdrawal of consent, death, or study termination by the sponsor, whichever occurs first, for a maximum of 5 years after enrollment.

### 6.2.8.9 Appropriateness of Measurements

The safety measures that aroused in this trial are considered standard procedures for evaluating the potential adverse effects of study medications.

Response is assessed by cystoscopy±biopsy and cytology, which is standard for evaluating response in NMIBC. The intervals of evaluation in this protocol are considered appropriate for disease management.

Immunogenicity is commonly assessed for biologics; therefore, standard tests are performed to detect the possible presence of specific antibodies to enfortumab vedotin. Pharmacokinetic assessments are also common in clinical studies to help characterize dose-exposure-response relationships.

### 6.2.9 Data Analysis Methods

### 6.2.9.1 Determination of Sample Size

Approximately 58 subjects is enrolled in this study. This includes approximately 18 subjects evaluated in dose-escalation and approximately 40 subjects evaluated in up to 2 expansion cohorts (approximately 20 subjects in each cohort).

The exact number of subjects needed to complete the dose escalation is unknown because it depends on the number of dose levels evaluated to reach the MTD and the number of subjects treated at each dose level.

No formal hypothesis testing is planned for the expansion cohort. Assuming an observed CR rate in the range of 30% to 50%, the 95% and 80% exact CIs with 20 subjects per cohort are summarized above in Table 11.

### 6.2.9.2 Study Endpoint Definitions

Study endpoints are presented in 6.1.3 Objectives. Endpoint definitions are presented in this section.

### (i) Complete Response Rate

CR rate is defined as the proportion of subjects achieving CR.

Subjects will be considered to have a CR when they have all of the following findings:
1. Cystoscopy: normal appearance of bladder. In case of abnormal appearance of bladder on cystoscopy, biopsies that are performed are negative or show low-grade Ta, any grade papillary urothelial neoplasm of low malignant potential, or any grade papilloma. If random bladder biopsies are performed, these biopsies should be negative or show low grade disease.
2. Urine cytology: negative.
   a. Urine cytology that is not conclusive should be evaluated
   b. Positive urine cytology should be further evaluated clinically by cystoscopy±biopsy and imaging
3. Imaging (if performed): normal or, if found abnormal, findings should support a CR in the bladder.

Due to the intravesical administration of the study treatment, a subject is considered to have a CR if they have negative cystoscopy with malignant urine cytology if cancer is found in the upper tract or prostatic urethra and random bladder biopsies are negative.

Subjects who do not have response assessment post-baseline are considered not achieving a CR.

### (ii) Duration of Complete Response

Duration of CR is defined as the time from first documented CR to the first evidence of recurrence, progression, or death due to any cause, whichever occurs first.

Subjects who achieve a CR, are alive and without disease recurrence and progression at the time of analysis will be censored at the last disease assessment. Detailed censoring rules are provided in the statistical analysis plan (SAP).

(iii) Rate of Cystectomy

Rate of cystectomy is defined as the proportion of subjects who subsequently undergo cystectomy.

### (iv) Progression-Free Survival

Progression-free survival (PFS) is defined as the time from start of study treatment to the first evidence of progression or death due to any cause, whichever occurs first. Subjects who are alive and without progression at the time of analysis are censored at the last disease assessment. Detailed censoring rules are provided in the SAP.

### (v) Cystectomy-Free Survival

Cystectomy-free survival (CFS) is defined as the time from start of study treatment to cystectomy or death due to any cause, whichever occurs first. Subjects who are alive and without cystectomy at the time of analysis are censored at the last known alive date. Detailed censoring rules are provided in the SAP.

### 6.2.9.3 Statistical and Analytical Plans

The high-level statistical and analytical plans are summarized below. More detailed and comprehensive plans will be provided in the SAP.

### (I) GENERAL CONSIDERATIONS

This is a phase 1 dose-escalation study with subsequent expansion cohorts. All analyses are descriptive.

Descriptive statistics (mean, median, standard deviation, minimum, maximum) are used to describe continuous variables. Frequencies and percentages are used to describe categorical variables.

### (a) Randomization and Blinding

This is an open-label dose-escalation and expansion study. Randomization is not utilized, and blinding is not applicable.

### (b) Adjustments for Covariates

Adjustments for covariates are not planned.

### (c) Handling of Dropouts and Missing Data

Missing data is be imputed unless otherwise specified. Missing AE start and stop dates are imputed for the purpose of calculating duration of events and TE status. Details on missing data handling are provided in the SAP.

### (d) Multicenter Studies

There are multiple centers in this study; however, it is not anticipated that any center will accrue enough subjects to warrant an analysis by center.

### (e) Multiple Comparisons and Multiplicity

No multiple comparisons are planned and no alpha adjustment is needed in this phase 1 study.

### (f) Data Transformations and Derivations

Time variables based on 2 dates (eg, start date and end date) are calculated as (end date−start date+1) (in days) unless otherwise specified in the analysis plan.

Baseline values used in all statistical analyses are the most recent non-missing measurement prior to the first dose of study drug unless otherwise specified in the analysis plan.

### (g) Analysis Sets

### All-Treated-Subjects Analysis Set

The all-treated-subjects (ATS) analysis set includes all subjects who receive any amount of enfortumab vedotin. The ATS analysis set is used for the analysis of safety and efficacy endpoints.

### DLT-Evaluable Analysis Set

The DE analysis set includes all treated subjects in dose escalation who either experienced a DLT or received at least 5 induction doses of enfortumab vedotin. The DE analysis set is used for the determination of MTD.

### (h) Examination of Subgroups

As exploratory analyses, subgroup analyses is conducted for selected endpoints. Subgroups may include but are not limited to the following:
Prior therapies
Disease subtype
Nectin-4 expression level

### (i) Timing of Analyses

The final analysis for this study occurs after all subjects have completed their treatment and the follow-up period or following study termination by the sponsor.

### (ii) Subject Disposition

An accounting of study subjects by disposition is tabulated and the number of subjects in each analysis set will be summarized. Subjects who discontinue study treatment and subjects who withdraw from the study are summarized with reason for discontinuation or withdrawal.

(iii) Subject Characteristics

Demographics and other baseline characteristics are summarized. Details will be provided in the SAP.

### (iv) Treatment Compliance

Study drug are administered intravesically at the investigational site. No summary of treatment compliance is planned.

### (v) Efficacy Analyses

All efficacy analyses are performed using the ATS analysis set.

The CR rate at any time on study and the CR rates at 3, 6, 12, 18, and 24 months are summarized along with the exact 95% CI.

Duration of CR are estimated using the Kaplan-Meier method. Only subjects who achieve a CR are included in the analysis.

PFS and CFS are estimated using the Kaplan-Meier method. Kaplan-Meier plots will be presented where appropriate. Detailed methodology is provided in the SAP.

Rate of cystectomy is summarized along with the exact 95% CI.

### (vi) Pharmacokinetic and Immunogenicity Analyses

Enfortumab vedotin concentrations in blood and urine are summarized with descriptive statistics at each PK sampling time point. Dose related PK parameters to be calculated may include, but are not limited to, AUC, Cmax, Tmax, t1/2, and Ctrough and are estimated by noncompartmental analyses and summarized by descriptive statistics. Additional analytes is evaluated as necessary. The relationship between PK and pharmacodynamics endpoints, safety, or efficacy is explored.

The incidence of ATA are summarized by descriptive statistics.

(vii) Biomarker Analyses

Relationships of biomarker parameters (eg, baseline values, absolute and relative changes from baseline) to antitumor activity, safety, and PK parameters are explored. Relationships and associated data that are determined to be of interest is summarized. Details are described separately in the SAP or biomarker analysis plan.

(viii) Patient Reported Outcomes Analyses

Patient reported outcomes are collected via subject interviews. The analysis of the transcripts that will be generated from these interviews are described in a separate document.

### (ix) Safety Analyses

All safety analyses are performed using the ATS analysis set.

### (a) Extent of Exposure

Duration of treatment, number of doses, total dose, and dose intensity are summarized. Dose modifications, including dose delay, skip, and reduction, are summarized. Details are provided in the SAP.

### (b) Adverse Events

An overview of AEs provides a tabulation of the incidence of all treatment-emergent adverse events (TEAEs), treatment-related TEAEs, Grade 3 or higher TEAEs, treatment-related Grade 3 or higher TEAEs, TE SAEs, treatment-related TE SAEs, TEAEs leading to death, treatment-related TEAEs leading to death, TEAEs leading to treatment discontinuation, and treatment-related TEAEs leading to treatment discontinuation. AEs are defined as treatment emergent if they are newly occurring or worsen following first dose of study treatment and on or before 30 days after last dose.

TEAEs are summarized by Medical Dictionary for Regulatory Activities (MedDRA) preferred term, severity, and relationship to study drug. In the event of multiple occurrences of the same AE with the same preferred term in 1 subject, the AE is counted once as the occurrence. TEAEs leading to dose modifications or treatment discontinuation are summarized in the same manner.

All TEAEs, Grade 3 or higher TEAEs, and TEAEs leading to treatment discontinuation are listed.

### (c) Dose-Limiting Toxicity

The number and percentage of subjects experiencing a DLT are summarized for the DE analysis set. Model-based estimate of the probability of DLT is presented along with the 95% credible interval for each dose level.

### (d) Deaths and Serious Adverse Events

SAEs are listed and summarized in the same manner as TEAEs. Events with a fatal outcome are listed.

### (e) Clinical Laboratory Results

Laboratory values (eg, chemistry, hematology, and urinalysis) are summarized by visit. Shifts from baseline to the maximum post-baseline NCI CTCAE grade are tabulated.

Laboratory values are listed with grade per NCI CTCAE and flagged when values are outside the normal reference range.

### (f) Other Safety Analyses

### Vital Signs

Vital signs measurements (systolic and diastolic blood pressure, heart rate, and temperature) are listed.

### ECOG Performance Status

Shifts from baseline to the best and worst post-baseline score is tabulated.

### ECG

ECG status (normal, abnormal clinically significant, or abnormal not clinically significant) are listed.

### (x) Interim Analyses

No formal interim analyses are planned. During the dose-escalation part of the study, data are evaluated by the sponsor and the SMC after each cohort to determine DLTs and inform dose-escalation decisions. The SMC monitors the trial for safety and DLTs on an ongoing basis.

The process for SMC decisions and the roles and responsibilities of the SMC are detailed in a separate document.

Interim data from the study is presented at scientific meetings such as the annual meetings of the American Society of Clinical Oncology.

### 6.2.10 List of Abbreviations and Definitions of Terms

ADC antibody-drug conjugate
AE adverse event
AESI adverse event of special interest
ALT alanine aminotransferase
ANC absolute neutrophil count
aPTT activated partial thromboplastin time
AST aspartate aminotransferase
ATA antitherapeutic antibody
ATS all-treated subjects
AUA American Urological Association
AUC area under the concentration-time curve
β-hCG beta human chorionic gonadotropin
BCG Bacillus Calmette-Guerin
BICR blinded independent central review
Cₘₐₓ maximum concentration
CrCl creatinine clearance
C_{trough} trough concentration
CBC complete blood count
CFS cystectomy-free survival
CI confidence interval
CIS carcinoma in situ
CR complete response
CRF case report form
CT computed tomography
CYP cytochrome P450
DE DLT-evaluable
DILI drug-induced liver injury
DLT dose-limiting toxicity
DOR duration of response
DU current dose is unacceptably toxic
EAU European Association of Urology
ECD extracellular domain
ECG electrocardiogram
ECOG Eastern Cooperative Oncology Group
eCRF electronic case report form
ELISA enzyme-linked immunosorbent assays
EOT end of treatment
FIH first-in-human
GFR glomerular filtration rate
HbA1c hemoglobin A1c
Hgb hemoglobin
HIV human immunodeficiency virus
HR hazard ratio
ICH International Council for Harmonisation
IEC independent ethics committee
Ig immunoglobulin
IND investigational new drug
RB institutional review board
IRR infusion-related reaction
IV intravenous
LDH lactate dehydrogenase
mAB monoclonal antibody
MDRD Modification of Diet in Renal Disease
MedDRA Medical Dictionary for Regulatory Activities
MMAE monomethylauristatin E
MRHD maximum recommended human dose
MRI magnetic resonance imaging
MTD maximum tolerated dose
mTPI modified toxicity probability interval
NCI CTCAE National Cancer Institute Common Terminology Criteria for Adverse Events
NMIBC non-muscle invasive bladder cancer
NOAEL no-observed-adverse-effect-level
NYHA New York Heart Association
ORR objective response rate
OS overall survival
PACS picture archiving and communication system
PCR polymerase chain reaction
PD-1 programmed cell death protein 1
PD-L1 programmed death-ligand 1
PFS progression-free survival
P-gp P-glycoprotein
PK pharmacokinetics
PK/PD pharmacokinetics/pharmacodynamics
PN peripheral neuropathy
PSA prostate-specific antigen
PT/PTT/INR prothrombin time/partial thromboplastin time/international normalized ratio
q1wk once a week
SAE serious adverse event
SAP statistical analysis plan
SJS Stevens-Johnson syndrome
SMC Safety Monitoring Committee
SUSAR suspected unexpected serious adverse reactions
t1/2 half-life
TAb total antibody
TE treatment-emergent
TEAE treatment-emergent adverse event
TEN toxic epidermal necrolysis
Tₘₐₓ time to maximum concentration
TURBT transurethral resection of the bladder tumor
UC urothelial cancer
ULN upper limit of normal
US United States
UTI urinary tract infection

What is claimed:

1. A method of treating bladder cancer in a human subject, comprising intravesically administering to the subject an effective amount of an antibody drug conjugate (ADC), wherein the ADC comprises an antibody or antigen binding fragment thereof that binds to 191P4D12 conjugated to one or more units of monomethyl auristatin E (MMAE).

2. The method of claim 1, wherein the bladder cancer is non-muscle invasive bladder cancer (NMIBC).

3. The method of claim 2, wherein the NMIBC has been histologically confirmed and is carcinoma in situ (CIS).

4. The method of claim 3, wherein the subject has papillary disease.

5. The method of claim 3, wherein the subject does not have papillary disease.

6. The method of any of claims 2 to 5, wherein the NMIBC has been histologically confirmed and wherein the predominant histologic component (>50%) is urothelial (transitional cell) carcinoma.

7. The method of any one of claims 1 to 6, wherein the subject has high-risk Bacillus Calmette-Guerin (BCG)-unresponsive disease.

8. The method of any one of claims 1 to 7, wherein the subject is ineligible for or refuses to undergo a radical cystectomy.

9. The method of any one of claims 1 to 8, wherein all visible papillary Ta/T1 tumors of the subject have completely resected within 60 days prior to the treatment.

10. The method of claim 9, wherein the subject has residual pure CIS.

11. The method of claim 9, wherein the subject does not have residual pure CIS.

12. The method of any one of claims 1 to 11, wherein the subject has an Eastern Cooperative Oncology Group (ECOG) Performance Status score of 0.

13. The method of any one of claims 1 to 11, wherein the subject has an Eastern Cooperative Oncology Group (ECOG) Performance Status score of 1.

14. The method of any one of claims 1 to 11, wherein the subject has an Eastern Cooperative Oncology Group (ECOG) Performance Status score of 2.

15. The method of claim 14, wherein the subject's glomerular filtration rate (GFR) is no less than 50 mL/min and the subject does not have New York Heart Association (NYHA) Class III heart failure.

16. The method of any one of claims 1 to 15, wherein the subject has one or more of the conditions selected from the group consisting of:
a. Absolute neutrophil count (ANC)≥1500/μL;
b. Hemoglobin (Hgb)≥10 g/dL;
c. Platelet count≥100,000/μL;
d. Serum bilirubin≤1.5×upper limit of normal (ULN) or ≤3×ULN for subjects with Gilbert's disease;
e. Calculated creatinine clearance (CrCl)≥30 mL/min (GFR can also be used in place of creatinine or CrCl). CrCl should be calculated using the Cockcroft-Gault method or Modification of Diet in Renal Disease (MDRD) equations. Subjects with an ECOG performance status of 2 must have GFR≥50 mL/min;
f. Alanine aminotransferase (ALT) and aspartate aminotransferase (AST)≤3×ULN; or
g. International normalized ratio (INR) or prothrombin time (PT), activated partial thromboplastin time (aPTT) or partial thromboplastin time (PTT)≤1.5 ULN unless subject is receiving anticoagulant therapy as long as PT or aPTT is within therapeutic range of intended use of anticoagulants.

17. The method of claim 16, wherein the subject has all of conditions (a) to (g) of claim 16.

18. The method of any one of claims 1 to 17, wherein the subject's estimated life expectancy is more than 2 years.

19. The method of any one of claims 1 to 18, wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23.

20. The method of any one of claims 1 to 19,
wherein the antibody or antigen binding fragment thereof comprises CDR-H1 comprising the amino acid sequence of SEQ ID NO:9, CDR-H2 comprising the amino acid sequence of SEQ ID NO:10, CDR-H3 comprising the amino acid sequence of SEQ ID NO:11; CDR-L1 comprising the amino acid sequence of SEQ ID NO:12, CDR-L2 comprising the amino acid sequence of SEQ ID NO:13, and CDR-L3 comprising the amino acid sequence of SEQ ID NO:14, or
wherein the antibody or antigen binding fragment thereof comprises CDR-H1 comprising the amino acid sequence of SEQ ID NO:16, CDR-H2 comprising the amino acid sequence of SEQ ID NO:17, CDR-H3 comprising the amino acid sequence of SEQ ID NO:18; CDR-L1 comprising the amino acid sequence of SEQ ID NO:19, CDR-L2 comprising the amino acid sequence of SEQ ID NO:20, and CDR-L3 comprising the amino acid sequence of SEQ ID NO:21.

21. The method of any one of claims 1 to 19,
wherein the antibody or antigen binding fragment thereof comprises CDR-H1 consisting of the amino acid sequence of SEQ ID NO:9, CDR-H2 consisting of the amino acid sequence of SEQ ID NO:10, CDR-H3 consisting of the amino acid sequence of SEQ ID NO:11; CDR-L1 consisting of the amino acid sequence of SEQ ID NO:12, CDR-L2 consisting of the amino acid sequence of SEQ ID NO:13, and CDR-L3 consisting of the amino acid sequence of SEQ ID NO:14, or
wherein the antibody or antigen binding fragment thereof comprises CDR-H1 consisting of the amino acid sequence of SEQ ID NO:16, CDR-H2 consisting of the amino acid sequence of SEQ ID NO:17, CDR-H3 consisting of the amino acid sequence of SEQ ID NO:18; CDR-L1 consisting of the amino acid sequence of SEQ ID NO:19, CDR-L2 consisting of the amino acid sequence of SEQ ID NO:20, and CDR-L3 consisting of the amino acid sequence of SEQ ID NO:21.

22. The method of any one of claims 1 to 21, wherein the antibody or antigen binding fragment thereof comprises a heavy chain variable region comprising the amino acid sequence of SEQ ID NO:22 and a light chain variable region comprising the amino acid sequence of SEQ ID NO:23.

23. The method of any one of claims 1 to 22, wherein the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

24. The method of any one of claims 1 to 23, wherein the antigen binding fragment is an Fab, F(ab′)2, Fv or scFv.

25. The method of any one of claims 1 to 24, wherein the antibody is a fully human antibody.

26. The method of any one of claims 1 to 25, wherein the antibody is an IgG1 and light chain is a kappa light chain.

27. The method of any one of claims 1 to 26, wherein the antibody or antigen binding fragment thereof is recombinantly produced.

28. The method of any one of claims 1 to 27, wherein the antibody or antigen binding fragment is conjugated to each unit of MMAE via a linker.

29. The method of claim 28, wherein the linker is an enzyme-cleavable linker, and wherein the linker forms a bond with a sulfur atom of the antibody or antigen binding fragment thereof.

30. The method of claim 28 or 29, wherein the linker has a formula of: -Aₐ-W_{w}-Y_{y}-; wherein -A- is a stretcher unit, a is 0 or 1; -W- is an amino acid unit, w is an integer ranging from 0 to 12; and -Y- is a spacer unit, y is 0, 1, or 2.

31. The method of claim 30, wherein the stretcher unit has the structure of Formula (1) below; the amino acid unit is valine-citrulline; and the spacer unit is a PAB group comprising the structure of Formula (2) below:

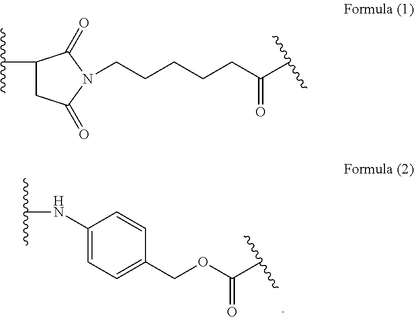 [SMILES: *C(=O)CCCCCN1C(=O)CC(*)C1=O ; *Nc1ccc(COC(*)=O)cc1]

32. The method of claim 30 or 31, wherein the stretcher unit forms a bond with a sulfur atom of the antibody or antigen binding fragment thereof; and wherein the spacer unit is linked to MMAE via a carbamate group.

33. The method of any one of claims 1 to 32, wherein the ADC comprises from 1 to 20 units of MMAE per antibody or antigen binding fragment thereof.

34. The method of any one of claims 1 to 33, wherein the ADC comprises from 1 to 10 units of MMAE per antibody or antigen binding fragment thereof.

35. The method of any one of claims 1 to 34, wherein the ADC comprises from 2 to 8 units of MMAE per antibody or antigen binding fragment thereof.

36. The method of any one of claims 1 to 35, wherein the ADC comprises from 3 to 5 units of MMAE per antibody or antigen binding fragment thereof.

37. The method of any one of claims 1 to 36, wherein the ADC has the following structure: 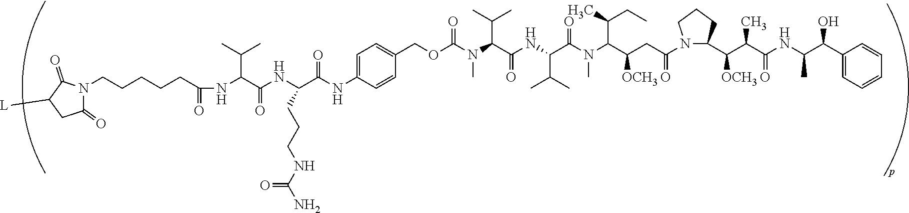
wherein L- represents the antibody or antigen binding fragment thereof and p is from 1 to 10.

38. The method of claim 37, wherein p is from 2 to 8.

39. The method of claim 37 or 38, wherein p is from 3 to 5.

40. The method of any one of claims 37 to 39, wherein p is from 3 to 4.

41. The method of any one of claims 37 to 40, wherein p is about 4.

42. The method of any one of claims 37 to 40, wherein the average p value of the effective amount of the antibody drug conjugates is about 3.8.

43. The method of any one of claims 1 to 42, wherein the ADC is formulated in a pharmaceutical composition comprising L-histidine, polysorbate-20 (TWEEN-20), and trehalose dehydrate.

44. The method of any one of claims 1 to 43, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.

45. The method of any one of claims 1 to 43, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

46. The method of any one of claims 1 to 45, wherein the effective amount of the ADC is a dose of between about 100 mg to about 1000 mg, between about 125 mg to about 950 mg, between about 125 mg to about 900 mg, between about 125 mg to about 850 mg, between about 125 mg to about 800 mg, or between about 125 mg to about 750 mg with a volume of instillation between about 10 mL to about 100 mL.

47. The method of any one of claims 1 to 46, wherein the effective amount of the ADC is a dose of between about 125 mg to about 750 mg with a volume of instillation of about 25 mL.

48. The method of any one of claims 1 to 47, wherein the effective amount of the ADC is a dose of about 125 mg with a volume of instillation of about 25 mL.

49. The method of any one of claims 1 to 47, wherein the effective amount of the ADC is a dose of about 250 mg with a volume of instillation of about 25 mL.

50. The method of any one of claims 1 to 47, wherein the effective amount of the ADC is a dose of about 500 mg with a volume of instillation of about 25 mL.

51. The method of any one of claims 1 to 47, wherein the effective amount of the ADC is a dose of about 750 mg with a volume of instillation of about 25 mL.

52. The method of any one of claims 1 to 51, wherein the maximal dwell time of each intravesical administration is about 90 minutes.

53. The method of any one of claims 1 to 51, wherein the maximal dwell time of each intravesical administration is about 120 minutes.

54. The method of any one of claims 1 to 51, wherein the dwell time of each intravesical administration is about 30, 40, 50, 60, 70, 80, 90, or 120 minutes.

55. The method of any one of claims 1 to 54, wherein the ADC is administered intravesically during two phases, wherein the two phases are an induction phase and a maintenance phase.

56. The method of claim 55, wherein the maintenance phase starts between six to ten weeks, between six to nine weeks, or between six to eight weeks after the induction phase.

57. The method of claim 55 or 56, wherein the ADC is administered intravesically once a week for six weeks during the induction phase.

58. The method of any one of claims 55 to 57, wherein the ADC is administered intravesically once a month for nine months during the maintenance phase.

59. The method of any one of claims 1 to 58, wherein the ADC has the following structure: 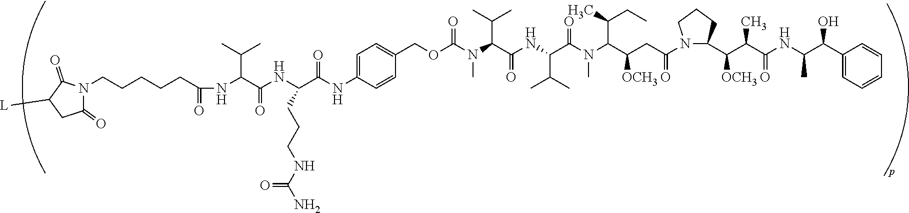
wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered intravesically at a dose of about 125 mg with a volume of instillation of about 25 mL and a maximum 90-minute dwell time, wherein the dose is administered intravesically once a week for six weeks during the induction phase and once a month for nine months during the maintenance phase, and wherein the maintenance phase starts between six to ten weeks after the induction phase.

60. The method of any one of claims 1 to 58, wherein the ADC has the following structure: 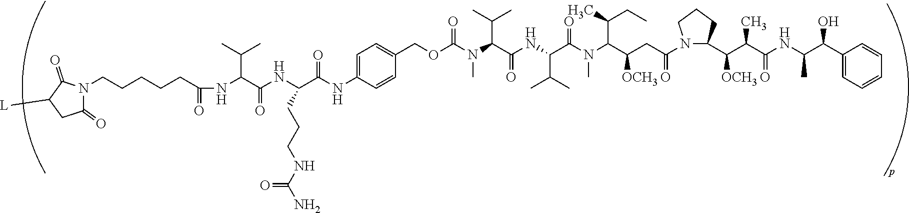
wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered intravesically at a dose of about 250 mg with a volume of instillation of about 25 mL and a maximum 90-minute dwell time, wherein the dose is administered intravesically once a week for six weeks during the induction phase and once a month for nine months during the maintenance phase, and wherein the maintenance phase starts between six to ten weeks after the induction phase.

61. The method of any one of claims 1 to 58, wherein the ADC has the following structure: 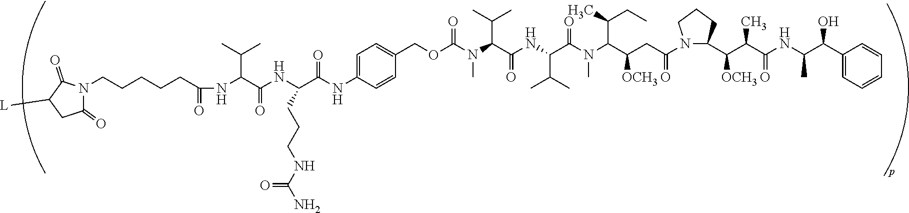
wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered intravesically at a dose of about 500 mg with a volume of instillation of about 25 mL and a maximum 90-minute dwell time, wherein the dose is administered intravesically once a week for six weeks during the induction phase and once a month for nine months during the maintenance phase, and wherein the maintenance phase starts between six to ten weeks after the induction phase.

62. The method of any one of claims 1 to 58, wherein the ADC has the following structure: 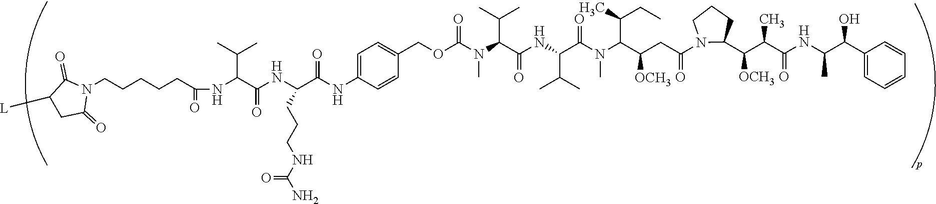
wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered intravesically at a dose of about 750 mg with a volume of instillation of about 25 mL and a maximum 90-minute dwell time, wherein the dose is administered intravesically once a week for six weeks during the induction phase and once a month for nine months during the maintenance phase, and wherein the maintenance phase starts between six to ten weeks after the induction phase.


SEQUENCE LISTING (23 sequences)

SEQ ID NO: 1 - DNA, 3464 bp, Homo sapiens
  source 1..3464: mol_type = other DNA; note = cDNA of 191P4D12
  CDS 264..1796: protein_id = 2; translation = MPLSLGAEMWGPEAWLLLLLLLASFTGRCPAGELETSDVVTVVL GQDAKLPCFYRGDSGEQVGQVAWARVDAGEGAQELALLHSKYGLHVSPAYEGRVEQPP PPRNPLDGSVLLRNAVQADEGEYECRVSTFPAGSFQARLRLRVLVPPLPSLNPGPALE EGQGLTLAASCTAEGSPAPSVTWDTEVKGTTSSRSFKHSRSAAVTSEFHLVPSRSMNG QPLTCVVSHPGLLQDQRITHILHVSFLAEASVRGLEDQNLWHIGREGAMLKCLSEGQP PPSYNWTRLDGPLPSGVRVDGDTLGFPPLTTEHSGIYVCHVSNEFSSRDSQVTVDVLD PQEDSGKQVDLVSASVVVVGVIAALLFCLLVVVVVLMSRYHRRKAQQMTQKYEEELTL TRENSIRRLHSHHTDPRSQPEESVGLRAEGHPDSLKDNSSCSVMSEEPEGRSYSTLTT VREIETQTELLSPGSGRAEEEEDQDEGIKQAMNHFVQENGTLRAKPTGNGIYINGRGH LV
ggccgtcgttgttggccacagcgtgggaagcagctctgggggagctcggagctcccgatcacggcttcttgggggtagctacggctgggtgtgtagaacggggccggggctggggctgggtcccctagtggagacccaagtgcgagaggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtggctcagacccaggtgtgcgggcatagctggagctggaatctgcctccggtgtgagggaacctgtctcctaccacttcggagccatgggggcaagtgtgaagcagccagtccctgggtcagccagaggcttgaactgttacagaagccctctgccctctggtggcctctgggcctgctgcatgtacatattttctgtaaatatacatgcgccgggagcttcttgcaggaatactgctccgaatcacttttaatttttttcttttttttttcttgccctttccattagttgtattttttatttatttttatttttatttttttttagagatggagtctcactatgttgctcaggctggccttgaactcctgggctcaagcaatcctcctgcctcagcctccctagtagctgggactttaagtgtacaccactgtgcctgctttgaatcctttacgaagagaaaaaaaaaattaaagaaagcctttagatttatccaatgtttactactgggattgcttaaagtgaggcccctccaacaccagggggttaattcctgtgattgtgaaaggggctacttccaaggcatcttcatgcaggcagccccttgggagggcacctgagagctggtagagtctgaaattagggatgtgagcctcgtggttactgagtaaggtaaaattgcatccaccattgtttgtgataccttagggaattgcttggacctggtgacaagggctcctgttcaatagtggtgttggggagagagagagcagtgattatagaccgagagagtaggagttgaggtgaggtgaaggaggtgctgggggtgagaatgtcgcctttccccctgggttttggatcactaattcaaggctcttctggatgtttctctgggttggggctggagttcaatgaggtttatttttagctggcccacccagatacactcagccagaatacctagatttagtacccaaactcttcttagtctgaaatctgctggatttctggcctaagggagaggctcccatccttcgttccccagccagcctaggacttcgaatgtggagcctgaagatctaagatcctaacatgtacattttatgtaaatatgtgcatatttgtacataaaatgatattctgtttttaaataaacagacaaaacttgaaaaa

SEQ ID NO: 2 - protein, 510 aa, Homo sapiens
  source 1..510: note = 191P4D12
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 3 - DNA, 1432 bp, Homo sapiens
  source 1..1432: mol_type = other DNA; note = cDNA of Ha22-2(2,4)6.1 heavy chain
  CDS 32..1432: protein_id = 4; translation = MELGLCWVFLVAILEGVQCEVQLVESGGGLVQPGGSLRLSCAAS GFTFSSYNMNWVRQAPGKGLEWVSYISSSSSTIYYADSVKGRFTISRDNAKNSLSLQM NSLRDEDTAVYYCARAYYYGMDVWGQGTTVTVSSASTKGPSVFPLAPSSKSTSGGTAA LGCLVKDYFPEPVTVSWNSGALTSGVHTFPAVLQSSGLYSLSSVVTVPSSSLGTQTYI CNVNHKPSNTKVDKRVEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSREEMTKNQVSLTCLVKGFY PSDIAVEWESNGQPENNYKTTPPVLDSDGSFFLYSKLTVDKSRWQQGNVFSCSVMHEA LHNHYTQKSLSLSPGK
ggtgatcagcactgaacacagaggactcaccatggagttggggctgtgctgggttttccttgttgctattttagaaggtgtccagtgtgaggtgcagctggtggagtctgggggaggcttggtacagcctggggggtccctgagactctcctgtgcagcctctggattcaccttcagtagctataacatgaactgggtccgccaggctccagggaaggggctggagtgggtttcatacattagtagtagtagtagtaccatatactacgcagactctgtgaagggccgattcaccatctccagagacaatgccaagaactcactgtctctgcaaatgaacagcctgagagacgaggacacggctgtgtattactgtgcgagagcatactactacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 4 - protein, 466 aa, Homo sapiens
  source 1..466: note = Ha22-2(2,4)6.1 heavy chain
MELGLCWVFLVAILEGVQCEVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 735 bp, Homo sapiens
  source 1..735: mol_type = other DNA; note = cDNA of Ha22-2(2,4)6.1 light chain
  CDS 25..735: protein_id = 6; translation = MDMRVPAQLLGLLLLWFPGSRCDIQMTQSPSSVSASVGDRVTIT CRASQGISGWLAWYQQKPGKAPKFLIYAASTLQSGVPSRFSGSGSGTDFTLTISSLQP EDFATYYCQQANSFPPTFGGGTKVEIKRTVAAPSVFIFPPSDEQLKSGTASVVCLLNN FYPREAKVQWKVDNALQSGNSQESVTEQDSKDSTYSLSSTLTLSKADYEKHKVYACEV THQGLSSPVTKSFNRGEC
agtcagacccagtcaggacacagcatggacatgagggtccccgctcagctcctggggctcctgctgctctggttcccaggttccagatgcgacatccagatgacccagtctccatcttccgtgtctgcatctgttggagacagagtcaccatcacttgtcgggcgagtcagggtattagcggctggttagcctggtatcagcagaaaccagggaaagcccctaagttcctgatctatgctgcatccactttgcaaagtggggtcccatcaaggttcagcggcagtggatctgggacagatttcactctcaccatcagcagcctgcagcctgaagattttgcaacttactattgtcaacaggctaacagtttccctcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - protein, 236 aa, Homo sapiens
  source 1..236: note = Ha22-2(2,4)6.1 light chain
MDMRVPAQLLGLLLLWFPGSRCDIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 466 aa, Homo sapiens; identical to SEQ ID NO: 4
  source 1..466: note = Ha22-2(2,4)6.1 heavy chain

SEQ ID NO: 8 - protein, 236 aa, Homo sapiens; identical to SEQ ID NO: 6
  source 1..236: note = Ha22-2(2,4)6.1 light chain

SEQ ID NO: 9 - protein, 5 aa, Homo sapiens
  source 1..5: note = CDR1 of heavy chain
SYNMN

SEQ ID NO: 10 - protein, 17 aa, Homo sapiens
  source 1..17: note = CDR2 of heavy chain
YISSSSSTIYYADSVKG

SEQ ID NO: 11 - protein, 8 aa, Homo sapiens
  source 1..8: note = CDR3 of heavy chain
AYYYGMDV

SEQ ID NO: 12 - protein, 11 aa, Homo sapiens
  source 1..11: note = CDR1 of light chain
RASQGISGWLA

SEQ ID NO: 13 - protein, 7 aa, Homo sapiens
  source 1..7: note = CDR2 of light chain
AASTLQS

SEQ ID NO: 14 - protein, 9 aa, Homo sapiens
  source 1..9: note = CDR3 of light chain
QQANSFPPT

SEQ ID NO: 15 - protein, 4 aa, synthetic construct
  source 1..4: note = linker
GFLG

SEQ ID NO: 16 - protein, 8 aa, synthetic construct
  source 1..8: note = VH CDR1 according to IMGT
GFTFSSYN

SEQ ID NO: 17 - protein, 8 aa, synthetic construct
  source 1..8: note = VH CDR2 according to IMGT
ISSSSSTI

SEQ ID NO: 18 - protein, 10 aa, synthetic construct
  source 1..10: note = VH CDR3 according to IMGT
ARAYYYGMDV

SEQ ID NO: 19 - protein, 6 aa, synthetic construct
  source 1..6: note = VL CDR1 according to IMGT
QGISGW

SEQ ID NO: 20 - skipped

SEQ ID NO: 21 - protein, 9 aa, synthetic construct
  source 1..9: note = VL CDR3 according to IMGT
QQANSFPPT

SEQ ID NO: 22 - protein, 117 aa, synthetic construct
  source 1..117: note = heavy chain variable region (VH), the 20th amino acid (glutamic acid) to the 136th amino acid (serine) of SEQ ID NO:7
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 23 - protein, 108 aa, synthetic construct
  source 1..108: note = light chain variable region (VL), the 23rd amino acid (aspartic acid) to the 130th amino acid (arginine) of SEQ ID NO:8
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKR